(19) United States
(12) Patent Application Publication
(10) Pub. No.: US 2023/0339977 A1
(43) Pub. Date: Oct. 26, 2023

(54) SUBSTITUTED HETEROARYL COMPOUND, AND COMPOSITION AND APPLICATION THEREOF

(71) Applicant: SUNSHINE LAKE PHARMA CO., LTD., Dongguan, Guangdong (CN)

(72) Inventors: Ning XI, Newbury Park, CA (US); Bin WANG, Dongguan (CN); Wuhong CHEN, Dongguan (CN); Peng CHEN, Dongguan (CN); Changlin BAI, Dongguan (CN); Wangjin XU, Dongguan (CN); Minxiong LI, Dongguan (CN); Shuying CHEN, Dongguan (CN)

(73) Assignee: SUNSHINE LAKE PHARMA CO., LTD., Dongguan, Guangdong (CN)

(21) Appl. No.: 18/020,981

(22) PCT Filed: Aug. 12, 2021
(86) PCT No.: PCT/CN2021/112211

§ 371 Date: Feb. 13, 2023

(30) Foreign Application Priority Data
Aug. 13, 2020 (CN) 202010812208.9

(51) Int. Cl. C07D 519/00; A61K 45/06

(52) U.S. Cl. CPC C07D 519/00; A61K 45/06

(57) ABSTRACT

A substituted heteroaryl compound, and a composition and application thereof. The compound is a compound as represented by formula (I) or a stereoisomer, a tautomer, an oxynitride, a solvate, a metabolite, a pharmaceutically acceptable salt or a prodrug of the compound as represented by formula (I). A drug composition contains the compound. The compound and the drug composition can adjust activity of a JAK kinase, particularly activity of TYK2, and are used for preventing, handling, treating and relieving diseases or disorder mediated by the activity of the JAK kinase.

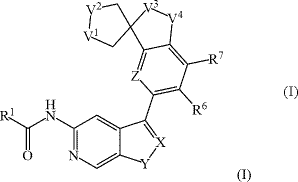

### CROSS-REFERENCE TO RELATED APPLICATION

This is a U.S. national stage application of the International Patent Application No. PCT/CN2021/112211, filed Aug. 12, 2021, which claims the priority and benefits of Chinese Patent Application No.202010812208.9, filed with the State Intellectual Property Office of China on Aug. 13, 2020, which is incorporated herein by reference in its entirety.

### FIELD OF THE INVENTION

The invention belongs to the field of medicine, and in particular relates to a class of novel substituted heteroaryl compounds as JAK kinase activity inhibitors, preparation methods thereof, pharmaceutical compositions containing the compounds, and application of the compounds and pharmaceutical compositions in the treatment of multiple diseases. More specifically, the compounds described herein may act as inhibitors of the activity or function of tyrosine kinase 2 (TYK2).

### BACKGROUND OF THE INVENTION

Janus kinase (JAK) is an intracellular non-receptor tyrosine kinase that transduce cytokine-mediated signals through the JAK-STAT pathway. The JAK family plays an important role in cytokine-dependent regulation of proliferation and cellular functions involved in immune responses. Cytokines bind to their receptors, causing receptor dimerization, which promotes mutual phosphorylation of JAKs, as well as phosphorylation of specific tyrosine motifs within cytokine receptors. STATs that recognize these phosphorylation motifs are recruited to receptors and then activated during the phosphorylation of JAK-dependent tyrosine. Due to activation, STATs dissociate from receptors, dimerize, and translocate to the nucleus, bind to specific DNA sites, and alter transcription.

Currently, there are four known mammalian JAK family members: JAK1 (Janus kinase-1), JAK2 (Janus kinase-2), JAK3 (Janus kinase, leukocyte, JAKL, L-JAK, and Janus kinase-3) and TYK2 (protein tyrosine kinase 2). Different members of the Janus kinase family are responsible for transmitting the signals of different cytokines and their receptors, JAK1, JAK2 and TYK2 are widely expressed, while JAK3 is reported to be preferentially expressed in natural killer (NK) cells and not expressed in other T cells.

TYK2 is associated with IFN-α (α-interferon), IL-6 (Interleukin-6), IL-10 (Interleukin-10), IL-12 (Interleukin-12) and IL-23 (Interleukin-23) signaling. Biochemical studies and knockout mice revealed an important role for TYK2 in immunity. TYK2-deficient mice can grow and reproduce, but have multiple immunodeficiencies, primarily hypersensitivity to infection and deficits in tumor surveillance. Conversely, the inhibition of TYK2 can improve resistance to allergy, autoimmune and inflammatory diseases. In particular, targeting TYK2 appears to be an innovative strategy for the treatment of IL-12, IL-23- or type I IFN-mediated diseases. Such diseases include, but are not limited to, rheumatoid arthritis, multiple sclerosis, lupus, psoriasis, psoriatic arthritis, inflammatory bowel disease, uveitis, sarcoidosis, and cancer (Shaw, M. et al., Proc. Natl. Acad. Sci., USA, 2003, 100, 11594-11599; Ortmann, R.A., and Shevach, E.M. Clin. Immunol, 2001, 98, 109-118; Watford et al, Immunol. Rev., 2004, 202: 139).

The European Commission recently approved Stelara (ustekinumab), a fully human monoclonal antibody targeting the p40 subunit shared by IL-12 and IL-23 cytokines, for the treatment of moderate-to-severe plaque psoriasis (Krueger et al., 2007, N. Engl. J. Med., 356:580-92; Reich et al., 2009, Nat. Rev. Drug Discov., 8:355-356). In addition, ABT-874, an antibody targeting the IL-12 and IL-23 pathways, is in clinical trials for the treatment of Crohn’s disease (Mannon et al., N.Engl. J. Med., 2004, 351 :2069-79).

Since the IL-12 and IL-23 signaling pathways are mediated by JAK2/TYK2 heterodimers via phosphorylation of STAT¾, the development of JAK2 and TYK2 inhibitors is of great interest to the scientific and medical communities. See, e.g., Liang et al., J. Med. Chem. (2013) 56:4521-4536. However, blocking JAK2 activity is considered problematic because JAK2 also regulates the signaling pathway of erythropoietin, and its inhibition is associated with undesired hematologic toxicities, such as anemia, neutropenia, and thrombocytopenia. See, e.g., Liang et al., J. Med. Chem. (2013) 56:4521-4536; Alabduaali, Hematology Rebies. (2009) 1: e1056-61.

Therefore, given the high sequence homology among JAK family kinase members, it is a significant challenge to develop selective TYK2 inhibitors that avoid the inhibition of JAK2 and/or JAK1.

### SUMMARY

The present invention provides a class of compounds that inhibit, adjust and/or regulate JAK kinase activity for the treatment of inflammatory diseases, proliferative diseases, autoimmune diseases, allergic diseases, inflammatory diseases, transplant rejection, and complications thereof. The present invention also provides methods for preparing these compounds, methods for using these compounds to treat the above-mentioned diseases in mammals, especially humans, and pharmaceutical compositions containing these compounds. The compound and the composition thereof of the present invention have better prospects for clinical application. Compared with the existing similar compounds, the compounds provided herein have better pharmacological activity, pharmacokinetic properties, physicochemical properties and/or lower toxicity. Specifically, the compounds provided herein show better inhibitory activity and optimized TYK2 selectivity to the target TYK2, and show good absorption and high bioavailability in pharmacokinetic tests in animals; and the compounds provided herein have no cardiotoxicity and are safe. Therefore, the compounds provided herein have better druggability.

Specifically:

In one aspect, the present invention relates to a compound having formula (I) or a stereoisomer, a geometric isomer, a tautomer, an N-oxide, a hydrate, a solvate, a metabolite, a pharmaceutically acceptable salt or a prodrug of the compound having formula (I),

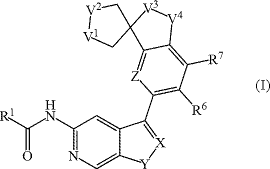

wherein:
X is N or CR^{a}; Z is N or CR^{e};
Y is NR^{b} or CR^{c}R^{d};
R¹ is —NH₂, C₁₋₆ alkyl, C₁₋₆ alkylamino, C₃₋₈ cycloalkyl, heterocyclyl consisting of 3-8 atoms, C₆₋₁₀ aryl or heteroaryl consisting of 5-12 atoms, wherein, the —NH₂, C₁₋₆ alkyl, C₁₋₆ alkylamino, C₃₋₈ cycloalkyl, heterocyclyl consisting of 3-8 atoms, C₆₋₁₀ aryl and heteroaryl consisting of 5-12 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy;
each of R⁶ and R⁷ is independently H, F, Cl, Br, I, —NO₂, CN, —OH, —NH₂, C₁₋₆ alkyl, C₁₋₆ haloalkyl, C₂₋₆ alkenyl, C₂₋₆ alkynyl, C₁₋₆ alkoxy, C₃₋₆ cycloalkyl, C₃₋₆ cycloalkyl-O-, C₁₋₆ alkylamino, C₁₋₆ alkyl-S(=O)₂-, C₁₋₆ alkyl-S-, heterocyclyl consisting of 3-8 atoms, C₆₋₁₀ aryl or heteroaryl consisting of 5-10 atoms, wherein, the —OH, —NH₂, C₁₋₆ alkyl, C₁₋₆ haloalkyl, C₂₋₆ alkenyl, C₂₋₆ alkynyl, C₁₋₆ alkoxy, C₃₋₆ cycloalkyl, C₃₋₆ cycloalkyl-O-, C₁₋₆ alkylamino, C₁₋₆ alkyl-S(=O)₂-, C₁₋₆ alkyl-S-, heterocyclyl consisting of 3-8 atoms, C₆₋₁₀ aryl and heteroaryl consisting of 5-10 atoms can be independently and optionally substituted with 1, 2, 3, 4 or 5 R^{X}; wherein the R^{X} has a meaning as described in the present invention.

Each of V¹, V², V³ and V⁴ is independently -(CR⁹R¹⁰)ₙ-, -(CR⁹R¹⁰)ₙ-O-, -(CR⁹R¹⁰)ₙ-S-, -(CR⁹R¹⁰)ₙ-NR¹¹-, -(CR⁹R¹⁰)ₙ -C(=O)-, -(CR⁹R¹⁰)ₙ-O-C(=O)-, -(CR⁹R¹⁰)ₙ-C(=O)-O-, -(CR⁹R¹⁰)ₙ-S(=O)- or -(CR⁹R¹⁰)ₙ-S(=O)₂-;
each of R⁹ and R¹⁰ is independently H, D, F, Cl, Br, I, —NO², CN, —OH, —NH₂, C₁₋₆ alkyl, C₁₋₆ haloalkyl, C₁₋₆ alkoxy or C₃ ₋₆ cycloalkyl, wherein, the —OH, —NH₂, C₁₋₆ alkyl, C₁₋₆ haloalkyl, C₁₋₆ alkoxy and C₃ ₋₆ cycloalkyl can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy; or
R⁹ and R¹⁰, together with the carbon atom to which they are attached, form C₃₋₆ cycloalkyl or heterocyclyl consisting of 3-6 atoms, wherein, the C₃₋₆ cycloalkyl and heterocyclyl consisting of 3-6 atoms can be independently and optionally substituted with 1, 2, 3, 4 or 5 substituents selected from F, Cl, Br, I, —NO₂, CN, —OH, —NH₂, oxo, C₁₋₃ alkyl, C₁₋₃ haloalkyl, C₁₋₃ alkoxy, C₁₋₃ haloalkoxy and C₁₋₃ hydroxyalkoxy;
R¹¹ is H, D, C₁₋₆ alkyl, C₁₋₆ alkyl-C(=O)-, C₁₋₆ alkyl-O-C(=O)-, C₁₋₆ haloalkyl or C₃₋₆ cycloalkyl, wherein, C₁₋₆ alkyl, C₁₋₆ alkyl-C(=O)-, C₁₋₆ alkyl-O-C(=O)-, C₁₋₆ haloalkyl and C₃₋₆ cycloalkyl can be independently and optionally substituted with 1, 2, 3, 4 or 5 substituents selected from F, Cl, Br, I, —NO₂, CN, —OH, —NH₂, oxo, C₁₋₃ alkyl, C₁₋₃ haloalkyl, C₁₋₃ alkoxy, C₁₋₃ haloalkoxy, C₁₋₃ hydroxyalkoxy and C₃₋₆ cycloalkyl;
each of R^{a}, R^{c} R^{d} and R^{e} is independently H, D, F, Cl, Br, I, —NO₂, CN, —OH, —NH₂, C₁₋₆ alkyl, C₁₋₆ haloalkyl, C₂₋₆ alkenyl, C₂₋₆ alkynyl, C₁₋₆ alkoxy, C₃₋₈ cycloalkyl, heterocyclyl consisting of 3-8 atoms, C₆₋₁₀ aryl or heteroaryl consisting of 5-12 atoms, wherein, the —OH, —NH₂, C₁₋₆ alkyl, C₁₋₆ haloalkyl, C₂₋₆ alkene, C₂₋₆ alkynyl, C₁₋₆ alkoxy, C₃₋₈ cycloalkyl, heterocyclyl consisting of 3-8 atoms, C₆₋₁₀ aryl and heteroaryl consisting of 5-12 atoms can be independently and optionally unsubstituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy;
R^{b} is H, D, C₁₋₆ alkyl, C₁₋₆ haloalkyl, C₂₋₆ alkenyl, C₂₋₆ alkynyl, C₁₋₆ alkoxy, C₃₋₈ cycloalkyl, heterocyclyl consisting of 3-8 atoms, C₆₋₁₀ aryl or heteroaryl consisting of 5-12 atoms, wherein, the C₁₋₆ alkyl, C₁₋₆ haloalkyl, C₂₋₆ alkenyl, C₂₋₆ alkynyl, C₁₋₆ alkoxy, C₃₋₈ cycloalkyl, heterocyclyl consisting of 3-8 atoms, C₆₋₁₀ aryl and heteroaryl consisting of 5-12 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy;
each R^{X} is independently F, Cl, Br, I, —NO₂, CN, —OH, —NH₂, C₁₋₆ alkyl, C₁₋₆ haloalkyl, C₂₋₆ alkenyl, C₂₋₆ alkynyl, C₁₋₆ alkoxy, C₁₋₆ alkylamino or C₃₋₆ cycloalkyl, wherein, the —OH, —NH₂, C₁₋₆ alkyl, C₁₋₆ haloalkyl, C₂₋₆ alkenyl, C₂₋₆ alkynyl, C₁₋₆ alkoxy, C₁₋₆ alkylamino and C₃₋₆ cycloalkyl can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ alkylamino, C₁₋₃ haloalkoxy and C₁₋₃ hydroxyalkoxy;
each n is independently 0, 1, or 2.

In some embodiments, each of V¹, V², V³ and V⁴ is independently -(CR⁹R¹⁰)-, -(CR⁹R¹⁰)₂-, —O—, -(CR⁹R¹⁰)-O-, —S—, -(CR⁹R¹⁰)-S-, -NR¹¹-, -(CR⁹R¹⁰)-NR¹¹-, —C(═O)—, -(CR⁹R¹⁰)-C(=O)-, —O—C(═O)—, -(CR⁹R¹⁰)-O-C(=O)-, —C(═O)—O—, -(CR⁹R¹⁰)-C(=O)-O-, —S(═O)—, -(CR⁹R¹⁰)-S(=O)-, —S(═O)₂— or -(CR⁹R¹⁰)-S(=O)₂-; wherein R⁹, R¹⁰ and R¹¹ have the meanings described in the present invention.

In some embodiments, the compounds of the present invention have the structure shown in formula (II):

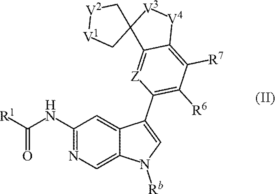

wherein, each Z, R¹, R⁶, R⁷, R^{b}, V¹, V², V³ and V⁴ has the meaning described in the present invention.

In some embodiments, R¹ is —NH₂, C₁₋₄ alkyl, C₁₋₄ alkylamino, C₃₋₆ cycloalkyl, heterocyclyl consisting of 3-6 atoms, C₆₋₁₀ aryl or heteroaryl consisting of 5-10 atoms, wherein, the -NH₂, C₁₋₄ alkyl, C₁₋₄ alkylamino, C₃₋₆ cycloalkyl, heterocyclyl consisting of 3-6 atoms, C₆₋₁₀ aryl and heteroaryl consisting of 5-10 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In some embodiments, R¹ is —NH₂, methyl, ethyl, n-propyl, isopropyl, n-butyl, isobutyl, sec-butyl, tert-butyl, N-methylamino, N-ethylamino, N,N-dimethylamino, N,N-diethylamino, N-ethylpropyl-2-amino, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein, the —NH₂, methyl, ethyl, n-propyl, isopropyl, n-butyl, isobutyl, sec-butyl, tert-butyl, N-methylamino, N-ethylamino, N,N-dimethylamino, N,N-diethylamino, N-ethylpropyl-2-amino, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and pyridazinyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, -OCH₂OH and -OCH₂CH₂OH.

In some embodiments, each R⁶ and R⁷ is independently H, F, Cl, Br, I, —NO₂, CN, —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, C₃₋₆ cycloalkyl—O—, C₁₋₄ alkylamino, C₁₋₄ alkyl—S(═O)₂—, C₁₋₄ alkyl—S—, heterocyclyl consisting of 3-6 atoms, C₆₋₁₀ aryl or heteroaryl consisting of 5-10 atoms, wherein the —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, C₃₋₆ cycloalkyl-O-, C₁₋₄ alkylamino, C₁₋₄ alkyl-S(=O)₂-, C₁₋₄ alkyl-S-, heterocyclyl consisting of 3-6 atoms, C₆₋₁₀ aryl and heteroaryl consisting of 5-10 atoms can be independently and optionally substituted with 1, 2, 3, 4 or 5 R^{X}; wherein the R^{X} has the meaning as described in the present invention.

In some embodiments, each R⁶ and R⁷ is independently H, F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl, cyclohexyl, cyclopropyl-O-, cyclobutyl-O-, cyclopentyl-O-, cyclohexyl-O-, —NH(CH₂)₃CH₃, CH₃(CH₂)₃S—, CH₃—S(═O)₂—, CH₃(CH₂)₃—S(═O)₂—, oxetanyl, azetidinyl, pyrrolidinyl, tetrahydrofuranyl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein the —OH, —NH₂, methyl, ethyl, n-propyl, isopropyl, n-butyl, isobutyl, sec-butyl, tert-butyl, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl, cyclohexyl, cyclopropyl-O-, cyclobutyl-O-, cyclopentyl-O-, cyclohexyl-O-, —NH(CH₂)₃CH₃, CH₃(CH₂)₃S—, CH₃—S(═O)₂—, CH₃(CH₂)₃—S(═O)₂—, oxetanyl, azetidinyl, pyrrolidinyl, tetrahydrofuranyl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and idazinyl can be independently and optionally substituted with 1, 2, 3, 4 or 5 R^{X}; wherein each R^{X} has the meaning as described in the present invention.

In some embodiments, each R⁹ and R¹⁰ is independently H, D, F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl or cyclohexyl, wherein, the —OH, —NH₂, methyl, ethyl, n-propyl, isopropyl, n-butyl, isobutyl, sec-butyl, tert-butyl, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl and cyclohexyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, —OCH₂OH and —OCH₂CH₂OH; or
R⁹ and R¹⁰, together with the carbon atom to which they are attached, form cyclopropyl, cyclobutyl, cyclopentyl, cyclohexyl, oxetanyl, azetidinyl, pyrrolidinyl, tetrahydrofuranyl, piperidinyl, piperazinyl or morpholinyl, wherein, each of the cyclopropyl, cyclobutyl, cyclopentyl, cyclohexyl, oxetanyl, azetidinyl, pyrrolidinyl, tetrahydrofuranyl, piperidinyl, piperazinyl or morpholinyl is independently unsubstituted or substituted with 1, 2, 3, 4 or 5 substituents selected from F, Cl, Br, I, —NO₂, CN, —OH, —NH₂, oxo, methyl, ethyl, n-propyl, isopropyl, trifluoromethyl, difluoromethyl, methoxy, ethoxy, isopropoxy, trifluoromethoxy, —OCH₂OH and —OCH₂CH₂OH;
R¹¹ is H, D, methyl, ethyl, n-propyl, isopropyl, n-butyl, isobutyl, sec-butyl, tert-butyl, CH₃CH₂—C(═O)—, CH₃CH₂—O—C(═O)—, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, cyclopropyl, cyclobutyl, cyclopentyl or cyclohexyl, wherein each of the methyl, ethyl, n-propyl, isopropyl, n-butyl, isobutyl, sec-butyl, tert-butyl, CH₃CH₂—C(═O)—, CH₃CH₂—O—C(═O)—, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, cyclopropyl, cyclobutyl, cyclopentyl and cyclohexyl is independently unsubstituted or substituted with 1, 2, 3, 4 or 5 substituents selected from F, Cl, Br, I, —NO₂, CN, —OH, —NH₂, oxo, methyl, ethyl, n-propyl, isopropyl, trifluoromethyl, difluoromethyl, methoxy, ethoxy, isopropoxy, trifluoromethoxy, —OCH₂OH, -OCH₂CH₂OH, cyclopropyl, cyclobutyl, cyclopentyl and cyclohexyl.

In some embodiments,

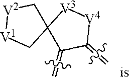

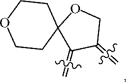

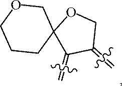

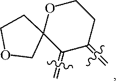

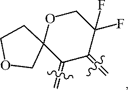

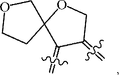

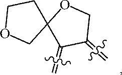

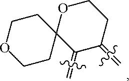

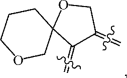

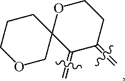

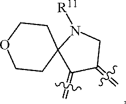

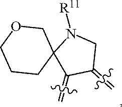

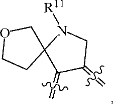

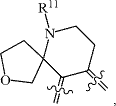

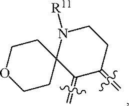

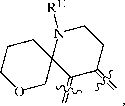

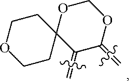

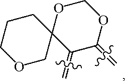

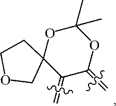

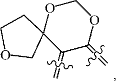

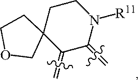

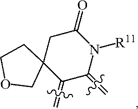

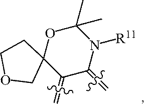

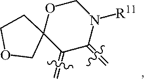

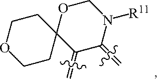

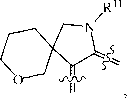

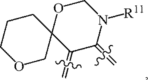

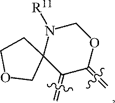

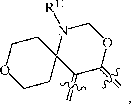

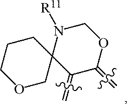

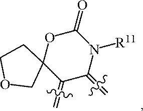

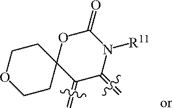

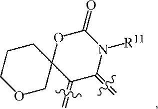

wherein R¹¹ is H, D, methyl, ethyl, n-propyl, isopropyl, n-butyl, isobutyl, sec-butyl, tert-butyl, CH₃CH₂—C(═O)—, CH₃CH₂—O—C(═O)—, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, cyclopropyl, cyclobutyl, cyclopentyl or cyclohexyl, wherein each of the methyl, ethyl, n-propyl, isopropyl, n-butyl, isobutyl, sec-butyl, tert-butyl, CH₃CH₂—C(═O)—, CH₃CH₂—O—C(═O)—, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, cyclopropyl, cyclobutyl, cyclopentyl and cyclohexyl is independently unsubstituted or substituted with 1, 2, 3, 4 or 5 substituents selected from F, Cl, Br, I, —NO₂, CN, —OH, —NH₂, oxo, methyl, ethyl, n-propyl, isopropyl, trifluoromethyl, difluoromethyl, methoxy, ethoxy, isopropoxy, trifluoromethoxy, —OCH₂OH, —OCH₂CH₂OH, cyclopropyl, cyclobutyl, cyclopentyl and cyclohexyl.

In some embodiments, each of R^{a}, R^{c} R^{d} and R^{e} is independently H, D, F, Cl, Br, I, —NO₂, CN, —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl or heteroaryl consisting of 5-10 atoms, wherein the —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl and heteroaryl consisting of 5-10 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy;

R^{b} is H, D, C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl or heteroaryl consisting of 5-10 atoms, wherein the C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl and heteroaryl consisting of 5-10 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In some embodiments, each of R^{a}, R^{c} R^{d} and R^{e} is independently H, D, F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein the —OH, —NH₂, methyl, ethyl, n-propyl, isopropyl, n-butyl, isobutyl, sec-butyl, tert-butyl, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and pyridazinyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, —OCH₂OH and —OCH₂CH₂OH;

R^{b} is H, D, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein the methyl, ethyl, n-propyl, isopropyl, n-butyl, isobutyl, sec-butyl, tert-butyl, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and pyridazinyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, —OCH₂OH and —OCH₂CH₂OH.

In some embodiments, each R^{x} is independently F, Cl, Br, I, —NO₂, CN, —OH, —NH₂, C₁₋₄ alkyl, C₁₋₄ haloalkyl, C₂₋₄ alkenyl, C₂₋₄ alkynyl, C₁₋₄ alkoxy, C₁₋₄ alkylamino or C₃₋₆ cycloalkyl, wherein the —OH, —NH₂, C₁₋₄ alkyl, C₁₋₄ haloalkyl, C₂₋₄ alkenyl, C₂₋₄ alkynyl, C₁₋₄ alkoxy, C₁₋₄ alkylamino and C₃₋₆ cycloalkyl can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ alkylamino, C₁₋₃ haloalkoxy and C₁₋₃ hydroxyalkoxy.

In some embodiments, each R^{x} is independently F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, —N(CH₃)₂, cyclopropyl, cyclobutyl, cyclopentyl or cyclohexyl, wherein the —OH, —NH₂, methyl, ethyl, n-propyl, isopropyl, n-butyl, isobutyl, sec-butyl, tert-butyl, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, —N(CH₃)₂, cyclopropyl, cyclobutyl, cyclopentyl and cyclohexyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, —N(CH₃)₂—, trifluoromethoxy, —OCH₂OH and —OCH₂CH₂OH.

In one aspect, the present invention relates to a pharmaceutical composition, which comprises the compound having formula (I) or formula (II) of the present invention, or a stereoisomer, a geometric isomer, a tautomer, an N-oxide, a hydrate, a solvate, a metabolite, a pharmaceutically acceptable salt or a prodrug thereof.

In some embodiments, the pharmaceutical composition disclosed herein further comprises at least one of pharmaceutically acceptable adjuvants, excipients, carriers and vehicles.

In other embodiments, the pharmaceutical composition of the present invention further comprises other therapeutic agents, the other therapeutic agents are selected from at least one of corticosteroids, rolipram, carvestatin, cytokine inhibitory anti-inflammatory drugs, interleukin-10, glucocorticoid, salicylate, nuclear translocation inhibitor, steroid antiviral agent, antiproliferative agent, antimalarial, TNF-a inhibitor.

In other aspect, provided herein is use of the compound or the pharmaceutical composition disclosed herein in the manufacture of a medicament for preventing, handling, treating and relieving TYK2-mediated diseases.

In some embodiments, the TYK2-mediated disease of the present invention is inflammatory disease, autoimmune disease, metabolic disease, destructive bone disease, proliferative disease, angiogenic disorder, sepsis, septic shock, Shigellosis, neurodegenerative disease, or retinitis.

In other embodiments, the TYK2-mediated disease of the present invention is pancreatitis, asthma, allergy, adult respiratory distress syndrome, chronic obstructive pulmonary disease, glomerulonephritis, rheumatoid arthritis, systemic lupus erythematosus, cutaneous lupus erythematosus, lupus nephritis, discoid lupus erythematosus, scleroderma, chronic thyroiditis, Graves disease, autoimmune gastritis, diabetes mellitus, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn’s disease, psoriasis, graft-versus-host disease, endotoxin-induced inflammatory response, tuberculosis, atherosclerosis, muscle degeneration, cachexia, psoriatic arthritis, Knight’s syndrome, gout, traumatic arthritis, rheumatoid arthritis, acute synovitis, pancreatic β-cell disease, disease characterized by massive neutrophil infiltration, rheumatoid spondylitis, gouty arthritis, cerebral malaria, chronic pulmonary inflammatory disease, silicosis, pulmonary sarcoidosis, bone resorption disease, allograft rejection, fever due to infection, myalgia due to infection, cachexia secondary to infection, keloid formation, scar tissue formation, fever, influenza, osteoporosis, osteoarthritis, acute myelogenous leukemia, chronic myelogenous leukemia, metastatic melanoma, Kaposi’s sarcoma, multiple myeloma, sepsis, septic shock, Shigellosis, Alzheimer’s disease, Parkinson’s disease, cerebral ischemia or neurodegenerative disease caused by traumatic injury, angiogenic disorders, viral diseases, CMV retinitis, AIDS, ARC, herpes, stroke, myocardial ischemia, ischemia in stroke heart attack, organ hypoxia, vascular proliferation, cardiac reperfusion injury, renal reperfusion injury, thrombosis, cardiac hypertrophy, coagulation-induced enzymatic platelet aggregation, endotoxemia, toxic shock syndrome, a disease state associated with prostaglandin endoperoxidase synthase-2, or pemphigus vulgaris.

In other aspect, provided herein is use of the compound or the pharmaceutical composition disclosed herein in the manufacture of a medicament for the activity of JAK kinase.

In some embodiments, the JAK kinase described herein is TYK2 kinase.

In yet another aspect, the present invention relates to methods for the preparation, separation and purification of compounds encompassed by formula (I) or formula (II).

The biological test results show that the compounds provided herein can be used as a better JAK kinase inhibitor, especially as a TYK2 kinase inhibitor.

Any embodiment disclosed herein can be combined with other embodiments as long as they are not contradictory to one another, even though the embodiments are described under different aspects of the invention. In addition, any technical feature in one embodiment can be applied to the corresponding technical feature in other embodiments as long as they are not contradictory to one another, even though the embodiments are described under different aspects of the invention.

The foregoing merely summarizes certain aspects disclosed herein and is not intended to be limiting in nature. These aspects and other aspects and embodiments are described more fully below.

### DETAILED DESCRIPTION OF THE INVENTION

### Definitions and General Terminology

Reference will now be made in detail to certain embodiments of the invention, examples of which are illustrated in the accompanying structures and formulas. The invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described herein. In the event that one or more of the incorporated literature, patents, and similar materials differs from or contradicts this application, including but not limited to defined terms, term usage, described techniques, or the like, this application controls.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Unless defined otherwise, all technical and scientific terms used herein have the same meaning as is commonly understood by one skilled in the art to which this invention belongs. All patents and publications referred to herein are incorporated by reference in their entirety.

As used herein, the following definitions shall apply unless otherwise indicated. For purposes of this invention, the chemical elements are identified in accordance with the Periodic Table of the Elements, CAS version, and the Handbook of Chemistry and Physics, 75th Ed. 1994. Additionally, general principles of organic chemistry are described in “Organic Chemistry”, Thomas Sorrell, University Science Books, Sausalito: 1999, and “March’s Advanced Organic Chemistry” by Michael B. Smith and Jerry March, John Wiley & Sons, New York: 2007, the entire contents of which are hereby incorporated by reference.

Reference throughout this specification to “an embodiment,” “some embodiments,” “an example,” “a specific example,” or “some examples,” means that a particular feature, structure, material, or characteristic described in connection with the embodiment or example is included in at least one embodiment or example of the present disclosure. In this specification, the schematic expression of the above terms must not address the same embodiments or examples. Furthermore, the particular features, structures, materials, or characteristics may be combined in any suitable manner in one or more embodiments or examples. In addition, those skilled in the art can integrate and combine different embodiments, examples or the features of them as long as they are not contradictory to one another.

The grammatical articles “a”, “an” and “the”, as used herein, are intended to include “at least one” or “one or more” unless otherwise indicated herein or clearly contradicted by the context. Thus, the articles are used herein to refer to one or more than one (i.e. at least one) of the grammatical objects of the article. By way of example, “a component” means one or more components, and thus, possibly, more than one component is contemplated and may be employed or used in an implementation of the described embodiments.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, “patient” refers to a human (including adults and children) or other animal. In one embodiment, “patient” refers to a human.

The term “comprise” or “contain” is an open expression, it means comprising the contents disclosed herein, but don’t exclude other contents.

“Stereoisomers” refers to compounds which have identical chemical constitution, but differ with regard to the arrangement of the atoms or groups in space. Stereoisomers include enantiomer, diastereomers, conformer (rotamer), geometric (cis/trans) isomer, atropisomer, etc.

“Chiral” refers to molecules which have the property of non-superimposability of the mirror image partner, while the term “achiral” refers to molecules which are superimposable on their mirror image partner.

“Enantiomers” refers to two stereoisomers of a compound which are non-superimposable mirror images of one another.

“Diastereomer” refers to a stereoisomer with two or more centers of chirality and whose molecules are not mirror images of one another. Diastereomers have different physical properties, e.g. melting points, boling points, spectral properties or biological activities. Mixture of diastereomers may separate under high resolution analytical procedures such as electrophoresis and chromatography such as HPLC.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994.

Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (-) are employed to designate the sign of rotation of plane-polarized light by the compound, with (-) or l meaning that the compound is levorotatory. A compound prefixed with (+) or d is dextrorotatory. A specific stereoisomer may be referred to as an enantiomer, and a mixture of such stereoisomers is called an enantiomeric mixture. A 50:50 mixture of enantiomers is referred to as a racemic mixture or a racemate, which may occur where there has been no stereoselection or stereospecificity in a chemical reaction or process.

Any asymmetric atom (e.g., carbon or the like) of the compound(s) disclosed herein can be present in racemic or enantiomerically enriched, for example the (R)-, (S)- or (R, S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)- configuration.

Depending on the choice of the starting materials and procedures, the compounds can be present in the form of one of the possible stereoisomers or as mixtures thereof, such as racemates and diastereoisomer mixtures, depending on the number of asymmetric carbon atoms. Optically active (R)- and (S)- isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration.

Any resulting mixtures of stereoisomers can be separated on the basis of the physicochemical differences of the constituents, into the pure or substantially pure geometric isomers, enantiomers, diastereomers, for example, by chromatography and/or fractional crystallization. Cis and trans isomers are diastereomer.

Any resulting racemates of final products or intermediates can be resolved into the optical antipodes by methods known to those skilled in the art, e.g., by separation of the diastereomeric salts thereof. Racemic products can also be resolved by chiral chromatography, e.g., high performance liquid chromatography (HPLC) using a chiral adsorbent. In particular, enantiomers may be prepared by asymmetric synthesis, see for example Jacques, et al., Enantiomers, Racemates and Resolutions (Wiley Interscience, New York, 1981); Principles of Asymmetric Synthesis (2^{nd} Ed. Robert E. Gawley, Jeffrey Aubé, Elsevier, Oxford, UK, 2012); Eliel, E.L. Stereochemistry of Carbon Compounds (McGraw-Hill, NY, 1962); Wilen, S.H. Tables of Resolving Agents and Optical Resolutions p. 268 (E.L. Eliel, Ed., Univ. of Notre Dame Press, Notre Dame, IN 1972); Chiral Separation Techniques: A Practical Approach (Subramanian, G Ed., Wiley-VCH Verlag GmbH & Co. KGaA, Weinheim, Germany, 2007).

The term “tautomer” or “tautomeric form” refers to structural isomers of different energies which are interconvertible via a low energy barrier. Where tautomerization is possible (e.g. in solution), a chemical equilibrium of tautomers can be reached. For example, proton tautomers (also known as prototropic tautomers) include interconversions via migration of a proton, such as keto-enol and imine-enamine isomerizations. Valence tautomers include interconversions by reorganization of some of the bonding electrons. A specific example of keto-enol tautomerization is the interconversion of pentane-2,4-dione and 4-hydroxypent-3-en-2-one tautomers. Another example of tautomerization is phenol-keto tautomerization. The specific example of phenol-keto tautomerisms is pyridin-4-ol and pyridin-4(1H)-one tautomerism. Unless otherwise stated, all tautomeric forms of the compounds disclosed herein are within the scope of the invention.

As described herein, compounds disclosed herein may optionally be substituted with one or more substituents, such as are illustrated generally below, or as exemplified by particular classes, subclasses, and species of the invention.

In general, the term “substituted” refers to the replacement of one or more substitutable hydrogen radicals in a given structure with the radical of a specified substituent. Unless otherwise indicated, a substituted group may have a substituent at each substitutable position of the group. When more than one position in a given structure can be substituted with more than one substituent selected from a specified group, the substituent may be either the same or different at each position.

The term “optional” or “optionally” refers to that the subsequently described event or circumstance may or may not occur, and that the description includes instances where the event or circumstance may or may not occur. For example, “heterocyclic group optionally substituted by an alkyl group” means that the alkyl may or may not be present, and the description includes the situation where the heterocyclic group is substituted by the alkyl group and the situation where the heterocyclic group is not substituted by the alkyl group.

The term “unsubstituted” refers to that the specified group bears no substituents.

The term “optionally substituted with” may be used interchangeably with the term “unsubstituted or substituted with”, i.e. the structures are unsubstituted or substituted with one or more substituents described herein.

Furthermore, what need to be explained is that the phrase “each...is independently” and “each of...and...is independently”, unless otherwise stated, should be broadly understood. The specific options expressed by the same symbol are independent of each other in different groups; or the specific options expressed by the same symbol are independent of each other in same groups.

At various places in the present specification, substituents of compounds disclosed herein are disclosed in groups or in ranges. It is specifically intended that the invention include each and every individual subcombination of the members of such groups and ranges. For example, the term “C1-C6 alkyl” is specifically intended to individually disclose methyl, ethyl, C3 alkyl, C4 alkyl, C5 alkyl, and C6 alkyl.

At various places in the present specification, linking substituents are described. Where the structure clearly requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “alkyl” or “alkyl group” refers to a saturated linear or branched-chain monovalent hydrocarbon group of 1-20 carbon atoms, wherein the alkyl group is optionally substituted with one or more substituents described herein. Unless otherwise stated, the alkyl group contains 1-20 carbon atoms. In some embodiments, the alkyl group contains 1-12 carbon atoms; in other embodiments, the alkyl group contains 2-12 carbon atoms; in other embodiments, the alkyl group contains 1-6 carbon atoms; in other embodiments, the alkyl group contains 2-6 carbon atoms; in still other embodiments, the alkyl group contains 1-4 carbon atoms; in yet other embodiments, the alkyl group contains 1-3 carbon atoms.

Some non-limiting examples of the alkyl group include, methyl (Me, —CH₃), ethyl (Et, —CH₂CH₃), n-propyl (n-Pr, —CH₂CH₂CH₃), isopropyl (i-Pr, —CH(CH₃)₂), n-butyl (n-Bu, —CH₂CH₂CH₂CH₃), isobutyl (i-Bu, —CH₂CH(CH₃)₂), sec-butyl (s-Bu, —CH(CH₃)CH₂CH₃), tert-butyl (t-Bu, —C(CH₃)₃), n-pentyl (—CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 2,2-dimethylpropyl (neopentyl, —CH₂C(CH₃)₂CH₃), 3-methyl-1-butyl (—CH₂CH₂CH(CH₃)₂), 2-methyl-1-butyl (—CH₂CH(CH₃)CH₂CH₃), n-hexyl (—CH₂CH₃CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, n-heptyl and n-octyl, etc.

The term “alkenyl” refers to linear or branched-chain monovalent hydrocarbon radical of 2 to 12 carbon atoms with at least one site of unsaturation, i.e., a carbon-carbon, sp² double bond, wherein the alkenyl radical may be optionally substituted independently with one or more substituents described herein, and includes radicals having “cis” and “trans” orientations, or alternatively, “E” and “Z” orientations. In some embodiments, the alkenyl contains 2 to 8 carbon atoms. In other embodiments, the alkenyl contains 2 to 6 carbon atoms. In still other embodiments, the alkenyl contains 2 to 4 carbon atoms. Some non-limiting examples of the alkenyl group include ethenyl or vinyl (—CH═CH₂), allyl (—CH₃CH═CH₂), propenyl (—CH═CHCH₂), and the like.

The term “alkynyl” refers to a linear or branched monovalent hydrocarbon radical of 2 to 12 carbon atoms with at least one site of unsaturation, i.e., a carbon-carbon, sp triple bond, wherein the alkynyl radical may be optionally substituted independently with one or more substituents described herein. In some embodiments, the alkynyl contains 2 to 8 carbon atoms. In other embodiments, the alkynyl contains 2 to 6 carbon atoms. In still other embodiments, the alkynyl contains 2 to 4 carbon atoms. Some non-limiting examples of the alkynyl group include ethynyl (—C≡CH), propargyl (—CH₂C≡CH), 1-propynyl (—C≡C—CH₃), 1-butynyl (—CH₂CH₂C≡CH), 2-alkynbutyl (—CH₂C≡CCH₃), 3-alkynbutyl (—C≡CCH₂CH₃), and the like.

The term “alkoxy” refers to an alkyl group, as previously defined, attached to the parent molecular moiety via an oxygen atom. Unless otherwise specified, the alkoxy group contains 1-12 carbon atoms. In one embodiment, the alkoxy group contains 1-6 carbon atoms. In other embodiment, the alkoxy group contains 1-4 carbon atoms. In still other embodiment, the alkoxy group contains 1-3 carbon atoms. The alkoxy group may be optionally substituted with one or more substituents disclosed herein.

Some non-limiting examples of the alkoxy group include, but are not limited to, methoxy (MeO, —OCH₃), ethoxy (EtO, —OCH₂CH₃), 1-propoxy (n-PrO, n-propoxy, —OCH₂CH₂CH₃), 2-propoxy (i-PrO, i-propoxy, —OCH(CH₃)₂), 1-butoxy (n-BuO, n-butoxy, —OCH₂CH₂CH₂CH₃), 2-methyl-l-propoxy (i-BuO, i-butoxy, —OCH₂CH(CH₃)₂), 2-butoxy (s-BuO, s-butoxy, —OCH(CH₃)CH₂CH₃), 2-methyl-2-propoxy (t-BuO, t-butoxy, —OC(CH₃)₃), 1-pentoxy (n-pentoxy, —OCH₂CH₂CH₂CH₂CH₃), 2-pentoxy (—OCH(CH₃)CH₂CH₂CH₃), 3-pentoxy (—OCH(CH₂CH₃)₂), 2-methyl-2-butoxy (—OC(CH₃)₂CH₂CH₃), 3-methyl-2-butoxy (—OCH(CH₃)CH(CH₃)₂), 3-methyl-1-butoxy (—OCH₂CH₂CH(CH₃)₂), 2-methyl-1-butoxy (—OCH₂CH(CH₃)CH₂CH₃), and the like.

The terms “hydroxyalkyl” and “hydroxyalkoxy” refer to an alkyl or alkoxy, as the case may be, is substituted with one or more hydroxy, wherein “hydroxy alkyl” and “hydroxyalkyl” are used interchangeably, examples of such include, but are not limited to, hydroxymethyl (—CH₂OH), hydroxyethyl (—CH₂CH₂OH, —CHOHCH₃), hydroxypropyl (—CH₂CH₂CH₂OH, —CH₂CHOHCH₃, —CHOHCH₂CH₃,), hydroxymethoxy (—OCH₂OH), and the like.

The term “haloalkoxy” refers to that an alkoxy group is substituted with one or more halogen atoms, wherein the alkoxy has the meaning described herein; examples of this include, but are not limited to, trifluoromethoxy (-OCF₃), and the like.

The term “haloalkyl” refers to an alkyl group is substituted with one or more halogen groups, wherein the alkyl is as defined herein. In some embodiments, the haloalkyl group contains 1-12 carbon atoms. In other embodiments, the haloalkyl group contains 1-10 carbon atoms. In other embodiments, the haloalkyl group contains 1-8 carbon atoms. In still other embodiments, the haloalkyl group contains 1-6 carbon atoms. In yet other embodiments, the haloalkyl group contains 1-4 carbon atoms and in still yet other embodiments, the haloalkyl group contains 1-3 carbon atoms. Such examples include, but are not limited to, difluoromethyl, trifluoromethyl, trifluoroethyl, and the like.

The term “cycloalkyl” refers to a monovalent or multivalent saturated ring having 3 to 12 carbon atoms as a monocyclic, bicyclic, or tricyclic ring system, In some embodiments, the cycloalkyl group contains 3-12 carbon atoms. In other embodiments, the cycloalkyl group contains 3-8 carbon atoms. In still other embodiments, the cycloalkyl group contains 4-7 carbon atoms. In other embodiments, the cycloalkyl group contains 3-6 carbon atoms. In some embodiments, the cycloalkyl group is a C₇₋₁₂ cycloalkyl containing 7-12 carbon atoms, which further contains a C₇₋₁₂ spirobicycloalkyl, a C₇₋₁₂ fused bicycloalkyl, and a C₇₋₁₂ bridged bicycloalkyl. In other embodiments, cycloalkyl group is a C₈₋₁₁ cycloalkyl containing 8-11 carbon atoms, which further contains a C₈₋₁₁ spirobicycloalkyl, a C₈₋₁₁ fused bicycloalkyl and a C₈₋₁₁ bridged bicycloalkyl. In some embodiments, C₃₋₆ cycloalkyl specifically refers to a ring containing 3-6 carbon atoms, including cyclopropyl, cyclobutyl, cyclopentyl and cyclohexyl. The cycloalkyl group may be optionally substituted with one or more substituents disclosed herein.

The terms “heterocyclyl” and “heterocycle” are used interchangeably herein, refer to a monovalent or polyvalent, saturated or partially unsaturated, non-aromatic monocyclic, bicyclic ring or tricyclic ring system containing 3-12 ring atoms, wherein at least one ring atom is selected from nitrogen, sulfur and oxygen atoms. Unless otherwise specified, the heterocyclyl group may be carbon or nitrogen linked, and a —CH₂— group can be optionally replaced by a —C(═O)— group. In which, the sulfur can be optionally oxygenized to S-oxide. and the nitrogen can be optionally oxygenized to N-oxide. Heterocyclyl includes saturated heterocyclyl (i.e.: heterocycloalkyl) and partially unsaturated heterocyclyl. In some embodiments, the heterocyclyl group is a heterocyclyl consisting of 3-8 atoms; in other embodiments, the heterocyclyl group is a heterocyclyl consisting of 3-6 atoms.

Some non-limiting examples of the heterocyclyl group include oxiranyl, azetidinyl, oxetanyl, thietanyl, pyrrolidinyl, 2-pyrrolinyl, 3-pyrrolinyl, pyrazolinyl, pyrazolidinyl, imidazolinyl, imidazolidinyl, tetrahydrofuranyl, dihydrofuranyl, tetrahydrothienyl, dihydrothienyl, 1,3-dioxolanyl, dithiolanyl, tetrahydropyranyl, dihydropyranyl, 2H-pyranyl, 4H-pyranyl, tetrahydrothiopyranyl, piperidinyl, morpholinyl, thiomorpholinyl, piperazinyl, dioxanyl, dithianyl, thioxanyl, homopiperazinyl, homopiperidinyl, oxepanyl, thiepanyl, oxazepinyl (e.g., 1,4-oxazepinyl, 1,2-oxazepinyl), diazepinyl (e.g., 1,4-diazepinyl, 1,2 -diazepinyl), dioxazinyl (e.g., 1,4-dioxazinyl, 1,2-dioxazinyl), thiazepinyl (e.g., 1,4-thiazepinyl, 1,2-thiazepinyl), indolinyl, 1,2,3,4-tetrahydroisoquinolyl, 1,3-benzodioxolyl, 2-oxa-5-azabicyclo[2.2.1]hept-5-yl, 2-azaspiro[4.4]nonyl, 1,6-dioxaspiro[4.4]nonyl, 2-azaspiro[4.5 ]decyl, 8-azaspiro[4.5]decyl, 7-azaspiro[4.5]decyl, 3-azaspiro[5.5]undecyl, 2-azaspiro[5.5] undecyl, octahydro-1H-isoindolyl, octahydrocyclopenta[c]pyrrolyl, hexahydrofuro[3,2-b]furyl and dodecahydroisoquinolyl, and the like. Some non-limiting examples of heterocyclyl wherein —CH₂— group is replaced by —C(═O)—include 1,1-dioxoisothiazolidinone-2-yl, pyrrolidin-2-one-1-yl, imidazolidin-2-one-1-yl, oxazolidin-2-one-3-yl, 2-oxopyrrolidinyl, oxo-1,3-thiazolidinyl, 2-piperidinonyl and 3,5-dioxopiperidinyl. Some non-limited examples of heterocyclyl wherein the sulfur atom is oxidized is sulfolanyl, 1,1-dioxothiomorpholinyl, 1,1-dioxotetrahydrothiophenyl and 1,1-dioxotetrahydro-2H-thiopyranyl, and the like. The heterocyclyl group may be optionally substituted with one or more substituents disclosed herein.

The term “s membered”, wherein s is an integer typically describes the number of ring-forming atoms in a moiety where the number of ring-forming atoms is s. For example, piperidinyl is an example of a 6 membered heterocycloalkyl and 1,2,3,4-tetrahydro-naphthalene is an example of a 10 membered carbocyclyl group.

The term “ring consisting of M-M¹ atoms” refers to that the cyclic group is composed of M-M₁ atoms, and the atoms include carbon atoms and/or O, N, S, P and other heteroatoms. For example, “heteroaryl consisting of 5-10 atoms” means that the heteroaryl is composed of 5, 6, 7, 8, 9 or 10 atoms.

The term “unsaturated” refers to a moiety having one or more units of unsaturation.

The term “heteroatom” refers to one or more of oxygen, sulfur, nitrogen, phosphorus and silicon, including any oxidized form of nitrogen, sulfur, or phosphorus; the quaternized form of any basic nitrogen; or a substitutable nitrogen of a heterocyclic ring, for example, N (as in 3,4-dihydro-2H-pyrrolyl), NH (as in pyrrolidinyl) or NR (as in N-substituted pyrrolidinyl).

The term “halogen” refers to fluorine(F), chlorine(Cl), bromine(Br) or iodine(I).

The term “aryl” refers to monocyclic, bicyclic and tricyclic carbocyclic ring systems having a total of 6-14 ring members, or 6-12 ring members, or 6-10 ring members, wherein at least one ring in the system is aromatic, wherein each ring in the system contains 3 to 7 ring members and that has a single point or multipoint of attachment to the rest of the molecule. The term “aryl” and “aromatic ring” can be used interchangeably herein. Examples of aryl ring may include phenyl, naphthyl and anthracene. The aryl group may be optionally and independently substituted with one or more substituents disclosed herein.

The term “heteroaryl” refers to aromatic monocyclic, bicyclic and tricyclic ring systems having a total of 5-12 ring members, or 5-10 ring members, or 5-6 ring members, wherein at least one ring member is selected from nitrogen, sulfur and oxygen, and wherein each ring in the system contains 5 to 7 ring members and that has a single point or multipoint of attachment to the rest of the molecule. The term “hetreroaryl” and “heteroaromatic ring” or “heteroaromatic compound” can be used interchangeably herein.

Examples of heteroaryl include, but are not limited to: benzimidazolyl, benzofuryl, benzothienyl, indolyl (such as 2-indolyl, 3-indolyl, 4-indolyl, 5-indolyl, 6-indolyl, 7-indolyl), purinyl, quinolinyl (such as 2-quinolinyl, 3-quinolinyl, 4-quinolinyl), isoquinolinyl (such as 1-isoquinolinyl, 3-isoquinolinyl, 4-isoquinolinyl), indazolyl (such as 3-indazolyl, 4-indazolyl, 5-indazolyl, 6-indazolyl, 7-indazolyl), imidazo[1,2-a]pyridyl, pyrazolo[1,5-a]pyridyl, pyrazolo[4,3-c]pyridyl, pyrazolo[3,4-b]pyridyl, pyrazolo[1,5-a]pyrimidinyl, imidazo[1,2-b]pyridazinyl, [1,2,4]triazolo[4,3-b]pyridazinyl, [1,2,4]triazolo[1,5-a]pyrimidinyl, [1,2,4]triazolo[1,5-a]pyridinyl, [1,2,4]triazolo[4,3-a]pyridyl, imidazo[1,2-c]pyrimidinyl, 1H-benzo[d][1,2,3]triazolyl, 3H-imidazo[4,5-b]pyridiyl, 1H-pyrrolo[2,3-b]pyridyl, 1H-benzo[d]imidazolyl, 1H-pyrazolo[3,2-b]pyridyl, [1,2,4]triazolo[1,5-a]pyridyl, purinyl, furyl (such as 2-furyl, 3-furyl), imidazolyl (such as 1-imidazolyl, 2-imidazolyl, 4-imidazolyl, 5-imidazolyl), isoxazolyl (such as 3-isoxazolyl, 4-isoxazolyl, 5-isoxazolyl), oxazolyl (such as 2-oxazolyl, 4-oxazolyl, 5-oxazolyl), pyrrolyl (such as 1-pyrrolyl, 2-pyrrolyl, 3-pyrrolyl), pyridyl (such as 2-pyridyl, 3-pyridyl, 4-pyridyl), pyridinone, pyrimidinyl (such as 2-pyrimidinyl, 4-pyrimidinyl, 5-pyrimidinyl), pyrimidinone, pyrimidinedione, pyridazinyl (such as 3-pyridazinyl, 4-pyridazinyl), pyrazinyl (such as 2-pyrazinyl, 3-pyrazinyl), thiazolyl (such as 2-thiazolyl, 4-thiazolyl, 5-thiazolyl), tetrazolyl (such as 5-tetrazolyl), triazolyl (such as 2-triazolyl and 5-triazolyl), thienyl (such as 2-thienyl, 3-thienyl), pyrazolyl (such as 1-pyrazolyl, 3-pyrazolyl, 4-pyrazolyl, 5-pyrazolyl), pyrazolone, isothiazolyl, oxadiazolyl (such as 1,2,3-oxadiazolyl, 1,2,5-oxadiazolyl, 1,2,4-oxadiazolyl), 1,2,3-triazolyl, 1,2,3-thiodiazolyl, 1,3,4-thiodiazolyl, 1,2,5-thiodiazolyl, pyrazinyl and 1,3,5-triazinyl, and the like.

The term “alkylamino” and “alkyl amino” are used interchangeably and include “N-alkylamino” and “N,N-dialkylamino”, wherein amino groups are independently substituted with one alkyl radical or two alkyl radicals, respectively. In some embodiments, the alkylamino radical is a lower alkylamino radical having one or two C₁₋₁₂ alkyl radicals attached to a nitrogen atom. In some other embodiments, the alkylamino radical is a lower alkylamino radical having one or two C₁₋₆ alkyl radicals attached to a nitrogen atom. In some other embodiments, the alkylamino radical is a lower alkylamino radical having one or two C₁₋₄ alkyl radicals attached to a nitrogen atom. In still some embodiments, the alkylamino radical is a lower alkylamino radical having one or two C₁₋₃ alkyl radicals attached to a nitrogen atom. Some non-limiting examples of suitable alkylamino radical include mono or dialkylamino. Some examples include, but not limited to, N-methylamino, N-ethylamino, N,N-dimethylamino, N,N-diethylamino, N-ethylpropyl-2-amino, and the like.

The term “prodrug” refers to a compound that is transformed in vivo into a compound of Formula (I) or (II). Such a transformation can be affected, for example, by hydrolysis of the prodrug form in blood or enzymatic transformation to the parent form in blood or tissue. Prodrugs of the compounds disclosed herein may be, for example, esters. Some common esters which have been utilized as prodrugs are phenyl esters, aliphatic (C₁₋₂₄) esters, acyloxymethyl esters, carbonates, carbamates and amino acid esters. For example, a compound disclosed herein that contains a hydroxy group may be acylated at this position in its prodrug form. Other prodrug forms include phosphates, such as, those phosphate compounds derived from the phosphonation of a hydroxy group on the parent compound. A thorough discussion of prodrugs is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, J. Rautio et al., Prodrugs: Design and Clinical Applications, Nature Review Drug Discovery, 2008, 7, 255-270, and S. J. Hecker et al., Prodrugs of Phosphates and Phosphonates, Journal of Medicinal Chemistry, 2008, 51, 2328-2345.

A “metabolite” is a product produced through metabolism in the body of a specified compound or salt thereof. The metabolites of a compound may be identified using routine techniques known in the art and their activities determined using tests such as those described herein. Such products may result for example from oxidation, reduction, hydrolysis, amidation, deamidation, esterification, deesterification, enzyme cleavage, and the like, of the administered compound. Accordingly, the invention includes metabolites of compounds disclosed herein, including metabolites produced by contacting a compound disclosed herein with a mammal for a sufficient time period.

A “pharmaceutically acceptable salts” refers to organic or inorganic salts of a compound disclosed herein. Pharmaceutically acceptable salts are well known in the art. For example, S. M. Berge et al., describe pharmaceutically acceptable salts in detail in J. Pharmaceutical Sciences, 1977, 66: 1-19, which is incorporated herein by reference. Some non-limiting examples of pharmaceutically acceptable and nontoxic salts include salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid and malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, laurylsulfate, malate, malonate, methanesulfonate, 2-naphthalenesulfonate, nicotinate, nitrate, oleate, palmitate, pamoate, pectinate, persulfate, 3-phenylpropionate, picrate, pivalate, propionate, stearate, thiocyanate, p-toluenesulfonate, undecanoate, valerate, and the like. Salts derived from appropriate bases include alkali metal, alkaline earth metal, ammonium and N⁺(C₁₋₄ alkyl)₄ salts. This invention also envisions the quaternization of any basic nitrogen-containing groups of the compounds disclosed herein. Water or oil soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, C1-8 sulfonate or aryl sulfonate.

The term “solvate” refers to an association or complex of one or more solvent molecules and a compound disclosed herein. Examples of solvents that form solvates include, but are not limited to, water, isopropanol, ethanol, methanol, DMSO, ethyl acetate, acetic acid and ethanolamine. The term “hydrate” refers to the complex where the solvent molecule is water.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

“Inflammatory disorder/disease” as used herein can refer to any disease, disorder, or syndrome in which an excessive or unregulated inflammatory response leads to excessive inflammatory symptoms, host tissue damage, or loss of tissue function. “Inflammatory disorder” also refers to a pathological state mediated by influx of leukocytes and/or neutrophil chemotaxis.

“Inflammation” as used herein refers to a localized, protective response elicited by injury or destruction of tissues, which serves to destroy, dilute, or wall off (sequester) both the injurious agent and the injured tissue. Inflammation is notably associated with influx of leukocytes and/or neutrophil chemotaxis. Inflammation can result from infection with pathogenic organisms and viruses and from noninfectious means such as trauma or reperfusion following myocardial infarction or stroke, immune response to foreign antigen, and autoimmune responses. Accordingly, inflammatory disorders amenable to treatment with the compounds disclosed herein encompass disorders associated with reactions of the specific defense system as well as with reactions of the nonspecific defense system.

“Specific defense system” refers to components of the immune system that respond to the presence of specific antigens. Examples of inflammation arising from specific defense system responses include classical responses to foreign antigens, autoimmune diseases, and delayed hypersensitivity responses (mediated by T-cells). Chronic inflammatory diseases, rejection of transplanted solid tissues and organs (such as rejection of kidney and bone marrow transplants), and graft-versus-host disease (GVHD) are other examples of inflammatory responses of specific defense systems.

“Autoimmune disease” as used herein refers to any group of disorders in which tissue injury is associated with humoral or cell-mediated responses to the body’s own constituents.

“Allergy” as used herein refers to any symptom, tissue damage, or loss of tissue function that produces allergy. “Arthritic disease” as used herein refers to any disease that is characterized by inflammatory lesions of the joints attributable to a variety of etiologies. “Dermatitis” as used herein refers to any of a large family of diseases of the skin that are characterized by inflammation of the skin attributable to a variety of etiologies. “Transplant rejection” as used herein refers to any immune reaction directed against grafted tissue, such as organs or cells (e.g., bone marrow), characterized by a loss of function of the grafted and surrounding tissues, pain, swelling, leukocytosis, and thrombocytopenia. The therapeutic methods of the present invention include methods for the treatment of disorders associated with inflammatory cell activation.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small- cell lung cancer, non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, testicular tumors, bladder cancer, hepatoma, breast cancer, colon cancer, rectum cancer, colorectum cancer, endometrium or uterus cancer, salivary gland cancer, kidney or kidney cancer, prostate cancer, vulvar cancer, thyroid cancer, medullary thyroid cancer, melanoma, retinoblastoma, hepatic carcinoma, anal cancer, penile cancer, acute myeloid leukemia, acute lymphoblastic leukemia, chronic myelogenous leukemia (CML), chronic lymphocytic leukemia, as well as head and neck cancer.

### Description of Compounds of the Invention

The present invention discloses a class of novel compounds which can be used as inhibitors of JAK kinase activity. Compounds thereof as JAK protein kinase inhibitors are useful in the treatment of diseases associated with JAK kinase activity, particularly TYK2 activity, such diseases include inflammatory diseases, autoimmune diseases, metabolic diseases, destructive bone diseases, proliferative disease, angiogenic disorder, sepsis, septic shock, shigellosis, neurodegenerative disease, or retinitis.

In one aspect, the present invention provides a compound having Formula (I) or a stereoisomer, a geometric isomer, a tautomer, an N-oxide, a hydrate, a solvate, a metabolite, a pharmaceutically acceptable salt or a prodrug thereof,

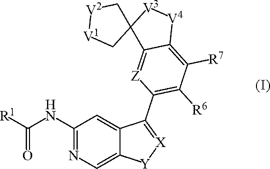

wherein, each of the X, Y, Z, R¹, R⁶, R⁷, V¹, V², V³ and V⁴ has the meaning described in the present invention.

In some embodiments, the compounds of the present invention have the structure shown in formula (II):

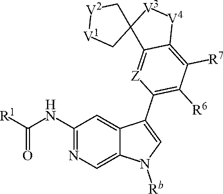

(II), wherein each of the Z, R¹, R⁶, R⁷, R^{b}, V¹, V², V³ and V⁴ has the meaning described in the present invention.

In some embodiments, X is N or CR^{a}; wherein the R^{a} has the meaning described herein.

In some embodiments, Z is N or CR^{e}; wherein the R^{e} has the meaning described herein.

In some embodiments, Y is NR^{b} or CR^{c}R^{d}; wherein each of the R^{b}, R^{c} and R^{d} has the meaning described herein.

In some embodiments, R¹ is —NH₂, C₁₋₆ alkyl, C₁₋₆ alkylamino, C₃₋₈ cycloalkyl, heterocyclyl consisting of 3-8 atoms, C₆₋₁₀ aryl or heteroaryl consisting of 5-12 atoms, wherein, the -NH₂, C₁₋₆ alkyl, C₁₋₆ alkylamino, C₃₋₈ cycloalkyl, heterocyclyl consisting of 3-8 atoms, C₆₋₁₀ aryl and heteroaryl consisting of 5-12 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R¹ is —NH₂, C₁₋₄ alkyl, C₁₋₄ alkylamino, C₃₋₆ cycloalkyl, heterocyclyl consisting of 3-6 atoms, C₆₋₁₀ aryl or heteroaryl consisting of 5-10 atoms, wherein, the -NH₂, C₁₋₄ alkyl, C₁₋₄ alkylamino, C₃₋₆ cycloalkyl, heterocyclyl consisting of 3-6 atoms, C₆₋₁₀ aryl and heteroaryl consisting of 5-10 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R¹ is —NH₂, methyl, ethyl, n-propyl, isopropyl, n-butyl, isobutyl, sec-butyl, tert-butyl, N-methylamino, N-ethylamino, N,N-dimethylamino, N,N-diethylamino, N-ethylpropyl-2-amino, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein, the —NH₂, methyl, ethyl, n-propyl, isopropyl, n-butyl, isobutyl, sec-butyl, tert-butyl, N-methylamino, N-ethylamino, N,N-dimethylamino, N,N-diethylamino, N-ethylpropyl-2-amino, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and pyridazinyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, —OCH₂OH and —OCH₂CH₂OH.

In some embodiments, R⁶ is H, F, Cl, Br, I, —NO₂, CN, —OH, —NH₂, C₁₋₆ alkyl, C₁₋₆ haloalkyl, C₂₋₆ alkenyl, C₂₋₆ alkynyl, C₁₋₆ alkoxy, C₃₋₆ cycloalkyl, C₃₋₆ cycloalkyl—O—, C₁₋₆ alkylamino, C₁₋₆ alkyl—S(═O)₂—, C₁₋₆ alkyl-S-, heterocyclyl consisting of 3-8 atoms, C₆₋₁₀ aryl or heteroaryl consisting of 5-10 atoms, wherein, the —OH, —NH₂, C₁₋₆ alkyl, C₁₋₆ haloalkyl, C₂₋₆ alkenyl, C₂₋₆ alkynyl, C₁₋₆ alkoxy, C₃₋₆ cycloalkyl, C₃₋₆ cycloalkyl-O-, C₁₋₆ alkylamino, C₁₋₆ alkyl-S(=O)₂-, C₁₋₆ alkyl-S-, heterocyclyl consisting of 3-8 atoms, C₆₋₁₀ aryl and heteroaryl consisting of 5-10 atoms can be independently and optionally substituted with 1, 2, 3, 4 or 5 R^{x}; wherein the R^{x} has a meaning as described in the present invention.

In other embodiments, R⁶ is H, F, Cl, Br, I, —NO₂, CN, —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, C₃₋₆ cycloalkyl-O-, C₁₋₄ alkylamino, C₁₋₄ alkyl-S(=O)₂-, C₁₋₄ alkyl-S-, heterocyclyl consisting of 3-6 atoms, C₆₋₁₀ aryl or heteroaryl consisting of 5-10 atoms, wherein, the —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, C₃₋₆ cycloalkyl-O-, C₁₋₄ alkylamino, C₁₋₄ alkyl-S(=O)₂-, C₁₋₄ alkyl-S-, heterocyclyl consisting of 3-6 atoms, C₆₋₁₀ aryl and heteroaryl consisting of 5-10 atoms can be independently and optionally substituted with 1, 2, 3, 4 or 5 R^{x}; wherein the R^{x} has a meaning as described in the present invention.

In other embodiments, R⁶ is H, F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, cyclopropyl-O-, cyclobutyl-O-, cyclopentyl-O-, cyclohexyl-O-, —NH(CH₂)₃CH₃, CH₃(CH₂)₃S—, CH₃—S(═O)₂—, CH₃(CH₂)₃—S(═O)₂—, oxetanyl, azetidinyl, pyrrolidinyl, tetrahydrofuranyl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein, the —OH, —NH₂, methyl, ethyl, n-propyl, isopropyl, n-butyl, isobutyl, sec-butyl, tert-butyl, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, cyclopropyl-O-, cyclobutyl-O-, cyclopentyl-O-, cyclohexyl-O-, —NH(CH₂)₃CH₃, CH₃(CH₂)₃S—, CH₃—S(═O)₂—, CH₃(CH₂)₃—S(═O)₂—, oxetanyl, azetidinyl, pyrrolidinyl, tetrahydrofuranyl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and idazinyl can be independently and optionally substituted with 1, 2, 3, 4 or 5 R^{x}; wherein each R^{x} has the meaning as described in the present invention.

In some embodiments, R⁷ is H, F, Cl, Br, I, —NO₂, CN, —OH, —NH₂, C₁₋₆ alkyl, C₁₋₆ haloalkyl, C₂₋₆ alkenyl, C₂₋₆ alkynyl, C₁₋₆ alkoxy, C₃₋₆ cycloalkyl, C₃₋₆ cycloalkyl-O-, C₁₋₆ alkylamino, C₁₋₆ alkyl-S(=O)₂-, C₁₋₆ alkyl-S-, heterocyclyl consisting of 3-8 atoms, C₆₋₁₀ aryl or heteroaryl consisting of 5-10 atoms, wherein, the —OH, —NH₂, C₁₋₆ alkyl, C₁₋₆ haloalkyl, C₂₋₆ alkenyl, C₂₋₆ alkynyl, C₁₋₆ alkoxy, C₃₋₆ cycloalkyl, C₃₋₆ cycloalkyl-O-, C₁₋₆ alkylamino, C₁₋₆ alkyl-S(=O)₂-, C₁₋₆ alkyl-S-, heterocyclyl consisting of 3-8 atoms, C₆₋₁₀ aryl and heteroaryl consisting of 5-10 atoms can be independently and optionally substituted with 1, 2, 3, 4 or 5 R^{x}; wherein the R^{x} has a meaning as described in the present invention.

In other embodiments, R⁷ is H, F, Cl, Br, I, —NO₂, CN, —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, C₃₋₆ cycloalkyl-O-, C₁₋₄ alkylamino, C₁₋₄ alkyl-S(=O)₂-, C₁₋₄ alkyl-S-, heterocyclyl consisting of 3-6 atoms, C₆₋₁₀ aryl or heteroaryl consisting of 5-10 atoms, wherein, the —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, C₃₋₆ cycloalkyl-O-, C₁₋₄ alkylamino, C₁₋₄ alkyl-S(=O)₂-, C₁₋₄ alkyl-S-, heterocyclyl consisting of 3-6 atoms, C₆₋₁₀ aryl and heteroaryl consisting of 5-10 atoms can be independently and optionally substituted with 1, 2, 3, 4 or 5 R^{x}; wherein the R^{x} has a meaning as described in the present invention.

In other embodiments, R⁷ is H, F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, cyclopropyl-O-, cyclobutyl-O-, cyclopentyl-O-, cyclohexyl-O-, —NH(CH₂)₃CH₃, CH₃(CH₂)₃S—, CH₃—S(═O)₂—, CH₃(CH₂)₃—S(═O)₂—, oxetanyl, azetidinyl, pyrrolidinyl, tetrahydrofuranyl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein, the —OH, —NH₂, methyl, ethyl, n-propyl, isopropyl, n-butyl, isobutyl, sec-butyl, tert-butyl, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-₂-yne-1-yl, but-1-yne-1-yl, methoxy, ethoxy, 1-propoxy, ₂-propoxy, 1-butoxy, ₂-methyl-1-propoxy, ₂-butoxy, ₂-methyl-₂-propoxy, cyclopropyl, cyclobutyl, cyclopentyl, cyclohexyl, cyclopropyl-O-, cyclobutyl-O-, cyclopentyl-O-, cyclohexyl-O-, —NH(CH₂)₃CH₃, CH₃(CH₂)₃S—, CH₃—S(═O)₂—, CH₃(CH₂)₃—S(═O)₂—, oxetanyl, azetidinyl, pyrrolidinyl, tetrahydrofuranyl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and idazinyl can be independently and optionally substituted with 1, 2, 3, 4 or 5 R^{x}; wherein each R^{x} has the meaning as described in the present invention.

In some embodiments, V¹ is -(CR⁹R¹⁰)ₙ-, -(CR⁹R¹⁰)ₙ-O-, -(CR⁹R¹⁰)ₙ-S-, -(CR⁹R¹⁰)ₙ-NR¹¹-, -(CR⁹R¹⁰)ₙ-C(=O)-, -(CR⁹R¹⁰)ₙ-O-C(=O)-, -(CR⁹R¹⁰)ₙ-C(=O)-O-, -(CR⁹R¹⁰)ₙ-S(=O)- or -(CR⁹R¹⁰)ₙ-S(=O)₂-; wherein R⁹, R¹⁰, R¹¹ and n have the meanings as described in the present invention.

In other embodiments, V¹ is —CH₂—, —O— or —CH₂—O—.

In some embodiments, V² is -(CR⁹R¹⁰)ₙ-, -(CR⁹R¹⁰)ₙ-O-, -(CR⁹R¹⁰)ₙ-S-, -(CR⁹R¹⁰)ₙ-NR¹¹-, -(CR⁹R¹⁰)ₙ -C(=O)-, -(CR⁹R¹⁰)ₙ-O-C(=O)-, -(CR⁹R¹⁰)ₙ-C(=O)-O-, -(CR⁹R¹⁰)ₙ-S(=O)- or -(CR⁹R¹⁰)ₙ-S(=O)₂- wherein R⁹, R¹⁰, R¹¹ and n have the meanings as described in the present invention.

In other embodiments, V² is —CH₂—, —(CH₂)₂—, —O— or —CH₂—O—.

In some embodiments, V³ is -(CR⁹R¹⁰)ₙ-, -(CR⁹R¹⁰)ₙ-O-, -(CR⁹R¹⁰)ₙ-S-, -(CR⁹R¹⁰)ₙ-NR¹¹-, -(CR⁹R¹⁰)ₙ -C(=O)-, -(CR⁹R¹⁰)ₙ-O-C(=O)-, -(CR⁹R¹⁰)ₙ-C(=O)-O-, -(CR⁹R¹⁰)ₙ-S(=O)- or -(CR⁹R¹⁰)ₙ-S(=O)₂-; wherein R⁹, R¹⁰, R¹¹ and n have the meanings as described in the present invention.

In some embodiments, V³ is —CH₂—, —O—, —CH₂—O—, —(CH₂)₂—, —CH₂—C(═O)—, —C(═O)—O—, —O—C(═O)—, —NHCH₃— or —CH₂—NHCH₃—.

In some embodiments, V⁴ is -(CR⁹R¹⁰)ₙ-, -(CR⁹R¹⁰)ₙ₋O-, -(CR⁹R¹⁰)ₙ₋S-, -(CR⁹R¹⁰)ₙ-NR¹¹-, -(CR⁹R¹⁰)ₙ -C(=O)-, -(CR⁹R¹⁰)ₙ₋O-C(=O)-, -(CR⁹R¹⁰)ₙ₋C(=O)-O-, -(CR⁹R¹°)ₙS(=O)- or -(CR⁹R¹⁰)ₙ₋S(=O)₂-; wherein R⁹, R¹⁰, R¹¹and n have the meanings as described in the present invention.

In some embodiments, V⁴ is —CH₂—, —(CH₂)₂—, —O—, —CH₂—O—, —C(CH₃)₂—O—, —CH₂—C(═O)—, —C(═O)—O—,

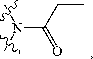

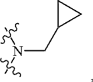

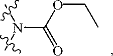

—N(CH₂CH₃)—, —C(CH₃)₂—NCH₃—, —CH₂CF₂—, —NCH₃— or —CH₂—NHCH₃—.

In some embodiments, R⁹ is H, D, F, Cl, Br, I, —NO², CN, —OH, —NH₂, C₁₋₆ alkyl, C₁₋₆ haloalkyl, C₁₋₆ alkoxy or C₃ ₋₆ cycloalkyl, wherein, the —OH, —NH₂, C₁₋₆ alkyl, C₁₋₆ haloalkyl, C₁₋₆ alkoxy and C₃ ₋₆ cycloalkyl can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R⁹ is H, D, F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl —CHF₂, —CHCl₂, —CF₃, —CH₂CH₂F, —CH₂CH₂Cl, —CH₂CHF₂, —CH₂CHCl₂, —CHFCH₂F, —CHCICH₂Cl, —CH₂CF₃, —CH(CF₃)₂, —CF₂CH₂CH₃, —CH₂CH₂CH₂F, —CH₂CH₂CHF₂, —CH₂CH₂CF₃, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl or cyclohexyl, wherein, the —OH, —NH₂, methyl, ethyl, n-propyl, isopropyl, n-butyl, isobutyl, sec-butyl, tert-butyl, CH₂F, —CH₂Cl CHF₂, —CHCl₂, —CH₂CH₂F, —CH₂CH₂Cl, —CH₂CHF₂, —CH₂CHCl₂, —CHFCH₂F, —CHCICH₂Cl, —CH₂CF₃, —CH(CF₃)₂, —CF₂CH₂CH₃, —CH₂CH₂CH₂F, —CH₂CH₂CHF₂, —CH₂CH₂CF₃, methoxy, ethoxy, 1-propoxy, 2-propoxy, 1-butoxy, 2-methyl-l-propoxy, 2-butoxy, 2-methyl-2- propoxy, cyclopropyl, cyclobutyl, cyclopentyl and cyclohexyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, —OCH₂OH and —OCH₂CH₂OH.

In some embodiments, R¹⁰ is H, D, F, Cl, Br, I, —NO², CN, —OH, —NH₂, C₁₋₆ alkyl, C₁₋₆ haloalkyl, C₁₋₆ alkoxy or C₃ ₋₆ cycloalkyl, wherein, the —OH, —NH₂, C₁₋₆ alkyl, C₁₋₆ haloalkyl, C₁₋₆ alkoxy and C₃ ₋₆ cycloalkyl can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R¹⁰ is H, D, F, Cl, Br, I, —NO2, CN, —OH, —NH2, methyl, ethyl, n-propyl, isopropyl, n-butyl, isobutyl, sec-butyl, tert-butyl, CH2F, —CH2Cl, —CHF2, —CHCl2, —CF3, —CH2CH2F, —CH2CH2Cl, —CH2CHF2, —CH2CHCl2, —CHFCH2F, —CHCICH2Cl, —CH2CF3, —CH(CF3)2, —CF2CH2CH3, —CH2CH2CH2F, —CH2CH2CHF2, —CH2CH2CF3, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl or cyclohexyl, wherein, the —OH, —NH2, methyl, ethyl, n-propyl, isopropyl, n-butyl, isobutyl, sec-butyl, tert-butyl, CH2F, —CH2Cl, —CHF2, —CHCl2, —CH2CH2F, —CH2CH2Cl, —CH2CHF2, —CH2CHCl2, —CHFCH2F, —CHCICH2Cl, —CH2CF3, —CH(CF3)2, —CF2CH2CH3, —CH2CH2CH2F, —CH2CH2CHF2, —CH2CH2CF3, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl and cyclohexyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, —OCH2OH and —OCH2CH2OH.

In some embodiments, R⁹ and R¹⁰, together with the carbon atom to which they are attached, form C₃₋₆ cycloalkyl or heterocyclyl consisting of 3-6 atoms, wherein, the C₃₋₆ cycloalkyl and heterocyclyl consisting of 3-6 atoms can be independently and optionally substituted with 1, 2, 3, 4 or 5 substituents selected from F, Cl, Br, I, —NO₂, CN, —OH, —NH₂, oxo, C₁₋₃ alkyl, C₁₋₃ haloalkyl, C₁₋₃ alkoxy, C₁₋₃ haloalkoxy and C₁₋₃ hydroxyalkoxy.

In other embodiments, R⁹ and R¹⁰, together with the carbon atom to which they are attached, form cyclopropyl, cyclobutyl, cyclopentyl, cyclohexyl, oxetanyl, azetidinyl, pyrrolidinyl, tetrahydrofuranyl, piperidinyl, piperazinyl or morpholinyl, wherein, each of the cyclopropyl, cyclobutyl, cyclopentyl, cyclohexyl, oxetanyl, azetidinyl, pyrrolidinyl, tetrahydrofuranyl, piperidinyl, piperazinyl or morpholinyl is independently unsubstituted or substituted with 1, 2, 3, 4 or 5 substituents selected from F, Cl, Br, I, —NO₂, CN, —OH, —NH₂, oxo, methyl, ethyl, n-propyl, isopropyl, trifluoromethyl, difluoromethyl, methoxy, ethoxy, isopropoxy, trifluoromethoxy, —OCH₂OH and —OCH₂CH₂OH.

In some embodiments, R¹¹ is H, D, C₁₋₆ alkyl, C₁₋₆ alkyl—C(═O)—, C₁₋₆ alkyl—O—C(═O)—, C₁₋₆ haloalkyl or C₃₋₆ cycloalkyl, wherein, C₁₋₆ alkyl, C₁₋₆ alkyl—C(═O)—, C₁₋₆ alkyl—O—C(═O)—, C₁₋₆ haloalkyl and C₃₋₆ cycloalkyl can be independently and optionally substituted with 1, 2, 3, 4 or 5 substituents selected from F, Cl, Br, I, —NO₂, CN, —OH, —NH₂, oxo, C₁₋₃ alkyl, C₁₋₃ haloalkyl, C₁₋₃ alkoxy, C₁₋₃ haloalkoxy, C₁₋₃ hydroxyalkoxy and C₃₋₆ cycloalkyl.

In other embodiments, R¹¹ is H, D, methyl, ethyl, n-propyl, isopropyl, n-butyl, isobutyl, sec-butyl, tert-butyl, CH₃CH₂—C(═O)—, CH₃CH₂—O—C(═O)—, CH₂F, —CH₂Cl CHF₂, —CHCl₂, —CF₃, —CH₂CH₂F, —CH₂CH₂Cl, —CH₂CHF₂, —CH₂CHCl₂, —CHFCH₂F, —CHCICH₂Cl, —CH₂CF₃, —CH(CF₃)₂, —CF₂CH₂CH₃, —CH₂CH₂CH₂F, —CH₂CH₂CHF₂, —CH₂CH₂CF₃, cyclopropyl, cyclobutyl, cyclopentyl or cyclohexyl, wherein, each of the methyl, ethyl, n-propyl, isopropyl, n-butyl, isobutyl, sec-butyl, tert-butyl, CH₃CH₂—C(═O)—, CH₃CH₂—O—C(═O)—, CH₂F, —CH₂Cl CHF₂, —CHCl₂, —CH₂CH₂F, —CH₂CH₂Cl, —CH₂CHF₂, —CH₂CHCl₂, —CHFCH₂F, —CHCICH₂Cl, —CH₂CF₃, —CH(CF₃)₂, —CF₂CH₂CH₃, —CH₂CH₂CH₂F, —CH₂CH₂CHF₂, —CH₂CH₂CF₃, cyclopropyl, cyclobutyl, cyclopentyl and cyclohexyl is independently unsubstituted or substituted with 1, 2, 3, 4 or 5 substituents selected from F, Cl, Br, I, —NO₂, CN, —OH, —NH₂, oxo, methyl, ethyl, n-propyl, isopropyl, trifluoromethyl, difluoromethyl, methoxy, ethoxy, isopropoxy, trifluoromethoxy, —OCH₂OH, —OCH₂CH₂OH, cyclopropyl, cyclobutyl, cyclopentyl and cyclohexyl.

In some embodiments,

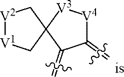

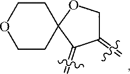

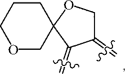

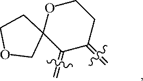

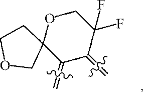

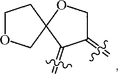

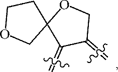

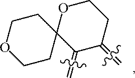

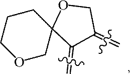

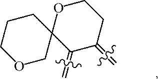

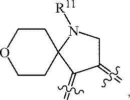

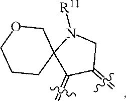

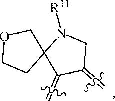

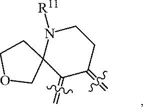

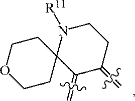

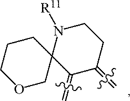

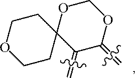

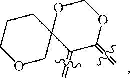

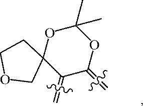

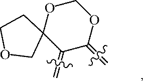

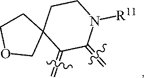

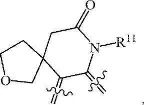

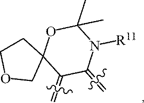

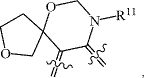

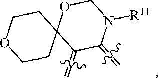

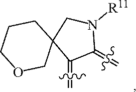

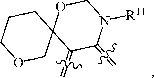

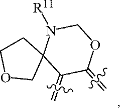

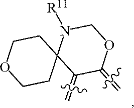

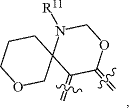

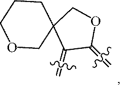

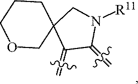

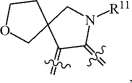

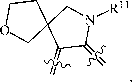

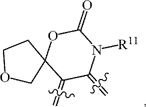

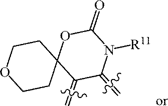

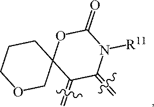

wherein, R¹¹ has the meaning as described in the present invention.

In some embodiments, R^{a} is H, D, F, Cl, Br, I, —NO₂, CN, —OH, —NH₂, C₁₋₆ alkyl, C₁₋₆ haloalkyl, C₂₋₆ alkenyl, C₂₋₆ alkynyl, C₁₋₆ alkoxy, C₃₋₈ cycloalkyl, heterocyclyl consisting of 3-8 atoms, C₆₋₁₀ aryl or heteroaryl consisting of 5-12 atoms, wherein, the —OH, —NH₂, C₁₋₆ alkyl, C₁₋₆ haloalkyl, C₂₋₆ alkene, C₂₋₆ alkynyl, C₁₋₆ alkoxy, C₃₋₈ cycloalkyl, heterocyclyl consisting of 3-8 atoms, C₆₋₁₀ aryl and heteroaryl consisting of 5-12 atoms can be independently and optionally unsubstituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R^{a} is H, D, F, Cl, Br, I, —NO₂, CN, —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl or heteroaryl consisting of 5-10 atoms, wherein, the —OH, —NH₂, C₁₋₄ alkyl, C₁₋₄ haloalkyl, C₂₋₄ alkene, C₂₋₄ alkynyl, C₁₋₄ alkoxy, C₃₋₆ cycloalkyl, heterocyclyl consisting of 3-6 atoms, C₆₋₁₀ aryl and heteroaryl consisting of 5-10 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R^{a} is H, D, F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl CHF₂, —CHCl₂, —CF₃, —CH₂CH₂F, —CH₂CH₂Cl, —CH₂CHF₂, —CH₂CHCl₂, —CHFCH₂F, —CHCICH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein, the —OH, —NH₂, methyl, ethyl, n-propyl, isopropyl, n-butyl, isobutyl, sec-butyl, tert-butyl, CH₂F, —CH₂Cl CHF₂, —CHCl₂, —CH₂CH₂F, —CH₂CH₂Cl, —CH₂CHF₂, —CH₂CHCl₂, —CHFCH₂F, —CHCICH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and pyridazinyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, —OCH₂OH and —OCH₂CH₂OH.

In some embodiments, R^{c} is H, D, F, Cl, Br, I, —NO₂, CN, —OH, —NH₂, C₁₋₆ alkyl, C₁₋₆ haloalkyl, C₂₋₆ alkenyl, C₂₋₆ alkynyl, C₁₋₆ alkoxy, C₃₋₈ cycloalkyl, heterocyclyl consisting of 3-8 atoms, C₆₋₁₀ aryl or heteroaryl consisting of 5-12 atoms, wherein, the —OH, —NH₂, C₁₋₆ alkyl, C₁₋₆ haloalkyl, C₂₋₆ alkene, C₂₋₆ alkynyl, C₁₋₆ alkoxy, C₃₋₈ cycloalkyl, heterocyclyl consisting of 3-8 atoms, C₆₋₁₀ aryl and heteroaryl consisting of 5-12 atoms can be independently and optionally unsubstituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R^{c} is H, D, F, Cl, Br, I, —NO₂, CN, —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl or heteroaryl consisting of 5-10 atoms, wherein, the —OH, —NH₂, C₁₋₄ alkyl, C₁₋₄ haloalkyl, C₂₋₄ alkene, C₂₋₄ alkynyl, C₁₋₄ alkoxy, C₃₋₆ cycloalkyl, heterocyclyl consisting of 3-6 atoms, C₆₋₁₀ aryl and heteroaryl consisting of 5-10 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R^{c} is H, D, F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl CHF₂, —CHCl₂, —CF₃, —CH₂CH₂F, —CH₂CH₂Cl, —CH₂CHF₂, —CH₂CHCl₂, —CHFCH₂F, —CHCICH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein, the —OH, —NH₂, methyl, ethyl, n-propyl, isopropyl, n-butyl, isobutyl, sec-butyl, tert-butyl, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHCICH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and pyridazinyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, —OCH₂OH and —OCH₂CH₂OH.

In some embodiments, R^{d} is H, D, F, Cl, Br, I, —NO₂, CN, —OH, —NH₂, C₁₋₆ alkyl, C₁₋₆ haloalkyl, C₂₋₆ alkenyl, C₂₋₆ alkynyl, C₁₋₆ alkoxy, C₃₋₈ cycloalkyl, heterocyclyl consisting of 3-8 atoms, C₆₋₁₀ aryl or heteroaryl consisting of 5-12 atoms, wherein, the —OH, —NH₂, C₁₋₆ alkyl, C₁₋₆ haloalkyl, C₂₋₆ alkene, C₂₋₆ alkynyl, C₁₋₆ alkoxy, C₃₋₈ cycloalkyl, heterocyclyl consisting of 3-8 atoms, C₆₋₁₀ aryl and heteroaryl consisting of 5-12 atoms can be independently and optionally unsubstituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R^{d} is H, D, F, Cl, Br, I, —NO₂, CN, —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl or heteroaryl consisting of 5-10 atoms, wherein, the —OH, —NH₂, C₁₋₄ alkyl, C₁₋₄ haloalkyl, C₂₋₄ alkene, C₂₋₄ alkynyl, C₁₋₄ alkoxy, C₃₋₆ cycloalkyl, heterocyclyl consisting of 3-6 atoms, C₆₋₁₀ aryl and heteroaryl consisting of 5-10 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R^{d} is H, D, F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl CHF₂, —CHCl₂, —CF₃, —CH₂CH₂F, —CH₂CH₂Cl, —CH₂CHF₂, —CH₂CHCl₂, —CHFCH₂F, —CHCICH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein, the —OH, —NH₂, methyl, ethyl, n-propyl, isopropyl, n-butyl, isobutyl, sec-butyl, tert-butyl, CH₂F, —CH₂Cl —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and pyridazinyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, —OCH₂OH and —OCH₂CH₂OH.

In some embodiments, R^{e} is H, D, F, Cl, Br, I, —NO₂, CN, —OH, —NH₂, C₁₋₆ alkyl, C₁₋₆ haloalkyl, C₂₋₆ alkenyl, C₂₋₆ alkynyl, C₁₋₆ alkoxy, C₃₋₈ cycloalkyl, heterocyclyl consisting of 3-8 atoms, C₆₋₁₀ aryl or heteroaryl consisting of 5-12 atoms, wherein, the —OH, —NH₂, C₁₋₆ alkyl, C₁₋₆ haloalkyl, C₂₋₆ alkene, C₂₋₆ alkynyl, C₁₋₆ alkoxy, C₃₋₈ cycloalkyl, heterocyclyl consisting of 3-8 atoms, C₆₋₁₀ aryl and heteroaryl consisting of 5-12 atoms can be independently and optionally unsubstituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R^{e} is H, D, F, Cl, Br, I, —NO₂, CN, —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl or heteroaryl consisting of 5-10 atoms, wherein, the —OH, —NH₂, C₁₋₄ alkyl, C₁₋₄ haloalkyl, C₂₋₄ alkene, C₂₋₄ alkynyl, C₁₋₄ alkoxy, C₃₋₆ cycloalkyl, heterocyclyl consisting of 3-6 atoms, C₆₋₁₀ aryl and heteroaryl consisting of 5-10 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R^{e} is H, D, F, Cl, Br, I, —NO₂, CN, —OH, —NH₂, methyl, ethyl, n-propyl, isopropyl, n-butyl, isobutyl, sec-butyl, tert-butyl, CH₂F, -CH₂Cl CHF₂, -CHCl₂, —CF₃, —CH₂CH₂F, —CH₂CH₂Cl, —CH₂CHF₂, —CH₂CHCl₂, —CHFCH₂F, —CHCICH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein, the —OH, —NH₂, methyl, ethyl, n-propyl, isopropyl, n-butyl, isobutyl, sec-butyl, tert-butyl, CH₂F, —CH₂Cl —CHF₂, —CHCl₂, —CH₂CH₂F, —CH₂CH₂Cl, —CH₂CHF₂, —CH₂CHCl₂, —CHFCH₂F, —CHCICH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-l-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and pyridazinyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, -OCH₂OH and -OCH₂CH₂OH.

In some embodiments, R^{b} is H, D, C₁₋₆ alkyl, C₁₋₆ haloalkyl, C₂₋₆ alkenyl, C₂₋₆ alkynyl, C₁₋₆ alkoxy, C₃₋₈ cycloalkyl, heterocyclyl consisting of 3-8 atoms, C₆₋₁₀ aryl or heteroaryl consisting of 5-12 atoms, wherein, the C₁₋₆ alkyl, C₁₋₆ haloalkyl, C₂₋₆ alkenyl, C₂₋₆ alkynyl, C₁₋₆ alkoxy, C₃₋₈ cycloalkyl, heterocyclyl consisting of 3-8 atoms, C₆₋₁₀ aryl and heteroaryl consisting of 5-12 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R^{b} is H, D, C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl or heteroaryl consisting of 5-10 atoms, wherein, the C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl and heteroaryl consisting of 5-10 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy.

In other embodiments, R^{b} is H, D, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein, the methyl, ethyl, n-propyl, isopropyl, n-butyl, isobutyl, sec-butyl, tert-butyl, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and pyridazinyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, -OCH₂OH and -OCH₂CH₂OH.

In some embodiments, R^{x} is F, Cl, Br, I, —NO₂, CN, —OH, —NH₂, C₁₋₆ alkyl, C₁₋₆ haloalkyl, C₂₋₆ alkenyl, C₂₋₆ alkynyl, C₁₋₆ alkoxy, C₁₋₆ alkylamino or C₃₋₆ cycloalkyl, wherein, the -OH, —NH₂, C₁₋₆ alkyl, C₁₋₆ haloalkyl, C₂₋₆ alkenyl, C₂₋₆ alkynyl, C₁₋₆ alkoxy, C₁₋₆ alkylamino and C₃₋₆ cycloalkyl can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ alkylamino, C₁₋₃ haloalkoxy and C₁₋₃ hydroxyalkoxy.

In other embodiments, R^{x} is F, Cl, Br, I, —NO₂, CN, —OH, —NH₂, C₁₋₄ alkyl, C₁₋₄ haloalkyl, C₂₋₄ alkenyl, C₂₋₄ alkynyl, C₁₋₄ alkoxy, C₁₋₄ alkylamino or C₃₋₆ cycloalkyl, wherein, the -OH, —NH₂, C₁₋₄ alkyl, C₁₋₄ haloalkyl, C₂₋₄ alkenyl, C₂₋₄ alkynyl, C₁₋₄ alkoxy, C₁₋₄ alkylamino and C₃₋₆ cycloalkyl can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ alkylamino, C₁₋₃ haloalkoxy and C₁₋₃ hydroxyalkoxy.

In other embodiments, R^{x} is F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, —N(CH₃)₂, cyclopropyl, cyclobutyl, cyclopentyl or cyclohexyl, wherein, the —OH, —NH₂, methyl, ethyl, n-propyl, isopropyl, n-butyl, isobutyl, sec-butyl, tert-butyl, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, —N(CH₃)₂, cyclopropyl, cyclobutyl, cyclopentyl and cyclohexyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, —N(CH₃)₂—, trifluoromethoxy, —OCH₂OH and —OCH₂CH₂OH.

In some embodiments, n is 0, 1 or 2.

In another aspect, provided herein is one of the following compounds or stereoisomers, geometric isomers, tautomers, N-oxides, solvates, hydrates, metabolites, esters, pharmaceutically acceptable salts or prodrugs thereof, but by no means limited to:

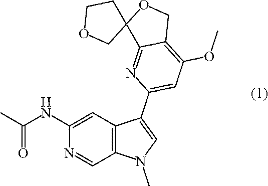 [SMILES: COc1cc(-c2cn(C)c3cnc(NC(C)=O)cc23)nc2c1COC21CCOC1]

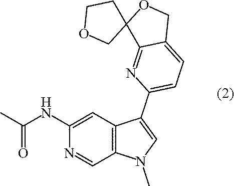 [SMILES: CC(=O)Nc1cc2c(-c3ccc4c(n3)C3(CCOC3)OC4)cn(C)c2cn1]

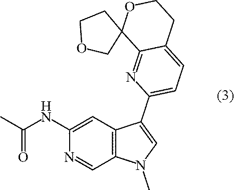 [SMILES: CC(=O)Nc1cc2c(-c3ccc4c(n3)C3(CCOC3)OCC4)cn(C)c2cn1]

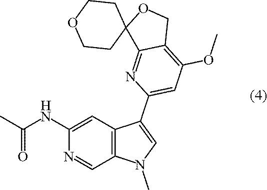 [SMILES: COc1cc(-c2cn(C)c3cnc(NC(C)=O)cc23)nc2c1COC21CCOCC1]

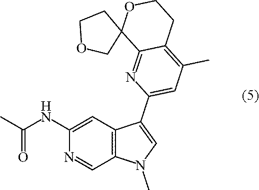 [SMILES: CC(=O)Nc1cc2c(-c3cc(C)c4c(n3)C3(CCOC3)OCC4)cn(C)c2cn1]

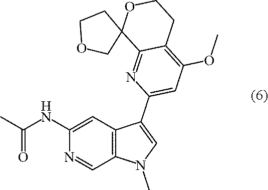 [SMILES: COc1cc(-c2cn(C)c3cnc(NC(C)=O)cc23)nc2c1CCOC21CCOC1]

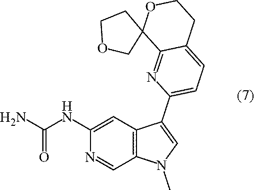 [SMILES: Cn1cc(-c2ccc3c(n2)C2(CCOC2)OCC3)c2cc(NC(N)=O)ncc21]

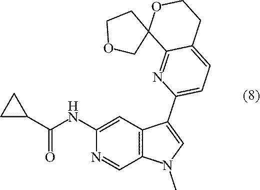 [SMILES: Cn1cc(-c2ccc3c(n2)C2(CCOC2)OCC3)c2cc(NC(=O)C3CC3)ncc21]

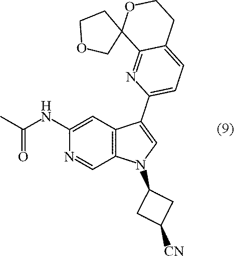 [SMILES: CC(=O)Nc1cc2c(-c3ccc4c(n3)C3(CCOC3)OCC4)cn([C@H]3C[C@@H](C#N)C3)c2cn1]

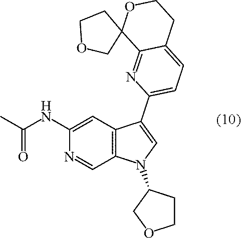 [SMILES: CC(=O)Nc1cc2c(-c3ccc4c(n3)C3(CCOC3)OCC4)cn([C@@H]3CCOC3)c2cn1]

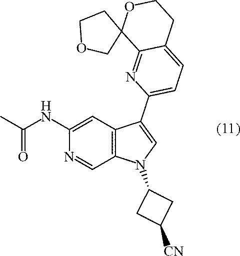 [SMILES: CC(=O)Nc1cc2c(-c3ccc4c(n3)C3(CCOC3)OCC4)cn([C@H]3C[C@H](C#N)C3)c2cn1]

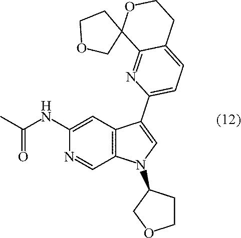 [SMILES: CC(=O)Nc1cc2c(-c3ccc4c(n3)C3(CCOC3)OCC4)cn([C@H]3CCOC3)c2cn1]

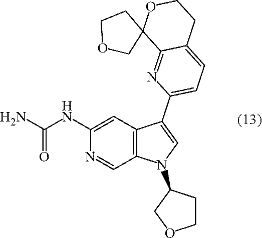 [SMILES: NC(=O)Nc1cc2c(-c3ccc4c(n3)C3(CCOC3)OCC4)cn([C@H]3CCOC3)c2cn1]

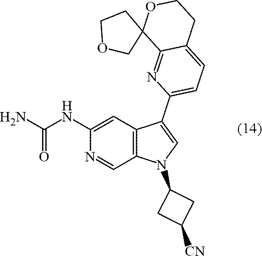 [SMILES: N#C[C@H]1C[C@@H](n2cc(-c3ccc4c(n3)C3(CCOC3)OCC4)c3cc(NC(N)=O)ncc32)C1]

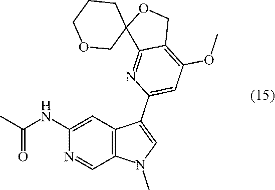 [SMILES: COc1cc(-c2cn(C)c3cnc(NC(C)=O)cc23)nc2c1COC21CCCOC1]

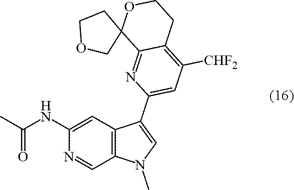 [SMILES: CC(=O)Nc1cc2c(-c3cc(C(F)F)c4c(n3)C3(CCOC3)OCC4)cn(C)c2cn1]

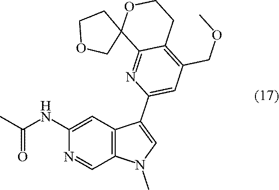 [SMILES: COCc1cc(-c2cn(C)c3cnc(NC(C)=O)cc23)nc2c1CCOC21CCOC1]

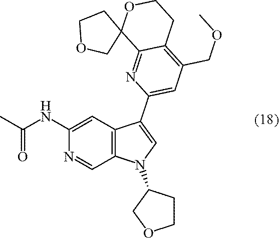 [SMILES: COCc1cc(-c2cn([C@@H]3CCOC3)c3cnc(NC(C)=O)cc23)nc2c1CCOC21CCOC1]

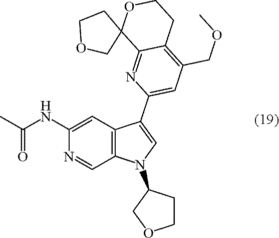 [SMILES: COCc1cc(-c2cn([C@H]3CCOC3)c3cnc(NC(C)=O)cc23)nc2c1CCOC21CCOC1]

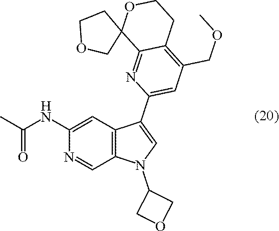 [SMILES: COCc1cc(-c2cn(C3COC3)c3cnc(NC(C)=O)cc23)nc2c1CCOC21CCOC1]

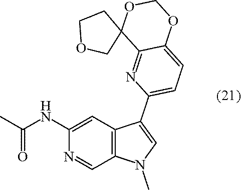 [SMILES: CC(=O)Nc1cc2c(-c3ccc4c(n3)C3(CCOC3)OCO4)cn(C)c2cn1]

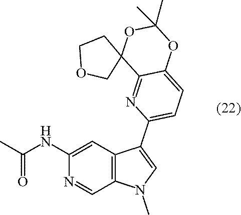 [SMILES: CC(=O)Nc1cc2c(-c3ccc4c(n3)C3(CCOC3)OC(C)(C)O4)cn(C)c2cn1]

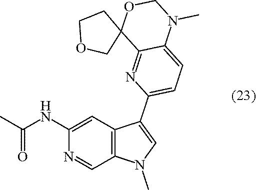 [SMILES: CC(=O)Nc1cc2c(-c3ccc4c(n3)C3(CCOC3)OCN4C)cn(C)c2cn1]

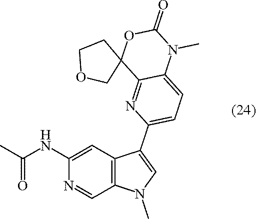 [SMILES: CC(=O)Nc1cc2c(-c3ccc4c(n3)C3(CCOC3)OC(=O)N4C)cn(C)c2cn1]

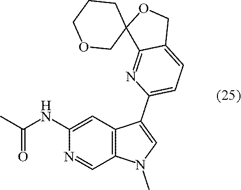 [SMILES: CC(=O)Nc1cc2c(-c3ccc4c(n3)C3(CCCOC3)OC4)cn(C)c2cn1]

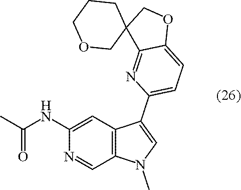 [SMILES: CC(=O)Nc1cc2c(-c3ccc4c(n3)C3(CCCOC3)CO4)cn(C)c2cn1]

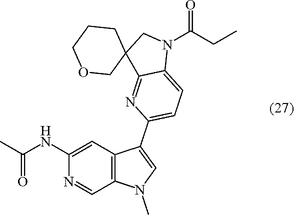 [SMILES: CCC(=O)N1CC2(CCCOC2)c2nc(-c3cn(C)c4cnc(NC(C)=O)cc34)ccc21]

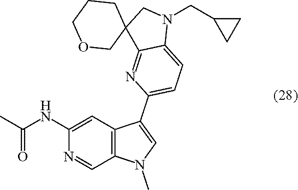 [SMILES: CC(=O)Nc1cc2c(-c3ccc4c(n3)C3(CCCOC3)CN4CC3CC3)cn(C)c2cn1]

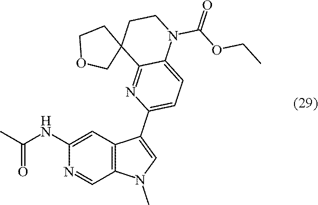 [SMILES: CCOC(=O)N1CCC2(CCOC2)c2nc(-c3cn(C)c4cnc(NC(C)=O)cc34)ccc21]

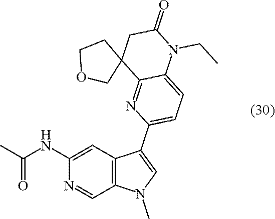 [SMILES: CCN1C(=O)CC2(CCOC2)c2nc(-c3cn(C)c4cnc(NC(C)=O)cc34)ccc21]

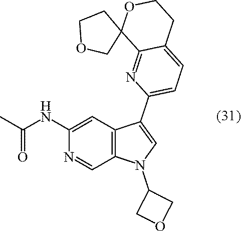 [SMILES: CC(=O)Nc1cc2c(-c3ccc4c(n3)C3(CCOC3)OCC4)cn(C3COC3)c2cn1]

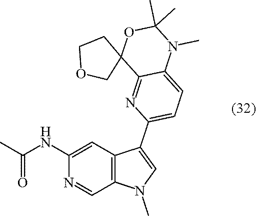 [SMILES: CC(=O)Nc1cc2c(-c3ccc4c(n3)C3(CCOC3)OC(C)(C)N4C)cn(C)c2cn1]

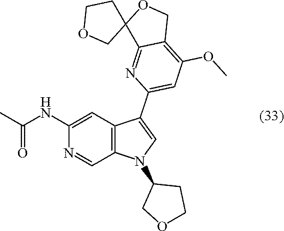 [SMILES: COc1cc(-c2cn([C@H]3CCOC3)c3cnc(NC(C)=O)cc23)nc2c1COC21CCOC1]

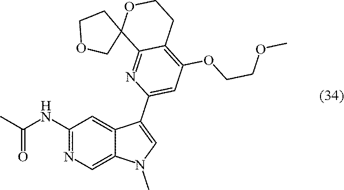 [SMILES: COCCOc1cc(-c2cn(C)c3cnc(NC(C)=O)cc23)nc2c1CCOC21CCOC1]

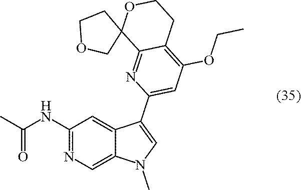 [SMILES: CCOc1cc(-c2cn(C)c3cnc(NC(C)=O)cc23)nc2c1CCOC21CCOC1]

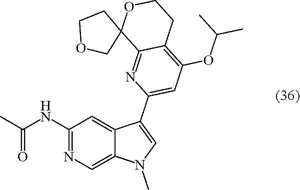 [SMILES: CC(=O)Nc1cc2c(-c3cc(OC(C)C)c4c(n3)C3(CCOC3)OCC4)cn(C)c2cn1]

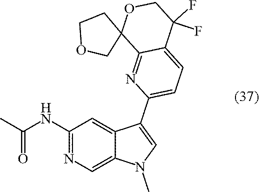 [SMILES: CC(=O)Nc1cc2c(-c3ccc4c(n3)C3(CCOC3)OCC4(F)F)cn(C)c2cn1]

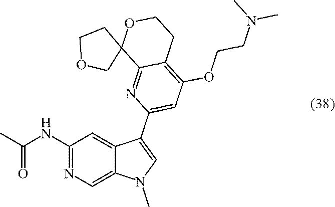 [SMILES: CC(=O)Nc1cc2c(-c3cc(OCCN(C)C)c4c(n3)C3(CCOC3)OCC4)cn(C)c2cn1]

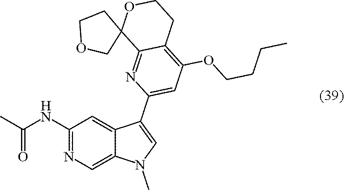 [SMILES: CCCCOc1cc(-c2cn(C)c3cnc(NC(C)=O)cc23)nc2c1CCOC21CCOC1]

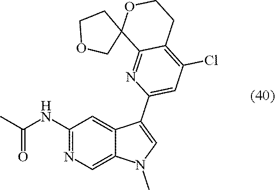 [SMILES: CC(=O)Nc1cc2c(-c3cc(Cl)c4c(n3)C3(CCOC3)OCC4)cn(C)c2cn1]

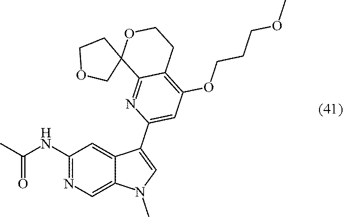 [SMILES: COCCCOc1cc(-c2cn(C)c3cnc(NC(C)=O)cc23)nc2c1CCOC21CCOC1]

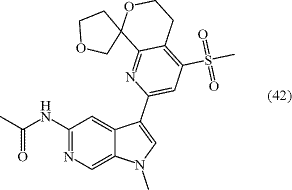 [SMILES: CC(=O)Nc1cc2c(-c3cc(S(C)(=O)=O)c4c(n3)C3(CCOC3)OCC4)cn(C)c2cn1]

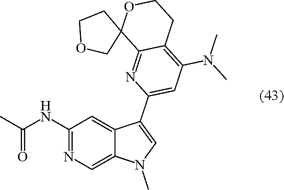 [SMILES: CC(=O)Nc1cc2c(-c3cc(N(C)C)c4c(n3)C3(CCOC3)OCC4)cn(C)c2cn1]

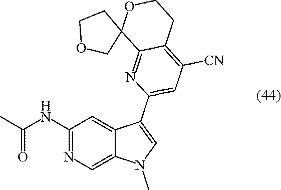 [SMILES: CC(=O)Nc1cc2c(-c3cc(C#N)c4c(n3)C3(CCOC3)OCC4)cn(C)c2cn1]

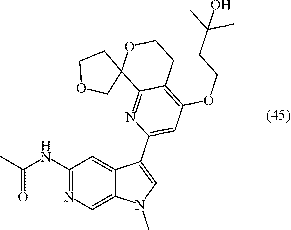 [SMILES: CC(=O)Nc1cc2c(-c3cc(OCCC(C)(C)O)c4c(n3)C3(CCOC3)OCC4)cn(C)c2cn1]

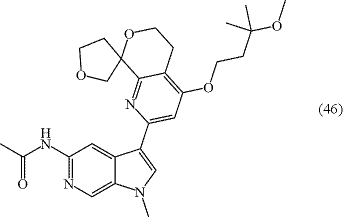 [SMILES: COC(C)(C)CCOc1cc(-c2cn(C)c3cnc(NC(C)=O)cc23)nc2c1CCOC21CCOC1]

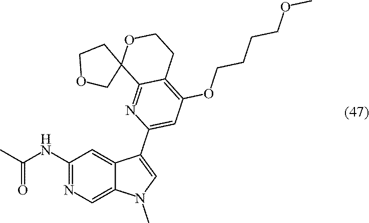 [SMILES: COCCCCOc1cc(-c2cn(C)c3cnc(NC(C)=O)cc23)nc2c1CCOC21CCOC1]

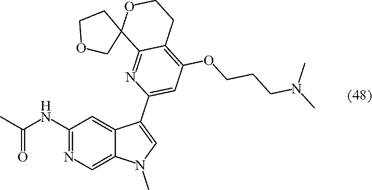 [SMILES: CC(=O)Nc1cc2c(-c3cc(OCCCN(C)C)c4c(n3)C3(CCOC3)OCC4)cn(C)c2cn1]

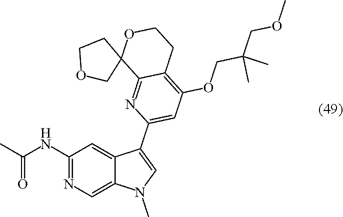 [SMILES: COCC(C)(C)COc1cc(-c2cn(C)c3cnc(NC(C)=O)cc23)nc2c1CCOC21CCOC1]

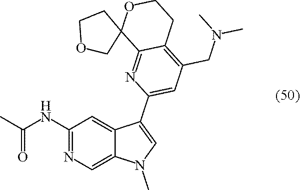 [SMILES: CC(=O)Nc1cc2c(-c3cc(CN(C)C)c4c(n3)C3(CCOC3)OCC4)cn(C)c2cn1]

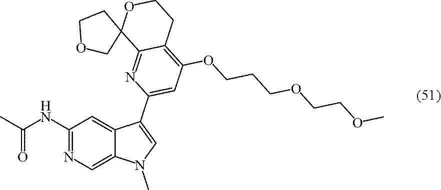 [SMILES: COCCOCCCOc1cc(-c2cn(C)c3cnc(NC(C)=O)cc23)nc2c1CCOC21CCOC1]

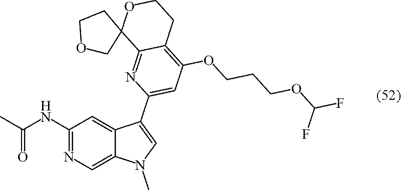 [SMILES: CC(=O)Nc1cc2c(-c3cc(OCCCOC(F)F)c4c(n3)C3(CCOC3)OCC4)cn(C)c2cn1]

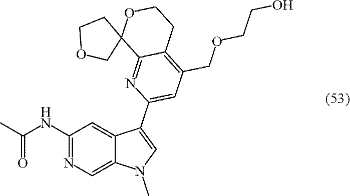 [SMILES: CC(=O)Nc1cc2c(-c3cc(COCCO)c4c(n3)C3(CCOC3)OCC4)cn(C)c2cn1]

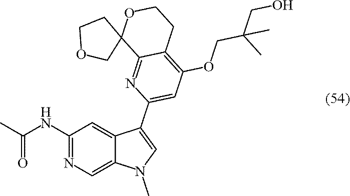 [SMILES: CC(=O)Nc1cc2c(-c3cc(OCC(C)(C)CO)c4c(n3)C3(CCOC3)OCC4)cn(C)c2cn1]

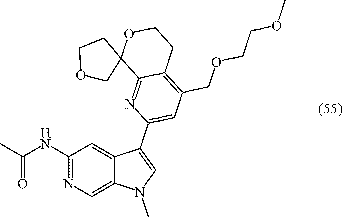 [SMILES: COCCOCc1cc(-c2cn(C)c3cnc(NC(C)=O)cc23)nc2c1CCOC21CCOC1]

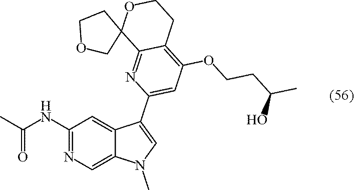 [SMILES: CC(=O)Nc1cc2c(-c3cc(OCC[C@@H](C)O)c4c(n3)C3(CCOC3)OCC4)cn(C)c2cn1]

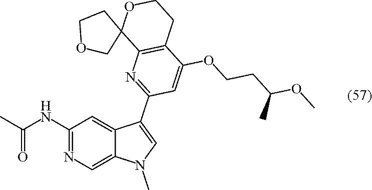 [SMILES: CO[C@@H](C)CCOc1cc(-c2cn(C)c3cnc(NC(C)=O)cc23)nc2c1CCOC21CCOC1]

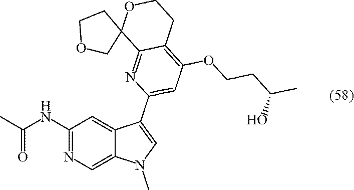 [SMILES: CC(=O)Nc1cc2c(-c3cc(OCC[C@H](C)O)c4c(n3)C3(CCOC3)OCC4)cn(C)c2cn1]

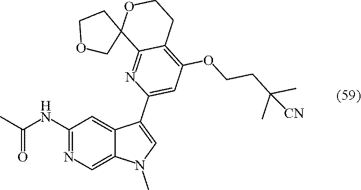 [SMILES: CC(=O)Nc1cc2c(-c3cc(OCCC(C)(C)C#N)c4c(n3)C3(CCOC3)OCC4)cn(C)c2cn1]

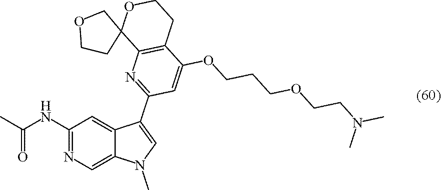 [SMILES: CC(=O)Nc1cc2c(-c3cc(OCCCOCCN(C)C)c4c(n3)C3(CCOC3)OCC4)cn(C)c2cn1]

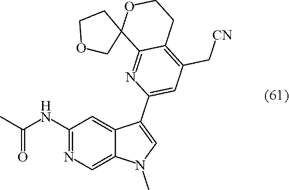 [SMILES: CC(=O)Nc1cc2c(-c3cc(CC#N)c4c(n3)C3(CCOC3)OCC4)cn(C)c2cn1]

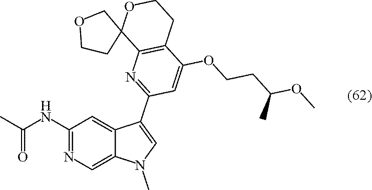 [SMILES: CO[C@@H](C)CCOc1cc(-c2cn(C)c3cnc(NC(C)=O)cc23)nc2c1CCOC21CCOC1]

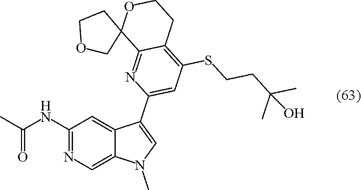 [SMILES: CC(=O)Nc1cc2c(-c3cc(SCCC(C)(C)O)c4c(n3)C3(CCOC3)OCC4)cn(C)c2cn1]

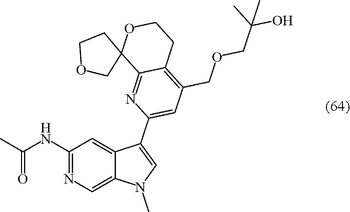 [SMILES: CC(=O)Nc1cc2c(-c3cc(COCC(C)(C)O)c4c(n3)C3(CCOC3)OCC4)cn(C)c2cn1]

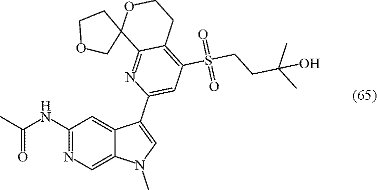 [SMILES: CC(=O)Nc1cc2c(-c3cc(S(=O)(=O)CCC(C)(C)O)c4c(n3)C3(CCOC3)OCC4)cn(C)c2cn1]

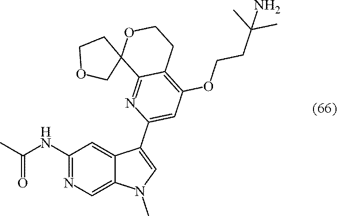 [SMILES: CC(=O)Nc1cc2c(-c3cc(OCCC(C)(C)N)c4c(n3)C3(CCOC3)OCC4)cn(C)c2cn1]

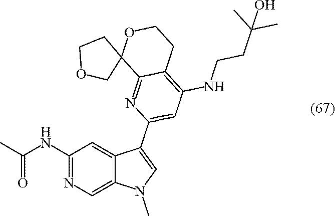 [SMILES: CC(=O)Nc1cc2c(-c3cc(NCCC(C)(C)O)c4c(n3)C3(CCOC3)OCC4)cn(C)c2cn1]

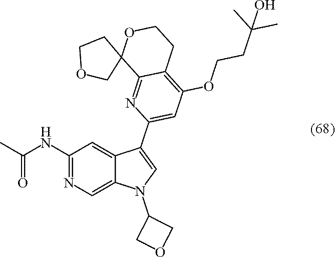 [SMILES: CC(=O)Nc1cc2c(-c3cc(OCCC(C)(C)O)c4c(n3)C3(CCOC3)OCC4)cn(C3COC3)c2cn1]

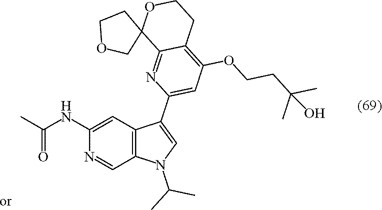

Unless otherwise specified, stereoisomers, geometric isomers, tautomers, N-oxides, hydrates, solvates, metabolites, pharmaceutically acceptable salts or prodrugs of the compounds having formula (I) or formula (II) are included within the scope of this invention.

The compounds disclosed herein may contain asymmetric or chiral centers and thus exist in different stereoisomeric forms. It is intended that all stereoisomeric forms of the compounds having formula (I) or formula (II) disclosed herein, including, but not limited to, diastereomers, enantiomers, atropisomers and geometric (or conformational) isomers, as well as mixtures thereof such as racemic mixtures, form part of the present invention.

In structures disclosed herein, when the stereochemistry of any particular chiral atom is not indicated, then all stereoisomers of the structure are contemplated and included in the invention as disclosed compounds. When stereochemistry is indicated by a solid wedge or a dashed line indicating a particular configuration, then the stereoisomers of that structure are identified and defined.

The compound having formula (I) or formula (II) can exist in different tautomer forms, and all these tautomers, such as the tautomers described herein, are all included within the scope of the present invention.

The compound having formula (I) or formula (II) may exist in the form of a salt. In some embodiments, the salt refers to a pharmaceutically acceptable salt. The term “pharmaceutically acceptable” means that the substance or composition must be chemically and/or toxicologically compatible with the other ingredients comprising the formulation and/or with the mammal being treated therewith. In other embodiments, the salt is not necessarily a pharmaceutically acceptable salt, but can be an intermediate for the preparation and/or purification of the compound having formula (I) or formula (II) and/or for separation of enantiomer of the compound having formula (I) or formula (II).

Pharmaceutically acceptable acid addition salts can be formed by reacting a compound having formula (I) or formula (II) with inorganic acids or organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, subsalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington’s Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Furthermore, the compounds disclosed herein, including their salts, can also be obtained in the form of their hydrates, or include other solvents such as ethanol, DMSO, and the like, used for their crystallization. The compounds of the present invention may inherently or by design form solvates with pharmaceutically acceptable solvents (including water); therefore, it is intended that the invention embrace both solvated and unsolvated forms.

Any formula given herein is also intended to represent isotopically unenriched forms as well as isotopically enriched forms of the compounds. Any formula given herein is also intended to represent isotopically unenriched forms as well as isotopically enriched forms of the compounds. Examples of isotopes that can be incorporated into compounds of the invention include isotopes of hydrogen, carbon, nitrogen, oxygen, phosphorous, fluorine, and chlorine, such as 2H (deuterium, D), 3H, 11C, 13C, 14C, 15N, 170, 180, 18F, 31P, 32P, 35S, 36C1, 125I, respectively.

In another aspect, the compounds of the invention include isotopically enriched compounds as defined herein, for example those into which radioactive isotopes, such as 3H, 14C and 18F, or those into which non-radioactive isotopes, such as 2H and 13C are present. Such isotopically enriched compounds are useful in metabolic studies (with 14C), reaction kinetic studies (with, for example 2H or 3H), detection or imaging techniques, such as positron emission tomography (PET) or single-photon emission computed tomography (SPECT) including drug or substrate tissue distribution assays, or in radioactive treatment of patients. In particular, an 18F-enriched compound may be particularly desirable for PET or SPECT studies. Isotopically-enriched compounds of Formula (I) or Formula (II) can generally be prepared by conventional techniques known to those skilled in the art or by processes analogous to those described in the accompanying Examples and Preparations using an appropriate isotopically-labeled reagent in place of the non-labeled reagent previously employed.

Further, substitution with heavier isotopes, particularly deuterium (i.e.,2H or D) may afford certain therapeutic advantages resulting from greater metabolic stability, for example increased in vivo half-life or reduced dosage requirements or an improvement in therapeutic index. for example, increased in vivo half-life or reduced dosage requirements or an improvement in therapeutic index. It is understood that deuterium in this context is regarded as a substituent of a compound of Formula (I) or (II). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). Pharmaceutically acceptable solvates in accordance with the invention include those wherein the solvent of crystallization may be isotopically substituted, e.g. D₂O, d₆-acetone, DMSO-d₆.

In other aspect, provided herein is an intermediate for preparing the compound having formula (I) or formula (II).

In another aspect, provided herein are methods for preparing, separating, and purifying the compounds having Formula (I) or (II).

In other aspect, provided herein is a pharmaceutical composition comprising the compound disclosed herein. In some embodiments, the pharmaceutical composition disclosed herein further comprises at least one of pharmaceutically acceptable adjuvants, excipients, carriers, and vehicles. In other embodiments, the pharmaceutical composition may be in the form of a liquid, solid, semi-solid, gel or spray.

In another aspect, provided herein is a method of treating a disease or disorder modulated by a JAK kinase comprising administering to a mammal an effective amount of the compound or pharmaceutical composition disclosed herein. In some embodiments, the disease or disorder is inflammatory disease, autoimmune disease, metabolic disease, destructive bone disease, proliferative disease, angiogenic disorder, sepsis, septic shock, shigellosis, neurodegenerative disease, or retinitis.

In another aspect, provided herein is the compound or pharmaceutical composition disclosed herein for use in treating a disease or disorder, the disease or disorder is inflammatory disease, autoimmune disease, metabolic disease, destructive bone disease, proliferative disease, angiogenic disorder, sepsis, septic shock, shigellosis, neurodegenerative disease, or retinitis.

In another aspect, provided herein is use of the compound or pharmaceutical composition disclosed herein in the manufacture of a medicament for treating a disease or disorder, the disease or disorder is inflammatory disease, autoimmune disease, metabolic disease, destructive bone disease, proliferative disease, angiogenic disorder, sepsis, septic shock, shigellosis, neurodegenerative disease, or retinitis.

In another aspect, provided herein is use of the compound or pharmaceutical composition disclosed herein in the manufacture of a medicament for inhibiting the activity of TYK2 kinase.

### Pharmaceutical Composition of the Compound of the Invention and Preparations and Administration

The present invention provides a pharmaceutical composition, which comprises the compound disclosed herein, or the compounds listed in the examples; and at least one of pharmaceutically acceptable adjuvants, excipients, carriers, and vehicles. The amount of the compound of the pharmaceutical composition disclosed herein refers to an amount which can be effectively detected to inhibit protein kinase of biology sample and patient.

It will also be appreciated that certain of the compounds of present invention can exist in free form for treatment, or where appropriate, as a pharmaceutically acceptable derivative or a prodrug thereof. According to the present invention, a pharmaceutically acceptable derivative or a prodrug includes, but is not limited to, pharmaceutically acceptable salts, esters, salts of such esters, or any other adduct or derivative which upon administration to a patient in need thereof is capable of providing, directly or indirectly, a compound as otherwise described herein, or a metabolite or residue thereof.

The pharmaceutical compositions of the invention may be prepared and packaged in bulk form wherein a safe and effective amount of a compound of Formula (I) or (II) disclosed herein can be extracted and then given to the patient, such as with powders or syrups. Alternatively, the pharmaceutical compositions of the invention may be prepared and packaged in unit dosage form wherein each physically discrete unit contains a safe and effective amount of a compound of Formula (I) or (II) disclosed herein.

“Pharmaceutically acceptable adjuvant” as used herein means a pharmaceutically acceptable material, composition or vehicle involved in giving form or consistency to the pharmaceutical composition. Each adjuvant must be compatible with the other ingredients of the pharmaceutical composition when commingled, such that interactions which would substantially reduce the efficacy of the compound of the invention when administered to a patient and would result in pharmaceutically unacceptable compositions are avoided. In addition, each adjuvant must of course be of sufficiently high purity to render it is pharmaceutically acceptable.

Suitable pharmaceutically acceptable adjuvant will vary depending upon the particular dosage form chosen. In addition, suitable pharmaceutically acceptable adjuvants may be chosen for a particular function that they may serve in the composition. For example, certain pharmaceutically acceptable adjuvants may be chosen for their ability to facilitate the production of uniform dosage forms. Certain pharmaceutically acceptable adjuvants may be chosen for their ability to facilitate the production of stable dosage forms. Certain pharmaceutically acceptable adjuvants may be chosen for their ability to facilitate the carrying or transporting the compound of the present invention once administered to the patient from one organ, or portion of the body, to another organ, or portion of the body. Certain pharmaceutically acceptable adjuvants may be chosen for their ability to enhance patient compliance.

Suitable pharmaceutically acceptable adjuvants include the following types of adjuvants: diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, humectants, chelating agents, plasticizers, viscosity increasing agents, antioxidants, preservatives, stabilizers, surfactants, and buffering agents. The skilled artisan will appreciate that certain pharmaceutically acceptable adjuvants may serve more than one function and may serve alternative functions depending on how much of the adjuvant is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington’s Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

In Remington: The Science and Practice of Pharmacy, 21st edition, 2005, ed. D.B. Troy, Lippincott Williams & Wilkins, Philadelphia, and Encyclopedia of Pharmaceutical Technology, eds. J. Swarbrick and J. C. Boylan, 1988-1999, Marcel Dekker, New York, the contents of each of which is incorporated by reference herein, are disclosed various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention.

The pharmaceutical compositions of the invention are prepared using techniques and methods known to those skilled in the art. Some of the methods commonly used in the art are described in Remington’s Pharmaceutical Sciences (Mack Publishing Company).

Therefore, another aspect of the present invention is related to a method for preparing a pharmaceutical composition, the pharmaceutical composition contains the compound disclosed herein and at least one of pharmaceutically acceptable adjuvant, excipient, carrier, vehicle, the method comprises mixing various ingredients. The pharmaceutical composition containing the compound disclosed herein can be prepared at for example environment temperature and under barometric pressure.

The compound of the invention will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, granules, and cachets; (2) parenteral administration such as sterile solutions, suspensions, and lyophilized powders; (3) transdermal administration such as transdermal patches; (4) rectal administration such as suppositories; (5) inhalation such as aerosols, solutions, and dry powders; and (6) topical administration such as creams, ointments, lotions, solutions, pastes, sprays, foams, and gels.

In some embodiments, the compounds disclosed herein can be prepared to oral. In other embodiments, the compounds disclosed herein can be prepared to inhalation. In other embodiments, the compounds disclosed herein can be prepared to nasal administration. In still other embodiments, the compounds disclosed herein can be prepared to transdermal administration. In still yet other embodiments, the compounds disclosed herein can be prepared to topical administration.

The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxy groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

Where appropriate, dosage unit formulations for oral administration can be microencapsulated. The formulation can also be prepared to prolong or sustain the release as for example by coating or embedding particulate material in polymers, wax, or the like.

The pharmaceutical compositions provided herein for oral administration may be also provided in the forms of liposomes, micelles, microspheres, or nanosystems. Miccellar dosage forms can be prepared as described in U.S. Pat. No. 6,350,458.

The pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The compounds disclosed herein can also be coupled to soluble polymers as targeted medicament carriers. Such polymers may encompass polyvinylpyrrolidone, pyran copolymer, polyhydroxypropylmethacrylamidophenol, polyhydroxyethylaspartamidophenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, polyorthoesters, polyacetals, polydihydroxypyrans, polycyanoacrylates and crosslinked or amphipathic block copolymers of hydrogels.

The pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action.

The pharmaceutical compositions provided herein may be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions provided herein may be formulated for single or multiple dosage administration. The single dosage formulations are packaged in an ampoule, a vial, or a syringe. The multiple dosage parenteral formulations must contain an antimicrobial agent at bacteriostatic or fungistatic concentrations. All parenteral formulations must be sterile, as known and practiced in the art.

In some embodiments, the pharmaceutical compositions are provided as ready-to-use sterile solutions. In other embodiments, the pharmaceutical compositions are provided as sterile dry soluble products, including lyophilized powders and hypodermic tablets, to be reconstituted with a vehicle prior to use. In yet other embodiments, the pharmaceutical compositions are provided as ready-to-use sterile suspensions. In yet other embodiments, the pharmaceutical compositions are provided as sterile dry insoluble products to be reconstituted with a vehicle prior to use. In still other embodiments, the pharmaceutical compositions are provided as ready-to-use sterile emulsions.

The pharmaceutical compositions provided herein may be formulated as immediate or modified release dosage forms, including delayed-, sustained, pulsed-, controlled, targeted-, and programmed-release forms.

The pharmaceutical compositions may be formulated as a suspension, solid, semi-solid, or thixotropic liquid, for administration as an implanted depot. In some embodiments, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

In another aspect, the pharmaceutical composition of the invention is prepared to a dosage form adapted for administration to a patient by inhalation, for example as a dry powder, an aerosol, a suspension, or a solution composition. In some embodiments, the invention is directed to a dosage form adapted for administration to a patient by inhalation as a dry powder. In other embodiments, the invention is directed to a dosage form adapted for administration to a patient by inhalation as a dry powder. Dry powder compositions for delivery to the lung by inhalation typically comprise a compound disclosed herein or a pharmaceutically acceptable salt thereof as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides. The finely divided powder may be prepared by, for example, micronisation and milling. Generally, the size-reduced (eg micronised) compound can be defined by a D₅₀ value of about 1 to about 10 microns (for example as measured using laser diffraction).

Aerosols may be formed by suspending or dissolving a compound disclosed herein or a pharmaceutically acceptable salt thereof in a liquified propellant. Suitable propellants include halocarbons, hydrocarbons, and other liquified gases. Representative propellants include: trichlorofluoromethane (propellant 11), dichlorofluoromethane (propellant 12), dichlorotetrafluoroethane (propellant 114), tetrafluoroethane (HFA-134a), 1,1-difluoroethane (HFA-152a), difluoromethane (HFA-32), pentafluoroethane (HFA-12), heptafluoropropane (HFA-227a), perfluoropropane, perfluorobutane, perfluoropentane, butane, isobutane, and pentane. Aerosols comprising a compound of formula (I) or a pharmaceutically acceptable salt thereof will typically be administered to a patient via a metered dose inhaler (MDI). Such devices are known to those skilled in the art.

The aerosol may contain additional pharmaceutically-acceptable excipients typically used with MDIs such as surfactants, lubricants, cosolvents and other excipients to improve the physical stability of the formulation, to improve valve performance, to improve solubility, or to improve taste.

Pharmaceutical compositions adapted for transdermal administration may be presented as discrete patches intended to remain in intimate contact with the epidermis of the patient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils. Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents, suspending agents or preservatives.

Topical preparations may be administered by one or more applications per day to the affected area; over skin areas occlusive dressings may advantageously be used. Continuous or prolonged delivery may be achieved by an adhesive reservoir system.

For treatments of the eye or other external tissues, for example mouth and skin, the compositions may be applied as a topical ointment or cream. When formulated in an ointment, the polymorph or salt of the invention may be employed with either a paraffinic or a water-miscible ointment base. Alternatively, the polymorph or salt of the invention may be formulated in a cream with an oil-in-water cream base or a water-in-oil base.

### Use of the Compounds and Pharmaceutical Compositions

The present invention provides that the compounds and pharmaceutical compositions disclosed herein can be used for treating, preventing or improving diseases or disorders mediated by TYK2 kinase or otherwise affected, especially for the manufacture of a medicament for treating, preventing or improving inflammatory diseases, autoimmune diseases, autologous diseases, metabolic diseases, destructive bone diseases, proliferative diseases, angiogenic disorders, sepsis, septic shock, shigellosis or neurodegenerative diseases or retinitis.

Specifically, the present invention provides a class of compounds disclosed herein or pharmaceutical compositions comprising the compounds for use in treating, preventing or improving diseases or disorders mediated by inappropriate TYK2 kinase activity or otherwise affected, the diseases or disorders are inflammatory diseases, autoimmune diseases, autologous diseases, metabolic diseases, destructive bone diseases, proliferative diseases, angiogenic disorders, sepsis, septic shock, shigellosis or neurodegenerative diseases or retinitis.

In some embodiments, such diseases or disorders include, but are not limited to: pancreatitis, asthma, allergies, adult respiratory distress syndrome, chronic obstructive pulmonary diseases, glomerulonephritis, rheumatoid arthritis, systemic lupus erythematosus, cutaneous lupus erythematosus, lupus nephritis, discoid lupus erythematosus, scleroderma, chronic thyroiditis, Graves disease, autoimmune gastritis, diabetes mellitus, autoimmune hemolytic anemia, autoimmune neutropenia thrombocytopenia, atopic dermatitis, chronic active hepatitis, myasthenia gravis, multiple sclerosis, inflammatory bowel disease, ulcerative colitis, Crohn’s disease, psoriasis, graft-versus-host disease, endotoxin induced inflammatory response, tuberculosis, atherosclerosis, muscle degeneration, cachexia, psoriatic arthritis, Knight syndrome, gout, traumatic arthritis, rheumatoid arthritis, acute synovitis, pancreatic β-cell disease, diseases characterized by massive neutrophil infiltration, rheumatoid spondylitis, gouty arthritis, cerebral malaria, chronic pulmonary inflammatory disease, silicosis, pulmonary sarcoid disease, bone resorption disease, concomitant allograft rejection, fever due to infection, myalgia due to infection, cachexia secondary to infection, keloid formation, scar tissue formation, fever, influenza, osteoporosis, osteoarthritis, acute bone marrow leukemia, chronic myelogenous leukemia, metastatic melanoma, Kaposi’s sarcoma, multiple myeloma, sepsis, septic shock, shigellosis, Alzheimer’s disease, Parkinson’s disease, cerebral ischemia or neurodegenerative disease caused by traumatic injury, angiogenic disorder, viral disease, CMV retinitis, AIDS, ARC, herpes, stroke, myocardial ischemia, ischemia in a stroke heart attack, organ hypoxia, vascular proliferation, cardiac reperfusion injury, renal reperfusion injury, thrombosis, cardiac hypertrophy, coagulation-induced enzyme platelet aggregation, endotoxemia, toxic shock syndrome, disease states associated with prostaglandin endoperoxidase synthase-2 or pemphigus vulgaris.

In other aspect, methods are provided herein for treating the diseases disclosed herein in a mammal suffering from (or at risk for), comprising administering to the mammal animal a therapeutically or prophylactically effective amount of one or more the pharmaceutical composition or compound disclosed herein.

In further method of treatment aspects, this invention provides methods of treating a mammal susceptible to or afflicted with a TYK2-mediated disease, comprising administering to the mammal a therapeutically or prophylactically effective amount of one or more the pharmaceutical composition or compound disclosed herein. In a particular embodiment, the TYK2-mediated disease is selected from inflammatory disease, autoimmune disease, autologous disease, metabolic disease, destructive bone disease, proliferative disease, angiogenic disorder, sepsis, septic shock, shigellosis, neurodegenerative disease, or retinitis.

In another aspect the present invention provides a compound of the invention or a composition containing the compound of the invention for use in the manufature of a medicine used in treatment or prevention of a TYK2-mediated disease. In a particular embodiment, the TYK2-mediated disease is selected from inflammatory disease, autoimmune disease, autologous disease, metabolic disease, destructive bone disease, proliferative disease, angiogenic disorder, sepsis, septic shock, shigellosis, neurodegenerative disease, or retinitis.

In another aspect, this invention provides methods of treating a mammal susceptible to or afflicted with an inflammatory, autoimmune or autologous disease, comprising administering to the mammal a therapeutically or prophylactically effective amount of one or more the pharmaceutical composition or compound disclosed herein. In a particular embodiment, the inflammatory disease is selected from, but not limited to Crohn’s disease, ulcerative colitis, asthma, graft-versus-host disease, allograft rejection, or chronic obstructive pulmonary disease; the autoimmune disease is selected from, but not limited to Graves disease, rheumatoid arthritis, systemic lupus erythematosus, cutaneous lupus, lupus nephritis, discoid lupus erythematosus, or psoriasis; autoinflammatory disease is selected from, but not limited to CAPS, TRAPS, FMF, adult Steele disease, systemic juvenile idiopathic arthritis, gout, or gouty arthritis.

### Therapeutic Methods

In some embodiments, the therapeutic methods disclosed herein comprise administrating to a patient in need of the treatment a safe and effective amount of the compound of the invention or the pharmaceutical composition containing the compound of the invention. Each example disclosed herein comprises the method of treating the above disorders or diseases comprising administrating to a patient in need of the treatment a safe and effective amount of the compound of the invention or the pharmaceutical composition containing the compound of the invention.

In some embodiments, the compound of the invention or the pharmaceutical composition thereof may be administered by any suitable route of administration, including both systemic administration and topical administration. Systemic administration includes oral administration, parenteral administration, transdermal administration and rectal administration. Parenteral administration refers to routes of administration other than enteral or transdermal, and is typically by injection or infusion. Parenteral administration includes intravenous, intramuscular, and subcutaneous injection or infusion. Topical administration includes application to the skin as well as intraocular, otic, intravaginal, inhaled and intranasal administration. In one embodiment, the compound of the invention or the pharmaceutical composition thereof may be administered orally. In other embodiments, the compound of the invention or the pharmaceutical composition thereof may be administered by inhalation. In a further embodiment, the compound of the invention or the pharmaceutical composition thereof may be administered intranasally.

In some embodiments, the compound of the invention or the pharmaceutical composition thereof may be administered once or according to a dosing regimen wherein a number of doses are administered at varying intervals of time for a given period of time. For example, doses may be administered one, two, three, or four times per day. In some embodiments, a dose is administered once per day. In still other embodiments, a dose is administered twice per day. Doses may be administered until the desired therapeutic effect is achieved or indefinitely to maintain the desired therapeutic effect. Suitable dosing regimens for the compound of the invention or the pharmaceutical composition thereof depend on the pharmacokinetic properties of that compound, such as absorption, distribution, and half-life, which can be determined by the skilled artisan. In addition, suitable dosing regimens, including the duration such regimens are administered, for the compound of the invention or the pharmaceutical composition thereof depend on the disorder being treated, the severity of the disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient’s response to the dosing regimen or over time as individual patient needs change.

The compounds of the present invention may be administered either simultaneously with, or before or after, one or more other therapeutic agents. The compounds of the present invention may be administered separately, by the same or different route of administration, or together in the same pharmaceutical composition as the other agents.

The pharmaceutical composition or combination of the present invention can be in unit dosage of about 1-1000 mg, or about 1-500 mg of active ingredients for a subject of about 50-70 kg. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., preferably aqueous solutions, and in vivo either enterally or parenterally, advantageously intravenously, e.g., as a suspension or in aqueous solution.

In some embodiments, a therapeutically effective dosage of the compound disclosed herein from about 0.1 mg to about 2,000 mg per day. The pharmaceutical compositions should provide a dosage of from about 0.1 mg to about 2000 mg of the compound. In a special embodiment, pharmaceutical dosage unit forms are prepared to provide from about 1 mg to about 2,000 mg, about 10 mg to about 1,000 mg of the active ingredient or a combination of essential ingredients per dosage unit form.

Additionally, the compounds of the invention may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of action of the compound in vivo; (b) modify the duration of action of the compound in vivo; (c) modify the transportation or distribution of the compound in vivo; (d) modify the solubility of the compound in vivo; and (e) overcome a side effect or other difficulty encountered with the compound. Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

### General Synthesis Steps

In order to describe the present invention, examples are listed below. However, it should be understood that the present invention is not limited to these examples, but only provides a method of practicing the present invention.

Generally, the compounds disclosed herein may be prepared by methods described herein, wherein the substituents are as defined for Formula (I) or Formula (II) above, except where further noted. The following non-limiting schemes and examples are presented to further exemplify the invention.

Persons skilled in the art will recognize that the chemical reactions described may be readily adapted to prepare a number of other compounds disclosed herein, and alternative methods for preparing the compounds disclosed herein are deemed to be within the scope disclosed herein. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds disclosed herein.

In the examples described below, unless otherwise indicated all temperatures are set forth in degrees Celsius. Reagents were purchased from commercial suppliers such as Aldrich Chemical Company, Arco Chemical Company and Alfa Chemical Company, and were used without further purification unless otherwise indicated. Common solvents were purchased from commercial suppliers such as Shantou XiLong Chemical Factory, Guangdong Guanghua Reagent Chemical Factory Co. Ltd., Guangzhou Reagent Chemical Factory, Tianjin YuYu Fine Chemical Ltd., Tianjin Fuchen Chemical Reagent Factory, Wuhan Xinhuayuan Technology Development Co., Ltd., Qingdao Tenglong Reagent Chemical Ltd., and Qingdao Ocean Chemical Factory.

Anhydrous THF, dioxane, toluene, and ether were obtained by refluxing the solvent with sodium. Anhydrous CH2Cl2 and CHCl3 were obtained by refluxing the solvent with CaH2. EtOAc, PE, hexane, DMAC and DMF were treated with anhydrous Na₂SO₄ prior to use.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography was conducted using a silica gel column. Silica gel (300-400 mesh) was purchased from Qingdao Ocean Chemical Factory.

1H NMR spectra were recorded using a Bruker 400 MHz or 600 MHz nuclear magnetic resonance spectrometer. ¹H NMR spectra were obtained by using CDCl₃, D₂O, DMSO-d₆, CD₃OD or acetone-d₆ solutions (reported in ppm), with TMS (0 ppm) or chloroform (7.26 ppm) as the reference standard. When peak multiplicities are reported, the following abbreviations are used: s (singlet), d (doublet), t (triplet), m (multiplet), br (broadened), dd (doublet of doublets), ddd (doublet of doublet of doublets), dddd (doublet of doublet of doublet of doublets), dt (doublet of triplets), tt (triplet of triplets). Coupling constants J, when given, were reported in Hertz (Hz).

The measurement conditions of low-resolution mass spectrometry (MS) data were: Agilent 6120 quadrupole HPLC-M (column model: Zorbax SB-C18, 2.1 × 30 mm, 3.5 µm, 6 min, flow rate: 0.6 mL/min. Mobile phase: 5% - 95% (CH₃CN with 0.1% formic acid) in (H₂O with 0.1% formic acid), using electrospray ionization (ESI) at 210 nm/254 nm with UV detection.

Pure compounds were detected using Agilent 1260 pre-HPLC or Calesep pump 250 pre-HPLC (column model: NOVASEP 50/80 mm DAC) with UV detection at 210 nm/254 nm.

The following abbreviations are used throughout the specification:

| | |
|---|---|
| h hour, hours | mL/ ml milliliter |
| DMF N,N-dimethylformamide | µL microlitre |
| CDC1₃ chloroform-d | MPa mega pascal |
| DMSO dimethylsulfoxide | NaCl sodium chloride |
| DMSO-d₆ dimethyl sulfoxide-d₆, | Pd₂(dba)₃ tris(dibenzylideneacetone)dipalladium |
| deuterated dimethyl sulfoxide | |
| THF tetrahydrofuran | Xantphos dimethylbisdiphenylphosphinoxanthene |
| H₂O water | NBS N-bromosuccinimide |
| g gram | LiHMDS lithium bis(trimethylsilyl)amide |
| mg milligram | NaHMDS sodium bis(trimethylsilyl)amide |
| M moles per liter | HCl hydrochloric acid |
| mM millimole per liter | hepes 4-Hydroxyethylpiperazine ethanesulfonic acid |
| mol mole | EDTA ethylenediaminetetraacetic acid |
| mmol millimole | |

PdCl₂dppf [1,1′-Bis(diphenylphosphino)ferrocene]palladium dichloride

The following reaction schemes describe the steps for preparing the compounds of the invention. Unless otherwise stated, each of X, V¹, V², V³, V⁴, R¹, R⁶, R⁷ and R^{b} has the definition as described in the present invention. -Lg represents a leaving group, such as -I, -Br, -OMs or -OTs.

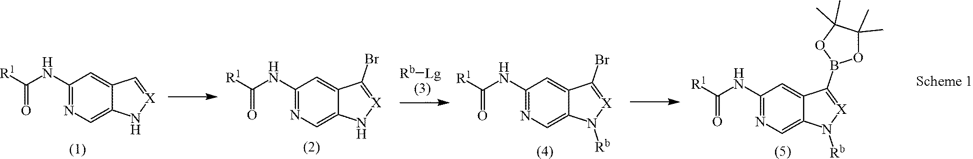

The compound shown in formula (5) can be prepared by this reaction scheme 1: the compound shown in formula (1) reacts under the effect of NBS to obtain the compound shown in formula (2). The compound shown in formula (2) and the compound shown in formula (3) react under the effect of sodium hydride to obtain the compound shown in formula (4). The compound shown in formula (4) reacts under the effect of bis(pinacolato) diboron and potassium acetate to obtain the compound shown in formula (5).

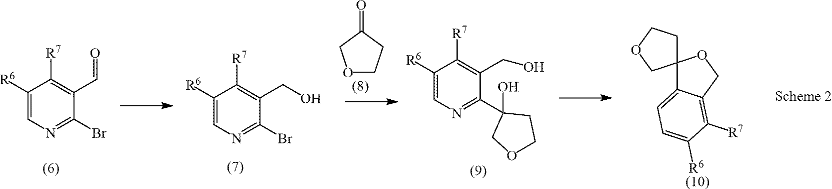

The compound shown in formula (10) can be prepared by the reaction scheme 2: the compound shown in formula (6) reacts under the effect of sodium borohydride to obtain the compound shown in formula (7). The compound shown in formula (7) and the compound shown in formula (8) react under the effect of n-butyllithium to obtain the compound shown in formula (9). The compound shown in formula (9) reacts under the effect of sodium bis(trimethylsilyl)amide and toluenesulfonyl chloride to obtain the compound shown in formula (10).

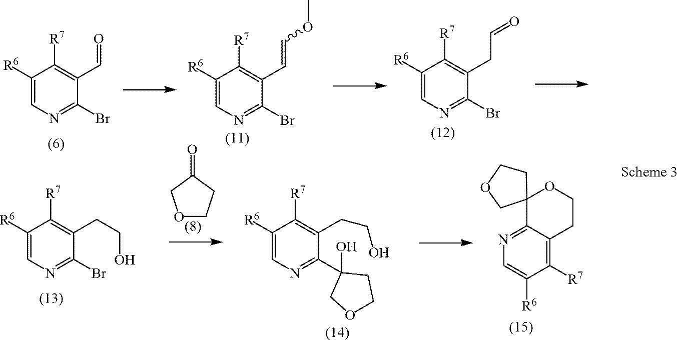

The compound shown in formula (15) can be prepared by this reaction scheme 3: the compound shown in formula (6) reacts under the effect of LiHMDS and chloride methoxymethyltriphenylphosphonium salt to obtain the compound shown in formula (11). The compound shown in formula (11) reacts with formic acid to obtain the compound shown in formula (12). The compound shown in formula (12) reacts under the effect of sodium borohydride to obtain the compound shown in formula (13). The compound shown in formula (13) and the compound shown in formula (8) react under the effect of n-butyllithium to obtain the compound shown in formula (14). The compound shown in formula (14) reacts under the effect of NaHMDS and p-toluenesulfonyl chloride to obtain the compound shown in formula (15).

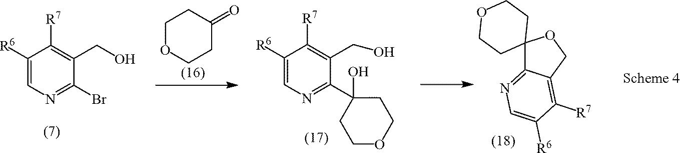

The compound shown in formula (18) can be prepared by this reaction scheme 4: the compound shown in formula (7) and the compound shown in formula (16) react under the effect of n-butyllithium to obtain the compound shown in formula (17). The compound shown in formula (17) reacts under the effect of sodium bis(trimethylsilyl)amide and p-toluenesulfonyl chloride to obtain the compound shown in formula (18).

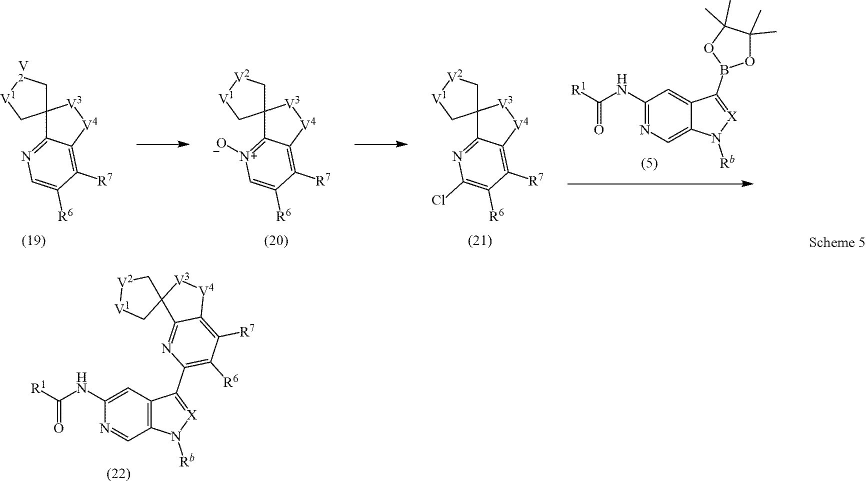

The compound shown in formula (22) can be prepared by the reaction scheme 5: the compound shown in formula (19) reacts under the effect of m-chloroperoxybenzoic acid to obtain the compound shown in formula (20). The compound shown in formula (20) reacts under the effect of phosphorus oxychloride to obtain the compound shown in formula (21). The compound shown in formula (21) and the compound shown in formula (5) react under the effect of Pd(dppf)Cl₂ and sodium carbonate to obtain the compound shown in formula (22).

The compounds, pharmaceutical compositions and uses thereof provided by the present invention will be further described below in conjunction with the examples.

### EXAMPLE

### Example 1 N-(3-(4′-methoxy-4,5-dihydro-2H,5′H-spiro[furan-3,7′-furo[3,4-b]pyridin]-2′ -yl)-1-methyl-1H-pyrrolo[2,3-c]pyridin-5-yl)acetamide

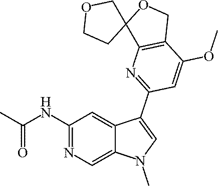 [SMILES: COc1cc(-c2cn(C)c3cnc(NC(C)=O)cc23)nc2c1COC21CCOC1]

### Step 1 Synthesis of N-(3-Bromo-1H-Pyrrolo[2,3-c]Pyridin-5-yl)Acetamide

N-(1H-pyrrolo[2,3-c]pyridin-5-yl)acetamide (3.50 g, 20.0 mmol) and DMF (20 mL) were added into a reaction flask, sonicated and stirred until the solution was clear, then NBS (3.81 g, 21.0 mmol) was added, the mixture was stirred at room temperature for 2 h, then the reaction was quenched by saturated sodium sulfite solution (2 mL), filtered and washed with methanol, the filtrates were combined and concentrated under reduced pressure. The obtained residue was purified by column chromatography (dichloromethane/methanol (v/v) = 17/3) to obtain the title compound as a yellow solid (4.75 g, yield 93.6%).

¹H NMR (400 MHz, DMSO-d₆) δ (ppm) 11.86 (s, 1H), 10.29 (s, 1H), 8.50 (s, 1H), 8.13 (s, 1H), 7.78 (d, J= 2.6 Hz, 1H), 2.09 (s, 3H).

### Step 2 Synthesis of N-(3-Bromo-1-Methyl-1H-Pyrrolo[2,3-c]Pyridin-5-yl)Acetamide

Under N₂, N-(3-bromo-1H-pyrrolo[2,3-c]pyridin-5-yl)acetamide (1.90 g, 7.48 mmol) and DMF (15 mL) were added into a reaction flask, the mixture was stirred to dissolve, then cooled to 0° C., sodium hydride (0.36 g, 9.00 mmol) was added and stirred for 15 min, then iodomethane (0.52 mL, 8.23 mmol) was added, and the mixture was warmed to room temperature and stirred and reacted for 2 h. The reaction was quenched by methanol (5 mL), and the solution was concentrated under reduced pressure, and the resulting residue was purified by column chromatography (dichloromethane/methanol (v/v) = 97:3) to obtain the title compound as a yellow-white flaky solid (1.12 g, yield 55.9%).

MS (ESI, pos. ion) m/z: 268.0 [M+H]⁺;

¹H NMR (400 MHz, DMSO-d₆) δ (ppm) 10.34 (s, 1H), 8.62 (s, 1H), 8.12 (s, 1H), 7.75 (s, 1H), 3.87 (s, 3H), 2.09 (s, 3H).

### Step 3 Synthesis of N-(1-Methyl-3-(4,4,5,5-Tetramethyl-1,3,2-Dioxaborolan-2-yl)-1H-Pyr rolo[2,3-c]Pyridin-5-yl)Acetamide

N-(3-bromo-1-methyl-1H-pyrrolo[2,3-c]pyridin-5-yl)acetamide (0.10 g, 0.37 mmol), molecular sieves (0.10 g), bis(pinacolato)diborane (0.29 g, 1.12 mmol) and potassium acetate (0.13 g, 1.30 mmol) were added into a reaction flask, under N₂, 1,4-dioxane (3 mL) was added, the mixture was bubbled with N₂ for 10 min, then Xantphos (18.1 mg, 0.03721 mmol) and Pd₂(dba)₃ (17.0 mg, 0.0180 mmol) were added to the flask. The reaction solution was stirred and reacted at 80° C. overnight under N₂, then methanol (5 mL) was added to dilute, the mixture was filtered through a celite pad, washed with a small amount of methanol, the filtrates were combined and concentrated under reduced pressure. The resulting residue was purified by column chromatography (dichloromethane/methanol (v/v) = 24/1) to obtain the title compound as a viscous yellow semi-solid (44 mg, yield 37.0%).

MS (ESI, pos. ion) m/z: 316.2 [M+H]⁺;

¹H NMR (400 MHz, DMSO-d₆) δ (ppm) 10.15 (s, 1H), 8.55 (s, 1H), 8.37 (s, 1H), 7.83 (s, 1H), 3.86 (s, 3H), 2.07 (s, 3H), 1.29 (s, 9H).

### Step 4 Synthesis of (2-Bromopyridin-3-yl)Methanol

2-Bromo-3-formylpyridine (5.58 g, 30.0 mmol) and anhydrous methanol (60 mL) were added into a reaction flask, then stirred to dissolve, under N₂, the mixture was cooled to 0° C., and sodium borohydride (1.38 g, 35.7 mmol) was added in two batches, the mixture was warmed to room temperature and stirred for 0.5 h. Saturated ammonium chloride (50 mL) was added to quench the reaction, and methanol was concentrated under reduced pressure. The resulting solution was extracted with ethyl acetate (50 mL×3), and the organic phases were combined, washed with saturated sodium chloride (50 mL), then the organic phases were collected and dried over anhydrous sodium sulfate, filtered, the filtrate was concentrated under reduced pressure, and then dried at 50° C. under vacuum to obtain the title compound as a white solid (5.47 g, yield 97.0%).

MS (ESI, pos. ion) m/z: 188.0 [M+H]⁺;

¹H NMR (400 MHz, CDCl₃) δ (ppm) 8.35 - 8.23 (m, 1H), 7.91 - 7.80 (m, 1H), 7.38 - 7.30 (m, 1H), 4.77 (d, J = 5.9 Hz, 2H), 2.38 (t, J = 6.0 Hz, 1H).

### Step 5 Synthesis of 3-(3-(hyDroxymethyl)Pyridin-2-yl)Tetrahydrofuran-3-Ol

(2-Bromopyridin-3-yl)-methanol (1.13 g, 6.01 mmol) and anhydrous toluene (10 mL) were added into a reaction flask, the mixture was concentrated under reduced pressure, under N₂, anhydrous THF (30 mL) was added and the mixture was cooled to -78° C. To the flask was added n-butyllithium (2.5 mol/L n-hexane solution, 4.9 mL, 12 mmol) dropwise and the mixture was stirred at -78° C. for 1 h, then tetrahydrofuran-3-one (0.62 g, 7.2 mmol) was added and stirred for 2 h, then the solution was warmed to room temperature and continued to stir for 30 min. The reaction was quenched by saturated ammonium chloride (30 mL), extracted with ethyl acetate (30 mL×3), the organic phases were combined, and washed with saturated sodium chloride (30 mL), then the organic phase was collected and dried over anhydrous sodium sulfate and filtered, the filtrate was concentrated under reduced pressure. The residue was purified by column chromatography (petroleum ether/ethyl acetate (v/v) = ¼) to obtain the title compound as viscous colorless oily liquid (0.21 g, yield 17.9%).

MS (ESI, pos. ion) m/z: 196.1 [M+H]⁺;

¹H NMR (400 MHz, CDCl₃) δ (ppm) 8.54 - 8.40 (m, 1H), 8.53 - 8.40 (m, 1H), 7.86 - 7.73 (m, 1H), 7.32 - 7.22 (m, 1H), 4.89 - 4.70 (m, 2H), 4.33 - 4.24 (m, 1H), 4.19 - 4.11 (m, 2H), 4.07 - 3.88 (m, 2H), 2.68 - 2.54 (m, 1H), 2.37 - 2.21 (m, 1H).

### Step 6 Synthesis of 4,5-Dihydro-2H,5′H- Spiro[Furan-3,7′-Furo[3,4-b]Pyridine]

3-(3-(Hydroxymethyl)pyridin-2-yl)tetrahydrofuran-3-ol (154.0 mg, 0.79 mmol) and anhydrous toluene (5 mL) were added into a reaction flask, the mixture was concentrated under reduced pressure, under N₂, anhydrous THF (8 mL) was added and the mixture was cooled to 0° C. To the flask was added sodium bis(trimethylsilyl)amide (2 mol/L THF solution, 0.87 mL, 1.7 mmol) dropwise, and the mixture was stirred at 0° C. for 10 min, then p-toluenesulfonyl chloride (184.0 mg, 0.95 mmol) was added and the mixture was continuously stirred for 1 h. The reaction was quenched by saturated ammonium chloride (10 mL), extracted with ethyl acetate (10 mL×3), the organic phases were combined, washed with saturated sodium chloride (10 mL) and filtered, the filtrate was concentrated under reduced pressure. The resulting residue was purified by column chromatography (dichloromethane/methanol (v/v) = 19/1) to obtain the title compound as yellow oil liquid (108.0 mg, yield 77.3%).

MS (ESI, pos. ion) m/z: 178.1 [M+H]⁺;

### Step 7 Synthesis of 4,5-Dihydro-2H,5′H-Spiro[Furan-3,7′-Furo[3,4-b]Pyridine]1′-Oxide

4,5-Dihydro-2H,5′H-spiro[furan-3,7′-furo[3,4-b]pyridine] (80.0 mg, 0.45 mmol), dichloromethane (5 mL) and m-chloroperoxybenzoic acid (137.0 mg, 0.67 mmol) were added into a flask, the mixture was stirred at room temperature for 4 h, then the reaction was quenched by saturated sodium sulfite (5 mL), and saturated sodium carbonate (5 mL) was added, the mixture was extracted with chloroform (10 mL× 3), the organic phases were combined, dried over anhydrous sodium sulfate and filtered, the filtrate was concentrated under reduced pressure. The resulting residue was purified by column chromatography (dichloromethane/methanol (v/v) = 19/1) to obtain the title compound as a white solid (50.0 mg, yield 57.3%).

MS (ESI, pos. ion) m/z: 194.1 [M+H]⁺;

¹H NMR (400 MHz, CDCl₃) δ (ppm) 8.10 (d, J = 6.4 Hz, 1H), 7.25 - 7.20 (m, 1H), 7.13 (d, J = 7.6 Hz, 1H), 5.09 (s, 2H), 4.39 (d, J = 9.3 Hz, 1H), 4.19 - 4.09 (m, 2H), 3.87 (d, J = 9.3 Hz, 1H), 3.09 - 2.95 (m, 1H), 2.15 - 2.00 (m, 1H).

### Step 8 Synthesis of 4′-Chloro-4,5-Dihydro-2H,5′H- Spiro[Furo-3,7′-Furo[3,4-b]Pyridine]

4,5-Dihydro-2H,5′H-spiro[furo-3,7′-furo[3,4-b]pyridine]1′-oxide (294.0 mg, 1.52 mmol) and phosphorus oxychloride (5 mL, 53.6 mmol) were added into a reaction flask, the mixture was heated to 110° C. and stirred for 5 h, the reaction solution was concentrated under reduced pressure, then saturated sodium carbonate (5 mL) and water (5 mL) were added to the flask, the mixture was extracted with ethyl acetate (10 mL ×3), the organic phases were combined, washed with saturated sodium chloride (10 mL), dried over anhydrous sodium sulfate and filtered, the filtrate was concentrated under reduced pressure. The resulting residue was purified by column chromatography (dichloromethane/methanol (v/v) = 49/1) to obtain the title compound as a yellow-white solid (180.0 mg, yield 56.0%).

MS (ESI, pos. ion) m/z: 194.1 [M+H]⁺;

¹H NMR (400 MHz, CDCl₃) δ (ppm) 8.43 (d, J = 5.4 Hz, 1H), 7.20 (d, J = 5.4 Hz, 1H), 5.12 (s, 2H), 4.13 (dd, J = 8.6, 5.5 Hz, 2H), 4.01 (q, J = 9.9 Hz, 2H), 2.49 - 2.36 (m, 1H), 2.35 - 2.23 (m, 1H).

### Step 9 Synthesis of 4′-Methoxy-4,5-Dihydro-2H,5′H-Spiro[Furan-3,7′-Furo[3,4-b]Pyridin e]

4′-Chloro-4,5-dihydro-2H,5′H-spiro[furo-3,7′-furo[3,4-b]pyridine] (20.0 mg, 0.0945 mmol) and a solution of sodium methoxide in methanol (2 mL, 11 mmol, 5.4 mol/L) were added to a reaction flask, the mixture was heated to 80° C. and stirred for 4 h, the reaction was quenched by saturated ammonium chloride (5 mL), then concentrated hydrochloric acid was added to adjust the pH to weak base. After extraction with ethyl acetate (10 mL × 3), the combined organic phases were washed with saturated sodium chloride (10 mL), and the organic phase was concentrated under reduced pressure to obtain the title compound as a white solid (19.0 mg, yield 97.0%).

MS (ESI, pos. ion) m/z: 208.1 [M+H]⁺;

¹H NMR (400 MHz, CDCl₃) δ (ppm) 8.43 (d, J = 5.7 Hz, 1H), 6.69 (d, J = 5.7 Hz, 1H), 5.06 (s, 2H), 4.12 (dd, J = 8.7, 5.5 Hz, 2H), 4.02 - 3.94 (m, 2H), 3.89 (s, 3H), 2.51 - 2.34 (m, 1H), 2.31 - 2.17 (m, 1H).

### Step 10 Synthesis of 4′-Methoxy-4,5-Dihydro-2H,5′H-Spiro[Furo-3,7′-Furo[3,4-b]Pyridine] 1′-Oxide

4′-Methoxy-4,5-dihydro-2H,5′H-spiro[furo-3,7′-furo[3,4-b]pyridine] (158.0 mg, 0.76 mmol), anhydrous dichloromethane (5 mL) and m-chloroperoxybenzoic acid (232.0 mg, 1.14 mmol) were added to a reaction flask, the mixture was stirred at room temperature overnight, the reaction was quenched by saturated sodium sulfite (5 mL) and stirred for 15 min, then saturated sodium carbonate (5 mL) was added. After extraction with chloroform (10 mL×3), the combined organic phases were dried over anhydrous sodium sulfate, filtered, and the filtrate was concentrated under reduced pressure, the obtained residue was purified by column chromatography (dichloromethane/methanol (v/v) = 19/1) to obtain the title compound as a white solid (124.0 mg, yield 72.9%).

MS (ESI, pos. ion) m/z: 224.1 [M+H]⁺;

¹H NMR (400 MHz, CDCl₃) δ (ppm) 8.07 (d, J = 7.0 Hz, 1H), 6.72 (d, J = 7.0 Hz, 1H), 5.09 - 4.99 (m, 2H), 4.43 (d, J = 9.2 Hz, 1H), 4.19 - 4.04 (m, 2H), 3.89 (s, 3H), 3.86 (d, J = 9.3 Hz, 1H), 3.12 - 2.97 (m, 1H), 2.13 - 2.00 (m, 1H).

### Step 11 Synthesis of 2′-Chloro-4′-Methoxy-4,5-Dihydro-2H,5′H-Spiro[Furan-3,7′-Furo[3,4 -b]Pyridine]

4′-Methoxy-4,5-dihydro-2H,5′H-spiro[furo-3,7′-furo[3,4-b]pyridine] 1′-oxide (90.0 mg, 0.40 mmol) and phosphorus oxychloride (6 mL, 64.37 mmol) were added to a reaction flask, the mixture was heated to 110° C. and stirred for 10 h, then concentrated in vacuum, and saturated sodium carbonate (5 mL) and water (5 mL) were added. After extraction with ethyl acetate (10 mL×3), the combined organic phase was washed with saturated sodium chloride (10 mL), then dried and filtered, the filtrate was concentrated under reduced pressure. The obtained residue was purified by column chromatography (dichloromethane/methanol (v/v) = 19/1) to obtain the title compound as a yellow-white solid (35.0 mg, yield 36.0%).

MS (ESI, pos. ion) m/z: 242.1 [M+H]⁺;

¹H NMR (400 MHz, CDCl₃) δ (ppm) 6.72 (s, 1H), 5.01 (s, 2H), 4.10 (dd, J = 8.8, 5.4 Hz, 2H), 4.02 - 3.93 (m, 2H), 3.90 (s, 3H), 2.49 - 2.36 (m, 1H), 2.28 - 2.17 (m, 1H).

### Step 12 Synthesis of N-(3-(4′-Methoxy-4,5-Dihydro-2H,5′H-Spiro[Furan-3,7′-Furo[3,4-b] Pyridin]-2′-yl)-1-Methyl-1H-Pyrrolo[2,3-c]Pyridin-5-yl)Acetamide

Under N₂, 2′-chloro-4′-methoxy-4,5-dihydro-2H,5′H-spiro[furan-3,7′-furo[3,4-b]pyridine] (65.0 mg, 0.27 mmol), N-(1-methyl-3-(4,4,5,5-tetramethyl-1,3,2-dioxaborolan-2-yl)-1H-pyrrolo[2,3-c]pyridin-5-yl)acetami de (84.8 mg, 0.27 mmol) and 1,4-dioxane (4 mL) were added to a reaction flask, the mixture was stirred and dissolved, then water (1 mL) and sodium carbonate (85.0 mg, 0.80 mmol) were added, the mixture was deoxygenated by bubbling N₂ for 10 min, then PdCl₂dppf (25.0 mg, 0.0341 mmol) was added. The solution was stirred at 80° C. overnight under N₂ and cooled to room temperature, the reaction was quenched by water (10 mL), after extraction with chloroform (10 mL×3), the combined organic phase was dried over anhydrous sodium sulfate, and concentrated under reduced pressure, and the resulting residue was purified by column chromatography (ethyl acetate/methanol (v/v) = 15/1) to obtain the title compound as a yellow solid (30 mg, yield 28.3%).

MS (ESI, pos. ion) m/z: 395.2[M+H]⁺;

¹H NMR (400 MHz, CDCl₃) δ (ppm) 8.98 (s, 1H), 8.42 (s, 1H), 8.24 (s, 1H), 7.92 (s, 1H), 7.16 (s, 1H), 5.09 (s, 2H), 4.03 (s, 3H), 4.27 - 4.01 (m, 4H), 3.93 (s, 3H), 2.61 - 2.46 (m, 1H), 2.34 - 2.23 (m, 1H), 2.23 (s, 3H).

¹³C NMR (151 MHz, CDCl₃) δ (ppm) 168.4, 161.9, 161.4, 156.6, 144.3, 134.4, 132.9, 130.3, 117.6, 116.2, 104.0, 102.2, 93.8, 77.8, 69.0, 68.4, 55.7, 39.8, 33.6, 29.8.

### Example 2 N-(3-(4,5-Dihydro-2H,5′H-Spiro[Furan-3,7′-Furo[3,4-b]Pyridin]-2′-yl)-1-Methyl-1H-Pyrrolo[2,3-c]Pyridin-5-yl)Acetamide

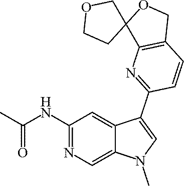 [SMILES: CC(=O)Nc1cc2c(-c3ccc4c(n3)C3(CCOC3)OC4)cn(C)c2cn1]

### Step 1 Synthesis of 2′-Chloro-4,5-Dihydro-2H,5′H- Spiro[Furo-3,7′-Furo[3,4-b]Pyridine]

4,5-Dihydro-2H,5′H-spiro[furo-3,7′-furo[3,4-b]pyridine]1′-oxide (294.0 mg, 1.52 mmol) and phosphorus oxychloride (5 mL, 53.6 mmol) were added to a reaction flask, the mixture was heated to 110° C. and stirred for 5 h, then concentrated under reduced pressure, to the solution were added saturated sodium carbonate (5 mL) and water (5 mL). After extraction with ethyl acetate (10 mL ×3), the combined organic phase was washed with saturated sodium chloride (10 mL), dried over anhydrous sodium sulfate and filtered, the filtrate was concentrated under reduced pressure. The obtained residue was purified by column chromatography (dichloromethane/methanol (v/ v) = 99/1) to obtain the title compound as a yellow-white solid (35.0 mg, yield 10.9%).

MS (ESI, pos. ion) m/z: 212.0 [M+H]⁺;

¹H NMR (400 MHz, CDCl₃) δ (ppm) 7.54 (d, J = 8.0 Hz, 1H), 7.28 (d, J = 3.7 Hz, 1H), 5.09 (s, 2H), 4.15 (dd, J = 8.7, 5.4 Hz, 2H), 4.03 (q, J = 9.7 Hz, 2H), 2.55 - 2.40 (m, 1H), 2.33 -2.22 (m, 1H).

### Step 2 Synthesis of N-(3-(4,5-Dihydro-2H,5′H-Spiro[Furan-3,7′-Furo[3,4-b]Pyridin]-2′-yl) -1-Methyl-1H-Pyrrolo[2,3-c]Pyridin-5-yl)Acetamide

Under N₂, 2′-chloro-4,5-dihydro-2H,5′H-spiro[furan-3,7′-furo[3,4-b]pyridine] (65.0 mg, 0.27 mmol), N-(1-methyl-3-(4,4,5,5-tetramethyl-1,3,2-dioxaborolan-2-yl)-1H-pyrrolidin[2,3-c]pyridin-5-yl)aceta mide (98.3 mg, 0.31 mmol) and 1,4-dioxane (4 mL), were added to a reaction flask and stirred to dissolve, then water (1 mL) and sodium carbonate (82.0 mg, 0.77 mmol) were added, the mixture was deoxygenated by N₂ bubbling for 10 min, then PdCl₂dppf (24.0 mg, 0.029 mmol) was added, the solution was stirred at 80° C. overnight under N₂ and cooled to room temperature. The resulting reaction was quenched by water (10 mL), after extraction with chloroform (3 × 10 mL), the combined organic phase was dried over anhydrous sodium sulfate, concentrated under reduced pressure, and the resulting residue was purified by column chromatography (ethyl acetate/methanol (v/v) = 15/1) to obtain the title compound as a yellow solid (30.4 mg, yield 35.9%).

MS (ESI, pos. ion) m/z: 365.2 [M+H]⁺;

¹H NMR (400 MHz, CDCl₃) δ (ppm) 9.00 (s, 1H), 8.59 (s, 1H), 8.40 (s, 1H), 7.89 (s, 1H), 7.66 - 7.52 (m, 2H), 5.10 (s, 2H), 4.27 - 4.05 (m, 4H), 3.92 (s, 3H), 2.66 - 2.51 (m, 1H), 2.37 - 2.28 (m, 1H), 2.24 (s, 3H).

¹³C NMR (151 MHz, CDCl₃) δ (ppm) 168.4, 160.4, 153.8, 144.2, 134.1, 133.0, 132.5, 129.9, 129.9, 129.2, 118.9, 115.8, 104.6, 93.2, 77.7, 69.5, 68.9, 39.8, 33.5, 24.7.

### Example 3 N-(1-Methyl-3-(4,5,5′,6′-Tetrahydro-2H-Spiro[Furan-3,8′-Pyrano[3,4-b]Pyridin e]-2′-yl)-1H-Pyrrolo[2,3-c]Pyridin-5-yl)Acetamide

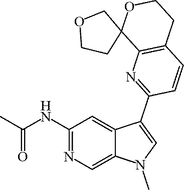 [SMILES: CC(=O)Nc1cc2c(-c3ccc4c(n3)C3(CCOC3)OCC4)cn(C)c2cn1]

### Step 1 Synthesis of 2-Bromo-3-(2-Methoxyethenyl)Pyridine

Methoxymethyltriphenylphosphonium chloride (4.42 g, 12.8 mmol) and anhydrous toluene (20 mL) were added to a reaction flask, the mixture was concentrated under reduced pressure, under N₂, anhydrous tetrahydrofuran (20 mL) was added and the solution was cooled to 0° C., then LiHMDS (12 mL, 12 mmol, 1 mol/L THF solution) was added dropwise. After the addition was completed, the mixture was stirred at 0° C. for 30 min, and then a solution of 2-bromopyridine-3-carbaldehyde (1.86 g, 10.0 mmol) in anhydrous THF (10 mL) was added and stirred at 0° C. for 3 h, the reaction was quenched by saturated ammonium chloride (30 mL), after extraction with petroleum ether/ethyl acetate mixture (30 mL×2, petroleum ether/ ethyl acetate (v/v) = 5/1), the combined organic phase was washed with water (50 mL) and saturated sodium chloride (50 mL), dried over anhydrous sodium sulfate and filtered, the filtrate was concentrated under reduced pressure. The resulting residue was purified by column chromatography (petroleum ether/ethyl acetate (v/v) = 9/1) to obtain the title compound as yellow oily liquid (1.77 g, yield 83.1%). Proton spectrum showed E/Z = 1:0.8.

NMR data of E-type products:

¹H NMR (400 MHz, CDCl₃) δ (ppm) 8.39 - 8.25 (m, 1H), 8.19 - 8.11 (m, 1H), 7.23 - 7.16 (m, 1H),6.36 (d, J = 7.2 Hz, 1H), 5.59 (d, J = 7.2 Hz, 1H), 3.83 (s, 3H).

NMR data of Z-type products:

¹H NMR (400 MHz, CDCl₃) δ (ppm) 8.31 - 8.06 (m, 1H), 7.64 - 7.56 (m, 1H), 7.20 - 7.12 (m, 1H), 6.99 (d, J = 12.9 Hz, 1H), 5.99 (d, J = 12.9 Hz, 1H), 3.75 (s, 3H).

### Step 2 Synthesis of 2-(2-Bromopyridin-3-yl)Acetaldehyde

2-Bromo-3-(2-methoxyethenyl)pyridine (1.75 g, 8.21 mmol) and anhydrous formic acid (10 mL) were added to a reaction flask, the mixture was heated to 60° C. and stirred overnight, and concentrated under reduced pressure, then dichloromethane (20 mL) was added, the solution was washed with saturated sodium bicarbonate (20 mL) and saturated sodium chloride (20 mL), then dried over anhydrous sodium sulfate and filtered, the filtrate was concentrated under reduced pressure, the resulting residue was purified by column chromatography (petroleum ether/ethyl acetate (v/v) = 4/1) to obtain the title compound as light yellow oily liquid (0.32 g, yield 19.0%).

MS (ESI, pos. ion) m/z: 200.0 [M+H]⁺;

¹H NMR (400 MHz, CDCl₃) δ (ppm) 9.81 (t, J = 1.3 Hz, 1H), 8.37 - 8.26 (m, 1H), 7.63 - 7.48 (m, 1H), 7.32 - 7.26 (m, 1H), 3.89 (d, J = 0.9 Hz, 2H).

### Step 3 Synthesis of 2-(2-Bromopyridin-3-yl)Ethanol

2-(2-Bromopyridin-3-yl)acetaldehyde (1.85 g, 9.25 mmol) and absolute ethanol (20 mL) were added to a reaction flask, under N₂, the mixture was cooled to 0° C., then sodium borohydride (0.43 g, 11 mmol) was added, and the mixture was heated to room temperature and stirred for 2 h, then the reaction was quenched by adding saturated ammonium chloride (20 mL) dropwise, ethanol was concentrated under reduced pressure. After extraction with ethyl acetate (20 mL×3), the resulting solution was washed with saturated sodium chloride (20 mL) and the organic phases were collected, dried over anhydrous sodium sulfate and filtered, the filtrate was concentrated under reduced pressure. The resulting residue was purified by column chromatography (petroleum ether/ethyl acetate (v/v) = 1/1) to obtain the title compound as colorless oily liquid (1.46 g, yield 78.1%).

MS (ESI, pos. ion) m/z: 202.0 [M+H]⁺;

¹H NMR (400 MHz, CDCl₃) δ (ppm) 8.28 - 8.16 (m, 1H), 7.65 - 7.55 (m, 1H), 7.24 - 7.15 (m, 1H), 3.93 (dd, J= 11.6, 6.2 Hz, 2H), 3.00 (t, J = 6.5 Hz, 2H).

### Step 2 Synthesis of 3-(3-(2-Hydroxyethyl)Pyridin-2-yl)Tetrahydrofuran-3-ol

2-(2-Bromopyridin-3-yl)ethanol (1.46 g, 7.23 mmol) was added to a reaction flask, under N₂, anhydrous THF (24 mL) was added and the mixture was cooled to -78° C., then n-butyllithium was added dropwise (5.9 mL, 15 mmol, 2.5 mol/L n-hexane solution), the solution was stirred at -78° C. for 1 h, then tetrahydrofuran-3-one (0.68 mL, 8.8 mmol) was added, and the mixture was warmed to room temperature and stirred for 90 min. The reaction was quenched by saturated ammonium chloride (20 mL), after extraction with ethyl acetate (20 mL×3), the organic phase was dried over anhydrous sodium sulfate, filtered, and the filtrate was concentrated under reduced pressure, the obtained residue was purified by column chromatography (petroleum ether/ethyl acetate (v/v) = ¼) to obtain the title compound as viscous colorless oily liquid (0.34 g, yield 22.0%).

MS (ESI, pos. ion) m/z: 210.1 [M+H]⁺;

¹H NMR (400 MHz, CDCl₃) δ (ppm) 8.38 (dd, J = 4.6, 1.5 Hz, 1H), 7.67 (dd, J = 7.7, 1.5 Hz, 1H),

7.23 (dd, J = 7.7, 4.7 Hz, 1H), 4.30 (d, J = 9.8 Hz, 1H), 4.21 - 4.06 (m, 2H), 3.99 - 3.88 (m, 3H), 3.12 - 2.96 (m, 2H), 2.54 - 2.46 (m, 1H), 2.22 - 2.15 (m, 1H).

### Step 5 Synthesis of 4,5,5′,6′-Tetrahydro-2H-Spiro[Furan-3,8′-Pyrano[3,4-b]Pyridine]

3-(3-(2-Hydroxyethyl)pyridin-2-yl)tetrahydrofuran-3-ol (105 mg, 0.50 mmol) was added to a reaction flask, under N₂, anhydrous THF (5 mL) was added and the mixture was cooled to 0° C., then NaEA/IIDS (0.55 mL, 1.1 mmol, 2 mol/L THF solution) was added dropwise and stirred at 0° C. for 10 min, then p-toluenesulfonyl chloride (117 mg, 0.60 mmol) was added and the mixture was stirred at 0° C. for 1 h. The resulting reaction was quenched by saturated ammonium chloride (10 mL), after extraction with ethyl acetate (10 mL×3), the combined organic phase was washed with saturated sodium chloride (10 mL) and filtered, the filtrate was concentrated under reduced pressure. The obtained residue was purified by column chromatography (dichloromethane/methanol (v/v) = 19/1) to obtain the title compound as yellow oily liquid (90.5 mg, yield 94.3%).

MS (ESI, pos. ion) m/z: 192.1 [M+H]⁺;

¹H NMR (400 MHz, CDCl₃) δ (ppm) 8.49 - 8.42 (m, 1H), 7.43 - 7.35 (m, 1H), 7.12 - 7.05 (m, 1H), 4.19 - 4.07 (m, 4H), 3.99 - 3.92 (m, 2H), 2.94 - 2.82 (m, 2H), 2.64 - 2.57 (m, 1H), 2.34 - 2.24 (m, 1H).

### Step 6 Synthesis of 4,5,5′,6′-Tetrahydro-2H Spiro[Furan-3,8′-Pyran[3,4-b]Pyridine]1′-Oxi de

4,5,5’,6′-Tetrahydro-2H-spiro[furan-3,8′-pyrano[3,4-b]pyridine] (985 mg, 5.15 mmol) and dichloromethane (25 mL) were added to a reaction flask, then m-chloroperoxybenzoic acid (1.57 g, 7.73 mmol) was added, the mixture was stirred at room temperature overnight. The reaction was quenched by saturated sodium sulfite (25 mL), stirred for 15 min, then saturated sodium carbonate (25 mL) was added, after extraction with chloroform (25 mL×3), the combined organic phase was dried over anhydrous sodium sulfate and filtered, the filtrate was concentrated under reduced pressure. The obtained residue was purified by column chromatography (dichloromethane/methanol (v/v)=19 /1) to obtain the title compound as a white solid (935.7 mg, yield 87.7%).

MS (ESI, pos. ion) m/z: 208.1 [M+H]⁺;

¹H NMR (400 MHz, CDCl₃) δ (ppm) 8.08 (d, J = 6.3 Hz, 1H), 7.18 - 7.11 (m, 1H), 7.10 - 7.04 (m, 1H), 4.50 (d, J = 8.8 Hz, 1H), 4.26 - 4.16 (m, 2H), 3.99 - 3.89 (m, 1H), 3.87 - 3.75 (m, 2H), 3.13 - 3.02 (m, 1H), 2.98 - 2.90 (m, 1H), 2.88 - 2.76 (m, 1H), 2.00 - 1.90 (m, 1H).

### Step 7 Synthesis of 2′-Chloro-4,5,5′,6′-Tetrahydro-2H-Spiro[Furan-3,8′-Pyrano[3,4-b]Pyri dine]

4,5,5’,6′-Tetrahydro-2H-spiro[furan-3,8′-pyrano[3,4-b]pyridine]1′-oxide (835 mg, 4.03 mmol) and phosphine oxychloride (15 mL, 160.9 mmol) were added to a reaction flask, the mixture was heated to 110° C. and stirred for 1 h, the resulting solution was concentrated under reduced pressure, 12 mL of saturated sodium bicarbonate was added, and the pH was adjusted to 7 with saturated sodium carbonate. After extraction with ethyl acetate (20 mL × 3), the combined organic phase was washed with saturated sodium chloride (20 mL), dried over anhydrous sodium sulfate and filtered, the filtrate was concentrated under reduced pressure, and the obtained residue was purified by column chromatography (dichloromethane/methanol (v/v) = 97:3) to obtain the title compound as a yellow-white solid (165 mg, yield 18.1%).

MS (ESI, pos. ion) m/z: 226.1 [M+H]⁺;

¹H NMR (400 MHz, CDCl₃) δ (ppm) 7.37 (d, J = 8.1 Hz, 1H), 7.13 (d, J = 8.0 Hz, 1H), 4.08 (mk, 4H), 4.00 - 3.86 (m, 2H), 2.94 - 2.76 (m, 2H), 2.65 - 2.56 (m, 1H), 2.31 - 2.19 (m, 1H).

### Step 8 Synthesis of N-(1-Methyl-3-(4,5,5′,6′-Tetrahydro-2H-Spiro[Furan-3,8′-Pyrano[3,4-b]Pyridine]-2′-yl)-1H-Pyrrolo[2,3-c]Pyridin-5-yl)Acetamide

Under N₂, 2′-chloro-4,5,5’,6′-tetrahydro-2H-spiro[furan-3,8′-pyrano[3,4-b]pyridine] (80 mg, 0.35 mmol), N-(1-methyl-3-(4,4,5,5-tetramethyl-1,3,2-dioxaborolan-2-yl)-1H-pyrrolo[2,3-c]pyridin-5-yl)acetami de (145 mg, 0.46 mmol) and 1,4-dioxane (8 mL) were added to a reaction flask and the mixture was stirred to dissolve, then water (2 mL) and sodium carbonate (113 mg, 1.07 mmol) were added, then PdCl₂dppf (30 mg, 0.04 mmol) was added, the mixture was deoxygenated by bubbling N₂ for 10 min and stirred at 80° C. overnight under N₂, then the heating was stopped, and the solution was cooled to room temperature. The reaction was quenched by water (10 mL), after extraction with chloroform (10 mL × 3), the combined organic phase was dried over anhydrous sodium sulfate and filtered, and the filtrate was concentrated under reduced pressure, the obtained residue was purified by column chromatography (dichloromethane/methanol (v/ v) = 91:9) to obtain the title compound as a yellow solid (78.5 mg, yield 58.5%).

MS (ESI, pos. ion) m/z: 379.2 [M+H]⁺;

¹H NMR (600 MHz, CDCl₃) δ (ppm) 8.99 (s, 1H), 8.40 (s, 1H), 8.30 (s, 1H), 7.79 (s, 1H), 7.49 (d, J = 8.0 Hz, 1H), 7.41 (d, J = 8.0 Hz, 1H), 4.36 - 4.11 (m, 4H), 4.06 - 3.95 (m, 2H), 3.91 (s, 3H), 2.96 - 2.80 (m, 3H), 2.38 - 2.30 (m, 1H), 2.23 (s, 3H).

¹³C NMR (151 MHz, CDCl₃) δ (ppm) 168.1, 155.5, 151.6, 144.0, 136.9, 133.5, 132.9, 132.6, 130.1, 126.8, 118.1, 115.9, 104.9, 87.0, 78.7, 68.7, 60.8, 40.8, 33.4, 28.4, 24.8.

### Example 4 N-(3-(4-Methoxy-2′,3′,5′,6′-Tetrahydro-5H-Spiro[Furo[3,4-b]Pyridine-7,4′-Pyr an]-2-yl)-1-m Ethyl-1H-Pyrrolo [2,3-c] Pyridin-5-yl)Acetamide

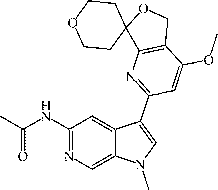 [SMILES: COc1cc(-c2cn(C)c3cnc(NC(C)=O)cc23)nc2c1COC21CCOCC1]

### Step 1 Synthesis of 4-(3-(Hydroxymethyl)Pyridin-2-yl)Tetrahydro-2H-Pyran-4-Ol

2-Bromo-3-hydroxymethylpyridine (5.64 g, 30 mmol) was added to a reaction flask, under N₂, anhydrous THF (100 mL) was added, then the mixture was cooled to -78° C., and n-butyllithium (25 mL, 63 mmol, 2.5 mol/L n-hexane solution) was added dropwise, and stirred at -78° C. for 1 h, then tetrahydropyran-4-one (3.60 g, 37 mmol) was added and stirred at -78° C. for 1 h, and then the solution was continued to stir at room temperature for 1 h. The reaction was quenched by saturated ammonium chloride (50 mL), after extraction with ethyl acetate (50 mL×3), the combined organic phase was dried over anhydrous sodium sulfate and filtered, and the filtrate was concentrated under reduced pressure. The obtained residue was purified by column chromatography (petroleum ether/ethyl acetate (v/v) = ¼) to obtain the title compound as viscous colorless oily liquid (1.66 g, yield 26.4%).

MS (ESI, pos. ion) m/z: 210.1 [M+H]⁺;

¹H NMR (400 MHz, CDCl₃) δ (ppm) 8.54 - 8.43 (m, 1H), 7.80 (dd, J = 7.7, 1.5 Hz, 1H), 7.26 - 7.21 (m, 1H), 4.92 (s, 2H), 4.03 - 3.85 (m, 4H), 2.51 - 2.32 (m, 2H), 1.63 - 1.48 (m, 2H).

### Step 2 Synthesis of 2′,3′,5′,6′-Tetrahydro-5H- Spiro[Furo[3,4-b]Pyridine-7,4′-Pyran]

4-(3-(Hydroxymethyl)pyridin-2-yl)tetrahydro-2H-pyran-4-ol (1.40 g, 6.69 mmol) was added to a reaction flask, under N₂, anhydrous THF (167 mL) was added and the mixture was cooled to 0° C., then sodium bis(trimethylsilyl)amide (7.4 mL, 15 mmol, 2 mol/L in THF) was added dropwise and stirred at 0° C. for 10 min, and then p-toluenesulfonyl chloride (1.56 g, 8.02 mmol) was added and stirred at 0° C. for 1 h. The reaction was quenched by saturated ammonium chloride (30 mL), after extraction with ethyl acetate (30 mL × 3), the combined organic phase was washed with saturated sodium chloride (30 mL) and filtered, and the filtrate was concentrated under reduced pressure. The resulting residue was purified by column chromatography (dichloromethane/methanol (v/v)=19/1) to obtain the title compound as yellow oily liquid (1.10 g, yield 86.0%).

MS (ESI, pos. ion) m/z: 192.1 [M+H]⁺;

¹H NMR (400 MHz, CDCl₃) δ (ppm) 8.50 (d, J = 4.8 Hz, 1H), 7.55 (d, J = 7.6 Hz, 1H), 7.20 - 7.14 (m, 1H), 5.08 (s, 2H), 4.03 - 3.95 (m, 2H), 3.90 - 3.81 (m, 2H), 2.27 - 2.11 (m, 2H), 1.66 - 1.58 (m, 2H).

### Step 3 Synthesis of 2′,3′,5′,6′-Tetrahydro-5H-Spiro[Furo[3,4-b]Pyridine-7,4′-Pyran]1-Oxi de

2′,3′,5′,6′-Tetrahydro-5H-spiro[furo[3,4-b]pyridine-7,4′-pyran] (290.5 mg, 1.52 mmol) dichloromethane (10 mL) and m-chloroperoxybenzoic acid (460.0 mg, 2.3 mmol, 85 mass%) were added to a reaction flask, the mixture was stirred at room temperature overnight, then the reaction was quenched by saturated sodium sulfite (10 mL) and stirred for 15 min, then saturated carbonic acid sodium (10 mL) was added. After extraction with chloroform (10 mL × 3), the combined organic phase was dried over anhydrous sodium sulfate and filtered, the filtrate was concentrated under reduced pressure. The obtained residue was purified by column chromatography (dichloromethane/methanol (v/ v) = 19/1) to obtain the title compound as a white solid (241.4 mg, yield 76.7%).

MS (ESI, pos. ion) m/z: 208.1 [M+H]⁺;

¹H NMR (400 MHz, CDCl₃) δ (ppm) 8.10 - 8.03 (m, 1H), 7.22 - 7.17 (m, 1H), 7.13 -7.08 (m, 1H), 5.09 (s, 2H), 3.99 - 3.89 (m, 2H), 3.85 - 3.77 (m, 2H), 3.06 - 2.90 (m, 2H), 1.54 -1.45 (m, 2H).

### Step 4 Synthesis of 4-Chloro-2′,3′,5′,6′-Tetrahydro-5H-Spiro[Furo[3,4-b]Pyridine-7,4′-Pyr an]

2′,3′,5′,6′-Tetrahydro-5H-spiro[furo[3,4-b]pyridine-7,4′-pyran]1-oxide (705.3 mg, 3.40 mmol) and phosphorus oxychloride (7 mL, 75.1 mmol) were added to a reaction flask, the mixture was heated to 110° C. and stirred for 1 h, then concentrated under reduced pressure, and saturated sodium bicarbonate (5 mL) was added, and then the pH was adjusted to 7 with saturated sodium carbonate, the solution was extracted with ethyl acetate (10 mL×3), the combined organic phase was washed with saturated sodium chloride (10 mL), dried over anhydrous sodium sulfate and filtered, the filtrate was concentrated under reduced pressure. The obtained residue was purified by column chromatography (dichloromethane/methanol (v/v) = 49/1) to obtain the title compound as a yellow-white solid product (542.4 mg, yield 71.0%).

MS (ESI, pos. ion) m/z: 226.1 [M+H]⁺;

¹H NMR (400 MHz, CDCl₃) δ (ppm) 8.40 (d, J = 5.4 Hz, 1H), 7.18 (d, J = 5.4 Hz, 1H), 5.10 (s, 2H), 4.04 - 3.94 (m, 2H), 3.90 - 3.80 (m, 2H), 2.22 - 2.11 (m, 2H), 1.70 - 1.59 (m, 2H).

### Step 5 Synthesis of 4-Methoxy-2′,3′,5′,6′-Tetrahydro-5H-Spiro[Furo[3,4-b]Pyridine-7,4′-P yran]

4-Chloro-2′,3′,5′,6′-tetrahydro-5H-spiro[furo[3,4-b]pyridine-7,4′-pyran] (542.4 mg, 2.40 mmol) and a solution of sodium methoxide in methanol (9 mL, 49 mmol, 5.4 mol/L) were added to a reaction flask, the mixture was heated to 80° C. and stirred for 5 h, then the reaction was quenched by saturated ammonium chloride (5 mL), and then concentrated hydrochloric acid was added to adjust the pH to weak base, after extraction with ethyl acetate (10 mL×3), the combined organic phase was washed with saturated sodium chloride (10 mL×3), dried over anhydrous sodium sulfate and filtered, and the organic phase was concentrated under reduced pressure to obtain the title compound as a white solid (450.3 mg, yield 84.7%).

MS (ESI, pos. ion) m/z: 222.1 [M+H]⁺.

### Step 6 Synthesis of 4-Methoxy-2′,3′,5′,6′-Tetrahydro-5H-Spiro[Furo[3,4-b]Pyridine-7,4′-P yran]1-Oxide

4-Methoxy-2′,3′,5′,6′-tetrahydro-5H-spiro[furo[3,4-b]pyridine-7,4′-pyran] (542.4 mg, 0.76 mmol), anhydrous dichloromethane (10 mL) and m-chloroperoxybenzoic acid (696.5 mg, 3.43 mmol, yield 85%) were added to a reaction flask, the mixture was stirred at room temperature overnight, then the reaction was quenched by saturated sodium sulfite (10 mL) and stirred for 15 min, and then saturated sodium carbonate (10 mL) was added. After extraction with 3×10 mL chloroform, the combined organic phase was dried over anhydrous sodium sulfate and filtered, and the filtrate was concentrated under reduced pressure, and the obtained residue was purified by column chromatography (dichloromethane/methanol (v/v) = 19/1) to obtain the title compound as a white solid (394.5 mg, yield 67.8%).

MS (ESI, pos. ion) m/z: 238.2 [M+H]⁺.

### Step 7 Synthesis of 2-Chloro-4-Methoxy-2′,3′,5′,6′-Tetrahydro-5H-Spiro[Furo[3,4-b]Pyrid ine-7,4′-Pyran]

4-Methoxy-2′,3′,5′,6′-tetrahydro-5H-spiro[furo[3,4-b]pyridine-7,4′-pyran]1-oxide (395.0 mg, 1.67 mmol) and phosphorus oxychloride (5 mL, 53.64 mmol) were added to a reaction flask, the mixture was heated to 110° C. and stirred for 8 h, the reaction solution was concentrated under reduced pressure, then saturated sodium bicarbonate (5 mL) was added, and the pH was adjusted to 7 with saturated sodium carbonate. After extraction with chloroform (10 mL × 3), the combined organic phase was washed with saturated sodium chloride (10 mL), drier over anhydrous sodium sulfate and filtered, the filtrate was concentrated under reduced pressure. The obtained residue was purified by column chromatography (dichloromethane/methanol (v/v) = 49/1) to obtain the title compound as a yellow-white solid (147.3 mg, yield 34.6%).

MS (ESI, pos. ion) m/z: 256.1 [M+H]⁺;

¹H NMR (400 MHz, CDCl₃) δ (ppm) 6.71 (s, 1H), 5.00 (s, 2H), 4.02 - 3.93 (m, 2H), 3.89 (s, 3H), 3.83 - 3.76 (m, 2H), 2.23 - 2.11 (m, 2H), 1.61 - 1.54 (m, 2H).

### Step 8 Synthesis of N-(3-(4-Methoxy-2′,3′,5′,6′-Tetrahydro-5H-Spiro[Furo[3,4-b]Pyridine -7,4′-Pyran]-2-yl)-1-Methyl-1H-Pyrrolo[2,3-c]Pyridin-5-yl)Acetamide

Under N₂, 2-chloro-4-methoxy-2′,3′,5′,6′-tetrahydro-5H-spiro[furo[3,4-b]pyridine-7,4′-pyran] (70.0 mg, 0.27 mmol), N-(1-methyl-3-(4,4,5,5-tetramethyl-1,3,2-dioxaborolan-2-yl)-1H-pyrrolo[2,3-c]pyridin-5-yl)acetami de (94.0 mg, 0.30 mmol) and 1,4-dioxane (8 mL) were added to a 50 mL two-neck round bottom flask, the mixture was stirred to dissolve, then water (2 mL) and sodium carbonate (87.0 mg, 0.82 mmol) were added, the solution was deoxygenated by bubbling N₂ for 10 min, then PdCl₂dppf (22.0 mg, 0.026 mmol) was added and deoxygenated by bubbling N₂ for 10 min. Under N₂, the mixture was stirred at 80° C. overnight, then the heating was stopped, the mixture was cooled to room temperature, the reaction was quenched by water (10 mL), after extraction with chloroform (10 mL × 3), the combined organic phase was dried over anhydrous sodium sulfate and filtered, and the filtrate was concentrated under reduced pressure. The obtained residue was purified by column chromatography (ethyl acetate/methanol (v/v) = 19/1) to obtain the title compound as a white solid (11.0 mg, yield 9.8%).

MS (ESI, pos. ion) m/z: 409.2 [M+H]⁺;

¹H NMR (400 MHz, CDCl₃) δ (ppm) 8.96 (s, 1H), 8.43 (s, 1H), 8.21 (s, 1H), 7.92 (s, 1H), 7.16 (s, 1H), 5.09 (s, 2H), 4.03 (s, 3H), 4.13 - 3.83 (m, 4H), 3.93 (s, 3H), 2.34 - 2.18 (m, 2H), 2.24 (s, 3H), 1.74 - 1.65 (m, 2H).

¹³C NMR (151 MHz, CDCl₃) δ (ppm) 168.3, 165.7, 161.4, 166.0, 144.1, 134.2, 132.7, 132.6, 130.3, 116.4, 116.2, 103.9, 101.9, 83.1, 67.5, 64.5, 55.5, 35.5, 33.4, 24.8.

### Example 5 N-(3-(4′-Methoxy-4,5,5′,6′-Tetrahydro-2H-Spiro[Furan-3,8′-Pyrano[3,4-b]Pyri din]-2′-yl)-1-Methyl-1H-Pyrrolo[2,3-c]Pyridin-5-yl)Acetamide

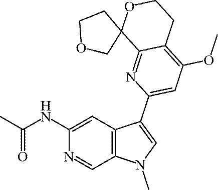 [SMILES: COc1cc(-c2cn(C)c3cnc(NC(C)=O)cc23)nc2c1CCOC21CCOC1]

### Step 1 Synthesis of 4′-Chloro-4,5,5′,6′-Tetrahydro-2H-Spiro[Furan-3,8′-Pyrano[3,4-b]Pyri dine]

To a 100 mL round bottom flask were added 4′-chloro-4,5,5′,6′-tetrahydro-2H-spiro[furan-3,8′-pyrano[3,4-b]pyridine]1′-oxide (2.11 g, 10.20 mmol) and phosphine oxychloride (28 mL, 300.40 mmol), then the mixture was heated to 110° C. and reacted for 1 h. After TLC monitoring showed that the reaction was completed, the mixture was concentrated in vacuum, then 10 mL of saturated sodium bicarbonate was added, the pH was adjusted to 7 with saturated sodium carbonate, after extraction with ethyl acetate (3 × 20 mL), the combined organic phase was washed with 20 mL of saturated sodium chloride, then dried and filtered, concentrated under reduced pressure, and the resulting residue was purified by column chromatography (dichloromethane/methanol (v/v) = 97/3) to obtain the title compound as a yellow-white solid (1.16 g, yield 50.50%).

MS (ESI, pos. ion) m/z: 226.2 [M+H]⁺.

### Step 2 Synthesis of 4′-Chloro-4,5,5′,6′-Tetrahydro-2H-Spiro[Furan-3,8′-Pyrano|[3,4-b]Pyri dine]1′-Oxide

4′-Chloro-4,5,5′,6′-tetrahydro-2H-spiro[furan-3,8′-pyrano[3,4-b]pyridine] (0.53 g, 2.33 mmol), dichloromethane (12 mL) and m-chloroperbenzoic acid (0.71 g, 3.50 mmol, 85 mass%) were added to a 100 mL flask, and the mixture was stirred overnight at room temperature. The reaction was quenched with 12 mL of saturated sodium sulfite, stirred for 15 min, then 12 mL of saturated sodium carbonate was added, after extraction with chloroform (3 × 20 mL), the combined organic phase was dried over anhydrous sodium sulfate and filtered, then concentrated under reduced pressure, and the resulting residue was purified by column chromatography(dichloromethane/methanol (v/v) = 95/5) to obtain the title compound as a white solid product (0.44 g, yield 78.11%).

MS (ESI, pos. ion) m/z: 242.0 [M+H]⁺.

### Step 3 Synthesis of 2′,4′-Dichloro-4,5,5′,6′-Tetrahydro-2H-Spiro[Furan-3,8′-Pyrano[3,4-b ]Pyridine]

To a 25 mL round bottom flask were added 4′-chloro-4,5,5′,6′-tetrahydro-2H-spiro[furan-3,8′-pyrano[3,4-b]pyridine]1′-oxide (0.44 g, 1.82 mmol) and phosphine oxychloride (5 mL, 53.64 mmol), the mixture was heated to 110° C. and reacted for 2 h. After the reaction was stopped, the resulting mixture was concentrated in vacuum, then saturated sodium bicarbonate (5 mL) was added, and the pH was adjusted to 7 with saturated sodium carbonate. After extraction with ethyl acetate (3 × 10 mL), the combined organic phase was dried over anhydrous sodium sulfate and filtered, then concentrated under reduced pressure, and the resulting residue was purified by column chromatography (petroleum ether/ethyl acetate (v/v) = 5/1) to obtain the title compound as a white solid (0.20 g, yield 40.00%).

MS (ESI, pos. ion) m/z: 260.1 [M+H]⁺.

### Step 4 Synthesis of 2′-Chloro-4′-Methoxy-4,5,5′,6′-Tetrahydro-2H-Spiro[Furan-3,8′-Pyra no[3,4-b]Pyridine]

To a 25 mL round bottom flask were added 2′,4′-dichloro-4,5,5′,6′-tetrahydro-2H-spiro[furan-3,8′-pyrano[3,4-b]pyridine] (0.087 g, 0.33 mmol), DMF (3 mL) and a solution of sodium methoxide in methanol (0.36 mL, 0.4 mmol, 1.1 M methanol solution), and the mixture was stirred overnight at room temperature. 3 mL of saturated ammonium chloride was added to quench the reaction, then 5 mL of water was added, after extraction with ethyl acetate (3 × 10 mL), the combined organic phase was washed with 10 mL of water and 10 mL of saturated sodium chloride, then the organic phase was separated and dried over anhydrous sodium sulfate, filtered and concentrated in vacuum, and the resulting residue was purified by column chromatography (petroleum ether/ethyl acetate (v/v) = 5/1) to obtain the title compound as colorless oily liquid (0.068 g, yield 85.00%).

MS (ESI, pos. ion) m/z: 256.2 [M+H]⁺.

### Step 5 Synthesis of N-(3-(4′-Methoxy-4,5,5′,6′-Tetrahydro-2H-Spiro[Furan-3,8′-Pyrano[3,4-b]Pyridine]-2′-yl)-1-Methyl-1H-Pyrrolo[2,3-c]Pyridin-5-yl)Acetamide

Under N₂, 2′-chloro-4′-methoxy-4,5,5′,6′-tetrahydro-2H-spiro[furan-3,8′-pyrano[3,4-b]pyridine] (89 mg, 0.35 mmol), N-[1-methyl-3-(4,4,5,5-tetramethyl-1,3,2-dioxaborolan-2-yl)-1H-pyrrolo[2,3-c]pyridin-5-yl]acetami de (126 mg, 0.40 mmol) and 1,4-dioxane (8 mL) were added to a 25 mL two-neck round bottom flask, the mixture was stirred to dissolve, then water (2 mL) was added, the system was a yellow turbid liquid, then sodium carbonate (111 mg, 1.05 mmol) was added and the mixture was deoxygenated by bubbling N₂ for 10 min, then PdCl₂dppf (29 mg, 0.03 mmol) was added and the mixture was deoxygenated by bubbling N₂ for 10 min. The flask was connected to a reflux condenser, and heated to 80° C. and reacted overnight under N₂. After the heating was stopped, the mixture was cooled to room temperature, then 10 mL of water was added to quench the reaction, after extraction with chloroform (3 × 10 mL), the combined organic phase was dried over anhydrous sodium sulfate, and the resulting residue was purified by column chromatography (ethyl acetate/methanol (v /v) = 91/9) to obtain the title compound as a yellow solid (77.0 mg, yield 54.20%).

MS (ESI, pos. ion) m/z: 409.2 [M+H]⁺;

¹H NMR (400 MHz, CDCl₃) δ 9.00 (s, 1H), 8.40 (s, 1H), 8.36 (s, 1H), 7.84 (s, 1H), 7.08 (s, 1H), 4.33 - 4.21 (m, 2H), 4.18 - 4.08 (m, 2H), 4.00 (s, 3H),4.05-3.86 (m, 2H),3.92 (s, 3H), 2.77 (d, J = 2.4 Hz, 1H), 2.35 - 2.26 (m, 1H), 2.23 (s, 3H).

### Example 6 N-(3-(4,5,5′,6′-Tetrahydro-2H-Spiro[Furan-3,8′-Pyrano[3,4-b]Pyridin]-2′-yl)-1-((R)-Tetrahydrofuran-3-yl)-1H-Pyrrolo[2,3-c]Pyridin-5-yl)Acetamide

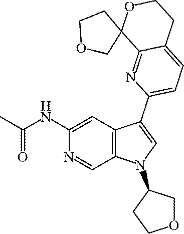 [SMILES: CC(=O)Nc1cc2c(-c3ccc4c(n3)C3(CCOC3)OCC4)cn([C@@H]3CCOC3)c2cn1]

### Step 1 Synthesis of (S)-Tetrahydrofuran-3-yl Methanesulfonate

Dichloromethane (15 mL, 100 mass%), (S)-tetrahydrofuran-3-ol (0.8 g, 5.7 mmol), methanesulfonyl chloride (1.2 g, 11 mmol) and pyridine (1.44 g, 19 mmol) were added to a 25 mL one-necked bottle at 0° C., the mixture was reacted at 60° C. for 2 h. The solvent was concentrated under reduced pressure, and the residual liquid was dissolved in 10 mL of ethyl acetate. After the insoluble solid was filtered off with suction, the solvent was concentrated under reduced pressure, and the crude product was purified by column chromatography (petroleum ether/ethyl acetate (v/v) = 2/ 1) to obtain the title compound as yellow liquid (680 mg, yield 45%).

¹H-NMR: (400 MHz, CDCl₃) δ 5.37 - 5.29 (m, 1H), 4.05 - 3.90 (m, 4H), 3.05 (s, 3H), 2.26 (dd, J = 7.6, 5.2 Hz, 2H).

### Step 2 Synthesis of (R)-N-(1-(Tetrahydrofuran-3-yl)-1H-Pyrrolo[2,3-c]Pyridin-5-yl)Acet amide

N,N-dimethylformamide (15 mL), (S)-tetrahydrofuran-3-yl methanesulfonate (600 mg, 3.6 mmol) and N-(1H-pyrrolo[2,3-c]pyridin-5-yl)acetamide (400 mg, 2.28 mmol) were added to a 50 mL one-necked bottle, the mixture was reacted at 80° C. for 5 h. The solvent was concentrated under reduced pressure, and the crude product was purified by column chromatography (dichloromethane/methanol (v/v) = 20/1) to obtain the title compound as a yellow solid (420.0 mg, yield 74.95%).

¹H NMR (400 MHz, CDCl₃) δ 8.49 (s, 1H), 8.41 (s, 1H), 7.40 (d, J = 3.1 Hz, 1H), 6.52 (d, J = 3.1 Hz, 1H), 5.31 (s, 1H), 5.12 (ddd, J = 10.7, 6.0, 2.9 Hz, 1H), 4.19 (dd, J = 14.5, 5.4 Hz, 2H), 4.12 - 4.05 (m, 1H), 3.96 (td, J = 8.7, 6.4 Hz, 1H), 2.57 (dt, J = 14.3, 8.1 Hz, 1H), 2.23 (s, 3H), 1.96 - 1.88 (m, 1H).

### Step 3 Synthesis of (R)-N-(3-Bromo-1-(Tetrahydrofuran-3-yl)-1H-Pyrrolo[2,3-c]Pyridin-5-yl)Acetamide

Dichloromethane (10 mL), N-bromosuccinimide (230 mg, 1.3 mmol) and (R)-N-(1-(tetrahydrofuran-3-yl)-1H-pyrrolo[2,3-c]pyridin-5-yl)acetamide (295 mg, 1.2 mmol) were added to a 50 mL one-necked bottle, the mixture was reacted at room temperature for 1 h. The resulting solution was poured into 200 mL of water, extracted with dichloromethane (3 × 50 mL), and dried. The solvent was concentrated under reduced pressure, and the crude product was purified by column chromatography (dichloromethane/methanol (v/v) = 20/1) to obtain the title compound as a yellow solid (340.0 mg, yield 87.20%).

¹H NMR (400 MHz, CDCl₃) δ 8.48 (s, 1H), 8.33 (s, 1H), 8.23 (s, 1H), 7.43 (s, 1H), 5.11 (td, J = 5.5, 2.8 Hz, 1H), 4.24 - 4.15 (m, 2H), 4.06 (dd, J = 10.2, 5.7 Hz, 1H), 3.95 (td, J = 8.8, 6.5 Hz, 1H), 2.59 (td, J = 14.1, 8.2 Hz, 1H), 2.24 (s, 3H), 2.18 (ddd, J = 13.7, 8.8, 3.2 Hz, 1H).

### Step 4 Synthesis of (R)-N-(1-(Tetrahydrofuran-3-yl)-3-(4,4,5,5,-Tetramethyl-1,3,2-Dioxab orolan-2-yl)-1H-Pyrrolo[2,3-c]Pyridin-5-yl)Acetamide

Under N₂, tetrahydrofuran (10 mL) was added to a 50 mL two-necked flask, and after cooling down to -78° C., n-butyllithium (2.5 mL, 6.3 mmol, 2.5 mmol/L) was added, then a solution of (R)-N-(3-bromo-1-(tetrahydrofuran-3-yl)-1H-pyrrolo[2,3-c]pyridin-5-yl)acetamide (340.0 mg, 1.049 mmol) in tetrahydrofuran (5 mL) was added slowly. The mixture was continuously stirred for 30 min. Then 2-isopropoxy-4,4,5,5-tetramethyl-1,3,2-dioxaborolane (1.23 g, 2 mmol) was added and the reaction was continued for 1 h. 10 mL of saturated ammonium chloride was added to quenched the reaction at 0° C., after extraction with ethyl acetate (2 × 20 mL), the solvent was concentrated under reduced pressure, and the crude product was purified by column chromatography (dichloromethane/methanol (v/v) = 20 /1) to obtain the title compound as a yellow solid (230.0 mg, yield 59.08%).

MS (ESI, pos. ion) m/z: 372.2 [M+H]⁺;

### Step 5 Synthesis of N-(3-(4,5,5′,6′-Tetrahydro-2H-Spiro[Furan-3,8′-Pyrano[3,4-b]Pyridin ]-2′-yl)-1-((R)-Tetrahydrofuran-3-yl)-1H-Pyrrolo[2,3-c]Pyridin-5-yl)Acetamide

1,4-Dioxane (8 mL), water (1 mL), PdCl₂dppf (25.5 mg, 0.04 mmol), potassium carbonate (60.3 mg, 0.41 mmol), (R)-N-(1-(tetrahydrofuran-3-yl)-3-(4,4,5,5,-tetramethyl-1,3,2-dioxaborolan-2-yl)-1H-pyrrolo[2,3-c] pyridin-5-yl)acetamide (88 mg, 0.24 mmol) and 2′-chloro-4,5,5′,6′-tetrahydro-2H-spiro[furan-3,8′-pyrano[3,4-b]pyridine] (40.0 mg, 0.18 mol) were added to a 25 mL one-necked flask, the mixture was reacted at 100° C. overnight. The solvent was concentrated under reduced pressure, and the crude product was purified by column chromatography (dichloromethane/methanol (v/v) = 20/1) to obtain the title compound as a yellow solid (60.0 mg, yield 77.9%).

MS (ESI, pos. ion) m/z: 435.2 [M+H]⁺;

¹H NMR (400 MHz, CDC13) δ 9.08 (s, 1H), 8.54 (s, 1H), 8.34 (s, 1H), 7.92 (s, 1H), 7.46 (d, J = 17.7 Hz, 2H), 5.16 (s, 1H), 4.36 (s, 1H), 4.26 (s, 5H), 4.11 (s, 1H), 4.00 (s, 3H), 2.91 (d, J = 16.7 Hz, 2H), 2.62 (s, 1H), 2.37 (s, 1H), 2.25 (s, 3H), 1.99 (s, 2H).

### Example 7 N-(3-(4′-(Difluoromethyl)-4,5,5′,6′-Tetrahydro-2H-Spiro[Furan-3,8′-Pyrano[3, 4-b]Pyridin]-2′-yl)-1-Methyl-1H-Pyrrolo[2,3-c]Pyridin-5-yl)Acetamide

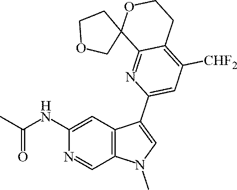 [SMILES: CC(=O)Nc1cc2c(-c3cc(C(F)F)c4c(n3)C3(CCOC3)OCC4)cn(C)c2cn1]

### Step 1 Synthesis of 4,5,5′,6′-Tetrahydro-2H-Spiro[Furan-3,8′-Pyrano[3,4-b]Pyridine]-4′-C arbaldehyde

(4,5,5′,6′-Tetrahydro-2H-spiro[furan-3,8′-pyrano[3,4-b]pyridin]-4′-yl)methanol (0.32 g, 1.45 mmol) and dichloromethane (20 mL) were added to a 50 mL round bottom flask, under N₂, Dess Martin oxidant (0.92 g, 2.17 mmol) was added, and the mixture was stirred at room temperature overnight. 5 mL of saturated sodium thiosulfate and 5 mL of saturated aqueous sodium bicarbonate were added dropwise and carefully, the mixture was continued to stir for 10 min, and then separated. The aqueous phase was extracted with dichloromethane (3 × 20 mL), washed with 20 mL of saturated sodium chloride, the organic phase was separated, then dried and filtered, and concentrated in vacuum, and the resulting residue was purified by column chromatography (dichloromethane/methanol (v/v)=20/1) to obtain the title compound as light yellow oily liquid (0.30 g, yield 94.9%).

MS (ESI, pos. ion) m/z: 220.2 [M+H]⁺;

¹H NMR (400 MHz, DMSO-d₆): δ (ppm) 10.26 (s, 1H), 8.75 (d, J = 4.8 Hz, 1H), 7.65 (d, J = 4.8 Hz, 1H), 4.04 - 3.91 (m, 6H), 3.25 (t, J = 5.6 Hz, 2H), 2.47 - 2.41 (m, 1H), 2.30 - 2.21 (m, 1H).

### Step 2 Synthesis of 4′-(Difluoromethyl)-4,5,5′,6′-Tetrahydro-2H-Spiro[Furan-3,8′-Pyrano [3,4-b]Pyridine]

4,5,5′,6′-Tetrahydro-2H-spiro[furan-3,8′-pyrano[3,4-b]pyridine]-4′-carbaldehyde (0.20 g, 0.91 mmol) was added to a 100 mL round bottom flask, under N₂, anhydrous DCM (25 mL) was added, then the mixture was cooled to -78° C. Diethylaminosulfur trifluoride (0.74 g, 4.56 mmol) was added dropwise, and after the addition was completed, the temperature was increased to 15° C. and stirring was continued for 4 h. The reaction was quenched by adding 20 mL of ice water, the mixture was extracted with dichloromethane (3 × 20 mL), dried over anhydrous sodium sulfate, filtered, and concentrated under reduced pressure, and the resulting residue was purified by column chromatography (petroleum ether/ethyl acetate (v/v) = 4/1) to obtain the title compound as a yellow solid (0.17 g, yield 77.0%).

MS (ESI, pos. ion) m/z: 242.2 [M+H]⁺;

¹H NMR (400 MHz, DMSO-d₆): δ (ppm) 8.63 (d, J = 4.8 Hz, 1H), 7.44 (d, J = 4.9 Hz, 1H), 7.22 (t, J = 54.0 Hz, 1H), 4.05 - 3.91 (m, 6H), 2.93 (s, 2H), 2.48 - 2.40 (m, 1H), 2.28 -2.20 (m, 1H).

### Step 3 Synthesis of 4′-(Difluoromethyl)-4,5,5′,6′-Tetrahydro-2H-Spiro[Furan-3,8′-Pyrano [3,4-b]Pyridine]-1′-Oxide

4′-(Difluoromethyl)-4,5,5′,6′-tetrahydro-2H-spiro[furan-3,8′-pyrano[3,4-b]pyridine] (0.16 g, 0.66 mmol), dichloromethane (25 mL) and m-chloroperoxybenzoic acid (0.20 g, 0.99 mmol, 85 mass%) were added to a 100 mL flask, and the mixture was stirred overnight at room temperature. The reaction was quenched with 25 mL of saturated sodium sulfite, then stirred for 15 min, and 25 mL of saturated sodium carbonate was added, after extraction with chloroform (3 × 25 mL), the combined organic phase was dried over anhydrous sodium sulfate, filtered, and concentrated under reduced pressure. The resulting residue was purified by column chromatography (dichloromethane/methanol (v/v) = 95/5) to obtain the title compound as a white solid (0.12 g, yield 70.0%).

MS (ESI, pos. ion) m/z: 258.2 [M+H]⁺.

### Step 4 Synthesis of 2′-Chloro-4′-(Difluoromethyl)-4,5,5′,6′-Tetrahydro-2H-Spiro[Furan-3, 8′-Pyrano[3,4-b]Pyridine]

4′-(Difluoromethyl)-4,5,5′,6′-tetrahydro-2H-spiro[furan-3,8′-pyrano[3,4-b]pyridine]-1′ -oxide (0.11 g, 0.43 mmol) and phosphine oxychloride (15 mL, 165 mmol) were added to a 50 mL round bottom flask, and the mixture was reacted under reflux for 1 h. After TLC monitoring showed that the reaction was completed, the mixture was concentrated in vacuum, then 12 mL of saturated sodium bicarbonate was added, and the pH was adjusted to 7 by saturated sodium carbonate, after extraction with ethyl acetate (3 × 20 mL), the combined organic phase was washed with 20 mL of saturated sodium chloride, then dried and filtered, concentrated under reduced pressure, and the resulting residue was purified by column chromatography (dichloromethane/methanol (v/v) = 97/3) to obtain the title compound as a yellow-white solid (0.084 g, yield 71.3%).

MS (ESI, pos. ion) m/z: 276.2 [M+H]⁺;

¹H NMR (400 MHz, DMSO-d₆): δ (ppm) 7.56 (s, 1H), 7.21 (t, J = 53.6 Hz, 1H), 4.02 - 3.88 (m, 6H), 2.90 (s, 2H), 2.41 - 2.33 (m, 1H), 2.29 - 2.22 (m, 1H).

### Step 5 Synthesis of N-(3-(4′-(Difluoromethyl)-4,5,5′,6′-Tetrahydro-2H-Spiro[Furan-3,8′-P yrano[3,4-b]Pyridin]-2′-yl)-1-Methyl-1H-Pyrrolo[2,3-c]Pyridin-5-yl)Acetamide

Under N₂, 2′-chloro-4′-(difluoromethyl)-4,5,5′,6′-tetrahydro-2H-spiro[furan-3,8′-pyrano[3,4-b]pyridine] (70 mg, 0.25 mmol), N-[1-methyl-3-(4,4,5,5-tetramethyl-1,3,2-dioxaborolan-2-yl)-1H-pyrrolo[2,3-c]pyridin-5-yl]acetami de (94 mg, 0.30 mmol), 1,4-dioxane (8 mL), water (2 mL) and potassium carbonate (70 mg, 0.51 mmol) were added to a 50 mL two-necked round bottom flask, the mixture was deoxygenated by bubbling N₂ for 10 min, then PdCl₂dppf (62 mg, 0.076 mmol) was added and deoxygenated by bubbling N₂ for 10 min. The flask was connected to a reflux condenser, and the reaction was refluxed overnight under N₂, then 10 mL of water was added to quench the reaction, after extraction with chloroform (3 × 10 mL), the combined organic phase was dried over anhydrous sodium sulfate, and the resulting residue was purified by column chromatography (dichloromethane/methanol (v /v) = 91/9) to obtain the title compound as a yellow solid (26 mg, yield 24.1%).

MS (ESI, pos. ion) m/z: 429.2 [M+H]⁺;

¹H NMR (400 MHz, DMSO-d₆): δ (ppm) 10.22 (s, 1H), 9.02 (s, 1H), 8.63 (s, 1H), 8.40 (s, 1H), 7.77 (s, 1H), 7.22 (s, 1H), 4.11 - 3.87 (m, 9H), 2.89 (s, 2H), 2.25 (s, 1H), 2.23 - 2.03 (m, 4H).

### Example 8 N-(3-(4′-(Methoxymethyl)-4,5,5′,6′-Tetrahydro-2H-Spiro[Furan-3,8′-Pyrano[3, 4-b]Pyridin]-2′-yl)-1-Methyl-1H-Pyrrolo[2,3-c]Pyridin-5-yl)Acetamide

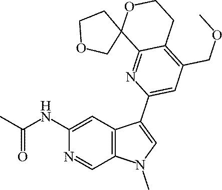 [SMILES: COCc1cc(-c2cn(C)c3cnc(NC(C)=O)cc23)nc2c1CCOC21CCOC1]

### Step 1 Synthesis of 4,5,5′,6′-Tetrahydro-2H-Spiro[Furan-3,8′-Pyrano[3,4-b]Pyridine]-4′-C arbonitrile

4′-Chloro-4,5,5′,6′-tetrahydro-2H-spiro[furan-3,8′-pyrano[3,4-b]pyridine] (0.90 g, 3.99 mmol), zinc cyanide (1.41 g, 11.96 mmol), DMF (15 mL), tri-tert-butylphosphine (2.42 g, 1.19 mmol, 10% in n-hexane), bis(tri-tert-butylphosphine)palladium (1.02 g, 1.99 mmol) were added to a 50 mL two-neck round bottom flask, the mixture was deoxygenated by bubbling N₂ for 10 min. The flask was connected to a reflux condenser, the mixture was reacted overnight at 120° C., then the reaction was quenched by 10 mL of water, after extraction with chloroform (3 × 10 mL), the combined organic phase was dried over anhydrous sodium sulfate, and the resulting residue was purified by column chromatography (petroleum ether/ethyl acetate (v/v) = 9/1) to obtain the title compound as yellow oily liquid (0.86 g, yield 100%).

MS (ESI, pos. ion) m/z: 217.2 [M+H]⁺;

### Step 2 Synthesis of 4,5,5′,6′-Tetrahydro-2H-Spiro[Furan-3,8′-Pyrano[3,4-b]Pyridine]-4′-C arboxylic Acid

4,5,5′,6′-Tetrahydro-2H-spiro[furan-3,8′-pyrano[3,4-b]pyridine]-4′-carbonitrile (0.90 g, 4.16 mmol) and concentrated hydrochloric acid (5 mL) were added to a 50 mL two-neck round bottom flask, then the flask was connected to a reflux condenser, the mixture was refluxed overnight, then the solvent was concentrated, then dichloromethane (20 mL) and 10 mL of saturated aqueous sodium bicarbonate were added, and separated, then the aqueous phase was taken. The pH of aqueous phase was adjusted to 1 with concentrated hydrochloric acid, after extraction with EtOAc (5 × 100 mL), the combined organic phase was washed with saturated brine (3 × 100 mL), dried over anhydrous sodium sulfate, and the solvent was concentrated to obtain the title compound as a white solid (0.58 g, yield 59.0%).

MS (ESI, pos. ion) m/z: 236.2 [M+H]⁺;

### Step 3 Synthesis of (4,5,5′,6′-Tetrahydro-2H-Spiro[Furan-3,8′-Pyrano[3,4-b]Pyridin]-4′-y 1)Methanol

4,5,5′,6′-Tetrahydro-2H-spiro[furan-3,8′-pyrano[3,4-b]pyridine]-4′-carboxylic acid (0.38 g, 1.62 mmol) and anhydrous THF (100 mL) were added to a 50 mL round bottom flask under N₂, then the mixture was cooled to 0° C. Lithium aluminum hydride (0.18 g, 4.85 mmol) was added and stirring was continued at 0° C. for 2 h. The reaction was quenched by adding 20 mL of saturated aqueous ammonium chloride, extracted with ethyl acetate (3 × 20 mL), dried over anhydrous sodium sulfate, filtered, and concentrated under reduced pressure, and the resulting residue was purified by column chromatography (dichloromethane/methanol (v/v)=97/3) to obtain the title compound as colorless oily liquid (0.27 g, yield 75.7%).

MS (ESI, pos. ion) m/z: 222.2 [M+H]⁺.

### Step 4 Synthesis of 4′-(Methoxymethyl)-4,5,5′,6′-Tetrahydro-2H-Spiro[Furan-3,8′-Pyran o[3,4-b]Pyridine]

(4,5,5′,6′-Tetrahydro-2H-spiro[furan-3,8′-pyrano[3,4-b]pyridin]-4′-yl)methanol (0.25 g, 1.13 mmol) and anhydrous THF (10 mL) were added to a 50 mL round bottom flask under N₂, then the mixture was cooled to 0° C. NaH (0.090 g, 2.26 mmol, 60%) was added, and the mixture was reacted at room temperature for 30 min. Then Iodomethane (0.32 g, 2.26 mmol) was added, and the mixture was reacted at room temperature. The reaction was quenched by adding 10 mL of water, extracted with ethyl acetate (3 × 20 mL), dried over anhydrous sodium sulfate, filtered, and concentrated under reduced pressure, and the resulting residue was purified by column chromatography (dichloromethane/methanol (v/v) = 97/3) to obtain the title compound as yellow oily liquid (0.18 g, yield 69.8%).

MS (ESI, pos. ion) m/z: 236.2 [M+H]⁺;

¹H NMR (400 MHz, CDCl₃): δ (ppm) 8.49 (d, J = 4.8 Hz, 1H), 7.22 (d, J = 4.9 Hz, 1H), 4.44 (s, 2H), 4.36 - 4.16 (m, 4H), 4.00 (dd, J = 11.2, 5.9 Hz, 2H), 3.48 (s, 3H), 2.81 (dd, J = 9.6, 5.3 Hz, 2H), 2.64 (dd, J = 8.7, 4.2 Hz, 1H), 2.36 - 2.27 (m, 1H).

### Step 5 Synthesis of 4′-(Methoxymethyl)-4,5,5′,6′-Tetrahydro-2H-Spiro[Furan-3,8′-Pyran 0[3,4-b]Pyridine] 1′-Oxide

4′-(Methoxymethyl)-4,5,5′,6′-tetrahydro-2H-spiro[furan-3,8′-pyrano[3,4-b]pyridine] (0.18 g, 0.76 mmol), dichloromethane (15 mL) and m-chloroperoxybenzoic acid (0.23 g, 1.15 mmol, 85 mass%) were added to a 100 mL flask, and the mixture was stirred overnight at room temperature. The reaction was quenched with 25 mL of saturated sodium sulfite, then stirred for 15 min, and 25 mL of saturated sodium carbonate was added, after extraction with chloroform (3 × 25 mL), the combined organic phase was dried over anhydrous sodium sulfate, filtered, and concentrated under reduced pressure, the resulting residue was purified by column chromatography (dichloromethane/methanol (v/v) = 95/5) to obtain the title compound as a white solid (0.18 g, yield 94.9%).

MS (ESI, pos. ion) m/z: 252.2 [M+H]⁺.

### Step 6 Synthesis of 2′-Chloro-4′-(Methoxymethyl)-4,5,5′,6′-Tetrahydro-2H-Spiro[Furan-3,8′-Pyrano[3,4-b]Pyridine]

4′-(Methoxymethyl)-4,5,5′,6′-tetrahydro-2H-spiro[furan-3,8′-pyrano[3,4-b]pyridine] 1′-oxide (0.18 g, 0.72 mmol) and phosphine oxychloride (15 mL, 165 mmol) were added to a 50 mL round bottom flask, and the mixture was refluxed for 1 h. The solution was concentrated in vacuum, then 12 mL of saturated sodium bicarbonate was added, the pH was adjusted to 7 with saturated sodium carbonate, after extraction with ethyl acetate (3 × 20 mL), the combined organic phase was washed with 20 mL of saturated sodium chloride, then dried and filtered, and concentrated under reduced pressure, the resulting residue was purified by column chromatography (dichloromethane/methanol (v/v) = 97/3) to obtain the title compound as a yellow-white solid (0.095 g, yield 49.4%).

MS (ESI, pos. ion) m/z: 270.2 [M+H]⁺;

### Step 7 Synthesis of N-(3-(4′-(Methoxymethyl)-4,5,5′,6′-Tetrahydro-2H-Spiro[Furan-3,8′-Pyrano[3,4-b]Pyridin]-2′-yl)-1-Methyl-1H-Pyrrolo[2,3-c]Pyridin-5-yl)Acetamide

Under N₂, 2′-chloro-4′-(methoxymethyl)-4,5,5′,6′-tetrahydro-2H-spiro[furan-3,8′-pyrano[3,4-b]pyridine] (50 mg, 0.19 mmol), N [1-methyl-3-(4,4,5,5-tetramethyl-1,3,2-dioxaborolan-2-yl)-1H-pyrrolo[2,3-c]pyridin-5-yl]acetami de (70 mg, 0.22 mmol), 1,4-dioxane (8 mL), water (2 mL) and potassium carbonate (51 mg, 0.37 mmol) were added to a 50 mL two-necked round bottom flask, the mixture was deoxygenated by bubbling N₂ for 10 min, then PdCl₂dppf (45 mg, 0.06 mmol) was added and deoxygenated by bubbling N₂ for 10 min. The flask was connected to a reflux condenser, and the mixture was refluxed overnight under N₂, then 10 mL of water was added to quench the reaction, after extraction with chloroform (3 × 10 mL), the combined organic phase was dried over anhydrous sodium sulfate, and the resulting residue was purified by column chromatography (dichloromethane/methanol (v /v) = 91/9) to obtain the title compound as a yellow solid (40 mg, yield 51.1%).

MS (ESI, pos. ion) m/z: 423.2 [M+H]⁺;

¹H NMR (400 MHz, DMSO-d₆): δ (ppm) 10.19 (s, 1H), 9.03 (s, 1H), 8.62 (s, 1H), 8.26 (s, 1H), 7.59 (s, 1H), 4.48 (s, 2H), 4.02 (dd, J = 18.3, 8.4 Hz, 4H), 3.90 (d, J = 19.6 Hz, 5H), 3.42 (s, 3H), 3.33 (s, 2H), 2.73 (d, J = 14.9 Hz, 1H), 2.22 (d, J = 5.5 Hz, 1H), 2.10 (s, 3H).

### Example 9 N-(1-Methyl-3-(1′-Methyl-1′,2′,4,5-Tetrahydro-2Hspiro[Furan-3,4′-Pyrido[3,2 -d][1,3]Oxazin]-6′-yl)-1H-Pyrrolo[2,3-c]Pyridin-5-yl)Acetamide

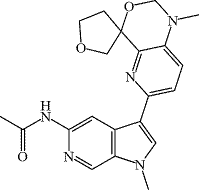 [SMILES: CC(=O)Nc1cc2c(-c3ccc4c(n3)C3(CCOC3)OCN4C)cn(C)c2cn1]

### Step 1 Synthesis of 2-Bromo-6-Chloro-N-Methylpyridin-3-Amine

2-Bromo-6-chloropyridin-3-amine (3.0 g, 14.4 mmol) and tetrahydrofuran (80 mL) were added to a 250 mL single-necked flask, then the mixture was cooled to 0° C., and then a solution of sodium bis(trimethylsilyl)amide (29 mL, 58.0 mmol, 2 mol/L) in tetrahydrofuran was added slowly, the resulting solution was stirred for 30 min, and potassium iodide (2.4 g, 16.9 mmol) was added dropwise, then the mixture was reacted at room temperature for 2 h. After the reaction was completed, 10 mL of methanol was added to quench the reaction, and extracted with ethyl acetate (3 × 20 mL), concentrated under reduced pressure, and the resulting residue was purified by column chromatography (petroleum ether/ethyl acetate (v/v) = 10/1) to obtain the title compound as red oily liquid (1.2 g, yield 36%).

MS (ESI, pos. ion) m/z: 221.0 [M+H]⁺.

### Step 2 Synthesis of 3-[6-Chloro-3-(Methylamino)Pyridin-2-yl]Tetrahydrofuran-3-Ol

2-Bromo-6-chloro-N-methylpyridin-3-amine (1.2 g, 5.4 mmol) and anhydrous tetrahydrofuran (20 mL) were added to a 100 mL single-necked flask in sequence, and then the solution was cooled to - 78° C., a solution of n-butyl lithium in n-hexane (4.8 mL, 12 mmol, 2.5 mol / L) was added slowly and dropwise, and continued to stir for 1 h, then tetrahydrofuran-3-one (0.56 g, 6.5 mmol) was added dropwise, and continued to stir for 2 h. The mixture was reacted at room temperature and stirred for 30 min, then 10 mL of saturated ammonium chloride was added to quench the reaction, then the mixture was extracted with ethyl acetate (3 × 20 mL), concentrated under reduced pressure, and the resulting residue was purified by column chromatography (petroleum ether/ethyl acetate (v/v)=1:1) to obtain the title compound as yellow oily liquid (0.5 g, yield 40%).

MS (ESI, pos. ion) m/z: 229.1 [M+H]⁺.

### Step 3 Synthesis of 6′-Chloro-1′-Methyl-1′,2′,4,5-Tetrahydro-2H-Spiro[Furan-3,4′-Pyrido[ 3,2-d][1,3]Oxazine]

3-[6-Chloro-3-(methylamino)pyridin-2-yl]tetrahydrofuran-3-ol (150 mg, 0.65 mmol), acetic acid (5 mL) and 40% formaldehyde solution (539 mg, 6.64 mmol) were added to a 10 mL single-necked bottle successively, the mixture was heated to 80° C. for reaction, after the reaction was completed, the solution was adjusted to basicity, then extracted with ethyl acetate (3 × 5 mL), the combined organic phase was concentrated under reduced pressure, and the resulting residue was purified by column chromatography (petroleum ether/ethyl acetate (v/v)=5/1) to obtain the title compound as a white solid (100 mg, yield 63%).

MS (ESI, pos. ion) m/z: 241.1 [M+H]⁺;

¹H NMR (400 MHz, CDCl₃) δ 7.09 (d, J = 8.6 Hz, 1H), 7.00 (d, J = 8.6 Hz, 1H), 4.63 - 4.51 (m, 2H), 4.20 - 4.00 (m, 4H), 2.89 (s, 3H), 2.67 (dt, J = 13.0, 8.8 Hz, 1H), 2.30 - 2.17 (m, 1H).

### Step 4 Synthesis of N-(1-Methyl-3-(1′-Methyl-1′,2′,4,5-Tetrahydro-2H-Spiro[Furan-3,4′-P yrido[3,2-d][1,3]Oxazin]-6′-yl)-1H-Pyrrolo[2,3-c]Pyridin-5-yl)Acetamide

6′-Chloro-1′-methyl-1′,2′,4,5-tetrahydro-2H-spiro[furan-3,4′-pyrido[3, 2-d][1,3]oxazine] (70 mg, 0.29 mmol), N-[1-methyl-3-(4,4,5,5-tetramethyl-1,3,2-dioxaborolan-2-yl)-1H-pyrrolo[2,3-c]pyridin-5-yl]acetami de (138 mg, 0.44 mmol), potassium phosphate (189 mg, 0.87 mmol), 2-dicyclohexylphosphine 2′, 4′, 6′-triisopropylbiphenyl (83 mg, 0.17 mmol), tris(benzhydrylethyleneacetone) dipalladium (80 mg, 0.08 mmol), 1,4-dioxane (6 mL) and water (0.5 mL) were added to a 25 mL two-necked bottle successively, the mixture was reacted at 100° C. for 3 h under N₂. After the reaction was completed, the solvent was concentrated, and the resulting residue was purified by column chromatography (dichloromethane/methanol (v/v) = 30:1) to obtain the title compound as a white solid (45 mg, yield 39%).

MS (ESI, pos. ion) m/z: 394.2 [M+H]⁺;

¹H NMR (400 MHz, CDCl₃) δ 9.00 (s, 1H), 8.41 (s, 1H), 8.28 (s, 1H), 7.70 (s, 1H), 7.51 (d, J = 8.5 Hz, 1H), 7.13 (d, J = 8.5 Hz, 1H), 4.72 - 4.56 (m, 2H), 4.38 - 4.29 (m, 2H), 4.24 (dd, J = 15.4, 8.0 Hz, 1H), 4.17 (d, J = 9.4 Hz, 1H), 3.92 (s, 3H), 3.01 - 2.95 (m, 1H), 2.94 (s, 3H), 2.40 - 2.30 (m, 1H), 2.25 (s, 3H).

### Example 10 N-(1-Methyl-3-(1′-Methyl-2′-Oxo-1′,2′,4,5-Tetrahydro-2H-Spiro[Furan-3,4′-Py rido[3,2-d][1,3]Oxazin]-6′-yl)-1H-Pyrrolo[2,3-c]Pyridin-5-yl)Acetamide

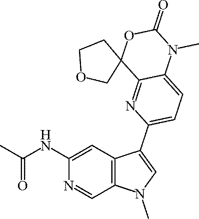 [SMILES: CC(=O)Nc1cc2c(-c3ccc4c(n3)C3(CCOC3)OC(=O)N4C)cn(C)c2cn1]

### Step 1 Synthesis of 6′-Chloro-1′-Methyl-4,5-Dihydro-2H-Spiro[Furan-3,4′-Pyrido[3,2-d][1,3]Oxazine]-2′(1′H)-One

3-[6-Chloro-3-(methylamino)pyridin-2-yl]tetrahydrofuran-3-ol (150 mg, 0.65 mmol), dichloromethane (10 mL) and DMF (20 mg) were added in sequence to a 25 mL single-necked bottle, and the mixture was cooled to 0° C., then triphosgene (77 mg, 0.26 mmol) dissolved in dichloromethane (2 mL) was slowly added, after the reaction was completed, the solution was adjusted to alkaline with 50 mL of saturated sodium carbonate solution, after extraction with ethyl acetate (3 × 20 mL), the organic phases were combined, and the solvent was concentrated, and the resulting residue was purified by column chromatography (petroleum ether/ethyl acetate (v/v) = 5/1) to obtain the title compound as a white solid (130 mg, yield 77.8%).

MS (ESI, pos. ion) m/z: 255.1 [M+H]⁺.

### Step 2 Synthesis of N-(1-Methyl-3-(1′-Methyl-2′-Oxo-1′,2′,4,5-Tetrahydro-2H-Spiro[Fura n-3,4′-Pyrido[ 3,2-d][1,3]Oxazin]-6′-yl)-1H-Pyrrolo[2,3-c]Pyridin-5-yl)Acetamide

6′-Chloro-1′-methyl-4,5-dihydro-2H-spiro[furan-3,4′-pyrido[3,2-d][1,3]oxazin]-2′(1′H )-one (50 mg, 0.19 mmol), N [1-methyl-3-(4,4,5,5-tetramethyl-1,3,2-dioxaborolan-2-yl)-1H-pyrrolo[2,3-c]pyridin-5-yl]acetami de (90 mg, 0.29 mmol), potassium carbonate (54 mg, 0.39 mmol), PdCl₂dppf (16 mg, 0.02 mmol), 1,4-dioxane (5 mL) and water (2 mL) were added in sequence to a 25 mL two-necked bottle, the mixture was reacted at 100° C. overnight under N₂. After the reaction was completed, the solvent was concentrated, and the resulting residue was purified by column chromatography (dichloromethane/methanol (v/v) = 30/1) to obtain the title compound as a white solid (36 mg, yield 45%).

MS (ESI, pos. ion) m/z: 408.1 [M+H]⁺;

¹H NMR (400 MHz, DMSO-d₆) δ 10.24 (s, 1H), 9.08 (s, 1H), 8.62 (s, 1H), 8.28 (s, 1H), 7.78 (d, J = 8.6 Hz, 1H), 7.58 (d, J = 8.7 Hz, 1H), 4.19 (dd, J = 11.7, 5.6 Hz, 2H), 4.16 - 4.09 (m, 1H), 4.01 (d, J = 10.2 Hz, 1H), 3.93 (s, 3H), 3.32 (s, 3H), 3.15 (dt, J = 16.1, 8.2 Hz, 1H), 2.46 -2.37 (m, 1H), 2.10 (s, 3H).

### Example 11 N-(3-(5′,6′-Dihydro-2′H,4′H,5H-Spiro[Furo[3,4-b]Pyridin-7,3′-Pyran]-2-yl)-1-Methyl-1H-Pyrrolo[2,3-c]Pyridin-5-yl)Acetamide

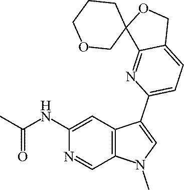 [SMILES: CC(=O)Nc1cc2c(-c3ccc4c(n3)C3(CCCOC3)OC4)cn(C)c2cn1]

### Step 1 Synthesis of 3-(3-(Hydroxymethyl)Pyridin-2-yl)Tetrahydro-2H-Pyran-3-ol

(2-Bromopyridin-3-yl)methanol (8.00 g, 42.54 mmol) was added to a 250 mL round bottom flask, then anhydrous THF (100 mL) was added under N₂, and the mixture was cooled to -78° C. A solution of n-butyllithium (37.0 mL, 93.60 mmol, 2.5 mol/L) in n-hexane was added to the flask dropwise, and the addition was finished in 30 min, the mixture was stirred at -78° C. for 1 h, then tetrahydropyran-3-one (4.73 mL, 51.05 mmol) was added, the mixture was kept warm and stirred for 30 min, and then continued stirring at room temperature for 90 min. The reaction was quenched by 20 mL of saturated aqueous ammonium chloride, extracted with ethyl acetate (3 × 20 mL), dried over anhydrous sodium sulfate, filtered, and concentrated under reduced pressure, the resulting residue was purified by column chromatography (petroleum ether/ethyl acetate (v/v)=1:3) to obtain the title compound as viscous colorless oily liquid (2.30 g, yield 25.8%).

MS (ESI, pos. ion) m/z: 210.2 [M+H]⁺.

### Step 2 Synthesis of 5′,6′-Dihydro-2′H,4′H,5H- Spiro[Furo[3,4-b]Pyridine-7,3′-Pyran]

3-(3-(Hydroxymethyl)pyridin-2-yl)tetrahydro-2H-pyran-3-ol (2.30 g, 11.0 mmol) was added to a 100 mL round bottom flask, and anhydrous THF (50 mL) was added under N₂, the mixture was cooled to 0° C. NaHMDS (12.1 mL, 24.2 mmol, 2 mol/L, THF solution) was added dropwise, and the mixture was stirred at 0° C. for 10 min after the addition. Then p-toluenesulfonyl chloride (2.51 g, 13.2 mmol) was added and the mixture was continued stirring at 0° C. for 1 h. 10 mL of saturated aqueous ammonium chloride was added to quench the reaction, after extraction with ethyl acetate (3 × 10 mL), the combined organic phase was washed with 10 mL of saturated sodium chloride. The mixture was filtered, and concentrated under reduced pressure, and the resulting residue was purified by column chromatography (dichloromethane/methanol (v/v) = 95/5) to obtain the title compound as yellow oily liquid (1.15 g, yield 54.7%).

MS (ESI, pos. ion) m/z: 192.2 [M+H]⁺;

¹H NMR (400 MHz, CDCl₃): δ (ppm) 8.49 (d, J = 4.7 Hz, 1H), 7.58 (d, J = 7.6 Hz, 1H), 7.21 (dd, J = 7.6, 4.9 Hz, 1H), 5.15 (d, J = 1.0 Hz, 2H), 4.09 - 4.01 (m, 1H), 3.80 - 3.74 (m, 2H), 3.67 - 3.57 (m, 1H), 2.14 (dd, J = 9.0, 2.9 Hz, 2H), 1.97 - 1.89 (m, 1H), 1.74 - 1.68 (m, 1H).

### Step 3 Synthesis of 5′,6′-Dihydro-2′H,4′H,5H-Spiro[Furo[3,4-b]Pyridine-7,3′-Pyran]1-Oxi de

5′,6′-Dihydro-2′H,4′H,5H-spiro[furo[3,4-b]pyridine-7,3′-pyran] (1.15 g, 6.01 mmol), dichloromethane (25 mL) and m-chloroperoxybenzoic acid (1.84 g, 9.02 mmol, 85 mass%) were added to a 100 mL flask, and the mixture was stirred overnight at room temperature. The reaction was quenched with 25 mL of saturated sodium sulfite, stirred for 15 min, then 25 mL of saturated sodium carbonate was added, after extraction with chloroform (3 × 25 mL), the combined organic phase was dried over anhydrous sodium sulfate, filtered and concentrated under reduced pressure, the resulting residue was purified by column chromatography (dichloromethane/methanol (v/v) = 95/5) to obtain the title compound as a white solid (1.00 g, yield 80.2%).

MS (ESI, pos. ion) m/z: 208.2 [M+H]⁺;

¹H NMR (400 MHz, CDCl₃): δ (ppm) 8.07 (d, J = 6.3 Hz, 1H), 7.26 - 7.20 (m, 1H), 7.13 (d, J = 7.6 Hz, 1H), 5.21 - 5.11 (m, 2H), 4.45 (d, J = 11.7 Hz, 1H), 4.04 (dd, J = 11.2, 4.5 Hz, 1H), 3.79 (dd, J = 11.6, 2.5 Hz, 1H), 3.68 - 3.60 (m, 1H), 2.99 - 2.85 (m, 1H), 2.34 - 2.04 (m, 1H), 1.89 - 1.74 (m, 2H).

### Step 4 Synthesis of 2-Chloro-5′,6′-Dihydro-2′H,4′H,5H-Spiro[Furo[3,4-b]Pyridine-7,3′-Py ran]

2′,4′,5′,6′-Tetrahydro-5H-spiro[furo[3,4-b]pyridine-7,3′-pyran]-1-oxide (0.90 g, 4.34 mmol) and phosphine oxychloride (15 mL, 165 mmol) were added to a 50 mL round bottom flask, and the mixture was refluxed for 1 h. After TLC monitoring showed that the reaction was completed, the resulting solution was concentrated in vacuum, then 12 mL of saturated sodium bicarbonate was added, and the pH was adjusted to 7 by saturated sodium carbonate, after extraction with ethyl acetate (3 × 20 mL), the combined organic phase was washed with 20 mL of saturated sodium chloride, then dried and filtered, and concentrated under reduced pressure, and the resulting residue was purified by column chromatography (dichloromethane/methanol (v/v)=97:3) to obtain the title compound as a yellow-white solid (130 mg, yield 13.0%).

MS (ESI, pos. ion) m/z: 226.2 [M+H]⁺;

¹H NMR (400 MHz, CDCl₃): δ (ppm) 7.54 (d, J = 8.0 Hz, 1H), 7.26 (d, J = 8.0 Hz, 1H), 5.13 (d, J = 2.6 Hz, 2H), 4.04 (dd, J = 7.4, 5.7 Hz, 1H), 3.80 - 3.75 (m, 2H), 3.60 (dd, J = 16.2, 6.8 Hz, 1H), 2.18 - 2.09 (m, 2H), 1.96 - 1.89 (m, 1H), 1.69 (dd, J = 9.2, 2.4 Hz, 1H).

### Step 5 Synthesis of N-(3-(5′,6′-Dihydro-2′H,4′H,5H-Spiro[Furo[3,4-b]Pyridin-7,3′-Pyran] -2-yl)-1-Methyl-1H-Pyrrolo[2,3-c]Pyridin-5-yl)Acetamide

Under N₂, 2-chloro-5′,6′-dihydro-2′H,4′H,SH-spiro[furo[3,4-b]pyridine-7,3′-pyran] (50 mg, 0.22 mmol), N-[1-methyl-3-(4,4,5,5-tetramethyl-1,3,2-dioxaborolan-2-yl)-1H-pyrrolo[2,3-c]pyridin-5-yl]acetami de (0.35 g, 0.33 mmol, 30%), 1,4-dioxane (8 mL), water (2 ml) and potassium carbonate (61 mg, 0.44 mmol) were added to a 50 mL two-necked round bottom flask, the mixture was deoxygenated by bubbling N₂ for 10 min, then PdCl₂dppf (54 mg, 0.066 mmol) was added and the mixture was deoxygenated by bubbling N₂ for 10 min. The flask was connected to a reflux condenser, the mixture was refluxed overnight under N₂, then 10 mL of water was added to quench the reaction, after extraction with chloroform (3 × 10 mL), the combined organic phase was dried over anhydrous sodium sulfate, concentrated under reduced pressure, and the resulting residue was purified by column chromatography (ethyl acetate/methanol (v/v) = 91/9) to obtain the title compound as a yellow solid (55 mg, yield 65.6%).

MS (ESI, pos. ion) m/z: 379.2 [M+H]⁺;

¹H NMR (400 MHz, DMSO-d₆): δ (ppm) 10.22 (s, 1H), 9.28 (s, 1H), 8.62 (s, 1H), 8.26 (s, 1H), 7.73 (d, J = 8.0 Hz, 1H), 7.64 (d, J = 8.0 Hz, 1H), 5.04 (s, 2H), 3.94 (s, 4H), 3.81 (d, J = 11.7 Hz, 1H), 3.69 (d, J = 11.6 Hz, 1H), 3.60 (t, J = 10.9 Hz, 1H), 2.23 (td, J = 13.2, 4.4 Hz, 1H), 2.12 (s, 3H), 1.96 (dd, J = 21.5, 8.8 Hz, 1H), 1.82 (d, J = 11.7 Hz, 1H), 1.64 (d, J = 12.6 Hz, 1H).

### Example 12 N-(3-(5′,6′-Dihydro-2′H,4′H,5H-Spiro[Furo[3,4-b]Pyridin-7,3′-Pyran]-2-yl)-1-Methyl-1H-Pyrrolo[2,3-c]Pyridin-5-yl)Acetamide

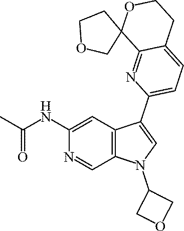 [SMILES: CC(=O)Nc1cc2c(-c3ccc4c(n3)C3(CCOC3)OCC4)cn(C3COC3)c2cn1]

### Step 1 Synthesis of N-(1-(Oxetan-3-yl)-1H-Pyrrolo[2,3-c]Pyridin-5-yl)Acetamide

N-(1H-pyrrolo[2,3-c]pyridin-5-yl)acetamide (2.00 g, 11.41 mmol) and DMF (15 mL) were added to a 100 mL round bottom flask, the mixture was stirred to dissolve, then cesium carbonate (7.42 g, 22.83 mmol) and 3-iodobutylene oxide (3.15 g, 17.12 mmol) were added, the mixture was heated to 100° C. and reacted for 2 h. The resulting solution was concentrated in vacuum, and the resulting residue was purified by column chromatography (dichloromethane/methanol (v/v) = 97/3) to obtain the title compound as a white solid (1.05 g, yield 39.8%).

MS (ESI, pos. ion) m/z: 232.2 [M+H]⁺;

¹H NMR (400 MHz, DMSO-d₆): δ (ppm) 10.21 (s, 1H), 8.68 (s, 1H), 8.25 (s, 1H), 7.95 (d, J = 3.1 Hz, 1H), 6.58 (d, J = 3.0 Hz, 1H), 5.87 - 5.78 (m, 1H), 5.06 (t, J = 7.3 Hz, 2H), 4.94 (t, J = 6.6 Hz, 2H), 2.08 (s, 3H).

### Step 2 Synthesis of N (3-Bromo-1-(Oxetan-3-yl)-1H Pyrrolo[2,3-c]Pyridin-5-yl)Acetamid e

N-(1-(oxetan-3-yl)-1H-pyrrolo[2,3-c]pyridin-5-yl)acetamide (0.95 g, 4.11 mmol), DMF (15 mL), and NBS (0.77 g, 4.31 mmol) were added to a 50 mL round bottom flask, and the mixture was stirred overnight at room temperature. Then 1 mL of water was added, the solution was concentrated under reduced pressure, and the obtained residue was purified by column chromatography (dichloromethane/methanol (v/v) = 95/5) to obtain the title compound as a red solid (1.17 g, yield 91.8%).

MS (ESI, pos. ion) m/z: 310.2 [M+H]⁺;

¹H NMR (400 MHz, DMSO-d₆): δ (ppm) 10.38 (s, 1H), 8.73 (s, 1H), 8.23 (s, 1H), 8.16 (s, 1H), 5.90 - 5.81 (m, 1H), 5.03 (t, J = 7.3 Hz, 2H), 4.95 (t, J = 6.7 Hz, 2H), 2.10 (s, 3H).

### Step 3 Synthesis of N-(1-(Oxetan-3-yl)-3-(4,4,5,5-Tetramethyl-1,3,2-Dioxaborolan-2-yl)-1 H-Pyrrolo[2,3-c]Pyridin-5-yl)Acetamide

Anhydrous THF (40 mL) was added to a 100 mL round bottom flask and then cooled to -78° C. A solution of n-butyllithium (3.6 mL, 9.03 mmol, 2.5 mol/L) in n-hexane was added, and the mixture was stirred for 10 min after the addition. Then N-(3-bromo-1-(oxetan-3-yl)-1H-pyrrolo[2,3-c]pyridin-5-yl)acetamide (0.70 g, 2.26 mmol) was added and the stirring was continued for 30 min. Then isopropanol pinacol borate (2.10 g, 11.28 mmol) was added and the stirring was continued for 1 h after the addition. Then the resulting solution was warmed to room temperature and stirred for 30 min. The reaction was quenched by adding 20 mL of saturated ammonium chloride, extracted with ethyl acetate (3 × 20 mL), dried over anhydrous sodium sulfate, filtered and concentrated under reduced pressure, and the resulting residue was purified by column chromatography (dichloromethane/methanol (v/v) = 95/5) to obtain the title compound as a yellow solid (0.070 g, yield 30.4%).

MS (ESI, pos. ion) m/z: 358.2 [M+H]⁺.

### Step 4 Synthesis of N-(1-(Oxetane-3-yl)-3-(4,5,5′,6′-Tetrahydro-2H-Spiro[Furan-3,8′-Pyra no[3,4-b]Pyridin]-2′-yl)-1H-Pyrrolo[2,3-c]Pyridin-5-yl)Acetamide

Under N₂, N (1-(oxetan-3-yl)-3-(4,4,5,5-tetramethyl-1,3,2-dioxaborolan-2-yl)-1H-pyrrolo[2,3-c]pyridin-5-yl)a cetamide (140 mg, 0.20 mmol, 50%), 2′-chloro-4,5,5′,6′-tetrahydro-2H-spiro[furan-3,8′-pyrano[3,4-b]pyridine] (30 mg, 0.13 mmol), 1,4-dioxane (8 mL), water (2 mL) and potassium carbonate (36 mg, 0.27 mmol) were added to a 50 mL two-necked round bottom flask, the mixture was deoxygenated by bubbling N₂ for 10 min, then PdCl₂dppf (32 mg, 0.039 mmol) was added and the mixture was deoxygenated by bubbling N₂ for 10 min. The flask was connected to a reflux condenser, the mixture was refluxed overnight under N₂, then 10 mL of water was added to quench the reaction, after extraction with chloroform (3 × 10 mL), the combined organic phase was dried over anhydrous sodium sulfate, and the resulting residue was purified by column chromatography (ethyl acetate/methanol (v/v) = 91/9) to obtain the title compound as a yellow solid (15 mg, yield 26.8%).

MS (ESI, pos. ion) m/z: 421.2 [M+H]⁺;

¹H NMR (400 MHz, DMSO-d₆): δ (ppm) 10.24 (s, 1H), 9.09 (s, 1H), 8.70 (d, J = 10.8 Hz, 2H), 7.74 (d, J = 8.1 Hz, 1H), 7.58 (d, J = 8.1 Hz, 1H), 5.94 - 5.84 (m, 1H), 5.09 (t, J = 7.2 Hz, 2H), 5.06 - 4.99 (m, 2H), 4.29 - 4.21 (m, 1H), 4.12 - 4.02 (m, 3H), 3.95 - 3.88 (m, 2H), 2.99 -2.84 (m, 2H), 2.10 (s, 3H), 1.30 - 1.22 (m, 2H).

### Example 13 N-(1-Methyl-3-(1′,2′,2′-Trimethyl-1′,2′,4,5-Tetrahydro-2H-Spiro[Furan-3,4′-P yrido[3,2-d][1,3]Oxazin]-6′-yl)-1H-Pyrrolo[2,3-c]Pyridin-5-yl)Acetamide

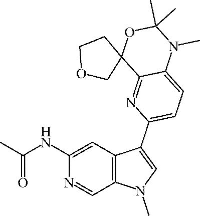 [SMILES: CC(=O)Nc1cc2c(-c3ccc4c(n3)C3(CCOC3)OC(C)(C)N4C)cn(C)c2cn1]

### Step 1 Synthesis of 6′-Chloro-1′,2′,2′-Trimethyl-1′,2′,4,5-Tetrahydro-2H-Spiro[Furan-3,4′-Pyrido [3,2-d] [1,3] Oxazine]

3-[6-Chloro-3-(methylamino)pyridin-2-yl]tetrahydrofuran-3-ol (100 mg, 0.44 mmol), acetic acid (5 mL) and acetone (5 mL) were added in sequence to a 10 mL single-necked bottle, the mixture was heated to 55° C., after the reaction was completed, the solution was adjusted to basicity with saturated sodium carbonate solution, after extraction with ethyl acetate (3 × 10 mL), the organic phases were combined, the solvent was concentrated, and the resulting residue was purified by column chromatography (petroleum ether/ethyl acetate = 5/1) to obtain the title compound as a white solid (60 mg, yield 51%).

MS (ESI, pos. ion) m/z: 269.1 [M+H]⁺.

### Step 2 Synthesis of N-(1-Methyl-3-(1′,2′,2′-Trimethyl-1′,2′,4,5-Tetrahydro-2H-Spiro[Fura n-3,4′-Pyrido[3,2-d][1,3]Oxazin]-6′-yl)-1H-Pyrrolo[2,3-c]Pyridin-5-yl)Acetamide

6′-Chloro-1′,2′,2′-trimethyl-1′,2′,4,5-tetrahydro-2H-spiro[furan-3,4′-pyrido[3,2-d][1,3] oxazine] (50 mg, 0.19 mmol), N [1-methyl-3-(4,4,5,5-tetramethyl-1,3,2-dioxaborolan-2-yl)-1H-pyrrolo[2,3-c]pyridin-5-yl]acetami de (87 mg, 0.28 mmol), potassium phosphate (121 mg, 0.56 mmol), 2-dicyclohexylphosphine 2′, 4′, 6′-triisopropylbiphenyl (53 mg, 0.11 mmol), tris(benzhydrylethyleneacetone) dipalladium (51 mg, 0.06 mmol), 1,4-dioxane (12 mL) and water (2 mL) were added in sequence to a 25 mL two-necked bottle, under N₂, the mixture was reacted overnight at 100° C., after the reaction was completed, the solvent was concentrated, and the resulting residue was purified by column chromatography (dichloromethane /methanol (v/v)=30/1) to obtain the title compound as a white solid (42 mg, yield 53%).

MS (ESI, pos. ion) m/z: 422.2 [M+H]⁺;

¹H NMR (400 MHz, CDCl₃) δ 8.93 (s, 1H), 8.39 (s, 1H), 8.12 (s, 1H), 7.70 (s, 1H), 7.50 (d, J = 8.4 Hz, 1H), 7.05 (d, J = 8.4 Hz, 1H), 4.22 (dt, J = 18.3, 8.0 Hz, 4H), 3.88 (d, J = 17.6 Hz, 3H), 2.80 (s, 3H), 2.44 (dd, J = 12.2, 6.2 Hz, 1H), 2.22 (s, 3H), 1.46 (d, J = 2.9 Hz, 6H), 0.86 (d, J = 10.5 Hz, 1H).

### Example 14 N-(3-(4′-Methoxy-4,5-Dihydro-2H,5′H-Spiro[Furan-3,7′-Furo[3,4-b]Pyridine]-2′-yl)-1-((R)-Tetrahydrofuran-3-yl)-1H-Pyrrolo[2,3-c]Pyridin-5-yl)Acetamide

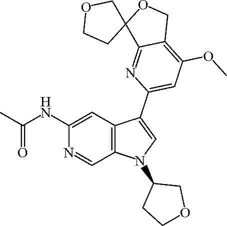 [SMILES: COc1cc(-c2cn([C@@H]3CCOC3)c3cnc(NC(C)=O)cc23)nc2c1COC21CCOC1]

### Step 1 Synthesis of N-(3-(4′-Methoxy-4,5-Dihydro-2H,5′H-Spiro[Furan-3,7′-Furo[3,4-b]P yridine]-2′-yl)-1H-Pyrrolo[2,3-c]Pyridin-5-yl)Acetamide

2′-chloro-4′-methoxy-4,5-dihydro-2H,5′H-spiro[furan-3,7′-furo[3,4-b]pyridine] (50 mg, 0.20 mmol), tert-butyl 5-acetamido-3-(4,4,5,5-tetramethyl-1,3,2-dioxborolan-2-yl)-1H-pyrrolo[2,3-c]pyridine-1-carboxylat e (124 mg, 0.31 mmol), potassium carbonate (57 mg, 0.41 mmol), tris(benzhydrylethyleneacetone) dipalladium (51 mg, 0.06 mmol), 1,4-dioxane (5 mL) and water (2 mL) were added in sequence to a 10 mL single-necked bottle, under N₂, the mixture was reacted overnight at 100° C., after the reaction was completed, the solvent was concentrated, and the crude product was purified by column chromatography (dichloromethane/methanol (v/v)=30/1) from the resulting residue to obtain the title compound as a white solid (70 mg, yield 90%).

MS (ESI, pos. ion) m/z: 381.2 [M+H]⁺;

¹H NMR (400 MHz, DMSO-d₆) δ 11.88 (s, 1H), 10.14 (s, 1H), 9.16 (s, 1H), 8.51 (s, 1H), 8.38 (d, J = 2.7 Hz, 1H), 7.35 (s, 1H), 4.99 (d, J = 12.8 Hz, 2H), 4.10 (td, J = 8.1, 3.5 Hz, 1H), 4.06 - 4.00 (m, 1H), 3.98 (d, J = 7.3 Hz, 3H), 3.91 (dd, J = 21.6, 9.4 Hz, 2H), 2.47 (d, J = 8.3 Hz, 1H), 2.24 - 2.14 (m, 1H), 2.09 (s, 3H).

### Step 2 Synthesis of N-(3-(4′-Methoxy-4,5-Dihydro-2H,5′H-Spiro[Furan-3,7′-Furo[3,4-b]P yridine]-2′-yl)-1-((R)-Tetrahydrofuran-3-yl)-1H-Pyrrolo[2,3-c]Pyridin-5-yl)Acetamide

N (3-(4′-methoxy-4,5-dihydro-2H,5′H-spiro[furan-3,7′-furo[3,4-b]pyridine]-2′-yl)-1H -pyrrolo[2,3-c]pyridin-5-yl)acetamide (50 mg, 0.13 mmol), (S)-tetrahydrofuran-3-yl methanesulfonate (39 mg, 0.19 mmol), cesium carbonate (100 mg, 0.26 mmol) and DMF (5 mL) were added in sequence to a 25 mL two-necked bottle, the mixture was reacted at 100° C., after the reaction was completed, the solvent was concentrated, and the resulting residue was purified by column chromatography (dichloromethane/methanol (v/v)=30/1) to obtain the title compound as a yellow solid (24 mg, yield 40%).

MS (ESI, pos. ion) m/z: 451.2 [M+H]⁺;

¹H NMR (400 MHz, DMSO-d₆) δ 10.21 (s, 1H), 9.19 (s, 1H), 8.72 (s, 1H), 8.39 (s, 1H), 7.38 (s, 1H), 5.41 (dd, J = 8.0, 4.0 Hz, 1H), 5.00 (s, 2H), 4.22 - 4.00 (m, 5H), 3.99 (s, 3H), 3.96 - 3.81 (m, 2H), 2.65 - 2.53 (m, 2H), 2.26 (d, J = 4.1 Hz, 1H), 2.22 - 2.13 (m, 1H), 2.06 (d, J = 26.0 Hz, 3H).

### Example 15 N-(3-(4′-(2-Methoxyethoxy)-4,5,5′,6′-Tetrahydro-2H-Spiro[Furan-3,8′-Pyrano [3,4-b]Pyridin]-2′-yl)-1-Methyl-1H-Pyrrolo[2,3-c]Pyridin-5-yl)Acetamide

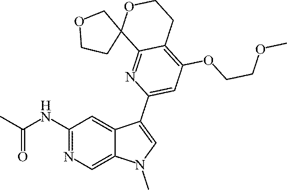 [SMILES: COCCOc1cc(-c2cn(C)c3cnc(NC(C)=O)cc23)nc2c1CCOC21CCOC1]

### Step 1 Synthesis of 2′-Chloro-4′-(2-Methoxyethoxy)-4,5,5′,6′-Tetrahydro-2H-Spiro[Furan-3,8′-Pyrano[3,4-b]Pyridine]

2′,4′-Dichloro-4,5,5′,6′-tetrahydro-2H-spiro[furan-3,8′-pyrano[3,4-b]pyridine] (83 mg, 0.32 mmol), DMF (3 mL), 2-methoxyethanol (49 mg, 0.60 mmol) and sodium hydride (15 mg, 0.38 mmol, 60% wt%) were added to a 25 mL round bottom flask, the mixture was stirred overnight at room temperature. 3 mL of saturated ammonium chloride was added to quench the reaction, then 5 mL of water was added, after extraction with ethyl acetate (3 × 10 mL), the combined organic phase was washed with 10 mL water and 10 mL saturated sodium chloride, then the organic phase was separated, dried over anhydrous sodium sulfate, filtered and concentrated in vacuum, and the resulting residue was purified by column chromatography (petroleum ether/ethyl acetate (v/v) = 5/1) to obtain the title compound as colorless oily liquid (75 mg, yield 78.00%).

MS (ESI, pos. ion) m/z: 300.1 [M+H]⁺.

### Step 2 Synthesis of N-(3-(4′-(2-Methoxyethoxy)-4,5,5′,6′-Tetrahydro-2H-Spiro[Furan-3,8′ -Pyrano[3,4-b]Pyridin]-2′-yl)-1-Methyl-1H-Pyrrolo[2,3-c]Pyridin-5-yl)Acetamide

Under N₂, 2′-chloro-4′-(2-methoxyethoxy)-4,5,5′,6′-tetrahydro-2H-spiro[furan-3,8′-pyrano[3,4-b]pyridine] (65 mg, 0.23 mmol), N-[1-methyl-3-(4,4,5,5-tetramethyl-1,3,2-dioxaborolan-2-yl)-1H-pyrrolo[2,3-c]pyridin-5-yl]acetami de (126 mg, 0.40 mmol) and 1,4-dioxane (8 mL) were added in sequence to a 50 mL two-necked round bottom bottle, the mixture was stirred to dissolve, then water (2 mL) was added, the solution was a yellow turbid liquid, then sodium carbonate (68 mg, 0.64 mmol) was added and the mixture was deoxygenated by N₂ bubbling for 10 min, then PdCl₂dppf (20 mg, 0.02 mmol) was added and the mixture was deoxygenated by N₂ bubbling for 10 min. The bottle was connected to a reflux condenser, the mixture was refluxed overnight at 80° C. under N₂. After the heating was stopped, the mixture was cooled to room temperature, then 10 mL of water was added to quench the reaction, after extraction with chloroform (3 × 10 mL), the combined organic phase was dried over anhydrous sodium sulfate, and the resulting residue was purified by column chromatography (ethyl acetate/methanol (v/v) = 91/9) to obtain the title compound as a yellow solid (40.0 mg, yield 40.80%).

MS (ESI, pos. ion) m/z: 453.2 [M+H]⁺;

¹H NMR (400 MHz, CDCl₃) δ 8.99 (s, 1H), 8.50 (s, 1H), 8.38 (s, 1H), 7.81 (s, 1H), 7.05 (s, 1H), 4.47 - 4.05 (m, 6H), 4.00 - 3.76 (m, 4H), 3.92 (s, 3H), 3.47 (s, 3H), 2.89 - 2.75 (m, 3H), 2.35 - 2.27 (m, 1H), 2.23 (s, 3H).

### Example 16 N-(3-(4′-Ethoxy-4,5,5′,6′-Tetrahydro-2H-Spiro[Furan-3,8′-Pyrano[3,4-b]Pyrid ine]-2′-yl)-1-Methyl-1H-Pyrrolo[2,3-c]Pyridin-5-yl)Acetamide

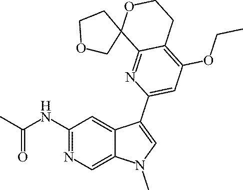 [SMILES: CCOc1cc(-c2cn(C)c3cnc(NC(C)=O)cc23)nc2c1CCOC21CCOC1]

### Step 1 Synthesis of 2′-Chloro-4′-Ethoxy-4,5,5′,6′-Tetrahydro-2H-Spiro[Furan-3,8′-Pyrano [3,4-b]Pyridine]

2′,4′-Dichloro-4,5,5′,6′-tetrahydro-2H-spiro[furan-3,8′-pyrano[3,4-b]pyridine] (90 mg, 0.34 mmol), DMF (4 mL) and absolute ethanol (2 mL) were added in sequence to a 25 mL one-necked bottle, the mixture was cooled to 0° C., then sodium hydride (11 mg, 0.48 mmol) was slowly added, after the addition was completed, the solution was reacted at room temperature. The reaction was quenched by 5 mL of saturated ammonium chloride solution, after extraction with ethyl acetate (3 × 20 mL), the organic phases were combined, the solvent was concentrated, and the resulting residue was purified by column chromatography (petroleum ether/ethyl acetate (v/v) = 10/1) to obtain the title compound as a yellow solid (69.0 mg, yield 74%).

MS (ESI, pos. ion) m/z: 270.1 [M+H]⁺.

### Step 2 Synthesis of N-(3-(4′-Ethoxy-4,5,5′,6′-Tetrahydro-2H-Spiro[Furan-3,8′-Pyrano[3,4 -b]Pyridine]-2′-yl)-1-Methyl-1H-Pyrrolo[2,3-c]Pyridin-5-yl)Acetamide

2′-Chloro-4′-ethoxy-4,5,5′,6′-tetrahydro-2H-spiro[furan-3,8′-pyrano[3,4-b]pyridine] (70 mg, 0.26 mmol), N-[1-methyl-3-(4,4,5,5-tetramethyl-1,3,2-dioxaborinane-2-yl)-1H-pyrrolo[2,3-c]pyridin-5-yl]aceta mide (160 mg, 0.31 mmol), potassium carbonate (71 mg, 0.52 mmol), PdCl₂dppf (21 mg, 0.02 mmol), 1,4-dioxane (5 mL) and water (2 mL) were added in sequence to a 25 mL two-necked bottle, under N₂, the mixture was reacted overnight at 100° C., after the reaction was completed, the solvent was concentrated, and the resulting residue was purified by column chromatography (dichloromethane/methanol (v/v) = 30/1) to obtain the title compound as a white solid (76 mg, yield 69%).

MS (ESI, pos. ion) m/z: 423.2 [M+H]⁺;

¹H NMR (400 MHz, DMSO-d₆) δ 10.18 (s, 1H), 9.03 (s, 1H), 8.60 (s, 1H), 8.29 (s, 1H), 7.20 (s, 1H), 4.22 (q, J = 6.9 Hz, 3H), 4.05 - 3.95 (m, 3H), 3.93 (s, 3H), 3.86 (dt, J = 11.6, 5.3 Hz, 2H), 2.85 (dt, J = 12.3, 8.6 Hz, 1H), 2.65 (d, J = 8.4 Hz, 2H), 2.21 - 2.11 (m, 1H), 2.09 (s, 3H), 1.40 (t, J = 6.9 Hz, 3H).

### Example 17 N-(3-(4′-Isopropoxy-4,5,5′,6′-Tetrahydro-2H-Spiro[Furan-3,8′-Pyrano[3,4-b]P yridine]-2′-yl)-1-Methyl-1H-Pyrrolo[2,3-c]Pyridin-5-yl)Acetamide

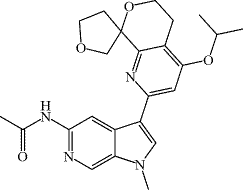 [SMILES: CC(=O)Nc1cc2c(-c3cc(OC(C)C)c4c(n3)C3(CCOC3)OCC4)cn(C)c2cn1]

### Step 1 Synthesis of 2′-Chloro-4′-Isopropoxy-4,5,5′,6′-Tetrahydro-2H-Spiro[Furan-3,8′-Py rano[3,4-b]Pyridine]

2′,4′-Dichloro-4,5,5′,6′-tetrahydro-2H-spiro[furan-3,8′-pyrano[3,4-b]pyridine] (90 mg, 0.34 mmol), DMF (4 mL) and isopropanol (2 mL) were added in sequence to a 25 mL one-necked bottle, the mixture was cooled to 0° C., then sodium hydride (11 mg, 0.48 mmol) was slowly added, after the addition was completed, the solution was reacted at room temperature. The reaction was quenched by 5 mL of saturated ammonium chloride solution, after extraction with ethyl acetate (3 × 20 mL), the organic phases were combined, the solvent was concentrated, and the resulting residue was purified by column chromatography (petroleum ether/ethyl acetate (v/v) = 10/1) to obtain the title compound as colorless oily liquid (72 mg, yield 73%).

MS (ESI, pos. ion) m/z: 284.1 [M+H]⁺;

### Step 2 Synthesis of N-(3-(4′-Isopropoxy-4,5,5′,6′-Tetrahydro-2H-Spiro[Furan-3,8′-Pyran o[3,4-b]Pyridine]-2′-yl)-1-Methyl-1H-Pyrrolo[2,3-c]Pyridin-5-yl)Acetamide

2′-Chloro-4′-isopropoxy-4,5,5′,6′-tetrahydro-2H-spiro[furan-3,8′-pyrano[3,4-b]pyridine], N-[1-methyl-3-(4,4,5,5-tetramethyl-1,3,2-dioxaborolan-2-yl)-1H-pyrrolo[2,3-c]pyridin-5-yl]acetamide (188 mg, 0.34 mmol), potassium carbonate (77 mg, 0.56 mmol), PdCl₂dppf (23 mg, 0.02 mmol), 1,4-dioxane (7 mL) and water (2 mL) were added in sequence to a 25 mL two-necked bottle, the mixture was reacted overnight at 100° C. under N₂. After the reaction was completed, the solvent was concentrated, and the resulting residue was purified by column chromatography (dichloromethane/methanol (v/v) = 30/1) to obtain the title compound as a white solid (56 mg, yield 45%).

MS (ESI, pos. ion) m/z: 437.2 [M+H]⁺;

¹H NMR (400 MHz, DMSO-d₆) δ 10.18 (s, 1H), 9.02 (s, 1H), 8.61 (s, 1H), 8.29 (s, 1H), 7.20 (s, 1H), 4.86 (dt, J = 12.0, 6.0 Hz, 1H), 4.20 (td, J = 8.3, 4.2 Hz, 1H), 3.98 (d, J = 8.4 Hz, 3H), 3.93 (s, 3H), 3.85 (dt, J = 11.7, 5.4 Hz, 2H), 2.84 (dt, J = 12.1, 8.6 Hz, 1H), 2.61 (s, 2H), 2.20 - 2.12 (m, 1H), 2.09 (s, 3H), 1.36 (d, J = 6.0 Hz, 6H).

### Example 18 N-(3-(5′,5′-Difluoro-4,5,5′,6′-Tetrahydro-2H-Spiro[Furan-3,8′-Pyrano[3,4-b]P yridine]-2′-yl)-1-Methyl-1H-Pyrrolo[2,3-c]Pyridin-5-yl)Acetamide

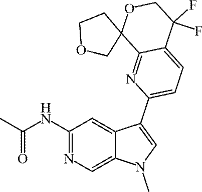 [SMILES: CC(=O)Nc1cc2c(-c3ccc4c(n3)C3(CCOC3)OCC4(F)F)cn(C)c2cn1]

### Step 1 Synthesis of 2-(2-Bromopyridin-3-yl)Acetic Acid

2-(2-Bromopyridin-3-yl)acetaldehyde (9.35 g, 46.70 mmol), DCM (50 mL), DMSO (26 mL), water (25 mL) and phosphoric acid (1.28 mL, 18.7 mmol, 85%) were added in sequence to a 250 mL round bottom flask, then aqueous solution (20 mL) of NaClO₂ (8.94 g, 79.5 mmol) was slowly added, the mixture was stirred at room temperature for 8 h. After suction filtration, the filter cake was taken to obtain the title compound as a white solid (5.56 g, yield 55.1%).

MS (ESI, pos. ion) m/z: 216.2 [M+H]⁺.

### Step 2 Synthesis of Methyl 2-(2-Bomopyridin-3-yl)Acetate

2-(2-Bromopyridin-3-yl)acetic acid (5.50 g, 25.45 mmol) was added to a 250 mL round-bottom flask, under N₂, anhydrous methanol (100 mL) was added, then concentrated sulfuric acid (1.38 mL, 25.45 mmol) was added dropwise, and the mixture was refluxed for 5 h after the addition. The solvent was concentrated, and the resulting residue was purified by column chromatography (petroleum ether/ethyl acetate (v/v) = 5/1) to obtain viscous colorless oily liquid (5.20 g, yield 88.8%).

MS (ESI, pos. ion) m/z: 230.2 [M+H]⁺;

¹H NMR (400 MHz, CDCl₃): δ (ppm) 8.31 (dd, J = 4.7, 1.7 Hz, 1H), 7.63 (dd, J = 7.5, 1.7 Hz, 1H), 7.27 (dd, J= 7.5, 4.7 Hz, 1H), 3.80 (s, 2H), 3.75 (s, 3H).

### Step 3 Synthesis of Methyl 2-(2-Bromopyridin-3-yl)-2,2-Difluoroacetate

Methyl 2-(2-bromopyridin-3-yl)acetate (4.10 g, 17.82 mmol) was added to a 250 mL round bottom flask, under N₂, anhydrous THF (100 mL) was added, and the mixture was cooled to -78° C. LiHMDS (71.3 mL, 71.30 mmol, 1 mol/L THF solution) was added dropwise, and after the addition, the mixture was stirred at -78° C. for 40 min, then N-fluorobisbenzenesulfonamide (22.48 g, 71.30 mmol) was added, and after the addition, the mixture was kept the temperature and stirred for 2 h, then heated to room temperature and continuously stirred for 2 h. The reaction was quenched by adding 20 mL of saturated aqueous ammonium chloride, extracted with ethyl acetate (3 × 20 mL), dried over anhydrous sodium sulfate, filtered, and concentrated under reduced pressure, and the resulting residue was purified by column chromatography (petroleum ether/ethyl acetate (v/v) =10/1) to obtain the title compound as yellow oily liquid (3.20 g, yield 67.5%).

MS (ESI, pos. ion) m/z: 266.2 [M+H]⁺;

¹H NMR (400 MHz, CDCl₃): δ (ppm) 8.52 (d, J = 4.0 Hz, 1H), 8.05 (dd, J = 7.8, 1.6 Hz, 1H), 7.45 (dd, J = 7.7, 4.8 Hz, 1H), 3.93 (s, 3H).

### Step 4 Synthesis of 2-(2-Bromopyridin-3-yl)-2,2-Difluoroethanol

Methyl-2-(2-bromopyridin-3-yl)-2,2-difluoroacetate (4.70 g, 17.66 mmol) and anhydrous methanol (100 mL) were added to a 250 mL round bottom flask, the mixture was cooled to 0° C. under N₂. Sodium borohydride (1.34 g, 35.33 mmol) was added, and the ice-water bath was removed after the addition, then the mixture was stirred at room temperature for 2 h. 20 mL of saturated aqueous ammonium chloride was added carefully and dropwise to quench the reaction, methanol was concentrated, after extraction with ethyl acetate (3 × 20 mL), the mixture was washed with 20 mL of saturated sodium chloride, then the organic phases were separated and dried, filtered and concentrated in vacuum, the resulting residue was purified by column chromatography (petroleum ether/ethyl acetate (v/v) = 2/1) to obtain the title compound as a white solid (3.74 g, yield 88.9%).

MS (ESI, pos. ion) m/z: 238.2 [M+H]⁺.

### Step 5 Synthesis of 3-(3-(1,1-Difluoro-2-Hydroxyethyl)Pyridin-2-yl)Tetrahydrofuran-3-ol

2-(2-Bromopyridin-3-yl)-2,2-difluoroethanol (2.50 g, 10.50 mmol) was added to a 250 mL round bottom flask, under N₂, anhydrous THF (40 mL) was added and the mixture was cooled to -78° C. N-butyllithium (10.3 mL, 23.10 mmol, 2.25 mol/L n-hexane solution) was added dropwise and the addition was completed in 30 min, the mixture was stirred at -78° C. for 20 min, then tetrahydrofuran-3-one (1.08 g, 12.60 mmol) was added and the mixture was kept stirring for 30 min, then heated to room temperature and continued stirring for 60 min. The reaction was quenched by 20 mL of saturated aqueous ammonium chloride, extracted with ethyl acetate (3 × 20 mL), dried over anhydrous sodium sulfate, filtered and concentrated under reduced pressure, and the resulting residue was purified by column chromatography (petroleum ether/ethyl acetate (v/v) =⅓) to obtain the title compound as viscous yellow oily liquid (0.45 g, yield 17.6%).

MS (ESI, pos. ion) m/z: 246.2 [M+H]⁺;

### Step 6 Synthesis of 5′,5′-Difluoro-4,5,5′,6′-Tetrahydro-2H-Spiro[Furan-3,8′-Pyrano[3,4-b] Pyridine]

3-(3-(1,1-Difluoro-2-hydroxyethyl)pyridin-2-yl)tetrahydrofuran-3-ol (0.95 g, 3.87 mmol) was added to a 100 mL round bottom flask, under N₂, anhydrous THF (10 mL) was added and the mixture was cooled to 0° C. NaHMDS (4.26 mL, 8.52 mmol, 2 mol/L in THF) was added dropwise, and the mixture was stirred at 0° C. for 10 min after the addition. Then p-toluenesulfonyl chloride (0.88 g, 4.65 mmol) was added and the mixture was continued stirring at 0° C. for 1 h. The reaction was quenched by 10 mL of saturated aqueous ammonium chloride, extracted with ethyl acetate (3 × 10 mL), the combined organic phase was washed with 10 mL of saturated sodium chloride. Then filtered and concentrated under reduced pressure, the resulting residue was purified by column chromatography (dichloromethane/methanol (v/v) = 95/5) to obtain the title compound as yellow oily liquid (0.29 g, yield 33.0%).

MS (ESI, pos. ion) m/z: 228.2 [M+H]⁺;

¹H NMR (400 MHz, CDCl₃): δ (ppm) 8.73 (d, J = 4.7 Hz, 1H), 8.01 (d, J = 7.9 Hz, 1H), 7.36 (dd, J = 7.9, 4.8 Hz, 1H), 4.23 - 4.13 (m, 6H), 2.70 - 2.60 (m, 1H), 2.47 - 2.37 (m, 1H).

### Step 7 Synthesis of 5′,5′-Difluoro-4,5,5′,6′-Tetrahydro-2H-Spiro[Furan-3,8′-Pyrano[3,4-b] Pyridine] 1′-Oxide

5′,5′-Difluoro-4,5,5′,6′-tetrahydro-2H-spiro[furan-3,8′-pyrano[3,4-b]pyridine] (0.29 g, 1.28 mmol), dichloromethane (25 mL) and m-chloroperoxybenzoic acid (0.39 g, 1.92 mmol, 85 mass%) were added to a 100 mL flask in sequence, the mixture was stirred overnight at room temperature. The reaction was quenched with 25 mL of saturated sodium sulfite, then stirred for 15 min, and 25 mL of saturated sodium carbonate was added, after extraction with chloroform (3 × 25 mL), the combined organic phase was dried over anhydrous sodium sulfate, filtered, and concentrated under reduced pressure, the resulting residue was purified by column chromatography (dichloromethane/methanol (v/v) = 95/5) to obtain the title compound as a white solid (0.25 g, yield 80.6%).

MS (ESI, pos. ion) m/z: 244.2 [M+H]⁺.

### Step 8 Synthesis of 2′-Chloro-5′,5′-Difluoro-4,5,5′,6′-Tetrahydro-2H-Spiro[Furan-3,8′-Pyr ano [3,4-b] Pyridine]

5′,5′-Difluoro-4,5,5′,6′-tetrahydro-2H-spiro[furan-3,8′-pyrano[3,4-b]pyridine] 1′-oxide (0.22 g, 0.90 mmol) and phosphine oxychloride (15 mL, 165 mmol) were added to a 50 mL round bottom flask, the mixture was refluxed for 1 h. The solution was concentrated in vacuum, 12 mL of saturated sodium bicarbonate was added, and the pH was adjusted to 7 with saturated sodium carbonate, after extraction with ethyl acetate (3 × 20 mL), the combined organic phase was washed with 20 mL of saturated sodium chloride, then dried and filtered, and concentrated under reduced pressure, the resulting residue was purified by column chromatography (petroleum ether/ethyl acetate (v/v) = 10/1) to obtain the title compound as a yellow-white solid (0.15 g, yield 64.4%).

MS (ESI, pos. ion) m/z: 262.2 [M+H]⁺;

¹H NMR (400 MHz, CDCl₃): δ (ppm) 7.96 (d, J = 8.3 Hz, 1H), 7.38 (d, J = 8.2 Hz, 1H), 4.23 - 4.10 (m, 6H), 2.71 - 2.51 (m, 1H), 2.44 - 2.37 (m, 1H).

### Step 9 Synthesis of N-(3-(5′,5′-Difluoro-4,5,5′,6′-Tetrahydro-2H-Spiro[Furan-3,8′-Pyrano [3,4-b]Pyridine]-2′-yl)-1-Methyl-1H-Pyrrolo[2,3-c]Pyridin-5-yl)Acetamide

Under N₂, 2′-chloro-5′,5′-difluoro-4,5,5′,6′-tetrahydro-2H-spiro[furan-3,8′-pyrano[3,4-b]pyridine] (40 mg, 0.15 mmol), N-[1-methyl-3-(4,4,5,5-tetramethyl-1,3,2-dioxaborolan-2-yl)-1H-pyrrolo[2,3-c]pyridin-5-yl]acetami de (0.082 g, 0.18 mmol, 70%), 1,4-dioxane (8 mL), water (2 mL) and potassium carbonate (42 mg, 0.31 mmol) were added to a 50 mL two-necked round bottom flask, the mixture was deoxygenated by bubbling N₂ for 10 min, then PdCl₂dppf (37 mg, 0.045 mmol) was added and the mixture was deoxygenated by bubbling N₂ for 10 min. The flask was connected to a reflux condenser, the resulting solution was refluxed overnight under N₂, then 10 mL of water was added to quench the reaction, after extraction with chloroform (3 × 10 mL), the combined organic phase was dried over anhydrous sodium sulfate, and the resulting residue was purified by column chromatography (ethyl acetate/methanol (v/v) = 91/9) to obtain the title compound as a yellow solid (30 mg, yield 47.4%).

MS (ESI, pos. ion) m/z: 415.2 [M+H]⁺;

¹H NMR (400 MHz, DMSO-d₆): δ (ppm) 10.27 (s, 1H), 9.08 (s, 1H), 8.65 (s, 1H), 8.45 (s, 1H), 8.07 (d, J = 8.4 Hz, 1H), 7.83 (d, J = 8.4 Hz, 1H), 4.31 (dd, J= 12.7, 8.2 Hz, 2H), 4.24 (d, J= 9.2 Hz, 2H), 3.96 (d, J = 7.0 Hz, 5H), 2.91 - 2.86 (m, 1H), 2.40 - 2.35 (m, 1H), 2.10 (s, 3H).

### Example 19 N-(3-(4′-(2-(Dimethylamino)Ethoxy)-4,5,5′,6′-Tetrahydro-2H-Spiro[Furan-3,8′ -Pyrano[3,4-b]Pyridine]-2′-yl)-1-Methyl-1H-Pyrrolo[2,3-c]Pyridin-5-yl)Acetamide

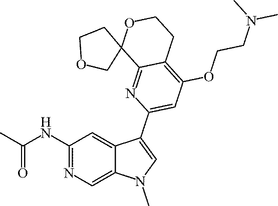 [SMILES: CC(=O)Nc1cc2c(-c3cc(OCCN(C)C)c4c(n3)C3(CCOC3)OCC4)cn(C)c2cn1]

### Step 1 Synthesis of 2-((2′-Chloro-4,5,5′,6′-Tetrahydro-2H-Spiro[Furan-3,8′-Pyrano[3,4-b] Pyridin]-4′-yl)Oxy)-N,N-Dimethylethylamine

2′,4′-Dichloro-4,5,5′,6′-tetrahydro-2H-spiro[furan-3,8′-pyrano[3,4-b]pyridine] (90 mg, 0.34 mmol), DMF (4 mL) and dimethylethanolamine (2 mL) were added to a 25 mL one-necked flask in sequence, the mixture was cooled to 0° C., then sodium hydride (11 mg, 0.48 mmol) was slowly added, and the mixture was reacted at room temperature after the addition. The reaction was quenched with 5 mL of saturated ammonium chloride solution, after extraction with ethyl acetate (3 × 20 mL), the organic phases were combined, the solvent was concentrated, the resulting residue was purified by column chromatography (dichloromethane/methanol (v/v) = 30/1) to obtain the title compound as oily liquid (70 mg, yield 64%).

MS (ESI, pos. ion) m/z: 313.1 [M+H]⁺.

### Step 2 Synthesis of N-(3-(4′-(2-(Dimethylamino)ethoxy)-4,5,5′,6′-Tetrahydro-2H-Spiro[F uran-3,8′-Pyrano[3,4-b]Pyridine]-2′-yl)-1-Methyl-1H-Pyrrolo[2,3-c]Pyridin-5-yl)Acetamide

2-((2′-Chloro-4,5,5′,6′-tetrahydro-2H-spiro[furan-3,8′-pyrano[3,4-b]pyridin]-4′-yl)oxy )-N,N-dimethylethylamine (70 mg, 0.22 mmol), N-[1-methyl-3-(4,4,5,5-tetramethyl-1,3,2-dioxaborolan-2-yl)-1H-pyrrolo[2,3-c]pyridin-5-yl]acetami de (160 mg, 0.26 mmol), potassium carbonate (61 mg, 0.44 mmol), PdCl₂dppf (18 mg, 0.02 mmol), 1,4-dioxane (5 mL) and water (2 mL) were added to a 25 mL two-necked flask in sequence, under N₂, the mixture was reacted overnight at 100° C., after the reaction was completed, the solvent was concentrated, the resulting residue was purified by column chromatography (dichloromethane/methanol (v/v) = 30/1) to obtain the title compound as a white solid (34 mg, yield 32%).

MS (ESI, pos. ion) m/z: 466.2 [M+H]⁺;

¹H NMR (400 MHz, DMSO-d₆) δ 10.18 (s, 1H), 9.04 (s, 1H), 8.61 (s, 1H), 8.34 (s, 1H), 7.27 (s, 1H), 4.40 (s, 2H), 4.23 (d, J= 3.7 Hz, 1H), 3.98 (dd, J= 8.7, 3.5 Hz, 3H), 3.93 (s, 3H), 3.91 - 3.80 (m, 2H), 3.15 (s, 2H), 2.87 (dd, J = 20.7, 8.5 Hz, 1H), 2.68 (s, 2H), 2.56 (s, 6H), 2.23 -2.13 (m, 1H), 2.09 (s, 3H).

### Example 20 N-(3-(4′-Butoxy-4,5,5′,6′-Tetrahydro-2H-Spiro[Furan-3,8′-pyrAno[3,4-b]Pyrid ine]-2′-yl)-1-Methyl-1H-Pyrrolo[2,3-c]Pyridin-5-yl)Acetamide

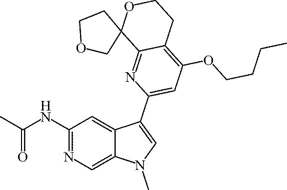 [SMILES: CCCCOc1cc(-c2cn(C)c3cnc(NC(C)=O)cc23)nc2c1CCOC21CCOC1]

### Step 1 Synthesis of 4′-Butoxy-2′-Chloro-4,5,5′,6′-Tetrahydro-2H-Spiro[Furan-3,8′-Pyrano [3,4-b]Pyridine]

2′,4′-Dichloro-4,5,5′,6′-tetrahydro-2H-spiro[furan-3,8′-pyrano[3,4-b]pyridine] (93 mg, 0.36 mmol), DMF (3 mL) and 1-butanol (53 mg, 0.80 mmol) were added to a 25 mL round bottom flask in sequence, then sodium hydride (20 mg, 0.50 mmol, 60% wt%) was added and the mixture was stirred overnight at room temperature. 3 mL of saturated ammonium chloride was added to quench the reaction, then 5 mL of water was added, after extraction with ethyl acetate (3 × 10 mL), the combined organic phase was washed with 10 mL of water and 10 mL of saturated sodium chloride, the organic phase was separated, dried over anhydrous sodium sulfate, filtered and concentrated, and the resulting residue was purified by column chromatography (petroleum ether/ethyl acetate (v/v) = 10/1) to obtain the title compound as colorless oily liquid (84 mg, yield 79.00%).

MS (ESI, pos. ion) m/z: 298.1 [M+H]⁺;

### Step 2 Synthesis of N-(3-(4′-Butoxy-4,5,5′,6′-Tetrahydro-2H-Spiro[Furan-3,8′-Pyrano[3,4 -b]Pyridine]-2′-yl)-1-Methyl-1H-Pyrrolo[2,3-c]Pyridin-5-yl)Acetamide

Under N₂, 4′-butoxy-2′-chloro-4,5,5′,6′-tetrahydro-2H-spiro[furan-3,8′-pyrano[3,4-b]pyridine] (66 mg, 0.22 mmol), N-[1-methyl-3-(4,4,5,5-tetramethyl-1,3,2-dioxaborolan-2-yl)-1H-pyrrolo[2,3-c]pyridin-5-yl]acetami de (164 mg, 0.52 mmol), potassium carbonate (61 mg, 0.44 mmol) and PdCl₂dppf (18 mg, 0.02 mmol) were added to a 50 mL two-neck round bottom flask, then vacuumized, and 1,4-dioxane (8 mL) was added under N₂, the mixture was stirred to dissolve most of the solids, then water (2 mL) was added and the mixture was deoxygenated by bubbling N₂ for 10 min. The flask was connected to a reflux condenser, under N₂, the mixture was heated to 100° C. and reacted overnight. After the heating was stopped, the mixture was cooled to room temperature, then 10 mL of water was added to quench the reaction, after extraction with chloroform (3 × 10 mL), the combined organic phase was dried over anhydrous sodium sulfate, and the resulting residue was purified by column chromatography (ethyl acetate/methanol (v/v) = 91/9) to obtain the title compound as a yellow solid (22.0 mg, yield 22.00%).

MS (ESI, pos. ion) m/z: 451.1 [M+H]⁺;

¹H NMR (400 MHz, CDCl₃) δ 9.00 (s, 1H), 8.40 (s, 1H), 8.28 (s, 1H), 7.79 (s, 1H), 7.03 (s, 1H), 4.30 - 4.22 (m, 2H), 4.21 - 4.13 (m, 4H), 4.03 - 3.94 (m, 2H), 3.91 (s, 3H), 2.92 -2.75 (m, 3H), 2.36 - 2.27 (m, 1H), 2.22 (s, 3H), 1.89 - 1.80 (m, 2H), 1.60 - 1.48 (m, 2H), 1.00 (t, J = 7.4 Hz, 3H).

### Example 21 N-(3-(4′-Chloro-4,5,5′,6′-Tetrahydro-2H-Spiro[Furan-3,8′-Pyrano[3,4-b]Pyridi ne]-2′-yl)-1-Methyl-1H-Pyrrolo[2,3-c]Pyridin-5-yl)Acetamide

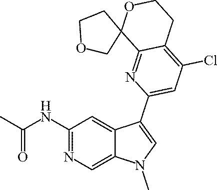 [SMILES: CC(=O)Nc1cc2c(-c3cc(Cl)c4c(n3)C3(CCOC3)OCC4)cn(C)c2cn1]

Under N₂, 2′,4′-dichloro-4,5,5′,6′-tetrahydro-2H-spiro[furan-3,8′-pyrano[3,4-b]pyridine] (52 mg, 0.2 mmol), N-[1-methyl-3-(4,4,5,5-tetramethyl-1,3,2-dioxaborolan-2-yl)-1H-pyrrolo[2,3-c]pyridin-5-yl]acetami de (99 mg, 0.22 mmol), potassium carbonate (55 mg, 0.40 mmol) and PdCl₂dppf (16 mg, 0.02 mmol) were added to a 25 mL two-neck round bottom flask in sequence, then vacuumized, and 1,4-dioxane (5 mL) was added under N₂, the mixture was stirred to dissolve most of the solids, then water (2 mL) was added and the mixture was deoxygenated by bubbling N₂ for 10 min. The flask was connected to a reflux condenser, under N₂, the mixture was heated to 100° C. and reacted overnight. After the heating was stopped, the mixture was cooled to room temperature, then 10 mL of water was added to quench the reaction, after extraction with chloroform (3 × 10 mL), the combined organic phase was dried over anhydrous sodium sulfate, and the resulting residue was purified by column chromatography (ethyl acetate/methanol (v/v) = 91/9) to obtain the title compound as a yellow solid (36.8 mg, yield 44.60%).

MS (ESI, pos. ion) m/z: 415.1 [M+H]⁺;

¹H NMR (400 MHz, CDCl₃) δ 9.04 (s, 1H), 8.45 (s, 1H), 8.39 (s, 1H), 7.77 (s, 1H), 7.53 (s, 1H), 4.42 - 4.31 (m, 1H), 4.30 - 4.14 (m, 3H), 4.09 - 3.97 (m, 2H), 3.92 (s, 3H), 2.91 (d, J = 2.9 Hz, 3H), 2.41 - 2.32 (m, 1H), 2.24 (s, 3H).

### Example 22 N-(3-(4′-(3-Methoxypropoxy)-4,5,5′,6′-Tetrahydro-2H-Spiro[Furan-3,8′-Pyra no[3,4-b]Pyridin]-2′-yl)-1-Methyl-1H-Pyrrolo[2,3-c]Pyridin-5-yl)Acetamide

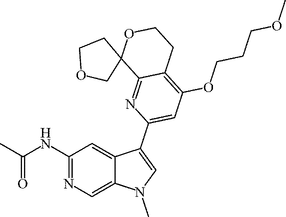 [SMILES: COCCCOc1cc(-c2cn(C)c3cnc(NC(C)=O)cc23)nc2c1CCOC21CCOC1]

### Step 1 Synthesis of 2′-Chloro-4′-(3-Methoxypropoxy)-4,5,5′,6′-Tetrahydro-2H-Spiro[fuRa n-3,8′-Pyrano[3,4-b]Pyridine]

2′,4′-Dichloro-4,5,5′,6′-tetrahydro-2H-spiro[furan-3,8′-pyrano[3,4-b]pyridine] (80 mg, 0.30 mmol), DMF (4 mL) and 3-methoxy-1-propanol (42 mg, 0.45 mmol) were added to a 25 mL one-neck flask in sequence, the mixture was cooled to 0° C., then sodium hydride (17 mg, 0.42 mmol) was slowly added, the solution was reacted at room temperature after the addition. 5 mL of saturated ammonium chloride solution was added to quench the reaction, after extraction with ethyl acetate (3 × 20 mL), the organic phases were combined, the solvent was concentrated, the resulting residue was purified by column chromatography (ethyl acetate/methanol (v/v) = 3/1) to obtain the title compound as yellow oily liquid (70 mg, yield 72%).

MS (ESI, pos. ion) m/z: 314.1 [M+H]⁺.

### Step 2 Synthesis of N-(3-(4′-(3-Methoxypropoxy)-4,5,5′,6′-Tetrahydro-2H-Spiro[Furan-3, 8′-Pyrano[3,4-b]Pyridin]-2′-yl)-1-Methyl-1H-Pyrrolo[2,3-c]pyRidin-5-yl)Acetamide

2′-Chloro-4′-(3-methoxypropoxy)-4,5,5′,6′-tetrahydro-2H-spiro[furan-3,8′-pyrano[3,4-b]pyridine] (70 mg, 0.22 mmol), N-[1-methyl-3-(4,4,5,5-tetramethyl-1,3,2-dioxaborolan-2-yl)-1H-pyrrolo[2,3-c]pyridin-5-yl]acetami de (98 mg, 0.44 mmol), potassium carbonate (61 mg, 0.44 mmol), PdCl₂dppf (18 mg, 0.02 mmol), 1,4-dioxane (5 mL) and water (2 mL) were added to a 25 mL two-neck flask in sequence, and the mixture was heated to 100° C. and reacted overnight under N₂. After the reaction was completed, the solvent was concentrated, and the resulting residue was purified by column chromatography (ethyl acetate/methanol (v/v) = 30/1) to obtain the title compound as a white solid (33 mg, yield 32%).

MS (ESI, pos. ion) m/z: 467.2 [M+H]⁺;

¹H NMR (400 MHz, DMSO-d₆) δ 10.19 (s, 1H), 9.05 (s, 1H), 8.61 (s, 1H), 8.32 (s, 1H), 7.22 (s, 1H), 4.20 (t, J = 6.1 Hz, 3H), 4.07 - 3.96 (m, 3H), 3.93 (s, 3H), 3.91 - 3.79 (m, 2H), 3.52 (t, J = 6.1 Hz, 2H), 3.34 (s, 6H), 3.27 (s, 3H), 2.87 (dd, J = 20.6, 8.6 Hz, 1H), 2.65 (s, 2H), 2.22 - 2.13 (m, 1H), 2.09 (s, 3H), 2.03 (dd, J = 12.2, 6.0 Hz, 2H).

### Example 23 N-(1-meThyl-3-(4′-(Methylsulfonyl)-4,5,5′,6′-Tetrahydro-2H-Spiro[Furan-3,8′-Pyrano[3,4-b]Pyridin]-2′-yl)-1H-Pyrrolo[2,3-c]Pyridin-5-yl)Acetamide

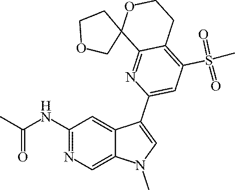 [SMILES: CC(=O)Nc1cc2c(-c3cc(S(C)(=O)=O)c4c(n3)C3(CCOC3)OCC4)cn(C)c2cn1]

### Step 1 Synthesis of 2′-Chloro-4′-(Methylthio)-4,5,5′,6′-Tetrahydro-2H-Spiro[Furan-3,8′-P yrano[3,4-b]Pyridine]

2′,4′-Dichloro-4,5,5′,6′-tetrahydro-2H-spiro[furan-3,8′-pyrano[3,4-b]pyridine] (78 mg, 0.30 mmol) and DMF (2 mL) were added to a 25 mL one-neck round bottom flask, then sodium methylthiolate (27 mg, 0.36 mmol) was added, the mixture was stirred overnight at room temperature. The reaction was quenched by 1 mL of water, concentrated in vacuum, and the resulting residue was purified by column chromatography (petroleum ether/ethyl acetate (v/v) = 10/1) to obtain the title compound as a white solid (0.055 g, yield 67.50%).

MS (ESI, pos. ion) m/z: 272.2 [M+H]⁺.

### Step 2 Synthesis of 2′-Chloro-4′-(Methylsulfonyl)-4,5,5′,6′-Tetrahydro-2H-Spiro[Furan-3, 8′-Pyrano[3,4-b]Pyridine]

2′-Chloro-4′-(methylthio)-4,5,5′,6′-tetrahydro-2H-spiro[furan-3,8′-pyrano[3,4-b]pyridi ne] (47 mg, 0.22 mmol), dichloromethane (5 mL) and m-chloroperoxybenzoic acid (84 mg, 0.41 mmol, purity 85%) were added to a 25 mL two-neck round bottom flask, the mixture was stirred at room temperature overnight. After the TLC spotting showed that the reaction was completed, 5 mL of saturated sodium sulfite was added to quench the reaction, and the mixture was stirred for 5 min, then 5 mL of saturated sodium carbonate was added. After extraction with chloroform (3 × 10 mL), the organic phases were combined, dried over anhydrous sodium sulfate and concentrated in vacuum to obtain the title compound as a white solid (52 mg, yield 99.90%).

MS (ESI, pos. ion) m/z: 304.1 [M+H]⁺.

### Step 3 Synthesis of N-(1-Methyl-3-(4′-(Methylsulfonyl)-4,5,5′,6′-Tetrahydro-2H-Spiro[Fu ran-3,8′-Pyrano[3,4-b]Pyridin]-2′-yl)-1H-Pyrrolo[2,3-c]Pyridin-5-yl)Acetamide

Under N₂, 2′-chloro-4′-(methylsulfonyl)-4,5,5′,6′-tetrahydro-2H-spiro[furan-3,8′-pyrano[3,4-b]pyridine] (60 mg, 0.2 mmol), N-[1-methyl-3-(4,4,5,5-tetramethyl-1,3,2-dioxaborolan-2-yl)-1H-pyrrolo[2,3-c]pyridin-5-yl]acetami de (100 mg, 0.22 mmol), potassium carbonate (55 mg, 0.40 mmol) and PdCl₂dppf (16 mg, 0.02 mmol) were added to a 25 mL two-neck round bottom flask, then vacuumized, and 1,4-dioxane (5 mL) was added under N₂, the mixture was stirred to dissolve most of the solids, then water (2 mL) was added and the mixture was deoxygenated by bubbling N₂ for 10 min. The flask was connected to a reflux condenser, under N₂, the mixture was heated to 100° C. and reacted overnight. After the heating was stopped, the mixture was cooled to room temperature, then 10 mL of water was added to quench the reaction, after extraction with chloroform (3 × 10 mL), the combined organic phase was dried over anhydrous sodium sulfate, and the resulting residue was purified by column chromatography (ethyl acetate/methanol (v /v) = 91/9) to obtain the title compound as a yellow solid (50.0 mg, yield 55.00%).

MS (ESI, pos. ion) m/z: 457.2 [M+H]⁺;

¹H NMR (400 MHz, CDCl₃) δ 9.14 (s, 1H), 8.64 (s, 1H), 8.40 (s, 1H), 8.04 (s, 1H), 7.85 (s, 1H), 4.51 - 4.39 (m, 1H), 4.31 - 4.18 (m, 3H), 4.09 - 3.99 (m, 2H), 3.94 (s, 3H), 3.36 -3.29 (m, 2H), 3.19 (s, 3H), 3.04 - 2.91 (m, 1H), 2.46 - 2.37 (m, 1H), 2.23 (s, 3H).

### Example 24 N-(3-(4′-(Dimethylamino)-4,5,5′,6′-Tetrahydro-2H-Spiro[Furan-3,8′-Pyrano[3,4-b]Pyridin]-2′-yl)-1-Methyl-1H-Pyrrolo[2,3-c]Pyridin-5-yl)Acetamide

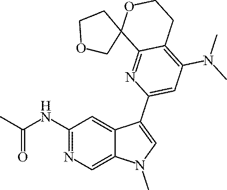 [SMILES: CC(=O)Nc1cc2c(-c3cc(N(C)C)c4c(n3)C3(CCOC3)OCC4)cn(C)c2cn1]

### Step 1 Synthesis of 2′-Chloro-N,N-Dimethyl-4,5,5′,6′-Tetrahydro-2H-Spiro[Furan-3,8′-Py rano[3,4-b]Pyridine]-4′-Amine

2′,4′-Dichloro-4,5,5′,6′-tetrahydro-2H-spiro[furan-3,8′-pyrano[3,4-b]pyridine] (78 mg, 0.30 mmol) and dimethylamine aqueous solution (5 mL, 40.4 mmol, 40 mass%) were added to a 10 mL one-neck round bottom flask, the mixture was stirred overnight at 50° C. The solution was concentrated in vacuum, and the resulting residue was purified by column chromatography (petroleum ether/ethyl acetate (v/v) = 4/1) to obtain the title compound as a white solid (54 mg, yield 67.00%).

MS (ESI, pos. ion) m/z: 269.1 [M+H]⁺.

### Step 2 Synthesis of N-(3-(4′-(Dimethylamino)-4,5,5′,6′-Tetrahydro-2H-Spiro[Furan-3,8′-P yrano[3,4-b]Pyridin]-2′-yl)-1-Methyl-1H-Pyrrolo[2,3-c]Pyridin-5-yl)Acetamide

Under N₂, 2′-chloro-N,N-dimethyl-4,5,5′,6′-tetrahydro-2H-spiro[furan-3,8′-pyrano[3,4-b]pyridine]-4′-amine (50 mg, 0.19 mmol), N-[1-methyl-3-(4,4,5,5-tetramethyl-1,3,2-dioxaborolan-2-yl)-1H-pyrrolo[2,3-c]pyridin-5-yl]acetami de (70.2 mg, 0.22 mmol), potassium carbonate (51 mg, 0.37 mmol) and PdCl₂dppf (16 mg, 0.02 mmol) were added to a 25 mL two-neck round bottom flask, then vacuumized, and 1,4-dioxane (5 mL) was added under N₂, the mixture was stirred to dissolve most of the solids, then water (2 mL) was added and deoxygenated by bubbling N₂ for 10 min. The flask was connected to a reflux condenser, under N₂, the mixture was heated to 100° C. and reacted overnight. After the heating was stopped, the mixture was cooled to room temperature, then 10 mL of water was added to quench the reaction, after extraction with chloroform (3 × 10 mL), the combined organic phase was dried over anhydrous sodium sulfate, and the resulting residue was purified by column chromatography (ethyl acetate/methanol (v/v) = 91/9) to obtain the title compound as a yellow solid (30.0 mg, yield 38.20%).

MS (ESI, pos. ion) m/z: 422.2 [M+H]⁺;

¹H NMR (400 MHz, CDCl₃) δ 8.99 (s, 1H), 8.40 (s, 1H), 8.15 (s, 1H), 7.83 (s, 1H), 7.12 (s, 1H), 4.33 - 4.07 (m, 4H), 3.92 (s, 3H), 3.94 - 3.82 (m, 2H), 2.92 (s, 6H), 2.83 (s, 3H), 2.36 - 2.26 (m, 1H), 2.23 (s, 3H).

### Example 25 N-(3-(4′-Cyano-4,5,5′,6′-Tetrahydro-2H-Spiro[Furan-3,8′-Pyrano[3,4-b]Pyridi ne]-2′-yl)-1-Methyl-1H-Pyrrolo[2,3-c]Pyridin-5-yl)Acetamide

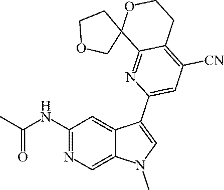 [SMILES: CC(=O)Nc1cc2c(-c3cc(C#N)c4c(n3)C3(CCOC3)OCC4)cn(C)c2cn1]

Under N₂, N-(3-(4′-chloro-4,5,5′,6′-tetrahydro-2H-spiro[furan-3,8′-pyrano[3,4-b]pyridine]-2′-yl)-1-methyl-1H-pyrrolo[2,3-c]pyridin-5-yl)acetamide (23 mg, 0.06 mmol), zinc cyanide (20 mg, 0.17 mmol) and bis(tri-tert-butylphosphine) palladium (14 mg, 0.03 mmol) were added to a 25 mL one-neck round bottom flask, then vacuumized, DMF (5 mL) was added under N₂, and the mixture was bubbled with N₂ for 10 min under stirring, then a solution of tri-tert-butylphosphine in n-hexane (0.04 mL, 0.02 mmol, 10 mass%) was added. The flask was connected to a reflux condenser, under N₂, the mixture was heated to 120° C. and reacted overnight. The resulting solution was concentrated in vacuum, the resulting residue was purified by column chromatography (ethyl acetate/methanol (v/v) = 91/9) to obtain the title compound as a yellow solid (16 mg, yield 71.20%).

MS (ESI, pos. ion) m/z: 404.2 [M+H]⁺;

¹H NMR (600 MHz, CDCl₃) δ 9.03 (s, 1H), 8.44 (s, 1H), 8.06 (s, 1H), 7.77 (s, 1H), 7.66 (s, 1H), 4.43 - 4.36 (m, 1H), 4.26 - 4.16 (m, 3H), 4.09 - 3.99 (m, 2H), 3.98 - 3.93 (m, 3H), 3.12 - 3.01 (m, 2H), 2.93 - 2.87 (m, 1H), 2.43 - 2.34 (m, 1H), 2.23 (s, 3H).

### Example 26 N-(3-(4′-(3-Hydroxy-3-Methylbutoxy)-4,5,5′,6′-Tetrahydro-2H-Spiro[Furan-3, 8′-Pyrano[3,4-b]Pyridin]-2′-yl)-1-Methyl-1H-Pyrrolo[2,3-c]Pyridin-5-yl)Acetamide

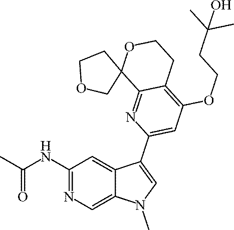 [SMILES: CC(=O)Nc1cc2c(-c3cc(OCCC(C)(C)O)c4c(n3)C3(CCOC3)OCC4)cn(C)c2cn1]

### Step 1 Synthesis of 4-((2′-Chloro-4,5,5′,6′-Tetrahydro-2H-Spiro[Furan-3,8′-Pyrano[3,4-b] Pyridin]-4′-yl)Oxy)-2-Methylbutan-2-ol

2′,4′-Dichloro-4,5,5′,6′-tetrahydro-2H-spiro[furan-3,8′-pyrano[3,4-b]pyridine] (83 mg, 0.32 mmol), DMF (3 mL), 3,3-dimethyl-1,3-diol (74 mg, 0.64 mmol) and sodium hydride (18 mg, 0.45 mmol, 60% wt%) were added to a 25 mL round bottom flask in sequence, the mixture was stirred overnight at room temperature. The reaction was quenched by water and concentrated in vacuum, and the resulting residue was purified by column chromatography (petroleum ether/ethyl acetate (v/v) = 1/1) to obtain the title compound as colorless oily liquid (80 mg, yield 76.00%).

MS (ESI, pos. ion) m/z: 328.1 [M+H]⁺;

### Step 2 Synthesis of N-(3-(4′-(3-Hydroxy-3-meThylbutoxy)-4,5,5′,6′-Tetrahydro-2H-Spiro[ Furan-3,8′-Pyrano[3,4-b]Pyridin]-2′-yl)-1-Methyl-1H-Pyrrolo[2,3-c]Pyridin-5-yl)Acetamide

Under N₂, 4-((2′-chloro-4,5,5′,6′-tetrahydro-2H-spiro[furan-3,8′-pyrano[3,4-b]pyridin]-4′-yl)oxy)-2-methylbuta n-2-ol (66 mg, 0.22 mmol), N-[1-methyl-3-(4,4,5,5-tetramethyl-1,3,2-dioxaborolan-2-yl)-1H-pyrrolo[2,3-c]pyridin-5-yl]acetami de (164 mg, 0.52 mmol), potassium carbonate (61 mg, 0.44 mmol) and PdCl₂dppf (18 mg, 0.02 mmol) were added to a 25 mL two-neck round bottom flask, then vacuumized, and 1,4-dioxane (8 mL) was added under N₂, the mixture was stirred to dissolve most of the solids, then water (2 mL) was added and deoxygenated by bubbling N₂ for 10 min. The flask was connected to a reflux condenser, under N₂, the mixture was heated to 100° C. and reacted overnight. After the heating was stopped, the mixture was cooled to room temperature, then 10 mL of water was added to quench the reaction, after extraction with chloroform (3 × 10 mL), the combined organic phase was dried over anhydrous sodium sulfate, and the resulting residue was purified by column chromatography (ethyl acetate/methanol (v/v) = 91/9) to obtain the title compound as a yellow solid (35.0 mg, yield 34.10%).

MS (ESI, pos. ion) m/z: 481.3 [M+H]⁺;

¹H NMR (400 MHz, CDCl₃) δ 8.99 (s, 1H), 8.41 (s, 1H), 8.14 (s, 1H), 7.87 (s, 1H), 7.23 (s, 1H), 4.46 (t, J = 7.3 Hz, 2H), 4.29 - 4.09 (m, 4H), 4.02 - 3.94 (m, 2H), 3.92 (s, 3H), 2.83 -2.69 (m, 3H), 2.36 - 2.25 (m, 1H), 2.22 (s, 3H), 2.14 (t, J = 7.3 Hz, 2H), 1.36 (s, 6H).

### Example 27 N-(3-(4′-(3-Methoxy-3-Methylbutoxy)-4,5,5′,6′-Tetrahydro-2H-Spiro[Furan-3, 8′-Pyrano[3,4-b]Pyridin]-2′-yl)-1-Methyl-1H-Pyrrolo[2,3-c]Pyridin-5-yl)Acetamide

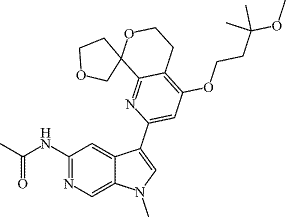 [SMILES: COC(C)(C)CCOc1cc(-c2cn(C)c3cnc(NC(C)=O)cc23)nc2c1CCOC21CCOC1]

### Step 1 Synthesis of 2′-Chloro-4′-(3-Methoxy-3-Methylbutoxy)-4,5,5′,6′-Tetrahydro-2H-Sp iro[furan-3,8′-Pyrano[3,4-b]Pyridine]

2′,4′-Dichloro-4,5,5′,6′-tetrahydro-2H-spiro[furan-3,8′-pyrano[3,4-b]pyridine] (70 mg, 0.27 mmol) and DMF (5 mL) were added to a 50 mL round bottom flask, under N₂, 3-methoxy-3-methylbutan-1-ol (63 mg, 0.54 mmol) and NaH (15 mg, 0.38 mmol, 60%) were added, the mixture was stirred overnight at room temperature. The solvent was concentrated, and the resulting residue was purified by column chromatography (petroleum ether/ethyl acetate (v/v) = 5/1) to obtain the title compound as a white solid (56 mg, yield 60.9%).

MS (ESI, pos. ion) m/z: 342.2 [M+H]⁺.

### Step 2 Synthesis of N-(3-(4′-(3-Methoxy-3-Methylbutoxy)-4,5,5′,6′-Tetrahydro-2H-Spiro[ Furan-3,8′-Pyrano[3,4-b]Pyridin]-2′-yl)-1-Methyl-1H-pYrrolo[2,3-c]pYridin-5-yl)Acetamide

Under N₂, 2′-chloro-4′-(3-methoxy-3-methylbutoxy)-4,5,5′,6′-tetrahydro-2H-spiro[furan-3,8′-pyrano[3,4-b]pyri dine] (55 mg, 0.16 mmol), N-[1-methyl-3-(4,4,5,5-tetramethyl-1,3,2-dioxaborolan-2-yl)-1H-pyrrolo[2,3-c]pyridin-5-yl]acetami de (61 mg, 0.19 mmol), 1,4-dioxane (8 mL), water (2 ml) and potassium carbonate (44 mg, 0.32 mmol) were added to a 50 mL two-neck round bottom flask, the mixture was deoxygenated by bubbling N₂ for 10 min, then PdCl₂dppf (39 mg, 0.048 mmol) was added and deoxygenated by bubbling N₂ for 10 min. The flask was connected to a reflux condenser, under N₂, the mixture was refluxed overnight, then 10 mL of water was added to quench the reaction, after extraction with chloroform (3 × 10 mL), the combined organic phase was dried over anhydrous sodium sulfate, and the resulting residue was purified by column chromatography (ethyl acetate/methanol (v/v) = 91/9) to obtain the title compound as a yellow solid (45 mg, yield 56.5%).

MS (ESI, pos. ion) m/z: 495.2 [M+H]⁺;

¹H NMR (400 MHz, DMSO-d₆): δ (ppm) 10.18 (s, 1H), 9.05 (s, 1H), 8.60 (s, 1H), 8.32 (s, 1H), 7.23 (s, 1H), 4.21 (d, J = 7.0 Hz, 2H), 3.98 (d, J = 7.6 Hz, 3H), 3.96 - 3.91 (m, 4H), 3.90 - 3.80 (m, 2H), 3.14 (s, 3H), 2.87 (dd, J = 21.0, 8.6 Hz, 1H), 2.63 (s, 2H), 2.21 - 2.12 (m, 1H), 2.09 (s, 3H), 1.99 (t, J = 6.7 Hz, 2H), 1.21 (s, 6H).

### Example 28 N-(3-(4′-(4-Methoxybutoxy)-4,5,5′,6′-Tetrahydro-2H-Spiro[Furan-3,8′-Pyran o[3,4-b]Pyridin]-2′-yl)-1-Methyl-1H-Pyrrolo[2,3-c]Pyridin-5-yl)Acetamide

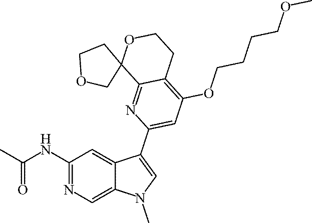 [SMILES: COCCCCOc1cc(-c2cn(C)c3cnc(NC(C)=O)cc23)nc2c1CCOC21CCOC1]

### Step 1 Synthesis of 2′-Chloro-4′-(4-Methoxybutoxy)-4,5,5′,6′-Tetrahydro-2H-Spiro[Furan -3,8′Pyrano[3,4-b]Pyridine]

2′,4′-Dichloro-4,5,5′,6′-tetrahydro-2H-spiro[furan-3,8′-pyrano[3,4-b]pyridine] (60 mg, 0.23 mmol), DMF (3 mL) and 4-methoxybutan-1-ol (34 mg, 0.32 mmol) were added to a 25 mL one-neck flask in sequence, the mixture was cooled to 0° C., then sodium hydride (12 mg, 0.32 mmol) was added slowly, the solution was reacted at room temperature after the addition. After the TLC plate showed that the reaction was completed, 5 mL of saturated ammonium chloride solution was added to quench the reaction, after extraction with ethyl acetate (3 × 20 mL), the organic phases were combined, and the solvent was concentrated, the resulting residue was purified by column chromatography (petroleum ether/ethyl acetate (v/v) = 3/1) to obtain the title compound as colorless oily liquid (50 mg, yield 66%).

MS (ESI, pos. ion) m/z: 316.2 [M+H]⁺;

### Step 2 Synthesis of N-(3-(4′-(4-Methoxybutoxy)-4,5,5′,6′-Tetrahydro-2H-Spiro[Furan-3,8 ‘-Pyrano[3,4-b]Pyridin]-2′-yl)-1-Methyl-1H-Pyrrolo[2,3-c]Pyridin-5-yl)Acetamide

2′-Chloro-4′-(4-methoxybutoxy)-4,5,5′,6′-tetrahydro-2H-spiro[furan-3,8′pyran[3,4-b]p yridine] (50 mg, 0.15 mmol), N-[1-methyl-3-(4,4,5,5-tetramethyl-1,3,2-dioxaborolan-2-yl)-1H-pyrrolo[2,3-c]pyridin-5-yl]acetami de (60 mg, 0.19 mmol), potassium carbonate (42 mg, 0.30 mmol), PdCl₂dppf (12 mg, 0.01 mmol), 1,4-dioxane (5 mL) and water (2 mL) were added to a 25 mL two-neck flask in sequence, the mixture was reacted at 100° C. overnight under N₂. After the reaction was completed, the solvent was concentrated, and the resulting residue was purified by column chromatography (dichloromethane /methanol (v/v) = 30/1) to obtain the title compound as a yellow solid (32 mg, yield 43%).

MS (ESI, pos. ion) m/z: 481.2 [M+H]⁺;

¹H NMR (400 MHz, DMSO-d₆) δ 10.19 (s, 1H), 9.04 (s, 1H), 8.61 (s, 1H), 8.30 (s, 1H), 7.21 (s, 1H), 4.22 (td, J = 8.4, 4.2 Hz, 1H), 4.16 (t, J = 6.2 Hz, 2H), 3.99 (q, J = 7.5 Hz, 3H), 3.93 (s, 3H), 3.91 - 3.79 (m, 2H), 3.43 - 3.39 (m, 2H), 3.23 (d, J = 8.1 Hz, 3H), 2.86 (dt, J = 17.1, 8.6 Hz, 1H), 2.64 (d, J = 2.7 Hz, 2H), 2.21 - 2.13 (m, 1H), 2.09 (s, 3H), 1.87 - 1.76 (m, 2H), 1.69 (dt, J = 13.0, 6.4 Hz, 2H).

### Example 29 N-(3-(4′-(3-(Dimethylamino)Propoxy)-4,5,5′,6′-Tetrahydro-2H-Spiro[Furan-3, 8′-Pyrano[3,4-b]Pyridin]-2′-yl)-1-Methyl-1H-Pyrrolo[2,3-c]Pyridin-5-yl)Acetamide

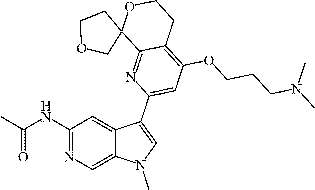 [SMILES: CC(=O)Nc1cc2c(-c3cc(OCCCN(C)C)c4c(n3)C3(CCOC3)OCC4)cn(C)c2cn1]

### Step 1 Synthesis of 3-((2′-Chloro-4,5,5′,6′-teTrahydro-2H-Spiro[Furan-3,8′-Pyrano[3,4-b] Pyridin]-4′-yl)Oxy)-N,N-Dimethylpropylamine

2′,4′-Dichloro-4,5,5′,6′-tetrahydro-2H-spiro[furan-3,8′-pyrano[3,4-b]pyridine] (60 mg, 0.23 mmol), DMF (3 mL) and 3-dimethylamino-1-propanol (38 mg, 0.32 mmol) were added to a 25 mL one-neck flask in sequence, the mixture was cooled to 0° C., then sodium hydride (12 mg, 0.32 mmol) was added slowly, the solution was reacted at room temperature after the addition. After the TLC plate showed that the reaction was completed, 5 mL of saturated ammonium chloride solution was added to quench the reaction, after extraction with ethyl acetate (3 × 20 mL), the organic phases were combined, and the solvent was concentrated to obtain the title compound as colorless oily liquid (50 mg, yield 66%).

MS (ESI, pos. ion) m/z: 327.2 [M+H]⁺.

### Step 2 Synthesis of N-(3-(4′-(3-(Dimethylamino)Propoxy)-4,5,5′,6′-Tetrahydro-2H-Spiro[ Furan-3,8′-Pyrano[3,4-b]Pyridin]-2′-yl)-1-Methyl-1H-Pyrrolo[2,3-c]Pyridin-5-yl)Acetamide

3-((2′-chloro-4,5,5′,6′-tetrahydro-2H-spiro[furan-3,8′-pyrano[3,4-b]pyridin]-4′-yl)oxy) -N,N-dimethylpropylamine (50 mg, 0.15 mmol), N-[1-methyl-3-(4,4,5,5-tetramethyl-1,3,2-dioxaborolan-2-yl)-1H-pyrrolo[2,3-c]pyridin-5-yl]acetami de (60 mg, 0.19 mmol), potassium carbonate (42 mg, 0.30 mmol), PdCl₂dppf (12 mg, 0.01 mmol), 1,4-dioxane (5 mL) and water (2 mL) were added to a 25 mL two-neck flask in sequence, the mixture was reacted at 100° C. overnight under N₂. After the reaction was completed, the solvent was concentrated, and the resulting residue was purified by column chromatography (dichloromethane /methanol (v/v) = 91/9) to obtain the title compound as a yellow solid (32 mg, yield 43%).

MS (ESI, pos. ion) m/z: 480.2 [M+H]⁺;

¹H NMR (400 MHz, DMSO-d₆) δ 10.19 (s, 1H), 9.05 (s, 1H), 8.60 (s, 1H), 8.30 (s, 1H), 7.30 (s, 1H), 7.21 (s, 1H), 6.69 (s, 1H), 4.26 - 4.15 (m, 3H), 3.99 (d, J = 7.7 Hz, 3H), 3.93 (s, 3H), 3.90 - 3.80 (m, 2H), 2.87 (dd, J = 20.8, 8.6 Hz, 1H), 2.65 (d, J = 2.6 Hz, 2H), 2.46 (t, J = 6.9 Hz, 2H), 2.21 (s, 6H), 2.09 (s, 3H), 1.99 - 1.89 (m, 2H), 1.76 (s, 4H).

### Example 30 N-(3-(4′-(3-Methoxy-2,2-Dimethylpropoxy)-4,5,5′,6′-Tetrahydro-2H-Spiro[Fu ran-3,8′-Pyrano[3,4-b]Pyridin]-2′-yl)-1-Methyl-1H-Pyrrolo[2,3-c]Pyridin-5-yl)Acetamide

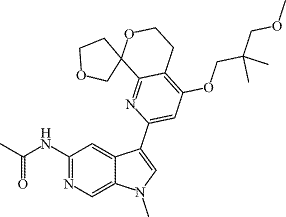 [SMILES: COCC(C)(C)COc1cc(-c2cn(C)c3cnc(NC(C)=O)cc23)nc2c1CCOC21CCOC1]

### Step 1 Synthesis of 2′-Chloro-4′-(3-Methoxy-2,2-Dimethylpropoxy)-4,5,5′,6′-Tetrahydro-2H-Spiro[Furan-3,8′-Pyrano[3,4-b]Pyridine]

2′,4′-Dichloro-4,5,5′,6′-tetrahydro-2H-spiro[furan-3,8′-pyrano[3,4-b]pyridine] (60 mg, 0.23 mmol), DMF(4 mL) and 3-methoxy-2,2-dimethylpropan-1-ol (71 mg, 0.57 mmol) were added to a 25 mL one-neck flask in sequence, the mixture was cooled to 0° C., then sodium hydride (12 mg, 0.32 mmol) was added slowly, the solution was reacted at room temperature after the addition. After the TLC plate showed that the reaction was completed, 5 mL of saturated ammonium chloride solution was added to quench the reaction, after extraction with ethyl acetate (3 × 20 mL), the organic phases were combined, and the solvent was concentrated, and the resulting residue was purified by column chromatography (petroleum ether/ethyl acetate (v/v) = 3/1) to obtain the title compound as colorless oily liquid (60 mg, yield 76%).

MS (ESI, pos. ion) m/z: 342.2 [M+H]⁺;

### Step 2 Synthesis of N-(3-(4′-(3-Methoxy-2,2-Dimethylpropoxy)-4,5,5′,6′-Tetrahydro-2H-S piro[Furan-3,8′-Pyrano[3,4-b]Pyridin]-2′-yl)-1-Methyl-1H-Pyrrolo[2,3-c]Pyridin-5-yl)Acetamid e

2′-Chloro-4′-(3-methoxy-2,2-dimethylpropoxy)-4,5,5′,6′-tetrahydro-2H-spiro[furan-3, 8′-pyrano[3,4-b]pyridine] (60 mg, 0.17 mmol), N [1-methyl-3-(4,4,5,5-tetramethyl-1,3,2-dioxaborolan-2-yl)-1H-pyrrolo[2,3-c]pyridin-5-yl]acetami de (66 mg, 0.20 mmol), potassium carbonate (48 mg, 0.34 mmol), PdCl₂dppf (14 mg, 0.01 mmol), 1,4-dioxane (5 mL) and water (2 mL) were added to a 25 mL two-neck flask in sequence, the mixture was reacted at 100° C. overnight under N₂. After the reaction was completed, the solvent was concentrated, and the resulting residue was purified by column chromatography (dichloromethane /methanol (v/v) = 30/1) to obtain the title compound as a yellow solid (70 mg, yield 80%).

MS (ESI, pos. ion) m/z: 495.2 [M+H]⁺;

¹H NMR (400 MHz, DMSO-d₆) δ 10.17 (s, 1H), 9.06 (s, 1H), 8.60 (s, 1H), 8.34 (s, 1H), 7.21 (s, 1H), 4.23 (d, J = 3.5 Hz, 1H), 3.99 (dd, J = 16.1, 10.1 Hz, 4H), 3.93 (s, 3H), 3.90 (d, J = 10.4 Hz, 3H), 3.27 (s, 3H), 3.25 (s, 2H), 2.88 (d, J = 12.0 Hz, 1H), 2.68 (d, J = 2.6 Hz, 2H), 2.24 - 2.13 (m, 1H), 2.09 (s, 3H), 1.05 (d, J = 20.1 Hz, 6H).

### Example 31 N-(3-(4′-((Dimethylamino)Methyl)-4,5,5′,6′-Tetrahydro-2H-Spiro[Furan-3,8′-Pyrano[3,4-b]Pyridin]-2′-yl)-1-Methyl-1H-Pyrrolo[2,3-c]Pyridin-5-yl)Acetamide

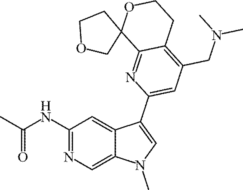 [SMILES: CC(=O)Nc1cc2c(-c3cc(CN(C)C)c4c(n3)C3(CCOC3)OCC4)cn(C)c2cn1]

### Step 1 Synthesis of 4′-(Hydroxymethyl)-4,5,5′,6′-Tetrahydro-2H-Spiro[Furan-3,8′-Pyrano [3,4-b]Pyridine]1′-Oxide

4,5,5′,6′-tetrahydro-2H-spiro[furan-3,8′-pyrano[3,4-b]pyridine]-4′-methanol (0.30 g, 1.36 mmol) and dichloromethane (20 mL) were added to a 50 mL round bottom flask, then m-chloroperbenzoic acid (0.41 g, 2.03 mmol, 85 mass%) was added under N₂, the mixture was stirred at room temperature overnight. 25 mL of saturated sodium sulfite was added to quench the reaction, then the solution was stirred for 15 min, 25 mL of saturated sodium carbonate was added, after extraction with chloroform (3 × 25 mL), the combined organic phase was dried over anhydrous sodium sulfate, filtered, and concentrated under reduced pressure, the resulting residue was purified by column chromatography (v/v) = 95/5) to obtain the title compound as a white solid (0.26 g, yield 80.8%).

MS (ESI, pos. ion) m/z: 238.2 [M+H]⁺;

### Step 2 Synthesis of 2′-Chloro-4′-(Chloromethyl)-4,5,5′,6′-Tetrahydro-2H-Spiro[Furan-3,8′ -Pyrano[3,4-b]Pyridine]

4′-(Hydroxymethyl)-4,5,5′,6′-tetrahydro-2H-spiro[furan-3,8′-pyrano[3,4-b]pyridine] 1′-oxide (0.26 g, 1.10 mmol) and phosphine oxychloride (15 mL, 165 mmol) were added to a 50 mL round bottom flask, and refluxed for 1 h. The mixture was concentrated in vacuum, then 12 mL of saturated sodium bicarbonate was added, the pH was adjusted to 7 with saturated sodium carbonate, after extraction with ethyl acetate (3 × 20 mL), the combined organic phase was washed with 20 mL of saturated sodium chloride, then dried and filtered, and concentrated under reduced pressure, the resulting residue was purified by column chromatography (dichloromethane/methanol (v/v) = 97/7) to obtain the title compound as a yellow-white solid (0.050 g, yield 16.6%).

MS (ESI, pos. ion) m/z: 274.2 [M+H]⁺;

¹H NMR (400 MHz, DMSO-d₆): δ (ppm) 7.52 (s, 1H), 4.80 (s, 2H), 4.02 - 3.88 (m, 6H), 2.87 - 2.82 (m, 2H), 2.37 - 2.31 (m, 1H), 2.27 - 2.18 (m, 1H).

### Step 3 Synthesis of 1-(2′-Chloro-4,5,5′,6′-Tetrahydro-2H-Spiro[Furan-3,8′-Pyrano[3,4-b]P yridin]-4′-yl)-N,N-Dimethylmethylamine

2′-Chloro-4′-(chloromethyl)-4,5,5′,6′-tetrahydro-2H-spiro[furan-3,8′-pyrano[3,4-b]pyr idine] (90 mg, 0.33 mmol), dimethylamine (0.33 mL, 0.66 mmol, 2 N in THF), THF (8 mL) and DIPEA (84 mg, 0.66 mmol) were added to a 50 mL two-neck round bottom flask under N₂, the mixture was heated to 30° C. and reacted overnight. The solvent was concentrated, and the resulting residue was purified by column chromatography (dichloromethane/methanol (v/v) = 40/1) to obtain the title compound as yellow oily liquid (60 mg, yield 64.6%).

MS (ESI, pos. ion) m/z: 283.2 [M+H]⁺;

1H NMR (400 MHz, DMSO-d₆): δ (ppm) 7.30 (s, 1H), 4.03 - 3.82 (m, 6H), 3.36 (s, 2H), 2.80 (t, J = 4.8 Hz, 2H), 2.37 (dd, J = 15.1, 6.4 Hz, 1H), 2.19 (d, J = 7.9 Hz, 7H).

### Step 4 Synthesis of N-(3-(4′-((Dimethylamino)Methyl)-4,5,5′,6′-Tetrahydro-2H-Spiro[Fur an-3,8′-Pyrano[3,4-b]Pyridin]-2′-yl)-1-Methyl-1H-Pyrrolo[2,3-c]Pyridin-5-yl)Acetamide

1-(2′-Chloro-4,5,5′,6′-tetrahydro-2H-spiro[furan-3,8′-pyrano[3,4-b]pyridin]-4′-yl)-N,N -dimethylmethylamine (60 mg, 0.21 mmol), N-[1-methyl-3-(4,4,5,5-tetramethyl-1,3,2-dioxaborolan-2-yl)-1H-pyrrolo[2,3-c]pyridin-5-yl]acetami de (0.12 g, 0.26 mmol, 70%), 1,4-dioxane (8 mL), water (2 mL) and potassium carbonate (58 mg, 0.42 mmol) were added to a 50 mL two-neck round bottom flask under N₂, the mixture was deoxygenated by bubbling N₂ for 10 min, then PdCl₂dppf (52 mg, 0.063 mmol) was added and deoxygenated by bubbling N₂ for 10 min. The flask was connected to a reflux condenser, under N₂, the mixture was refluxed overnight, then 10 mL of water was added to quench the reaction, after extraction with chloroform (3 × 10 mL), the combined organic phase was dried over anhydrous sodium sulfate, and the resulting residue was purified by column chromatography (ethyl acetate/methanol (v/v) = 91/9) to obtain the title compound as a yellow solid (55 mg, yield 59.5%).

MS (ESI, pos. ion) m/z: 436.2 [M+H]⁺;

¹H NMR (400 MHz, DMSO-d₆): δ (ppm) 10.21 (s, 1H), 9.02 (s, 1H), 8.62 (s, 1H), 8.26 (s, 1H), 7.33 (s, 1H), 4.03 - 3.89 (m, 8H), 2.85 (s, 3H), 2.34 (d, J = 6.0 Hz, 3H), 2.22 (d, J= 5.6 Hz, 2H), 2.09 (s, 4H), 2.00 (d, J = 7.5 Hz, 1H), 1.76 (s, 3H).

### Example 32 N-(3-(4′-(3-(2-Methoxyethoxy)Propoxy)-4,5,5′,6′-Tetrahydro-2H-Spiro[Furan -3,8′-Pyrano[3,4-b]Pyridin]-2′-yl)-1-Methyl-1H-Pyrrolo[2,3-c]Pyridin-5-yl)Acetamide

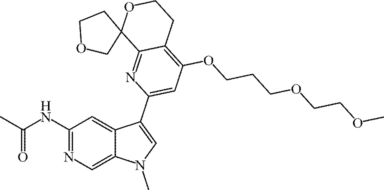 [SMILES: COCCOCCCOc1cc(-c2cn(C)c3cnc(NC(C)=O)cc23)nc2c1CCOC21CCOC1]

### Step 1 Synthesis of 2′-Chloro-4′-(3-(2-Methoxyethoxy)Propoxy)-4,5,5′,6′-Tetrahydro-2H-Spiro[Furan-3,8′-Pyrano[3,4-b]Pyridine]

2′,4′-Dichloro-4,5,5′,6′-tetrahydro-2H-spiro[furan-3,8′-pyrano[3,4-b]pyridine] (85 mg, 0.33 mmol) and anhydrous DMF (3 mL) were added to a flask, the mixture was stirred evenly, then 3-(2-methoxyethoxy)propan-1-ol (93 mg, 0.69 mmol) and sodium hydride (18 mg, 0.45 mmol, 60 mass%) were added under N₂, the solution was heated to 50° C. and stirred overnight, then 10 drops of water were added to quench the reaction. The mixture was concentrated in vacuum, and the resulting residue was purified by column chromatography (petroleum ether/ethyl acetate (v/ v) = 5/1) to obtain the title compound as colorless transparent oil (73 mg, yield 62.2%).

MS (ESI, pos. ion) m/z: 358.2 [M+H]⁺;

¹H NMR (400 MHz, CDCl₃) δ (ppm) 6.68 (s, 1H), 4.16 - 4.06 (m, 5 H), 3.98 - 3.85 (m, 2 H), 3.71 - 3.59 (m, 5 H), 3.56-3.54 (m, 2 H), 3.39 (s, 3 H), 2.73 - 2.56 (m, 3 H), 2.27 - 2.19 (m, 1 H), 2.16 - 2.06 (m, 2 H).

### Step 2 Synthesis of N-(3-(4′-(3-(2-Methoxyethoxy)Propoxy)-4,5,5′,6′-Tetrahydro-2H-Spir o[furan-3,8′-Pyrano[3,4-b]Pyridin]-2′-yl)-1-Methyl-1H-Pyrrolo[2,3-c]Pyridin-5-yl)Acetamide

2′-Chloro-4′-(3-(2-methoxyethoxy)propoxy)-4,5,5′,6′-tetrahydro-2H-spiro[furan-3,8′-pyrano[3,4-b]pyridine] (58 mg, 0.16 mmol), N-[1-methyl-3-(4,4,5,5-tetramethyl-1,3,2-dioxaborolan-2-yl)-1H-pyrrolo[2,3-c]pyridin-5-yl]acetami de (86 mg, 0.20 mmol), potassium carbonate (45 mg, 0.33 mmol) and PdCl₂dppf (15 mg, 0.018 mmol) were added to a round bottom flask, the mixture was purged with N₂, then 1,4-dioxane (4 mL) and water (1.5 mL) were added, and the mixture was purged with N₂ again and heated to 100° C. and stirred for 3 h. Then the solution was cooled to room temperature, concentrated under reduced pressure, the resulting residue was purified by column chromatography (dichloromethane/methanol (v/v) = 91/9) to obtain the title compound as a yellow semi-solid (52.0 mg, yield 62.83%).

MS (ESI, pos. ion) m/z: 511.4 [M+H]⁺;

¹H NMR (400 MHz, CDCl₃) δ (ppm) 9.04 (s, 1H), 8.43 (s, 1H), 8.17 (s, 1H), 7.81 (s, 1H), 7.05 (s, 1H), 4.38 - 4.11 (m, 7 H), 3.94 (s, 3 H), 3.77 - 3.53 (m, 7 H), 3.39 (s, 3 H), 2.98 -2.71 (m, 3 H), 2.41 - 2.29 (m, 1H), 2.24 (s, 3 H), 2.21 - 2.14 (m, 2 H).

### Example 33 N-(3-(4′-(3-(Difluoromethoxy)Propoxy)-4,5,5′,6′-Tetrahydro-2H-Spiro[Furan-3,8′-Pyrano[3,4-b]Pyridin]-2′-yl)-1-Methyl-1H-Pyrrolo[2,3-c]Pyridin-5-yl)Acetamide

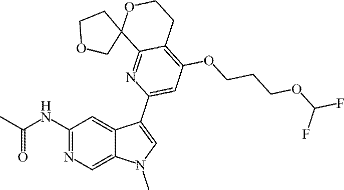 [SMILES: CC(=O)Nc1cc2c(-c3cc(OCCCOC(F)F)c4c(n3)C3(CCOC3)OCC4)cn(C)c2cn1]

### Step 1 Synthesis of 2′-Chloro-4′-(3-(Difluoromethoxy)Propoxy)-4,5,5′,6′-Tetrahydro-2H-S piro[Furan-3,8′-Pyrano[3,4-b]Pyridine]

2′,4′-Dichloro-4,5,5′,6′-tetrahydro-2H-spiro[furan-3,8′-pyrano[3,4-b]pyridine] (50 mg, 0.19 mmol), DMF (4 mL) and 3-(difluoromethoxy)propan-1-ol (35 mg, 0.26 mmol) were added to a 25 mL one-neck flask, the mixture was cooled to 0° C., then sodium hydride (10 mg, 0.25 mmol) was added slowly, and the solution was reacted at room temperature after the addition. 5 mL of saturated ammonium chloride solution was added to quench the reaction, after extraction with ethyl acetate (3 × 20 mL), the organic phases were combined, the solvent was concentrated, the resulting residue was purified by column chromatography (petroleum ether/ethyl acetate (v/v) = 3/1) to obtain the title compound as colorless oily liquid (40 mg, yield 59%).

MS (ESI, pos. ion) m/z: 350.1 [M+H]⁺.

### Step 2 Synthesis of N-(3-(4′-(3-(Difluoromethoxy)Propoxy)-4,5,5′,6′-Tetrahydro-2H-Spir o[furan-3,8′-Pyrano[3,4-b]Pyridin]-2′-yl)-1-Methyl-1H-Pyrrolo[2,3-c]Pyridin-5-yl)Acetamide

2′-Chloro-4′-(3-(difluoromethoxy)propoxy)-4,5,5′,6′-tetrahydro-2H-spiro[furan-3,8′-p yrano[3,4-b]pyridine] (40 mg, 0.11 mmol), N-[1-methyl-3-(4,4,5,5-tetramethyl-1,3,2-dioxaborolan-2-yl)-1H-pyrrolo[2,3-c]pyridin-5-yl]acetami de (70 mg, 0.16 mmol), potassium carbonate (31 mg, 0.23 mmol), PdCl₂dppf (9 mg, 0.01 mmol), 1,4-dioxane (5 mL) and water (2 mL) were added to a 25 mL two-neck flask, the mixture was reacted at 100° C. under N₂ overnight. After the reaction was completed, the solvent was concentrated and the resulting residue was purified by column chromatography (dichloromethane/methanol (v/v) = 30/1) to obtain the title compound as a yellow solid (27 mg, yield 47%).

MS (ESI, pos. ion) m/z: 503.2 [M+H]⁺;

¹H NMR (400 MHz, DMSO-d₆) δ 10.19 (s, 1H), 9.05 (s, 1H), 8.61 (s, 1H), 8.32 (s, 1H), 7.24 (s, 1H), 6.71 (t, J = 76.1 Hz, 1H), 4.24 (t, J= 5.9 Hz, 3H), 4.10 - 3.96 (m, 5H), 3.93 (s, 3H), 3.86 (ddd, J = 17.6, 11.7, 6.0 Hz, 2H), 2.87 (dd, J = 20.9, 8.6 Hz, 1H), 2.66 (s, 2H), 2.15 (ddd, J= 21.6, 10.7, 5.1 Hz, 3H), 2.09 (s, 3H).

### Example 34 N-(3-(4′-(3-Hydroxypropoxy)-4,5,5′,6′-Tetrahydro-2H-Spiro[Furan-3,8′-Pyran o[3,4-b]Pyridin]-2′-yl)-1-Methyl-1H-Pyrrolo[2,3-c]Pyridin-5-yl)Acetamide

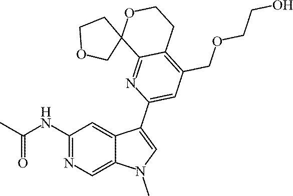 [SMILES: CC(=O)Nc1cc2c(-c3cc(COCCO)c4c(n3)C3(CCOC3)OCC4)cn(C)c2cn1]

### Step 1 Synthesis of 3-((2′-Chloro-4,5,5′,6′-Tetrahydro-2H-Spiro[Furan-3,8′-Pyran[3,4-b]P yridin]-4′-yl)Oxy)Propanol

2′,4′-Dichloro-4,5,5′,6′-tetrahydro-2H-spiro[furan-3,8′-pyrano[3,4-b]pyridine] (50 mg, 0.19 mmol), DMF(2 mL) and 1,3-propanediol (22 mg, 0.29 mmol) were added to a 25 mL one-neck flask in sequence, the mixture was cooled to 0° C., then sodium hydride (10 mg, 0.27 mmol) was added slowly, the solution was reacted at room temperature after the addition. After the TLC plate showed that the reaction was completed, 5 mL of saturated ammonium chloride solution was added to quench the reaction, after extraction with ethyl acetate (3 × 20 mL), the organic phases were combined, and the solvent was concentrated, and the resulting residue was purified by column chromatography (petroleum ether/ethyl acetate (v/v) =3/1) to obtain the title compound as colorless oily liquid (50 mg, yield 87%).

MS (ESI, pos. ion) m/z: 300.2 [M+H]⁺.

### Step 2 Synthesis of N-(3-(4′-(3-Hydroxypropoxy)-4,5,5′,6′-Tetrahydro-2H-Spiro[Furan-3, 8′-Pyrano[3,4-b]Pyridin]-2′-yl)-1-Methyl-1H-Pyrrolo[2,3-c]Pyridin-5-yl)Acetamide

3-((2′-Chloro-4,5,5′,6′-tetrahydro-2H-spiro[furan-3,8′-pyrano[3,4-b]pyridin]-4′-yl)oxy )propanol (50 mg, 0.16 mmol), N-[1-methyl-3-(4,4,5,5-tetramethyl-1,3,2-dioxaborolan-2-yl)-1H-pyrrolo[2,3-c]pyridin-5-yl]acetami de (100 mg, 0.31 mmol), potassium carbonate (46 mg, 0.33 mmol), PdCl₂dppf (13 mg, 0.02 mmol), 1,4-dioxane (8 mL) and water (2 mL) were added to a 25 mL two-neck flask in sequence, the mixture was reacted at 100° C. overnight under N₂. After the reaction was completed, the solvent was concentrated, and the resulting residue was purified by column chromatography (dichloromethane /methanol (v/v) = 91/9) to obtain the title compound as a yellow solid (32 mg, yield 42%).

MS (ESI, pos. ion) m/z: 453.2 [M+H]⁺;

¹H NMR (400 MHz, DMSO-d₆) δ 10.20 (s, 1H), 9.04 (s, 1H), 8.60 (s, 1H), 8.32 (s, 1H), 7.23 (s, 1H), 4.69 (s, 1H), 4.22 (t, J = 6.0 Hz, 3H), 4.05 - 3.95 (m, 3H), 3.92 (s, 3H), 3.91 -3.77 (m, 2H), 3.60 (t, J = 6.0 Hz, 2H), 2.87 (dd, J = 20.8, 8.7 Hz, 1H), 2.62 (d, J = 17.0 Hz, 2H), 2.23 - 2.12 (m, 1H), 2.09 (s, 3H), 1.99 - 1.86 (m, 2H).

### Example 35 N-(3-(4′-(3-Hydroxy-2,2-Dimethylpropoxy)-4,5,5′,6′-Tetrahydro-2H-Spiro[Fur an-3,8′-Pyrano[3,4-b]Pyridin]-2′-yl)-1-Methyl-1H-Pyrrolo[2,3-c]Pyridin-5-yl)Acetamide

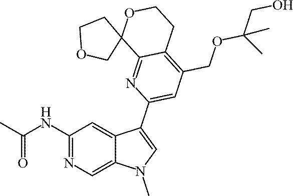 [SMILES: CC(=O)Nc1cc2c(-c3cc(COC(C)(C)CO)c4c(n3)C3(CCOC3)OCC4)cn(C)c2cn1]

### Step 1 Synthesis of 3-((2′-Chloro-4,5,5′,6′-Tetrahydro-2H-Spiro[Furan-3,8′-Pyrano[3,4-b] Pyridin]-4′-yl)Oxy)-2,2-Dimethylpropanol

2′,4′-Dichloro-4,5,5′,6′-tetrahydro-2H-spiro[furan-3,8′-pyrano[3,4-b]pyridine] (50 mg, 0.19 mmol), DMF (1 mL) and 2,2-dimethyl-1,3-propanediol (30 mg, 0.29 mmol) were added to a 25 mL one-neck flask in sequence, the mixture was cooled to 0° C., then sodium hydride (11 mg, 0.28 mmol) was added slowly, the solution was reacted at room temperature after the addition. After the TLC plate showed that the reaction was completed, 5 mL of saturated ammonium chloride solution was added to quench the reaction, after extraction with ethyl acetate (3 × 20 mL), the organic phases were combined, and the solvent was concentrated, the resulting residue was purified by column chromatography (petroleum ether/ethyl acetate (v/v) = 3/1) to obtain the title compound as colorless oily liquid (60 mg, yield 95%).

MS (ESI, pos. ion) m/z: 328.1 [M+H]⁺.

### Step 2 Synthesis of N-(3-(4′-(3-Hydroxy-2,2-Dimethylpropoxy)-4,5,5′,6′-Tetrahydro-2H-S piro[Furan-3,8′-Pyrano[3,4-b]Pyridin]-2′-yl)-1-Methyl-1H-Pyrrolo[2,3-c]Pyridin-5-yl)Acetamid e

3-((2′-Chloro-4,5,5′,6′-tetrahydro-2H-spiro[furan-3,8′-pyrano[3,4-b]pyridin]-4′-yl)oxy )-2,2-dimethylpropanol (70 mg, 0.21 mmol), N-[1-methyl-3-(4,4,5,5-tetramethyl-1,3,2-dioxaborolan-2-yl)-1H-pyrrolo[2,3-c]pyridin-5-yl]acetami de (120 mg, 0.25 mmol), potassium carbonate (59 mg, 0.43 mmol), PdCl₂dppf (17 mg, 0.02 mmol), 1,4-dioxane (8 mL) and water (2 mL) were added to a 25 mL two-neck flask in sequence, the mixture was reacted at 100° C. overnight under N₂. After the reaction was completed, the solvent was concentrated, and the resulting residue was purified by column chromatography (dichloromethane /methanol (v/v) = 91/9) to obtain the title compound as a yellow solid (60 mg, yield 58%).

MS (ESI, pos. ion) m/z: 481.2 [M+H]⁺;

¹H NMR (400 MHz, DMSO-d₆) δ 10.18 (s, 1H), 9.06 (s, 1H), 8.60 (s, 1H), 8.33 (s, 1H), 7.21 (s, 1H), 4.71 (s, 1H), 4.23 (d, J = 2.9 Hz, 1H), 3.99 (d, J = 7.5 Hz, 3H), 3.92 (s, 3H), 3.89 (d, J = 12.1 Hz, 4H), 3.33 (d, J = 4.7 Hz, 2H), 2.97 - 2.79 (m, 1H), 2.68 (s, 2H), 2.24 - 2.13 (m, 1H), 2.09 (s, 3H), 0.98 (s, 6H).

### Example 36 N-(3-(4′-((2-Methoxyethoxy)Methyl)-4,5,5′,6′-Tetrahydro-2H-Spiro[Furan-3,8 ‘-Pyrano[3,4-b]Pyridin]-2′-yl)-1methyl-1H-Pyrrolo[2,3-c]Pyridin-5-yl)Acetamide

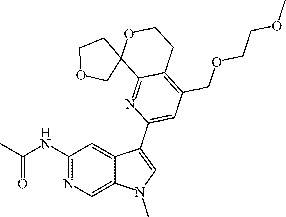 [SMILES: COCCOCc1cc(-c2cn(C)c3cnc(NC(C)=O)cc23)nc2c1CCOC21CCOC1]

### Step 1 Synthesis of 4′-((2-Methoxyethoxy)Methyl)-4,5,5′,6′-Tetrahydro-2H-Spiro[Furan-3,8′-Pyrano[3,4-b]Pyridine]

(4,5,5′,6′-Tetrahydro-2H-spiro[furo-3,8′-pyrano[3,4-b]pyridin]-4′-yl)methanol (0.12 g, 0.54 mmol) and DMF (5 mL) were added to a 50 mL round bottom flask, under N₂, 2-methoxyethyl-4-methylbenzenesulfonic acid (0.19 g, 0.81 mmol) and NaH (0.043 g, 1.08 mmol, 60%) were added, the mixture was stirred at room temperature for 2 h. The solvent was concentrated, and the resulting residue was purified by column chromatography (petroleum ether/ethyl acetate (v/v) =1/1) to obtain the title compound as yellow oily liquid (0.14 g, yield 89.1%).

MS (ESI, pos. ion) m/z: 280.2 [M+H]⁺.

### Step 2 Synthesis of 4′-((2-Methoxyethoxy)Methyl)-4,5,5′,6′-Tetrahydro-2H-Spiro[Furan-3,8′-Pyrano[3,4-b]Pyridine] 1′-Oxide

4′-((2-Methoxyethoxy)methyl)-4,5,5′,6′-tetrahydro-2H-spiro[furan-3,8′-pyrano[3,4-b] pyridine] (0.13 g, 0.46 mmol), dichloromethane (25 mL) and m-chloroperbenzoic acid (0.14 g, 0.70 mmol, 85 mass%) were added to a 100 mL flask, the mixture was stirred overnight at room temperature. 25 mL of saturated sodium sulfite was added to quench the reaction, then the solution was stirred for 15 min, 25 mL of saturated sodium carbonate was added, after extraction with chloroform (3 × 25 mL), the combined organic phase was dried over anhydrous sodium sulfate, filtered, and concentrated under reduced pressure, the resulting residue was purified by column chromatography (v/v) = 95/5) to obtain the title compound as a white solid (0.11 g, yield 80.4%).

MS (ESI, pos. ion) m/z: 296.2 [M+H]⁺;

¹H NMR (400 MHz, DMSO-d₆): δ (ppm) 8.11 (d, J = 6.6 Hz, 1H), 7.31 (d, J = 6.6 Hz, 1H), 4.53 (d, J = 11.1 Hz, 3H), 4.44 - 4.24 (m, 2H), 3.97 (dd, J = 11.2, 5.9 Hz, 1H), 3.90 - 3.81 (m, 2H), 3.72 (dd, J = 5.5, 3.3 Hz, 2H), 3.62 (dd, J = 5.6, 3.3 Hz, 2H), 3.42 (s, 3H), 3.29 - 3.09 (m, 1H), 2.96 - 2.80 (m, 1H), 2.76 - 2.56 (m, 1H), 1.98 - 1.88 (m, 1H).

### Step 3 Synthesis of 2′-Chloro-4′-((2-Methoxyethoxy)Methyl)-4,5,5′,6′-Tetrahydro-2H-Spi ro[furan-3,8′-Pyrano[3,4-b]Pyridine]

4′-((2-Methoxyethoxy)methyl)-4,5,5′,6′-tetrahydro-2H-spiro[furan-3,8′-pyrano[3,4-b] pyridine] 1′-oxide (0.11 g, 0.37 mmol) and phosphine oxychloride (15 mL, 165 mmol) were added to a 50 mL round bottom flask, and the mixture was refluxed for 1 h. After TLC monitoring showed that the reaction was completed, the resulting solution was concentrated in vacuum, then 12 mL of saturated sodium bicarbonate was added, and the pH was adjusted to 7 by saturated sodium carbonate, after extraction with ethyl acetate (3 × 20 mL), the combined organic phase was washed with 20 mL of saturated sodium chloride, then dried and filtered, and concentrated under reduced pressure, and the resulting residue was purified by column chromatography (dichloromethane/methanol (v/v)=97/7) to obtain the title compound as a yellow-white solid (0.052 g, yield 44.5%).

MS (ESI, pos. ion) m/z: 314.2 [M+H]⁺;

### Step 4 Synthesis of N-(3-(4′-((2-Methoxyethoxy)Methyl)-4,5,5′,6′-Tetrahydro-2H-Spiro[F uran-3,8′-Pyrano[3,4-b]Pyridin]-2′-yl)-1methyl-1H-Pyrrolo[2,3-c]Pyridin-5-yl)Acetamide

Under N₂, 2′-chloro-4′-((2-methoxyethoxy)methyl)-4,5,5′,6′-tetrahydro-2H-spiro[furan-3,8′-pyrano[3,4-b]pyrid ine] (50 mg, 0.16 mmol), N-[1-methyl-3-(4,4,5,5-tetramethyl-1,3,2-dioxaborolan-2-yl)-1H-pyrrolo[2,3-c]pyridin-5-yl]acetami de (60 mg, 0.19 mmol), 1,4-dioxane (8 mL), water (2 mL) and potassium carbonate (44 mg, 0.32 mmol) were added to a 50 mL two-necked round bottom flask, the mixture was deoxygenated by bubbling N₂ for 10 min, then PdCl₂dppf (39 mg, 0.047 mmol) was added and deoxygenated by bubbling N₂ for 10 min. The flask was connected to a reflux condenser, the mixture was refluxed overnight under N₂, then 10 mL of water was added to quench the reaction, after extraction with chloroform (3 × 10 mL), the combined organic phase was dried over anhydrous sodium sulfate, and the resulting residue was purified by column chromatography (ethyl acetate/methanol (v/v) = 91/9) to obtain the title compound as a yellow solid (33 mg, yield 44.4%).

MS (ESI, pos. ion) m/z: 467.2 [M+H]⁺;

¹H NMR (400 MHz, DMSO-d₆): δ (ppm) 9.09 (s, 1H), 8.42 (s, 1H), 8.32 (s, 1H), 7.80 (s, 1H), 7.59 (s, 1H), 4.61 (s, 2H), 4.38 (s, 1H), 4.34 - 4.24 (m, 3H), 4.03 (d, J = 4.1 Hz, 2H), 3.93 (s, 3H), 3.67 (s, 2H), 3.44 (s, 3H), 3.00 - 2.80 (m, 3H), 2.38 (d, J = 5.8 Hz, 1H), 2.24 (s, 3H).

### Example 37 N-(3-(4′-((R)-3-Hydroxybutoxy)-4,5,5′,6′-Tetrahydro-2H-Spiro[Furan-3,8′-Py rano[3,4-b]Pyridin]-2′-yl)-1-Methyl-1H-Pyrrolo[2,3-c]Pyridin-5-yl)Acetamide

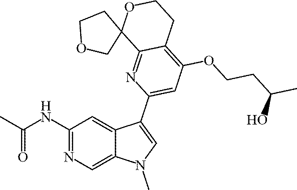 [SMILES: CC(=O)Nc1cc2c(-c3cc(OCC[C@@H](C)O)c4c(n3)C3(CCOC3)OCC4)cn(C)c2cn1]

### Step 1 Synthesis of Tert-Butyl 5-Acetamido-3-Bromo-1H-Pyrrolo[2,3-c]Pyridine-1-Carbo xylate

N-(3-Bromo-1H-pyrrolo[2,3-c]pyridin-5-yl)acetamide (3.25 g, 12.80 mmol), ahydrous acetonitrile (64 mL), Boc anhydride (3.37 g, 15.2 mmol) and 4-(dimethylamino)pyridine (156 mg, 1.28 mmol) were added to a 250 mL one-neck round bottom flask, the mixture was stirred at room temperature for 24 h. The solution was concentrated, and the resulting residue was purified by column chromatography (dichloromethane/methanol (v/v) = 97/3) to obtain the title compound as a white solid (4.53 g, yield 100.00%).

MS (ESI, pos. ion) m/z: 354.0 [M+H]⁺;

### Step 2 Synthesis of Tert-Butyl 5-Acetamido-3-(4,4,5,5-Tetramethyl-1,3,2-Dioxaborolan-2-yl)-1H-Pyrrolo[2,3-c]Pyridine-1-Carboxylate

Tert-butyl 5-acetamido-3-bromo-1H-pyrrolo[2,3-c]pyridine-1-carboxylate (5.46 g, 15.4 mmol), bipinacol borate (7.99 g, 30.8 mmol) and potassium acetate (3.09 g, 30.9 mmol) were added to a 250 mL round bottom flask, under N₂, anhydrous 1,4-dioxane (60 mL) and PdCl₂dppf (1.26 g, 1.54 mmol) were added, then the mixture was continued to bubble with N₂ for 10 min, the flask was connected a reflux condenser, and the solution was stirred at 100° C. for 3.5 h. The resulting solution was filtered through a celite pad, washed with 20 mL of ethyl acetate, the combined filtrates were concentrated, and the resulting residue was purified by column chromatography (petroleum ether/ethyl acetate (v/v) = 1/1) to obtain the title compound as a light yellow solid (4.09 g, yield 66.10%).

MS (ESI, pos. ion) m/z: 402.2 [M+H]⁺;

### Step 3 Synthesis of (2R)-4-((2′-Chloro-4,5,5′,6′-Tetrahydro-2H-Spiro[Furan-3,8′-Pyrano[3,4-b]Pyridine]-4′-yl)Oxy)Butan-2-Ol

2′,4′-Dichloro-4,5,5′,6′-tetrahydro-2H-spiro[furan-3,8′-pyrano[3,4-b]pyridine] (78 mg, 0.30 mmol), DMF (3 mL), 3,3-dimethyl-1,3-diol (74 mg, 0.82 mmol) and sodium hydride (17 mg, 0.42 mmol, 60% wt%) were added to a 25 mL round bottom flask, the mixture was stirred overnight at room temperature. The reaction was quenched by adding water, concentrated in vacuum, and the resulting residue was purified by column chromatography (petroleum ether/ethyl acetate (v/v) = 1/1) to obtain the title compound as colorless oily liquid (87 mg, yield 92.00%).

MS (ESI, pos. ion) m/z: 314.1 [M+H]⁺.

### Step 4 Synthesis of N-(3-(4′-((R)-3-Hydroxybutoxy)-4,5,5′,6′-Tetrahydro-2H-Spiro[Furan -3,8′-Pyrano[3,4-b]Pyridin]-2′-yl)-1H Pyrrolo[2,3-c]Pyridin-5-yl)Acetamide

Under N₂, (2R)-4-((2′-chloro-4,5,5′,6′-tetrahydro-2H-spiro[furan-3,8′-pyrano[3,4-b]pyridine]-4′-yl)oxy)butan-2 -ol (87 mg, 0.28 mmol), tert-butyl 5-acetamido-3-(4,4,5,5-tetramethyl-1,3,2-dioxaborolan-2-yl)-1H-pyrrolo[2,3-c]pyridine-1-carboxyl ate (145 mg, 0.36 mmol), potassium carbonate (46 mg, 0.33 mmol) and PdCl₂dppf (23 mg, 0.03 mmol) were added to a 25 mL two-neck round bottom flask, then vacuumized, and 1,4-dioxane (5 mL) was added under N₂, the mixture was stirred to dissolve most of the solids, then water (2 mL) was added and deoxygenated by bubbling N₂ for 10 min. The flask was connected to a reflux condenser, under N₂, the mixture was heated to 100° C. and reacted overnight. After the heating was stopped, the mixture was cooled to room temperature, concentrated in vacuum, then methanol was added, and the resulting residue was purified by column chromatography (dichloromethane/methanol (v/v) = 91/9) to obtain the title compound as a light yellow solid (94 mg, yield 74.90%).

MS (ESI, pos. ion) m/z: 453.1 [M+H]⁺.

### Step 5 Synthesis of N-(3-(4′-((R)-3-Hydroxybutoxy)-4,5,5′,6′-Tetrahydro-2H-Spiro[Furan -3,8′-Pyrano[3,4-b]Pyridin]-2′-yl)-1-Methyl-1H-Pyrrolo[2,3-c]Pyridin-5-yl)Acetamide

N-(3-(4′-((R)-3-hydroxybutoxy)-4,5,5′,6′-tetrahydro-2H-spiro[furan-3,8′-pyrano[3,4-b ]pyridin]-2′-yl)-1H-pyrrolo[2,3-c]pyridin-5-yl)acetamide (94 mg, 0.21 mmol), acetonitrile (5 mL), cesium carbonate (81 mg, 0.25 mmol) and methyl iodide (35 mg, 0.25 mmol) were added to a 25 mL round bottom flask, the mixture was stirred overnight at room temperature. The mixture was concentrated, and the resulting residue was purified by column chromatography (dichloromethane/methanol (v/v) = 95/5) to obtain the title compound as a light yellow solid (64 mg, yield 66.00%).

MS (ESI, pos. ion) m/z: 467.2 [M+H]⁺;

¹H NMR (400 MHz, CDCl₃) δ 8.95 (s, 1H), 8.39 (s, 1H), 8.33 (s, 1H), 7.85 (s, 1H), 7.20 (s, 1H), 4.51 - 4.31 (m, 2H), 4.28 - 4.05 (m, 5H), 4.02 - 3.93 (m, 2H), 3.90 (s, 3H), 3.43 (s, 1H), 2.78 (dt, J= 10.5, 7.2 Hz, 3H), 2.37 - 2.26 (m, 1H), 2.23 (s, 3H), 2.16 - 2.04 (m, 2H), 1.33 (d, J= 6.2 Hz, 3H).

### Example 38 N-(3-(4′-((S)-3-Methoxybutoxy)-4,5,5′,6′-Tetrahydro-2H-Spiro[Furan-3,8′-Py rano[3,4-b]Pyridin]-2′-yl)-1-Methyl-1H-Pyrrolo[2,3-c]Pyridin-5-yl)Acetamide

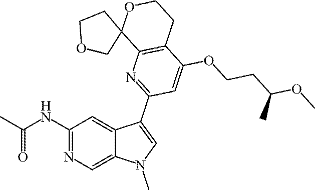 [SMILES: CO[C@@H](C)CCOc1cc(-c2cn(C)c3cnc(NC(C)=O)cc23)nc2c1CCOC21CCOC1]

### Step 1 Synthesis of (2S)-4-((2′-Chloro-4,5,5′,6′-Tetrahydro-2H-Spiro[Furan-3,8′-Pyrano[3,4-b]Pyridine]-4′-yl)Oxy)Butyl-2-Ol

(S)-1,3-Butanediol (8 mg, 0.09 mmol) and anhydrous DMF (2 mL) were added to a round bottom flask, then sodium hydride (10 mg, 0.25 mmol, 60 mass%) was added under stirring at room temperature, the mixture was stirred at room temperature for 10 min after the addition, then 2′,4′-dichloro-4,5,5′,6′-tetrahydro-2H-spiro[furan-3,8′-pyrano[3,4-b]pyridine] (15 mg, 0.06 mmol) was added, the mixture was stirred overnight after the addition, the reaction was quenched by adding 5 drops of water, the mixture was concentrated and the resulting residue was purified by column chromatography (petroleum ether/ethyl acetate (v/v) = 10/1) to obtain the title compound as a colorless oily substance (14 mg, yield 75.9%).

MS (ESI,pos.ion) m/z: 314.10 [M+H]⁺.

### Step 2 Synthesis of 2′-Chloro-4′-((S)-3-Methoxybutoxy)-4,5,5′,6′-Tetrahydro-2H-Spiro[Fu ran-3,8′-Pyrano [3,4-b]Pyridine]

(2S)-4-((2′-Chloro-4,5,5′,6′-tetrahydro-2H-spiro[furan-3,8′-pyrano[3,4-b]pyridine]-4′-yl)oxy)butyl-2-ol (14 mg, 0.05 mmol) and anhydrous THF (3 mL) were added to a flask, the mixture was stirred well at 0° C., under N₂, sodium hydride (3 mg, 0.13 mmol, 60 mass%) and methyl iodide (15 mg, 0.11 mmol) were added, then the solution was heated to 60° C. and stirred for 9 h, then the reactionwas quenched with 5 mL of water and diluted with 50 mL of ethyl acetate, the mixture was stirred for 5 min, and separated, the aqueous phase was extracted with 20 mL of ethyl acetate, the organic phase was dried over anhydrous sodium sulfate, concentrated and the resulting residue was purified by column chromatography (petroleum ether/ethyl acetate (v/v) = 5/1) to obtain the title compound as colorless transparent oil (13 mg, yield 88.89%).

¹H NMR (400 MHz, CDCl₃) δ 6.68 (s, 1H), 4.24 - 4.04 (m, 6 H), 4.01 - 3.85 (m, 2 H), 3.59 - 3.43 (m, 1 H), 3.35 (s, 3 H), 2.78 - 2.56 (m, 3 H), 2.29 - 2.18 (m, 1 H), 2.02 - 1.91 (m, 2 H), 1.23 (d, J = 6.1 Hz, 3 H).

### Step 3 Synthesis of N-(3-(4′-((S)-3-Methoxybutoxy)-4,5,5′,6′-Tetrahydro-2H-Spiro[Furan -3,8′-Pyrano[3,4-b]Pyridin]-2′-yl)-1-Methyl-1H-Pyrrolo[2,3-c]Pyridin-5-yl)Acetamide

2′-Chloro-4′-((S)-3-methoxybutoxy)-4,5,5′,6′-tetrahydro-2H-spiro[furan-3,8′-pyrano[3,4-b]pyridine] (36 mg, 0.11 mmol), N-[1-methyl-3-(4,4,5,5-tetramethyl-1,3,2-dioxaborolan-2-yl)-1H-pyrrolo[2,3-c]pyridin-5-yl]acetami de (59 mg, 0.13 mmol), potassium carbonate (35 mg, 0.25 mmol) and PdCl₂dppf (15 mg, 0.02 mmol) were added to a round bottom flask, the mixture was purged with N₂, then 1,4-dioxane (4 mL) and water (1.5 mL) were added, the mixture was purged with N₂ again and heated to 100° C. and stirred for 3.5 h. Then the solution was cooled to room temperature, concentrated under reduced pressure, the resulting residue was purified by column chromatography (dichloromethane/methanol (v/v) = 30/1) to obtain the title compound as a yellow semi-solid (20 mg, yield 37.89%).

MS (ESI,pos.ion) m/z: 481.2 [M+H]⁺;

¹H NMR (400 MHz, CDCl₃) δ (ppm) 9.05 (s, 1H), 8.43 (s, 1H), 8.22 (s, 1H), 7.80 (s, 1H), 7.06 (s, 1H), 4.32-4.16 (m, 6 H), 4.07 - 3.96 (m, 2 H), 3.94 (s, 3 H), 3.69 - 3.53 (m, 1 H), 3.38 (s, 3 H), 2.98 - 2.73 (m, 3 H), 2.40 - 2.30 (m, 1 H), 2.24 (s, 3 H), 2.09-1.99 (m, 2 H), 1.27 (d, J= 5.3 Hz, 4 H).

### Example 39 N-(3-(4′-((S)-3-Hydroxybutoxy)-4,5,5′,6′-Tetrahydro-2H-Spiro[Furan-3,8′-Pyr ano[3,4-b]Pyridin]-2′-yl)-1-Methyl-1H-Pyrrolo[2,3-c]Pyridin-5-yl)Acetamide

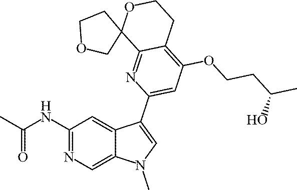 [SMILES: CC(=O)Nc1cc2c(-c3cc(OCC[C@H](C)O)c4c(n3)C3(CCOC3)OCC4)cn(C)c2cn1]

### Step 1 Synthesis of N-(3-(4′-((S)-3-Hydroxybutoxy)-4,5,5′,6′-Tetrahydro-2H-Spiro[Furan-3,8′-Pyrano[3,4-b]Pyridin]-2′-yl)-1H-Pyrrolo[2,3-c]Pyridin-5-yl)Acetamide

Under N₂, (2S)-4-((2′-chloro-4,5,5′,6′-tetrahydro-2H-spiro[furan-3,8′-pyrano[3,4-b]pyridine]-4′-yl)oxy)butan-2 -ol (68 mg, 0.22 mmol), tert-butyl 5-acetamido-3-(4,4,5,5,-tetramethyl-1,3,2-dioxaborolan-2-yl)-1H-pyrrolo[2,3-c]pyridine-1-carboxyl ate (113 mg, 0.28 mmol), potassium carbonate (60 mg, 0.43 mmol) and PdCl₂dppf (18 mg, 0.02 mmol) were added to a 25 mL two-neck round bottom flask, then vacuumized, and 1,4-dioxane (5 mL) was added under N₂, the mixture was stirred to dissolve most of the solids, then water (2 mL) was added and deoxygenated by bubbling N₂ for 10 min. The flask was connected to a reflux condenser, under N₂, the mixture was heated to 100° C. and reacted overnight. After the heating was stopped, the mixture was cooled to room temperature, concentrated in vacuum, and the resulting residue was purified by column chromatography (ethyl acetate/methanol (v /v) = 91/9) to obtain the title compound as a brown solid (70 mg, yield 71.40%).

MS (ESI,pos.ion) m/z: 453.1 [M+H]⁺.

### Step 2 Synthesis of N-(3-(4′-((S)-3-Hydroxybutoxy)-4,5,5′,6′-Tetrahydro-2H-Spiro[Furan-3,8′-Pyrano[3,4-b]Pyridin]-2′-yl)-1-Methyl-1H-Pyrrolo[2,3-c]Pyridin-5-yl)Acetamide

N-(3-(4′-((S)-3-Hydroxybutoxy)-4,5,5′,6′-tetrahydro-2H-spiro[furan-3,8′-pyrano[3,4-b ]pyridin]-2′-yl)-1H-pyrrolo[2,3-c]pyridin-5-yl)acetamide (70 mg, 0.16 mmol), acetonitrile (5 mL), cesium carbonate (65 mg, 0.25 mmol) and methyl iodide (29 mg, 0.20 mmol) were added to a 25 mL round bottom flask, the mixture was stirred overnight at room temperature. The mixture was concentrated, and the resulting residue was purified by column chromatography (dichloromethane/methanol (v/v) = 95/5) to obtain the title compound as a light yellow solid (54 mg, yield 74.80%).

MS (ESI,pos.ion) m/z: 467.3 [M+H]⁺;

¹H NMR (400 MHz, CDCl₃) δ 8.95 (s, 1H), 8.40 (s, 1H), 7.85 (s, 1H), 7.20 (s, 1H), 4.49 - 4.33 (m, 2H), 4.27 - 4.16 (m, 3H), 4.15 - 4.09 (m, 2H), 4.01 - 3.93 (m, 2H), 3.91 (s, 3H), 2.80 - 2.69 (m, 3H), 2.34 - 2.26 (m, 1H), 2.23 (s, 3H), 2.15 - 2.04 (m, 2H), 1.33 (d, J = 6.3 Hz, 3H).

### Example 40 N-(3-(4′-(3-Cyano-3-Methylbutoxy)-4,5,5′,6′-Tetrahydro-2H-Spiro[Furan-3,8′-Pyrano[3,4-b]Pyridin]-2′-yl)-1-Methyl-1H-Pyrrolo[2,3-c]Pyridin-5-yl)Acetamide

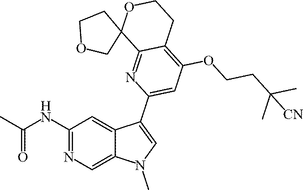 [SMILES: CC(=O)Nc1cc2c(-c3cc(OCCC(C)(C)C#N)c4c(n3)C3(CCOC3)OCC4)cn(C)c2cn1]

### Step 1 Synthesis of 4-((2′-Chloro-4,5,5′,6′-Tetrahydro-2H-Spiro[Furan-3,8′-Pyrano[3,4-b] Pyridin]-4′-yl)Oxy)-2,2-Dimethylbutyronitrile

2′,4′-Dichloro-4,5,5′,6′-tetrahydro-2H-spiro[furan-3,8′-pyrano[3,4-b]pyridine] (60 mg, 0.23 mmol), DMF (3 mL), 4-hydroxy-2,2-bismethyl-butyronitrile (91 mg, 0.80 mmol) and sodium hydride (12 mg, 0.30 mmol, 60% wt%) were added to a 25 mL round bottom flask, the mixture was stirred overnight at room temperature. The reaction was quenched by adding water, concentrated, and the resulting residue was purified by column chromatography (petroleum ether/ethyl acetate (v/v) = 1/1) to obtain the title compound as colorless oily liquid (58 mg, yield 74.70%).

MS (ESI, pos.ion) m/z: 339.1 [M+H]⁺.

### Step 2 Synthesis of N-(3-(4′-(3-Cyano-3-Methylbutoxy)-4,5,5′,6′-Tetrahydro-2H-Spiro[Fu ran-3,8′-Pyran [3,4-b]Pyridin]-2′-yl)-1H-Pyrrolo[2,3-c]Pyridin-5-yl)Acetamide

Under N₂, 4-((2′-chloro-4,5,5′,6′-tetrahydro-2H-spiro[furan-3,8′-pyrano[3,4-b]pyridin]-4′-yl)oxy)-2,2-dimethyl butyronitrile (58 mg, 0.17 mmol), tert-butyl 5-acetamido-3-(4,4,5,5,-tetramethyl-1,3,2-dioxaborolan-2-yl)-1H-pyrrolo[2,3-c]pyridine-1-carboxyl ate (99 mg, 0.22 mmol), potassium carbonate (47 mg, 0.34 mmol) and PdCl₂dppf (14 mg, 0.02 mmol) were added to a 25 mL two-neck round bottom flask, then vacuumized, and 1,4-dioxane (5 mL) was added under N₂, the mixture was stirred to dissolve most of the solids, then water (2 mL) was added and deoxygenated by bubbling N₂ for 10 min. The flask was connected to a reflux condenser, under N₂, the mixture was heated to 100° C. and reacted overnight. After the heating was stopped, the mixture was cooled to room temperature, concentrated in vacuum, and the resulting residue was purified by column chromatography (ethyl acetate/methanol (v/v) = 91/9) to obtain the title compound as a brown solid (50.0 mg, yield 61.10%).

MS (ESI, pos.ion) m/z: 476.2 [M+H]⁺.

### Step 3 Synthesis of N-(3-(4′-(3-Cyano-3-Methylbutoxy)-4,5,5′,6′-Tetrahydro-2H-Spiro[Fu ran-3,8′-Pyran[3,4-b]Pyridin]-2′-yl)-1-Methyl-1H-Pyrrolo[2,3-c]Pyridin-5-yl)Acetamide

N-(3-(4′-(3-Cyano-3-methylbutoxy)-4,5,5′,6′-tetrahydro-2H-spiro[furan-x3,8′-pyran[3, 4-b]pyridin]-2′-yl)-1H-pyrrolo[2,3-c]pyridin-5-yl)acetamide (50 mg, 0.11 mmol), acetonitrile (5 mL), cesium carbonate (44 mg, 0.14 mmol) and methyl iodide (22 mg, 0.16 mmol) were added to a 25 mL round bottom flask, the mixture was stirred overnight at room temperature. The mixture was concentrated, and the resulting residue was purified by column chromatography (dichloromethane/methanol (v/v) = 95/5) to obtain the title compound as a light yellow solid (40 mg, yield 77.70%).

MS (ESI, pos.ion) m/z: 490.2 [M+H]⁺;

¹H NMR (400 MHz, CDCl₃) δ 9.01 (s, 1H), 8.42 (s, 1H), 8.11 (s, 1H), 7.82 (s, 1H), 7.06 (s, 1H), 4.39 (t, J = 5.8 Hz, 2H), 4.33 - 4.09 (m, 4H), 4.03 - 3.94 (m, 2H), 3.92 (s, 3H), 2.92 -2.70 (m, 3H), 2.36 - 2.27 (m, 1H), 2.21 (s, 3H), 2.20 - 2.09 (m, 1H), 1.50 (s, 6H).

### Example 41 N-(3-(4′-(3-(2-(Dimethylamino)Ethoxy)Propoxy)-4,5,5′,6′-Tetrahydro-2H-Spir o[Furan-3,8′-Pyrano[3,4-b]Pyridin]-2′-yl)-1-Methyl-1H-Pyrrolo[2,3-c]Pyridin-5-yl)Acetamide

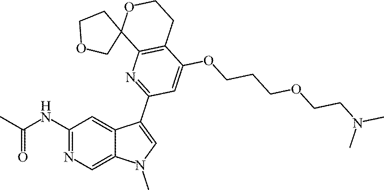 [SMILES: CC(=O)Nc1cc2c(-c3cc(OCCCOCCN(C)C)c4c(n3)C3(CCOC3)OCC4)cn(C)c2cn1]

### Step 1 Synthesis of 2-(3-((2′-Chloro-4,5,5′,6′-Tetrahydro-2H-Spiro[Furan-3,8′-Pyrano[3,4 -b]Pyridine]-4′-yl)Oxy)Propoxy)-N,N-Dimethylethylamine

3-(2-(Dimethylamino)ethoxy)propyl-1-ol (75 mg, 0.51 mmol) and anhydrous DMF (2 mL) were added to a round bottom flask, the mixture was stirred at room temperature, then sodium hydride (32 mg, 0.80 mmol) was added under N₂, the mixture was stirred for 30 min, then 2′,4′-dichloro-4,5,5′,6′-tetrahydro-2H-spiro[furan-3,8′-pyrano[3,4-b]pyridine] (55.3 mg, 0.21 mmol) was added, the mixture was heated to 100° C. and stirred for 6 h, then cooled to room temperature, the reaction was quenched by adding 0.1 mL of water, concentrated in vacuum, and the resulting residue was purified by column chromatography (petroleum ether/ethyl acetate (v/v) = 1/1) to obtain the title compound as yellow oily substance (39 mg, yield 49.5%).

MS (ESI, pos.ion) m/z: 371.2 [M+H]⁺.

### Step 2 Synthesis of N-(3-(4′-(3-(2-(Dimethylamino)Ethoxy)Propoxy)-4,5,5′,6′-Tetrahydro -2H-Spiro[Furan-3,8′-Pyrano[3,4-b]Pyridin]-2′-yl)-1-Methyl-1H-Pyrrolo[2,3-c]Pyridin-5-yl)Acet amide

2-(3-((2′-Chloro-4,5,5′,6′-tetrahydro-2H-spiro[furan-3,8′-pyrano[3,4-b]pyridine]-4′-yl) oxy)propoxy)-N,N-dimethylethylamine (39 mg, 0.11 mmol), N-(1-methyl-3-(4,4,5,5-tetramethyl-1,3,2-dioxaborolan-2-yl)-1H-pyrrolo[2,3-c]pyridin-5-yl)acetami de (51.5 mg, 0.11 mmol), PdCl₂dppf (13 mg, 0.02 mmol) and potassium carbonate (32.1 mg, 0.23 mmol) were added to a round bottom flask, the mixture was purged with N₂, then 1,4-dioxane (4 mL) and water (1.5 mL) were added, and the mixture was purged with N₂ again for 5 min, and heated to 100° C. and stirred for 3 h. Then the solution was cooled to room temperature, concentrated under reduced pressure, the resulting residue was purified by column chromatography (dichloromethane/methanol (v/v) = 97/3) to obtain the title compound as a yellow solid (13 mg, yield 23.61%).

MS (ESI, pos.ion) m/z: 262.8 [M+H]⁺;

¹H NMR (400 MHz, CDCl₃) δ (ppm) 8.97 (s, 1H), 8.46 (s, 1H), 8.40 (s, 1H), 7.89 (s, 1 H), 7.07 (s, 1 H), 4.31 - 4.08 (m, 6 H), 4.02 - 3.93 (m, 2 H), 3.91 (s, 3 H), 3.79 - 3.76 (t, J=4.0 Hz, 2 H), 3.68 (t, J = 6.1 Hz, 2 H), 3.05 - 2.92 (m, 1 H), 2.90 - 2.69 (m, 3 H), 2.64 (s, 3 H), 2.35-2.26 (m, 2 H), 2.22 (s, 3 H), 2.17 - 2.10 (m, 2 H).

### Example 42 N-(3-(4′-(Cyanomethyl)-4,5,5′,6′-Tetrahydro-2H-Spiro[Furan-3,8′-Pyrano[3,4-b]Pyridin]-2′-yl)-1-Methyl-1H-Pyrrolo[2,3-c]Pyridin-5-yl)Acetamide

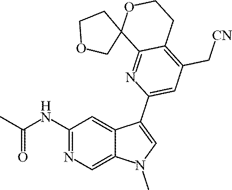 [SMILES: CC(=O)Nc1cc2c(-c3cc(CC#N)c4c(n3)C3(CCOC3)OCC4)cn(C)c2cn1]

### Step 1 Synthesis of 2-(2′-Chloro-4,5,5′,6′-Tetrahydro-2H-Spiro[Furan-3,8′-Pyrano[3,4-b]P yridin]-4′-yl)Acetonitrile

Trimethylsilyl cyanide (54 mg, 0.55 mmol) and acetonitrile (5 mL) were added to a 50 mL round bottom flask, under N₂, TBAF (0.55 mL, 0.55 mmol, 1 mol/L in THF) was added, the mixture was stirred at room temperature for 1 h. Then 2′-chloro-4′-(chloromethyl)-4,5,5′,6′-tetrahydro-2H-spiro[furan-3,8′-pyrano[3,4-b]pyridine] (50 mg, 0.18 mmol) was added, the mixture was stirred at room temperature overnight. The solvent was concentrated, 3 mL of water was added, the mixture was extracted with EtOAc (3 × 5 mL), the organic phases were combined, and the resulting residue was purified by column chromatography (petroleum ether/ethyl acetate (v/v) = 1/1) to obtain the title compound as a white solid (15 mg, yield 31.5%).

MS (ESI, pos.ion) m/z: 265.2 [M+H]⁺.

### Step 2 Synthesis of N-(3-(4′-(Cyanomethyl)-4,5,5′,6′-Tetrahydro-2H-Spiro[Furan-3,8′-Pyr ano[3,4-b]Pyridin]-2′-yl)-1-Methyl-1H-Pyrrolo[2,3-c]Pyridin-5-yl)Acetamide

Under N₂, 2-(2′-chloro-4,5,5′,6′-tetrahydro-2H-spiro[furan-3,8′-pyrano[3,4-b]pyridin]-4′-yl) acetonitrile (30 mg, 0.11 mmol), N-(1-methyl-3-(4,4,5,5-tetramethyl-1,3,2-dioxaborolan-2-yl)-1H-pyrrolo[2,3-c]pyridin-5-yl)acetami de (61 mg, 0.14 mmol, 70%), 1,4-dioxane (8 mL), water (2 mL) and potassium carbonate (31 mg, 0.23 mmol) were added to a 50 mL two-neck round bottom flask, the mixture was deoxygenated by N₂ bubbling for 10 min, then PdCl₂dppf (28 mg, 0.034 mmol) was added and deoxygenated by N₂ bubbling for 10 min. The flask was connected to a reflux condenser, refluxed for 3 h under N₂ again, then quenched by adding 10 mL of water, after extraction with chloroform (3 × 10 mL), the combined organic phase was dried over anhydrous sodium sulfate, and the resulting residue was purified by column chromatography (ethyl acetate /methanol (v/v) = 91/9) to obtain the title compound as a yellow solid (3.5 mg, yield 7.4%).

MS (ESI, pos.ion) m/z: 418.2 [M+H]⁺;

¹H NMR (400 MHz, CDCl₃): δ (ppm) 9.11 (s, 1H), 8.44 (s, 1H), 8.19 (s, 1H), 7.84 (s, 1H), 7.59 (s, 1H), 4.45 - 4.25 (m, 4H), 4.06 (dd, J = 10.1, 5.5 Hz, 2H), 3.96 (s, 3H), 3.73 (s, 2H), 2.96 (d, J = 12.7 Hz, 1H), 2.84 - 2.79 (m, 1H), 2.43 - 2.35 (m, 2H), 2.26 (s, 3H).

### Example 43 N-(3-(4′-((R)-3-Methoxybutoxy)-4,5,5′,6′-Tetrahydro-2H-Spiro[Furan-3,8′-Py rano[3,4-b]Pyridin]-2′-yl)-1-Methyl-1H-Pyrrolo[2,3-c]Pyridin-5-yl)Acetamide

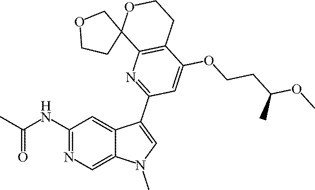 [SMILES: CO[C@@H](C)CCOc1cc(-c2cn(C)c3cnc(NC(C)=O)cc23)nc2c1CCOC21CCOC1]

### Step 1 Synthesis of 2′-Chloro-4′-((R)-3-Methoxybutoxy)-4,5,5′,6′-Tetrahydro-2H-Spiro[Fu ran-3,8′-Pyrano[3,4-b]Pyridine]

(2R)-4-((2′-Chloro-4,5,5′,6′-tetrahydro-2H-spiro[furan-3,8′-pyrano[3,4-b]pyridine]-4′-yl)oxy)butyl-2-ol (50 mg, 0.16 mmol) and anhydrous DMF (3 mL) were added to a flask, the mixture was stirred well at room temperature, under N₂, sodium hydride (20 mg, 0.83 mmol, 60 mass%) and methyl iodide (115 mg, 0.81 mmol) were added, then the solution was heated to 50° C. and stirred overnight, then the reaction was quenched with 5 mL of water and diluted with 50 mL of ethyl acetate, the mixture was stirred for 5 min, and separated, the aqueous phase was extracted with 20 mL of ethyl acetate, the organic phase was dried over anhydrous sodium sulfate, concentrated and the resulting residue was purified by column chromatography (petroleum ether/ethyl acetate (v/v) = 2/1) to obtain the title compound as colorless transparent oil (40 mg, yield 76.58%).

MS (ESI,pos.ion) m/z: 328.3 [M+H]⁺;

¹H NMR (400 MHz, CDCl₃) δ ppm 6.68 (s, 1H), 4.20 - 4.04 (m, 6 H), 4.00 - 3.84 (m, 2 H), 3.60 - 3.44 (m, 1 H), 3.34 (s, 3 H), 2.76 - 2.56 (m, 3 H), 2.26-2.20 (m, 1H), 2.01 - 1.90 (m, 2 H), 1.23 (d, J= 6.2 Hz, 3 H).

### Step 2 Synthesis of N-(3-(4′-((R)-3-Methoxybutoxy)-4,5,5′,6′-Tetrahydro-2H-Spiro[Furan -3,8′-Pyrano[3,4-b]Pyridin]-2′-yl)-1H-Pyrrolo[2,3-c]Pyridin-5-yl)Acetamide

2′-Chloro-4′-((R)-3-methoxybutoxy)-4,5,5′,6′-tetrahydro-2H-spiro[furan-3,8′-pyrano[3,4-b]pyridine] (63 mg, 0.19 mmol), tert-butyl 5-acetamido-3-(4,4,5,5,-tetramethyl-1,3,2-dioxaborolan-2-yl)-1H-pyrrolo[2,3-c]pyridine-1-carboxyl ate (112.1 mg, 0.25 mmol), potassium carbonate (54.1 mg, 0.39 mmol) and PdCl₂dppf (17.1 mg, 0.02 mmol) were added to a round bottom flask, the mixture was purged with N₂, then 1,4-dioxane (5 mL) and water (2 mL) were added, the mixture was purged with N₂ again and heated to 100° C. and stirred overnight. Then the solution was cooled to room temperature, concentrated under reduced pressure, the resulting residue was purified by column chromatography (dichloromethane/methanol (v/v) = 97/3) to obtain the title compound as a yellow solid (36 mg, yield 40.15%).

MS (ESI,pos.ion) m/z: 467.2 [M+H]⁺.

### Step 3 Synthesis of N-(3-(4′-((R)-3-Methoxybutoxy)-4,5,5′,6′-Tetrahydro-2H-Spiro[Furan -3,8′-Pyrano[3,4-b]Pyridin]-2′-yl)-1-Methyl-1H-Pyrrolo[2,3-c]Pyridin-5-yl)Acetamide

N-(3-(4′-((R)-3-methoxybutoxy)-4,5,5′,6′-tetrahydro-2H-spiro[furan-3,8′-pyran[3,4-b] pyridin]-2′-yl)-1H-pyrrolo[2,3-c]pyridin-5-yl)acetamide (36 mg, 0.08 mmol), cesium carbonate (20.1 mg, 0.15 mmol) and acetonitrile (5 mL) were added dropwise to a flask, then methyl iodide (30.2 mg, 0.21 mmol) was added using a syringe. Under N₂, the solution was stirred in an oil bath at 35° C. overnight, cooled and concentrated in vacuum, the resulting residue was purified by column chromatography (dichloromethane/methanol (v/v) = 97/3) to obtain the title compound as a yellow solid (25 mg, yield 67.41%).

MS (ESI, pos.ion) m/z: 481.3 [M+H]⁺;

¹H NMR (400 MHz, CDCl₃) δ ppm 9.05 (s, 1H), 8.42 (s, 1H), 8.32 (s, 1H), 7.80 (s, 1H), 7.06 (s, 1H), 4.37 - 4.13 (m, 6 H), 4.07 - 3.97 (m, 2 H), 3.93 (s, 3 H), 3.69 - 3.51 (m, 1 H), 3.37 (s, 3 H), 2.94 - 2.86 (m, 1H), 2.82 - 2.75 (m, 2 H), 2.37 - 2.31 (m, 1 H), 2.24 (s, 3 H), 2.09 - 1.99 (m, 3 H), 1.27 (d, J = 5.8 Hz, 3 H).

### Example 44 N-(3-(4′-((3-Hydroxy-3-Methylbutyl)Thio)-4,5,5′,6′-Tetrahydro-2H-Spiro[Fura n-3,8′-Pyrano[3,4-b]Pyridin]-2′-yl)-1-Methyl-1H-Pyrrolo[2,3-c]Pyridin-5-yl)Acetamide

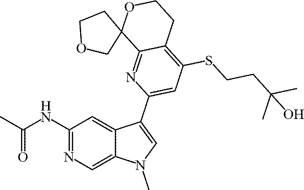 [SMILES: CC(=O)Nc1cc2c(-c3cc(SCCC(C)(C)O)c4c(n3)C3(CCOC3)OCC4)cn(C)c2cn1]

### Step 1 Synthesis of 4-((2′-Chloro-4,5,5′,6′-Tetrahydro-2H-Spiro[Furan-3,8′-Pyrano[3,4-b] Pyridin]-4′-yl)Thio)-2-Methylbutan-2-Ol

2′,4′-Dichloro-4,5,5′,6′-tetrahydro-2H-spiro[furan-3,8′-pyrano[3,4-b]pyridine] (52 mg, 0.20 mmol), DMF (2 mL), 2-methyl-4-mercapto-butan-2-ol (31 mg, 0.26 mmol) and sodium hydride (11 mg, 0.28 mmol, 60% wt%) were added to a 25 mL round bottom flask, the mixture was stirred overnight at room temperature. The reaction was quenched by adding water, concentrated, and the resulting residue was purified by column chromatography (petroleum ether/ethyl acetate (v/v) = 1/1) to obtain the title compound as colorless oily liquid (48 mg, yield 76.00%).

MS (ESI, pos.ion) m/z: 344.2 [M+H]⁺.

### Step 2 Synthesis of N-(3-(4′-((3-Hydroxy-3-Methylbutyl)Thio)-4,5,5′,6′-Tetrahydro-2H-Sp iro[Furan-3,8′-Pyrano[3,4-b]Pyridin]-2′-yl)-1H-Pyrrolo[2,3-c]Pyridin-5-yl)Acetamide

Under N₂, 4-((2′-chloro-4,5,5′,6′-tetrahydro-2H-spiro[furan-3,8′-pyrano[3,4-b]pyridin]-4′-yl)thio)-2-methylbuta n-2-ol (48 mg, 0.14 mmol), tert-butyl 5-acetamido-3-(4,4,5,5,-tetramethyl-1,3,2-dioxaborolan-2-yl)-1H-pyrrolo[2,3-c]pyridine-1-carboxyl ate (79 mg, 0.20 mmol), potassium carbonate (39 mg, 0.28 mmol) and PdCl₂dppf (11 mg, 0.01 mmol) were added to a 25 mL two-neck round bottom flask, then vacuumized, and 1,4-dioxane (5 mL) was added under N₂, the mixture was stirred to dissolve most of the solids, then water (2 mL) was added and deoxygenated by bubbling N₂ for 10 min. The flask was connected to a reflux condenser, under N₂, the mixture was heated to 100° C. and reacted overnight. After the heating was stopped, the mixture was cooled to room temperature and concentrated, then methanol was added, and silica gel was mixed, and the resulting residue was purified by column chromatography (dichloromethane/methanol (v/v) = 91/9) to obtain the title compound as a brown solid (50 mg, yield 74.20%).

MS (ESI, pos.ion) m/z: 483.2 [M+H]⁺.

### Step 3 Synthesis of N-(3-(4′-((3-Hydroxy-3-Methylbutyl)Thio)-4,5,5′,6′-Tetrahydro-2H-Sp iro[Furan-3,8′-Pyrano[3,4-b]Pyridin]-2′-yl)-1-Methyl-1H-Pyrrolo[2,3-c]Pyridin-5-yl)Acetamide

N-(3-(4′-((3-Hydroxy-3-methylbutyl)thio)-4,5,5′,6′-tetrahydro-2H-spiro[furan-3,8′-pyr ano[3,4-b]pyridin]-2′-yl)-1H-pyrrolo[2,3-c]pyridin-5-yl)acetamide (50 mg, 0.10 mmol), acetonitrile (3 mL), cesium carbonate (44 mg, 0.13 mmol) and methyl iodide (22 mg, 0.15 mmol) were added to a 25 mL round bottom flask, the mixture was stirred overnight at room temperature. The mixture was concentrated, and the resulting residue was purified by column chromatography (dichloromethane/methanol (v/v) = 95/5) to obtain the title compound as a light yellow solid (26 mg, yield 50.50%).

MS (ESI, pos.ion) m/z: 497.2 [M+H]⁺;

¹H NMR (400 MHz, CDCl₃) δ 9.02 (s, 1H), 8.53 (s, 1H), 8.42 (s, 1H), 8.02 (s, 1H), 7.61 (s, 1H), 4.81 (s, 1H), 4.36 - 4.13 (m, 4H), 4.05 - 3.99 (m, 2H), 3.94 (s, 3H), 3.43 - 3.30 (m, 2H), 2.85 - 2.70 (m, 3H), 2.37 - 2.30 (m, 1H), 2.26 (s, 3H), 2.05 - 1.89 (m, 2H), 1.35 (s, 6H).

### Example 45 N-(3-(4′-((2-Hydroxy-2-Methylpropoxy)Methyl)-4,5,5′,6′-Tetrahydro-2H-Spir o[Furan-3,8′-Pyrano[3,4-b]Pyridin]-2′-yl)-1-Methyl-1H-Pyrrolo[2,3-c]Pyridin-5-yl)Acetamide

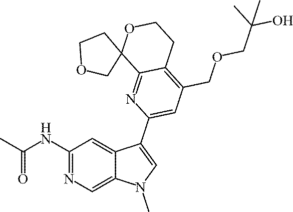 [SMILES: CC(=O)Nc1cc2c(-c3cc(COCC(C)(C)O)c4c(n3)C3(CCOC3)OCC4)cn(C)c2cn1]

### Step 1 Synthesis of 1-((2′-Chloro-4,5,5′,6′-Tetrahydro-2H-Spiro[Furan-3,8′-Pyrano[3,4-b] Pyridin]-4′-yl)Methoxy)-2-Methylpropan-2-ol

2′-Chloro-4′-(chloromethyl)-4,5,5′,6′-tetrahydro-2H-spiro[furan-3,8′-pyrano[3,4-b]pyr idine] (50 mg, 0.18 mmol), 2-methylpropane-1,2-diol (50 mg, 0.55 mmol) and DMF (2 mL) were added to a 50 mL round bottom flask, under N₂, cesium carbonate (12 mg, 0.36 mmol) was added, the mixture was reacted at room temperature for 5 h. The solvent was concentrated, and the resulting residue was purified by column chromatography (petroleum ether/ethyl acetate (v/v) = ⅕) to obtain the title compound as yellow oily liquid (28 mg, yield 46.8%).

MS (ESI, pos.ion) m/z: 328.2 [M+H]⁺.

### Step 2 Synthesis of N-(3-(4′-((2-Hydroxy-2-Methylpropoxy)Methyl)-4,5,5′,6′-Tetrahydro-2H-Spiro[Furan-3,8′-Pyrano[3,4-b]Pyridin]-2′-yl)-1-Methyl-1H-Pyrrolo[2,3-c]Pyridin-5-yl)Aceta mide

Under N₂, 1-((2′-chloro-4,5,5′,6′-tetrahydro-2H-spiro[furan-3,8′-pyrano[3,4-b]pyridin]-4′-yl)methoxy)-2-methy lpropan-2-ol (60 mg, 0.18 mmol), N-(1-methyl-3-(4,4,5,5-tetramethyl-1,3,2-dioxaborolan-2-yl)-1H-pyrrolo[2,3-c]pyridin-5-yl)acetami de (700 mg, 0.22 mmol), 1,4-dioxane (8 mL), water (2 mL) and potassium carbonate (50 mg, 0.37 mmol) were added to a 50 mL two-neck round bottom flask, the mixture was deoxygenated by bubbling N₂ for 10 min, then PdCl₂dppf (44 mg, 0.054 mmol) was added and deoxygenated by bubbling N₂ for 10 min. The flask was connected to a reflux condenser, the mixture was refluxed for 3 h under N₂, then 10 mL of water was added to quench the reaction, after extraction with chloroform (3 × 10 mL), the combined organic phase was dried over anhydrous sodium sulfate, and the resulting residue was purified by column chromatography (ethyl acetate/methanol (v/v) = 91/9) to obtain the title compound as a yellow solid (23.0 mg, yield 26.1%).

MS (ESI, pos.ion) m/z: 481.2 [M+H]⁺;

¹H NMR (400 MHz, DMSO-d₆): δ (ppm) 10.20 (s, 1H), 9.01 (s, 1H), 8.62 (s, 1H), 8.20 (s, 1H), 7.65 (s, 1H), 4.56 (s, 2H), 4.53 (s, 1H), 4.24 (s, 1H), 4.07 - 3.98 (m, 3H), 3.94 (s, 4H), 3.31 (s, 3H), 2.91 - 2.67 (m, 3H), 2.21 (s, 1H), 2.09 (s, 3H), 1.15 (s, 6H).

### Example 46 N-(3-(4′-((3-Hydroxy-3-Methylbutyl)Sulfonyl)-4,5,5′,6′-Tetrahydro-2H-Spiro[ Furan-3,8′-Pyrano[3,4-b]Pyridin]-2′-yl)-1-Methyl-1H-Pyrrolo[2,3-c]Pyridin-5-yl)Acetamide

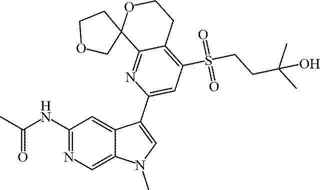 [SMILES: CC(=O)Nc1cc2c(-c3cc(S(=O)(=O)CCC(C)(C)O)c4c(n3)C3(CCOC3)OCC4)cn(C)c2cn1]

### Step 1 Synthesis of 4-((2′-Chloro-4,5,5′,6′-Tetrahydro-2H-Spiro[Furan-3,8′-Pyrano[3,4-b] Pyridin]-4′-yl)Sulfonyl)-2-Methylbutan-2-ol

4-((2′-Chloro-4,5,5′,6′-tetrahydro-2H-spiro[furan-3,8′-pyrano[3,4-b]pyridin]-4′-yl)thio )-2-methylbutan-2-ol (90 mg, 0.26 mmol), DCM (5 mL) and m-chloroperbenzoic acid (128 mg, 0.63 mmol, 85 wt%) were added to a 25 mL round bottom flask, the mixture was stirred overnight at room temperature. After the TLC spotting showed that the reaction was completed, 5 mL of saturated sodium sulfite was added to quench the reaction, and the mixture was stirred for 5 min, then 5 mL of saturated sodium carbonate was added. After extraction with chloroform (3 × 10 mL), the organic phases were combined, dried over anhydrous sodium sulfate and concentrated in vacuum to obtain the title compound as a white solid (95.4 mg, yield 97.00%).

MS (ESI, pos.ion) m/z: 376.0 [M+H]⁺.

### Step 2 Synthesis of N-(3-(4′-((3-Hydroxy-3-Methylbutyl)Sulfonyl)-4,5,5′,6′-Tetrahydro-2 H-Spiro[Furan-3,8′-Pyrano[3,4-b]Pyridin]-2′-yl)-1H-Pyrrolo[2,3-c]Pyridin-5-yl)Acetamide

Under N₂, 4-((2′-chloro-4,5,5′,6′-tetrahydro-2H-spiro[furan-3,8′-pyrano[3,4-b]pyridin]-4′-yl)sulfonyl)-2-methyl butan-2-ol (95 mg, 0.25 mmol), tert-butyl 5-acetamido-3-(4,4,5,5-tetramethyl-1,3,2-dioxaborolan-2-yl)-1H-pyrrolo[2,3-c]pyridine-1-carboxyl ate (131 mg, 0.33 mmol), potassium carbonate (70 mg, 0.51 mmol) and PdCl₂dppf (21 mg, 0.03 mmol) were added to a 25 mL two-neck round bottom flask, then vacuumized, and 1,4-dioxane (5 mL) was added under N₂, the mixture was stirred to dissolve most of the solids, then water (2 mL) was added and deoxygenated by bubbling N₂ for 10 min. The flask was connected to a reflux condenser, under N₂, the mixture was heated to 100° C. and reacted overnight. After the heating was stopped, the mixture was cooled to room temperature and concentrated, then methanol was added, and silica gel was mixed, and the resulting residue was purified by column chromatography (dichloromethane/methanol (v/v) = 91/9) to obtain the title compound as a brown solid (31 mg, yield 23.80%).

MS (ESI, pos.ion) m/z: 515.1 [M+H]⁺.

### Step 3 Synthesis of N-(3-(4′-((3-Hydroxy-3-Methylbutyl)Sulfonyl)-4,5,5′,6′-Tetrahydro-2 H-Spiro[Furan-3,8′-Pyrano[3,4-b]Pyridin]-2′-yl)-1-Methyl-1H-Pyrrolo[2,3-c]Pyridin-5-yl)Aceta mide

N-(3-(4′-((3-Hydroxy-3-methylbutyl)sulfonyl)-4,5,5′,6′-tetrahydro-2H-spiro[furan-3,8′ -pyrano[3,4-b]pyridin]-2′-yl)-1H-pyrrolo[2,3-c]pyridin-5-yl)acetamide (31.0 mg, 0.10 mmol), acetonitrile (3 mL), cesium carbonate (26 mg, 0.08 mmol) and methyl iodide (13 mg, 0.09 mmol) were added to a 25 mL round bottom flask, the mixture was stirred overnight at room temperature. The mixture was concentrated, and the resulting residue was purified by column chromatography (dichloromethane/methanol (v/v) = 95/5) to obtain the title compound as a light yellow solid (22 mg, yield 69.10%).

MS (ESI, pos.ion) m/z: 529.1 [M+H]⁺;

¹H NMR (400 MHz, CDCl₃) δ 9.03 (s, 1H), 8.44 (s, 1H), 8.29 (s, 1H), 7.99 (s, 1H), 7.88 (s, 1H), 4.41 - 4.31 (m, 1H), 4.29 - 4.17 (m, 3H), 4.09 - 3.98 (m, 2H), 3.95 (s, 3H), 3.53 - 3.44 (m, 2H), 3.38 - 3.27 (m, 2H), 2.92 - 2.81 (m, 1H), 2.46 - 2.34 (m, 1H), 2.23 (s, 3H), 2.08 - 1.98 (m, 2H), 1.29 (s, 6H).

### Example 47 N-(3-(4′-(3-Amino-3-Methylbutoxy)-4,5,5′,6′-Tetrahydro-2H-Spiro[Furan-3,8′ -Pyrano[3,4-b]Pyridin]-2′-yl)-1-Methyl-1H-Pyrrolo[2,3-c]Pyridin-5-yl)Acetamide

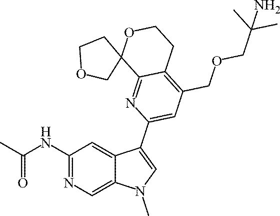 [SMILES: CC(=O)Nc1cc2c(-c3cc(COCC(C)(C)N)c4c(n3)C3(CCOC3)OCC4)cn(C)c2cn1]

### Step 1 Synthesis of 4-((2′-Chloro-4,5,5′,6′-Tetrahydro-2H-Spiro[Furan-3,8′-Pyran[3,4-b]P yridin]-4′-yl)Oxy)-2-Methylbutan-2-Amine

2′,4′-Dichloro-4,5,5′,6′-tetrahydro-2H-spiro[furan-3,8′-pyrano[3,4-b]pyridine] (78 mg, 0.30 mmol), DMF (3 mL), 3-amino-3-methyl-butan-1-ol (62 mg, 0.60 mmol) and sodium hydride (16 mg, 0.40 mmol, 60% wt%) were added to a 25 mL round bottom flask, the mixture was stirred overnight at room temperature. The reaction was quenched by water, concentrated, and the resulting residue was purified by column chromatography (dichloromethane/methanol (v/v) = 91/9) to obtain the title compound as colorless oily liquid (91 mg, yield 92.60%).

MS (ESI, pos.ion) m/z: 327.1 [M+H]⁺.

### Step 2 Synthesis of N-(3-(4′-(3-Amino-3-Methylbutoxy)-4,5,5′,6′-Tetrahydro-2H-Spiro[Fu ran-3,8′-Pyrano[3,4-b]Pyridin]-2′-yl)-1-Methyl-1H-Pyrrolo[2,3-c]Pyridin-5-yl)Acetamide

Under N₂, 4-((2′-chloro-4,5,5′,6′-tetrahydro-2H-spiro[furan-3,8′-pyran[3,4-b]pyridin]-4′-yl)oxy)-2-methylbutan -2-amine (63 mg, 0.19 mmol), N-(1-methyl-3-(4,4,5,5-tetramethyl-1,3,2-dioxaborolan-2-yl)-1H-pyrrolo[2,3-c]pyridin-5-yl)acetami de (79 mg, 0.25 mmol), potassium carbonate (53 mg, 0.39 mmol), PdCl₂dppf (16 mg, 0.02 mmol) and 1,4-dioxane (5 mL) were added to a 50 mL two-neck round bottom flask, the mixture was stirred to dissolve, then water (2 mL) was added, the system was a yellow turbid liquid, then deoxygenated by bubbling N₂ for 10 min. The flask was connected to a reflux condenser, and heated to 100° C. and reacted overnight under N₂. After the heating was stopped, the mixture was cooled to room temperature, then concentrated, and the resulting residue was purified by column chromatography (dichloromethane/methanol (v/v) = 4/1) to obtain the title compound as a brown solid (69 mg, yield 74.60%).

MS (ESI, pos.ion) m/z: 480.8 [M+H]⁺;

¹H NMR (400 MHz, DMSO-d₆) δ 10.19 (s, 1H), 9.05 (s, 1H), 8.61 (s, 1H), 8.31 (s, 1H), 7.26 (s, 1H), 4.33 - 4.18 (m, 3H), 4.06 - 3.78 (m, 9H), 2.91 - 2.79 (m, 1H), 2.68 - 2.59 (m, 2H), 2.22 - 2.12 (m, 1H), 2.09 (s, 3H), 1.99 - 1.90 (m, 2H), 1.23 (s, 6H).

### Example 48 N-(3-(4′-((3-Hydroxy-3-Methylbutyl)Amino)-4,5,5′,6′-Tetrahydro-2H-Spiro[Fu ran-3,8′-Pyrano[3,4-b]Pyridin]-2′-yl)-1-Methyl-1H-Pyrrolo[2,3-c]Pyridin-5-yl)Acetamide

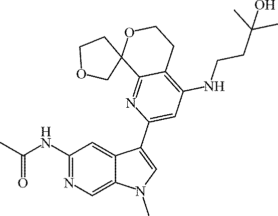 [SMILES: CC(=O)Nc1cc2c(-c3cc(NCCC(C)(C)O)c4c(n3)C3(CCOC3)OCC4)cn(C)c2cn1]

N-(3-(4′-Chloro-4,5,5′,6′-tetrahydro-2H-spiro[furan-3,8′-pyrano[3,4-b]pyridine]-2′-yl) -1-methyl-1H-pyrrolo[2,3-c]pyridin-5-yl)acetamide (22 mg, 0.05 mmol) and 4-amino-2-methyl-butan-2-ol (13 mg, 0.13 mmol) were added to a 25 mL one-neck round bottom flask, then vacuumized, DMF (5 mL) and sodium tert-butoxide (25 mg, 0.26 mmol) was added under N₂, and the mixture was bubbled with N₂ for 10 min under stirring, then bis(tri-tert-butylphosphine)palladium (14 mg, 0.03 mmol) was added. The flask was connected to a reflux condenser, under N₂, the mixture was heated to 120° C. and reacted 24 h. The mixture was cooled to room temperature, concentrated, then 20 mL of chloroform was added, washed with water (3 × 10 mL) and the aqueous phases were combined, after extraction with 10 mL of chloroform, the combined organic phase was dried over anhydrous sodium sulfate, filtered, and the resulting residue was purified by column chromatography (dichloromethane/methanol (v/v) = 85/15) to obtain the title compound as a yellow solid (16 mg, yield 63.00%).

MS (ESI, pos.ion) m/z: 480.2 [M+H]⁺;

¹H NMR (400 MHz, CDCl₃) δ 8.98 (s, 1H), 8.39 (s, 1H), 8.19 (s, 1H), 7.86 (s, 1H), 6.96 (s, 1H), 4.55 (s, 1H), 4.32 - 3.96 (m, 6H), 3.91 (s, 3H), 3.61 - 3.39 (m, 3H), 2.86 - 2.71 (m, 1H), 2.60 - 2.41 (m, 2H), 2.31 - 2.25 (m, 1H), 2.23 (s, 3H), 2.05 - 1.95 (m, 2H), 1.34 (s, 6H).

### Example 49 N-(3-(4′-(3-Hydroxy-3-Methylbutoxy)-4,5,5′,6′-Tetrahydro-2H-Spiro[Furan-3, 8′-Pyrano[3,4-b]Pyridin]-2′-yl)-1-(Oxetan-3-yl)-1H-Pyrrolo[2,3-c]Pyridin-5-yl)Acetamide

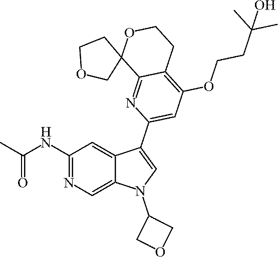 [SMILES: CC(=O)Nc1cc2c(-c3cc(OCCC(C)(C)O)c4c(n3)C3(CCOC3)OCC4)cn(C3COC3)c2cn1]

### Step 1 Synthesis of N-(3-(4′-(3-Hydroxy-3-Methylbutoxy)-4,5,5′,6′-Tetrahydro-2H-Spiro[ Furan-3,8′-Pyrano[3,4-b]Pyridin]-2′-yl)-1H-Pyrrolo[2,3-c]Pyridin-5-yl)Acetamide

Under N₂, 4-((2′-chloro-4,5,5′,6′-tetrahydro-2H-spiro[furan-3,8′-pyrano[3,4-b]pyridin]-4′-yl)oxy)-2-methylbuta n-2-ol (485 mg, 1.48 mmol), tert-butyl 5-acetamido-3-(4,4,5,5,-tetramethyl-1,3,2-dioxaborolan-2-yl)-1H-pyrrolo[2,3-c]pyridine-1-carboxyl ate (700 mg, 1.75 mmol), potassium carbonate (371 mg, 2.68 mmol) and PdCl₂dppf (112 mg, 0.13 mmol) were added to a 25 mL two-neck round bottom flask, then vacuumized, and 1,4-dioxane (15 mL) was added under N₂, the mixture was stirred to dissolve most of the solids, then water (6 mL) was added and deoxygenated by bubbling N₂ for 10 min. The flask was connected to a reflux condenser, under N₂, the mixture was heated to 100° C. and reacted overnight. After the heating was stopped, the mixture was cooled to room temperature, then concentrated, and the resulting residue was purified by column chromatography (dichloromethane/methanol (v/v) = 91/9) to obtain the title compound as a yellow solid 575 mg, yield 83.31%).

MS (ESI, pos.ion) m/z: 467.3 [M+H]⁺.

### Step 2 Synthesis of N-(3-(4′-(3-hydroxy-3-methylbutoxy)-4,5,5′,6′-tetrahydro-2H-Spiro[ Furan-3,8′-Pyrano|[3,4-b|Pyridin]-2′-yl)-1-(Oxetan-3-yl)-1H-Pyrrolo|2,3-c]Pyridin-5-yl)Acetami de

N-(3-(4′-(3-Hydroxy-3-methylbutoxy)-4,5,5′,6′-tetrahydro-2H-spiro[furan-3,8′-pyrano [3,4-b]pyridin]-2′-yl)-1H-pyrrolo[2,3-c]pyridin-5-yl)acetamide (38.0 mg, 0.08 mmol), cesium carbonate (40 mg, 0.12 mmol) and 3-iodooxetane (19 mg, 0.10 mmol) were added to a 25 mL round bottom flask, the mixture was stirred at 80° C. for 24h. Then the mixture was concentrated, the resulting residue was purified by column chromatography (dichloromethane/methanol (v/v) = 95/5) to obtain the title compound as a yellow solid (18 mg, yield 42.00%).

MS (ESI, pos.ion) m/z: 523.2 [M+H]⁺;

¹H NMR (400 MHz, CDCl₃) δ 9.04 (s, 1H), 8.64 (s, 1H), 8.23 (s, 1H), 8.14 (s, 1H), 7.24 (s, 1H), 5.70 - 5.53 (m, 1H), 5.33 - 5.20 (m, 2H), 5.19 - 5.06 (m, 2H), 4.53 - 4.39 (m, 2H), 4.30 - 4.08 (m, 4H), 4.05 - 3.85 (m, 2H), 2.85 - 2.68 (m, 3H), 2.36 - 2.27 (m, 1H), 2.23 (s, 3H), 2.16 - 2.08 (m, 2H), 1.36 (s, 6H).

### Example 50 N-(3-(4′-(3-Hydroxy-3-Methylbutoxy)-4,5,5′,6′-Tetrahydro-2H-Spiro[Furan-3, 8′-Pyrano[3,4-b]Pyridin]-2′-yl)-1-Isopropyl-1H-Pyrrolo[2,3-c]Pyridin-5-yl)Acetamide

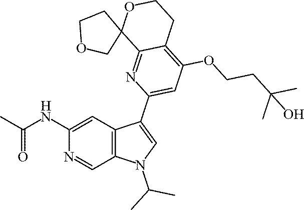 [SMILES: CC(=O)Nc1cc2c(-c3cc(OCCC(C)(C)O)c4c(n3)C3(CCOC3)OCC4)cn(C(C)C)c2cn1]

Under N₂, N-(3-(4′-(3-hydroxy-3-methylbutoxy)-4,5,5′,6′-tetrahydro-2H-spiro[furan-3,8′-pyrano[3,4-b]pyridin] -2′-yl)-1H-pyrrolo[2,3-c]pyridin-5-yl)acetamide (35 mg, 0.08 mmol), cesium carbonate (37 mg, 0.11 mmol) and acetonitrile (2 mL) were added to a 25 mL two-neck round bottom flask, the mixture was stirred for 5 min, then 2-iodopropane (26 mg, 0.15 mmol) was added, the mixture was stirred at 80° C. for 48 h. The solution was concentrated under reduced pressure, and the resulting residue was purified by column chromatography (dichloromethane/methanol (v/v) = 95/5) to obtain the title compound as a light yellow solid (26 mg, yield 68%).

MS (ESI, pos.ion) m/z: 467.3 [M+H]⁺;

¹H NMR (400 MHz, CDCl₃) δ 9.00 (s, 1H), 8.48 (s, 1H), 8.17 (s, 1H), 7.97 (s, 1H), 7.23 (s, 1H), 4.86 - 4.66 (m, 1H), 4.54 - 4.38 (m, 2H), 4.32 - 4.08 (m, 4H), 4.04 - 3.85 (m, 2H), 3.49 - 3.31 (m, 1H), 2.88 - 2.67 (m, 3H), 2.35 - 2.27 (m, 1H), 2.22 (s, 3H), 2.15 - 2.10 (m, 1H), 1.64 (d, J= 6.7 Hz, 6H), 1.36 (s, 6H).

### Biological Assay

The analytical LC/MS/MS system includes an Agilent 1200 series vacuum degasser, a binary syringe pump, an orifice plate autosampler, a column oven, and an Agilent G6430 triple quadrupole mass spectrometer with an electrospray ionization (ESI) source. Quantitative analysis was carried out in MRM mode, and the parameters of MRM conversion are shown in Table A:

TABLE A

| | |
|---|---|
| Multiple reaction detection scan | 490.2→383.1 |
| Fragmentation voltage | 230 V |
| Capillary voltage | 55 V |
| Temperature of drying gas | 350° C. |
| Atomizer | 0.28 MPa |
| Flow rate of drying gas | 10 L/min |

Agilent XDB-C18, 2.1 × 30 mm, 3.5 µM column was used to analysis, 5 µL sample was injected. Analytical conditions: Mobile phases were 0.1% formic acid in water (A) and 0.1% formic acid in methanol (B). The flow rate was 0.4 mL/min. The mobile phase gradient is shown in Table B:

TABLE B

| | |
|---|---|
| Time | Gradient of mobile phase B |
| 0.5 min | 5% |
| 1.0 min | 95% |
| 2.2 min | 95% |
| 2.3 min | 5% |
| 5.0 min | Terminate |

In addition, Agilent 6330 series LC/MS/MS spectrometer was used for analysis, equipped with G1312A binary syringe pump, G1367A automatic sampler and G1314C UV detector; LC/MS/MS spectrometer used ESI radiation source. Optimum analysis was achieved using standards with appropriate cation model processing and MRM conversion for each analyte. A Capcell MP-C18 column, 100 × 4.6 mm I.D., 5 µM (Phenomenex, Torrance, California, USA) was used during the analysis. Mobile phase was 5 mM ammonium acetate, 0.1% methanol in water (A): 5 mM ammonium acetate, 0.1% methanol in acetonitrile (B) (70/30, v/v); flow rate was 0.6 mL/min; column temperature was maintained at room temperature; 20 µL of sample was injected.

### Example A Stability in Human and Rat Liver Microsomes

The stability of the compounds of the present invention in human and rat liver microsomes can be tested by the following two methods:

### Method 1:

Human or rat liver microsomes were incubated in polypropylene tubes in duplicate wells. A typical incubation mixture included human or rat liver microsomes (0.5 mg protein/mL), target compound (5 µM), and a total volume of 200 µL of NADPH (1.0 mM) potassium phosphate buffer (PBS, 100 mM, pH 7.4), the compound was dissolved in DMSO and diluted with PBS, the final concentration of the DMSO solution was 0.05%. Human or rat liver microsomes were incubated at 37° C. in an air-connected water bath. After pre-incubation for 3 minutes, protein was added to start the reaction. The same volume of ice-cold acetonitrile was added to terminate the reaction at different time points (0, 5, 10, 15, 30 and 60 min). Samples were stored at -80° C. until LC/MS/MS analysis.

The linear concentration range of each target compound was determined, and then, the concentration of the target compounds in the human or rat liver microsomes incubation mixture was determined by LC/MS/MS method.

Parallel incubations were performed using denatured microsomes as a negative control and dextromethorphan (70 µM) as a positive control. Negative control was incubated at 37° C., the reaction was terminated at different time points (0, 15 and 60 minutes); positive control was incubated at 37° C., the reaction was terminated at different time points (0, 5, 10, 15, 30 and 60 minutes). Positive and negative control samples were used in each assay to ensure the integrity of the microsomal incubation system.

### Method 2:

In addition, the stability data of the compounds of the present invention in human or rat liver microsomes can also be obtained from the following tests:

Human or rat liver microsomes were incubated in polypropylene tubes in duplicate wells. A typical incubation mixture included human or rat liver microsomes (final concentration: 0.5 mg protein/mL), target compound (final concentration: 1.5 µM) and a total volume of 30 µL of K-buffer solution (containing 1.0 mM EDTA, 100 mM, pH 7.4). Compounds were dissolved in DMSO and diluted with K-buffer solution, the final concentration of DMSO was 0.2%. After pre-incubation for 10 min, 15 µL of NADPH (final concentration: 2 mM) was added to carry out the enzymatic reaction, and the whole experiment was carried out in an incubation tube at 37° C. 135 µL of acetonitrile (with IS) was added to terminate the reaction at various time points (0, 15, 30 and 60 min). The mixture was centrifuged at 4000 rpm for 10 min to remove protein and the supernatant was collected for LC-MS/MS analysis.

In the above experiments, ketanserin (1 µM) was selected as a positive control, which was incubated at 37° C., and the reaction was terminated at different time points (0, 15, 30 and 60 min). Positive control sample was used in each assay to ensure the integrity of the microsomal incubation system.

### Data Analysis

For each reaction, in vivo intrinsic hepatic clearance CLᵢₙₜ is extrapolated by plotting the compound concentration (expressed as a percentage) in human or rat liver microsomal incubations as a percentage relative to time zero (ref.: Naritomi Y, Terashita S, Kimura S, Suzuki A, Kagayama A, Sugiyama Y. Prediction of human hepatic clearance from in vivo animal experiments and in vitro metabolic studies with liver microsomes from animals and humans. Drug Metabolism and Disposition 2001, 29: 1316-1324.). The results are shown in Table 1, Table 1 shows the experimental results of the stability of the compounds provided in the examples of the present invention in human and rat liver microsomes.

Table 1 Experimental results of the stability of the compounds provided in the examples of the present invention in human and rat liver microsomes

| Example No. | Human | | Rat | |
|---|---|---|---|---|
| | T_{½} (min) | CLᵢₙₜ (mL/min/kg) | T_{½} (min) | CLᵢₙₜ (mL/min/kg) |
| Example 1 | 115.4 | 15.1 | 153.5 | 16.2 |
| Example 2 | 48.22 | 36.0 | 161.4 | 15.4 |

As can be seen from Table 1, when the compounds of the present invention were incubated in human and rat liver microsomes, the compounds exhibited appropriate stability.

### Example B Pharmacokinetic Evaluation of Mice, Rats, Dogs and Monkeys After Intravenous Injection and Oral Quantification of Compounds of the Present Invention

The present invention evaluates the pharmacokinetic studies of the compounds of the present invention in mice, rats, dogs or monkeys. The compounds of the present invention were administered in the form of aqueous solution or 2% HPMC + 1% Tween-80 aqueous solution, 5% DMSO + 5% saline solution, 4% MC or capsule. For intravenous administration, animals were dosed at about 0.5, 0.6, 1 or 2 mg/kg. The oral doses (p.o.) were 5 or 10 mg/kg for rats and mice, and 10 mg/kg for dogs and monkeys. Bloods (0.3 mL) were taken at time points 0.25, 0.5, 1.0, 2.0, 3.0, 4.0, 6.0, 8.0, 12 and 24 h and centrifuged at 3,000 or 4,000 rpm for 10 min. Plasma solutions were collected and stored at -20° C. or -70° C. until LC/MS/MS analysis as described above. The results show that when the compounds of the present invention are administered intravenously or orally, the compounds exhibit good pharmacokinetic properties, including good absorption and good oral bioavailability. Table 2 shows the results. Table 2 is the experimental results of the pharmacokinetic characteristics of the compounds provided in the examples of the present invention in rats.

Table 2 The experimental results of the pharmacokinetic characteristics of the compounds provided in the examples of the present invention in rats

| Example No. | | PK | | | F (%) |
|---|---|---|---|---|---|
| | dose (mg/kg) | T_{½} (h) | Cₘₐₓ ng/mL | AUCₗₐₛₜ (ng.h/mL) | |
| Example | PO 5 | 3.11 | 2650 | 8810 | 106.7 |
| 1 | IV 1 | 1.24 | 1170 | 1650 | NA |
| Example 2 | PO 5 | 2.71 | 1990 | 9430 | 164.9 |
| | IV 1 | 0.971 | 1010 | 1140 | NA |
| Example 26 | PO 5 | 3.62 | 612 | 2290 | 98.5 |
| | IV 2 | 1.65 | 289 | 494 | NA |

As can be seen from Table 2, when the compounds provided herein are administered intravenously or orally, the compounds of the present invention show good pharmacokinetic properties, including good absorption (AUCₗₐₛₜ) and good oral bioavailability (F).

### Example C Kinase Activity Experiments

The utility of the compounds disclosed in this invention as protein kinase inhibitors can be evaluated by the following experiments.

### General Description of Kinase Experiments

Kinase experiments are performed by detecting myelin basic protein (MBP) incorporated into γ-³³P-ATP. 20 µg/ml of MBP (Sigma #M-1891) in tris-buffered saline (TBS; 50 mM Tris pH 8.0, 138 mM NaCl, 2.7 mM KCl) were prepared and coated high-binding white 384-well plate (Greiner) with 60 µL per well. The plate was incubated at 4° C. for 24 h. Then washed with 100 µL TBS 3 times. Kinase reactions were performed in a total volume of 34 µL in Kinase Buffer (5 mM Hepes pH 7.6, 15 mM NaCl, 0.01% Bovine Serum Albumin (Sigma #I-5506), 10 mM MgCl₂, 1 mM DTT, 0.02% TritonX-100). Compounds were dissolved in DMSO and added to the wells at a final concentration of 1% DMSO. Each data was determined twice, and the determination of each compound was carried out at least twice. For example, the final enzyme concentration was 10 nM or 20 nM. The reaction was started by adding unlabeled ATP (10 µM) and γ-³³P-labeled ATP (2 × 10⁶ cpm per well, 3000 Ci/mmol). The reaction was carried out with shaking at room temperature for 1 h. The 384-well plate was washed with 7 × PBS, and then 50 µL of scintillation fluid was added to each well. Results were checked with a Wallac Trilux counter. For those skilled in the art, this is only one of many detection methods, and other methods are also acceptable.

IC₅₀ of inhibition and/or inhibition constant Kᵢ can be obtained by the above test method. IC₅₀ is defined as the concentration of the compound that inhibits 50% of the enzyme activity under the test conditions. IC50 values were estimated by plotting 10 concentration points using ½ log dilutions (e.g., plot a typical curve across the following compound concentrations: 3 µM, 1 µM, 0.3 µM, 0.1 µM, 0.03 µM, 0.01 µM, 0.003 µM, 0.001 µM, 0.0003 µM, 0 µM).

### JAK1 (h)

JAK1 (h) was incubated in the presence of 20 mM Tris/HCl pH 7.5, 0.2 mM EDTA, 500 µM GEEPLYWSFPAKKK, 10 mM magnesium acetate and [γ-³³P-ATP] (specific activity was about 500 cpm/pmol, 10 µM or K_{M} value). The reaction was started after adding the MgATP mixture. After incubation at room temperature for 40 min, a 3% phosphoric acid solution was added to terminate the reaction. 10 µL of the reaction solution was spotted onto a P30 filter, washed 3 times in 5 min with 75 mM phosphoric acid, and stored in methanol solution immediately prior to drying and scintillation counting.

### JAK2 (h)

JAK2 (h) was incubated in the presence of 8 mM MOPS pH 7.0, 0.2 mM EDTA, 100 µM KTFCGTPEYLAPEVRREPRILSEEEQEMFRDFDYIADWC, 10 mM magnesium acetate and [γ-³³P-ATP] (specific activity was about 500 cpm/pmol, 10 µM or K_{M} value). The reaction was started after adding the MgATP mixture. After incubation at room temperature for 40 min, a 3% phosphoric acid solution was added to terminate the reaction. 10 µL of the reaction solution was spotted onto a P30 filter, washed 3 times in 5 min with 75 mM phosphoric acid, and stored in methanol solution immediately prior to drying and scintillation counting.

### JAK3 (h)

JAK3 (h) was incubated in the presence of 8 mM MOPS pH 7.0, 0.2 mM EDTA, 500 µM GGEEEEYFELVKKKK, 10 mM magnesium acetate and [γ-³³P-ATP] (specific activity was about 500 cpm/pmol, 10 µM or K_{M} value). The reaction was started after adding the MgATP mixture. After incubation at room temperature for 40 min, a 3% phosphoric acid solution was added to terminate the reaction. 10 µL of the reaction solution was spotted onto a P30 filter, washed 3 times in 5 min with 75 mM phosphoric acid, and stored in methanol solution immediately prior to drying and scintillation counting.

### TYK2 (h)

TYK2 (h) was incubated in the presence of 8 mM MOPS pH 7.0, 0.2 mM EDTA, 250 µM GGMEDIYFEFMGGKKK, 10 mM magnesium acetate and [γ-³³P-ATP] (specific activity was about 500 cpm/pmol, 10 uM or K_{M} value). The reaction was started after adding the MgATP mixture. After incubation at room temperature for 40 min, a 3% phosphoric acid solution was added to terminate the reaction. 10 µL of the reaction solution was spotted onto a P30 filter, washed 3 times in 5 min with 75 mM phosphoric acid, and stored in methanol solution immediately prior to drying and scintillation counting.

The kinase experiment in the present invention was performed by Millipore, UK (Millipore UK Ltd, Dundee Technology Park, Dundee DD2 1SW, UK).

Alternatively, the kinase activity of compounds can be detected by KINOMEscan™, which is based on quantitative detection of compounds using an active site-directed competitive binding assay. The experiment was carried out by combining with three compounds, namely DNA labeling enzyme, immobilized ligand and detection compound, and the ability of compounds to compete with immobilized ligands was tested by qPCR with DNA labeling.

In most experiments, kinase-tagged T7 phage strains were cultivated from E. coli hosts derived from BL21 strains. After infecting E. coli in the logarithmic growth phase with T7 phages, they were shaken and incubated at 32° C. until lysed, and the lysed products were centrifuged and filtered, the cell debris was removed, and the remaining kinases were transferred to HEK-293 cells for qPCR detection with DNA markers. Streptavidin-coated particles were treated with biotinylated small molecule ligands for 30 min at room temperature to generate affinity resins for kinase experiments. After the ligand particles were blocked with excess biotin, the unbound ligand was washed with blocking solution (SEABLOCK™ (Pierce), 1% bovine serum albumin, 0.05% Tween-20, 1 mM DTT) to reduce non-specific binding. Binding reactions were performed by binding kinase, ligand affinity particles and test compounds in 1× binding buffer (20% SEABLOCK™, 0.17× phosphate buffered saline, 0.05% Tween 20, 6 mM DTT), all reactions were carried out in a 96-well plate, the final volume of the reaction was 0.135 mL. The mixture was shaken and incubated at room temperature for 1 h, washing buffer (1× phosphate buffer solution, 0.05% Tween 20) was added to wash the affinity particles, elution buffer was added to resuspend (1× phosphate buffered saline, 0.05% Tween 20, 0.5 µM non-biotinylated affinity ligand), the mixture was incubated with shaking at room temperature for 30 min, and the concentration of kinase in the eluate was detected by qPCR. Kinase activity experiments described herein were performed at the KINOMEscan™ division of DiscoveRx Corporation (Albrae St. Fremont, CA 94538, USA).

It can be seen from the experimental results that the compounds of the present invention have significant inhibitory activity on TYK2 kinase in the kinase experiment.

### Example D Cell Activity Experiments

The utility of the compounds disclosed in the present invention as TYK2 inhibitors can be evaluated by the following experiments.

### Effect of TYK2 Inhibitor on P-STAT3 Expression in PBMC Induced by IFN-α

Whole blood was collected from healthy subjects, anticoagulated with heparin sodium, and PBMC (human peripheral blood mononuclear cells) were extracted. PBMC were seeded in 96-well culture plates at a density of 1*106/well. The initial concentration of the positive drug BMS986165 was 1000 nM, after diluting 10 times, the positive drug BMS986165 was diluted 8 concentrations by 3 times; the concentration of the other tested compounds was 333 nM. Positive wells and negative wells were added with the same volume of DMSO as the compound, and the final concentration of DMSO was 0.5%. After the compound was added, the cells were incubated at 37° C. for 1 h, and then stimulated by the addition of IFN-α at a final concentration of 5000 U/mL. An equal volume of medium was added to the negative wells and incubated for 15 min. The cells were centrifuged to remove the supernatant, and lysed, and the P-STAT3 was measured according to the instructions of the ELISA kit, the absorbance was read at 450 nm, and the IC₅₀ value or inhibition rate was calculated.

### Inhibitory Effect of TYK2 Inhibitor on TF-1 Cell Proliferation

TF-1 cells were seeded in a 96-well culture plate at a density of 20,000/well with 80 µL per well, the blank well was an equal amount of complete medium, and 10 µL of the compound dilution solution was added to the 96-well plate B1-11, C1-11, E1-11, F1-11, positive and negative wells were added with 5% DMSO medium equal to the volume of the compound, and the final concentration of DMSO was 0.5%. After 30 min, GM-CSF with a final concentration of 2 ng/mL was added to rows B, C, and D (positive wells), and the remaining wells (rows E, F, and G) were added with the same volume of medium as cytokines (negative wells). After incubation for 44 h at 37° C. in 5% CO₂, alamar blue was added, the plate was incubated for 4 h, the fluorescence intensity of each well under the conditions of 540 nM, 580 nM wavelength was measured, and the IC₅₀ value was calculated.

### Inhibitory Effect of TYK2 Inhibitor on CTLL-2, P-STAT3, TF-1 Cell Proliferation

CTLL-2, P-STAT3 and TF-1 cells were seeded in a 96-well culture plate at a density of 20,000/well with 90 µL per well, and the blank well was an equal amount of complete medium. 10 ul of different compound dilutions were added to each plate row BC, DE, and FG, and 5% DMSO medium with equal volume of the compound was added to positive wells and negative wells, and the final concentration of DMSO was 0.5%. After incubating in a 37° C. incubator for 20 h, alamar blue was added, the plate was incubated for 4 h, the fluorescence intensity of each well under the conditions of 540 nM, 580 nM wavelength was measured, and the IC₅₀ value was calculated. The results are shown in Table 3. Table 3 shows the results of the cell inhibitory activity experiments of the compounds provided in the examples of the present invention.

Table 3 P-STAT3, CTLL-2 and TF-1 cell inhibition experiment results of the compounds of the present invention

| Example No. | IC₅₀ (nM) | | |
|---|---|---|---|
| | P-STAT3 (h) | CTLL-2 (h) | TF-1 (h) |
| Example 2 | 95.75 | > 10000 | 7563 |
| Example 3 | 21.41 | > 10000 | 5292 |

It can be seen from the experimental results that the compounds of the present invention have significant inhibitory activity on P-STAT3 cells in the cell inhibition experiment, and have no inhibitory activity on CTLL-2 and TF-1 cells, so the compounds of the present invention have good TYK2 selectivity inhibition.

### Example E Cell Level Activity Test Method of JAK1/TYK2

IFN-α can induce the phosphorylation of STAT3 downstream of JAK1/TYK2 in PBMC, and by measuring the level of STAT3 phosphorylation, the activity of the compound on JAK1/TYK2 at the cell level can be reflected.

Whole blood was collected from healthy subjects, anticoagulated with heparin sodium, and PBMC (human peripheral blood mononuclear cells) were extracted. PBMC were seeded in a 96-well culture plates at a density of 1*106/well. Positive wells and negative wells were added with the same volume of DMSO as the compound, and the final concentration of DMSO was 0.5%. The compounds were incubated with the cells at 37° C. for 1 h, and then stimulated by adding IFN-α at a final concentration of 5000 U/mL, an equal volume of medium was added to the negative wells, and incubated for 15 min. The cells were centrifuged to remove the supernatant, then lysed, and P-STAT3 was measured according to the instructions of the ELISA kit, the absorbance was read at 450 nm, and the IC₅₀ value or inhibition rate was calculated. The results are shown in Table 4. Table 4 shows the experimental results of the activity of the compound provided in the examples of the present invention on JAK1/TYK2 cell level.

Table 4 shows the results of activity experiments on JAK1/TYK2 cell level

| | | | |
|---|---|---|---|
| compound | IC₅₀ nM (JAK 1 /TYK2) | compound | IC₅₀ nM (JAK 1 /TYK2) |
| Example 2 | 95.75 | Example 19 | 115 |
| Example 3 | 21.41 | Example 20 | 64.86 |
| Example 5 | 177.9 | Example 21 | 36.88 |
| Example 7 | 14.09 | Example 22 | 7.23 |
| Example 8 | 41.17 | Example 23 | 103.6 |
| Example 9 | 113.6 | Example 24 | 29.94 |
| Example 10 | >1000 | Example 25 | 45.52 |
| Example 11 | >1000 | Example 26 | 110 |
| Example 13 | 104.1 | Example 27 | 13.10 |
| Example 15 | 48.64 | Example 28 | 12.94 |
| Example 17 | 125.7 | Example 29 | 93.12 |
| Example 18 | 19.6 | Control compound | 52.66 |
| | | 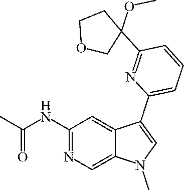 [SMILES: COC1(c2cccc(-c3cn(C)c4cnc(NC(C)=O)cc34)n2)CCOC1] | |

It can be seen from the experimental results that the compounds of the present invention have better inhibitory activity on the JAK1/TYK2 cell level.

### Example F Cell Level Activity Test Method of JAK2/TYK2

TYK2 belongs to the JAK family, and can accept the ligand acting on the coupled receptor signal to regulate downstream signal activator of transcription (STAT) phosphorylation. Phosphorylation of STAT can regulate the expression of downstream related genes, leading to changes in physiological functions such as cell proliferation and differentiation. IL-12 mediates IFNγ expression in NK92 cells through JAK2/TYK2.

Therefore, by inhibiting TYK2 activity, inhibition of this cascade pathway leads to a decrease in IFNγ expression. However, IL-2 can induce NK92 proliferation and produce IFNγ through receptor-coupled JAK⅓, so the influence of IL-2 needs to be ruled out. In this experiment, the activity of compounds on JAK2/TYK2 was evaluated by detecting the expression of IFNγ at various compound concentrations.

The test compound was dissolved in DMSO, prepared into a 20 mM stock solution, and stored at -20° C. for future use. The mother solution was diluted 10 times with DMSO to make a 2 mM solution, then diluted with medium to an initial concentration of 105 nM, and then diluted 3-fold with medium containing 5% DMSO to obtain a prepared concentration gradient of 105 nM, 33333.3 nM, 11111.1 nM, 3703.70 nM, 1234.57 nM, 411.523 nM, 137.174 nM, 45.7247 nM, 15.2416 nM; 10 ul of the above-mentioned concentration of drugs were added into a 96-well plate to obtain the final concentrations of 104 nM, 3333.3 nM, 1111.1 nM, 370.4 nM, 123.5 nM, 41.1 nM, 13.7 nM, 4.6 nM, 1.52 nM;

NK92 cells were resuscitated and cultured, 16 h before the experiment, the medium was replaced with interleukin-free medium. The mixture was centrifuged and IL-12 medium was added to resuspend the cells, seeded 95 ul in a 96-well plate at a density of 20,000 cells/well, 10 ul of the above gradual dilution was added and incubated for 24 h, centrifuged to take the supernatant, diluted 3-fold with pure water, and the IFNγ concentration of the supernatant was detected by ELISA, the IC₅₀ value was calculated.

The results are shown in Table 5. Table 5 shows the experimental results of the activity of the compounds provided in the examples of the present invention on the JAK2/TYK2 cell level.

Table 5 shows the results of activity experiments on the JAK2/TYK2 cell level

| compound | IC₅₀ nM (JAK2/TYK2) | compound | IC₅₀ nM (JAK2/TYK2) |
|---|---|---|---|
| Example 30 | 102 | Example 40 | 152 |
| Example 32 | 30.6 | Example 42 | 9.6 |
| Example 33 | 38.6 | Example 43 | 26.4 |
| Example 34 | 34.3 | Example 44 | 40.2 |
| Example 35 | 20.4 | Example 45 | 111.2 |
| Example 36 | 52.9 | Example 48 | 115 |
| Example 37 | 20.0 | Example 49 | 68.12 |
| Example 38 | 27.7 | Example 50 | 47 |
| Example 39 | 11.0 | | |

It can be seen from the experimental results that the compounds of the present invention have better inhibitory activity on the JAK2/TYK2 cell level.

Finally, it should be noted that there are other ways to implement the invention. Accordingly, the examples of the present invention will be described as illustrations, but not limited to the described contents of the present invention. It can be understood that the above-mentioned embodiments are exemplary, and should not be construed as limitations on the present invention, and those skilled in the art may make changes, alternatives, and modifications to the above-mentioned embodiments within the scope of the present invention. Modifications within the scope of the invention or equivalents added in the claims are also possible. All publications or patents cited in the present invention shall be regarded as reference documents of the present invention.

What is claimed is:

1. A compound having formula (I) or a stereoisomer, a geometric isomer, a tautomer, an N-oxide, a hydrate, a solvate, a metabolite, a pharmaceutically acceptable salt or a prodrug thereof, 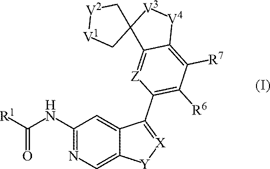
wherein:
X is N or CR^{a}, Z is Nor CR^{e};
Y is NR^{b} or CR^{c}R^{d};
R¹ is —NH₂, C₁₋₆ alkyl, C₁₋₆ alkylamino, C₃₋₈ cycloalkyl, heterocyclyl consisting of 3-8 atoms, C₆₋₁₀ aryl or heteroaryl consisting of 5-12 atoms, wherein, the —NH₂, C₁₋₆ alkyl, C₁₋₆ alkylamino, C₃₋₈ cycloalkyl, heterocyclyl consisting of 3-8 atoms, C₆₋₁₀ aryl and heteroaryl consisting of 5-12 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy;
each of R⁶ and R⁷ is independently H, F, Cl, Br, I, —NO₂, CN, —OH, —NH₂, C₁₋₆ alkyl, C₁₋₆ haloalkyl, C₂₋₆ alkenyl, C₂₋₆ alkynyl, C₁₋₆ alkoxy, C₃₋₆ cycloalkyl, C₃₋₆ cycloalkyl-O-, C₁₋₆ alkylamino, C₁₋₆ alkyl-S(=O)₂-, C₁₋₆ alkyl-S-, heterocyclyl consisting of 3-8 atoms, C₆₋₁₀ aryl or heteroaryl consisting of 5-10 atoms, wherein, the —OH, —NH₂, C₁₋₆ alkyl, C₁₋₆ haloalkyl, C₂₋ ₆ alkenyl, C₂₋₆ alkynyl, C₁₋₆ alkoxy, C₃₋₆ cycloalkyl, C₃₋₆ cycloalkyl-O-, C₁₋₆ alkylamino, C₁₋₆ alkyl-S(=O)₂-, C₁₋₆ alkyl-S-, heterocyclyl consisting of 3-8 atoms, C₆₋₁₀ aryl and heteroaryl consisting of 5-10 atoms can be independently and optionally substituted with 1, 2, 3, 4 or 5 R^{x};
each of V¹, V², V³ and V⁴ is independently -(CR⁹R¹⁰)ₙ₋, -(CR⁹R¹⁰)ₙ₋O-, -(CR⁹R ¹⁰ )ₙ₋S-, (CR⁹R¹⁰)ₙ₋NR¹¹-, -(CR⁹R¹⁰)ₙ-C(=O)-, -(CR⁹R^{1O})ₙ-O-C(=O)-, -(CR⁹R¹⁰)ₙ-C(=O)-O-, -(CR⁹R¹⁰)ₙ-S(=O)- or -(CR⁹R¹⁰)ₙ-S(=O)₂-;
each of R⁹ and R¹⁰ is independently H, D, F, Cl, Br, I, —NO₂, CN, —OH, —NH₂, C₁₋₆ alkyl, C₁₋₆ haloalkyl, C₁₋₆ alkoxy or C₃₋₆ cycloalkyl, wherein, the —OH, —NH₂, C₁₋₆ alkyl, C₁₋₆ haloalkyl, C₁₋₆ alkoxy and C₃₋₆ cycloalkyl can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy; or
R⁹ and R¹⁰, together with the carbon atom to which they are attached, form C₃₋₆ cycloalkyl or heterocyclyl consisting of 3-6 atoms, wherein, the C₃₋₆ cycloalkyl and heterocyclyl consisting of 3-6 atoms can be independently and optionally substituted with 1, 2, 3, 4 or 5 substituents selected from F, Cl, Br, I, —NO₂, CN, —OH, —NH₂, oxo, C₁₋₃ alkyl, C₁₋₃ haloalkyl, C₁₋₃ alkoxy, C₁₋₃ haloalkoxy and C₁₋₃ hydroxyalkoxy;
R ¹¹ is H, D, C₁₋₆ alkyl, C₁₋₆ alkyl-C(=O)-, C₁₋₆ alkyl-O-C(=O)-, C₁₋₆ haloalkyl or C₃₋₆ cycloalkyl, wherein, C₁₋₆ alkyl, C₁₋₆ alkyl-C(=O)-, C₁₋₆ alkyl-O-C(=O)-, C₁₋₆ haloalkyl and C₃₋₆ cycloalkyl can be independently and optionally substituted with 1, 2, 3, 4 or 5 substituents selected from F, Cl, Br, I, —NO₂, CN, —OH, —NH₂, oxo, C₁₋₃ alkyl, C₁₋₃ haloalkyl, C₁₋₃ alkoxy, C₁₋₃ haloalkoxy, C₁₋₃ hydroxyalkoxy and C₃₋₆ cycloalkyl;
each of R^{a}, R^{c}, R^{d} and R^{e} is independently H, D, F, Cl, Br, I, —NO₂, CN, —OH, —NH₂, C₁₋₆ alkyl, C₁₋₆ haloalkyl, C₂₋₆ alkenyl, C₂₋₆ alkynyl, C₁₋₆ alkoxy, C₃₋₈ cycloalkyl, heterocyclyl consisting of 3-8 atoms, C₆₋₁₀ aryl or heteroaryl consisting of 5-12 atoms, wherein, the —OH, —NH₂, C₁₋₆ alkyl, C₁₋₆ haloalkyl, C₂₋₆ alkenyl, C₂₋₆ alkynyl, C₁₋₆ alkoxy, C₃₋₈ cycloalkyl, heterocyclyl consisting of 3-8 atoms, C₆₋₁₀ aryl and heteroaryl consisting of 5-12 atoms can be independently and optionally unsubstituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy;
R^{b} is H, D, C₁₋₆ alkyl, C₁₋₆ haloalkyl, C₂₋₆ alkenyl, C₂₋₆ alkynyl, C₁₋₆ alkoxy, C₃₋₈ cycloalkyl, heterocyclyl consisting of 3-8 atoms, C₆₋₁₀ aryl or heteroaryl consisting of 5-12 atoms, wherein, the C₁₋₆ alkyl, C₁₋₆ haloalkyl, C₂₋₆ alkenyl, C₂₋₆ alkynyl, C₁₋₆ alkoxy, C₃₋₈ cycloalkyl, heterocyclyl consisting of 3-8 atoms, C₆₋₁₀ aryl and heteroaryl consisting of 5-12 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy;
each R^{X} is independently F, Cl, Br, I, —NO₂, CN, —OH, —NH₂, C₁₋₆ alkyl, C₁₋₆ haloalkyl, C₂₋₆ alkenyl, C₂₋₆ alkynyl, C₁₋₆ alkoxy, C₁₋₆ alkylamino or C₃₋₆ cycloalkyl, wherein, the —OH, —NH₂, C₁₋₆ alkyl, C₁₋₆ haloalkyl, C₂₋₆ alkenyl, C₂₋₆ alkynyl, C₁₋₆ alkoxy, C₁₋₆ alkylamino and C₃₋₆ cycloalkyl can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ alkylamino, C₁₋₃ haloalkoxy and C₁₋₃ hydroxyalkoxy;
each n is independently 0, 1, or 2.

2. The compound of claim 1, R¹ is —NH₂, C₁₋₄ alkyl, C₁₋₄ alkylamino, C₃₋₆ cycloalkyl, heterocyclyl consisting of 3-6 atoms, C₆₋₁₀ aryl or heteroaryl consisting of 5-10 atoms, wherein, the —NH₂, C₁₋₄ alkyl, C₁₋₄ alkylamino, C₃₋₆ cycloalkyl, heterocyclyl consisting of 3-6 atoms, C₆₋₁₀ aryl and heteroaryl consisting of 5-10 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy;
each R⁶ and R⁷ is independently H, F, Cl, Br, I, —NO₂, CN, —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, C₃₋₆ cycloalkyl-O-, C₁₋₄ alkylamino, C₁₋₄ alkyl-S(=O)₂-, C₁₋₄ alkyl—S—, heterocyclyl consisting of 3-6 atoms, C₆₋₁₀ aryl or heteroaryl consisting of 5-10 atoms, wherein, the —OH, —NH₂, C₁₋₄ alkyl, C₁₋₄ haloalkyl, C₂₋ ₄ alkenyl, C₂₋₄ alkynyl, C₁₋₄ alkoxy, C₃₋₆ cycloalkyl, C₃₋₆ cycloalkyl-O-, C₁₋₄ alkylamino, C₁₋₄ alkyl-S(=O)₂-, C₁₋₄ alkyl-S-, heterocyclyl consisting of 3-6 atoms, C₆₋₁₀ aryl and heteroaryl consisting of 5-10 atoms can be independently and optionally substituted with 1, 2, 3, 4 or 5 R^{x}.

3. The compound of claim 1, R¹ is —NH₂, methyl, ethyl, n-propyl, isopropyl, n-butyl, isobutyl, sec-butyl, tert-butyl, N-methylamino, N-ethylamino, N,N-dimethylamino, N,N-diethylamino, N-ethylpropyl-2-amino, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein, the —NH₂, methyl, ethyl, n-propyl, isopropyl, n-butyl, isobutyl, sec-butyl, i-butyl, N-methylamino, N-ethylamino, N,N-dimethylamino, N,N-diethylamino, N-ethylpropyl-2-amino, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and pyridazinyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, —OCH₂OH and -OCH₂CH₂OH;
each R⁶ and R⁷ is independently H, F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, cyclopropyl-O-, cyclobutyl-O-, cyclopentyl-O-, cyclohexyl-O-, —NH(CH₂)₃CH₃, CH₃(CH₂)₃S—, CH₃—S(═O)₂—, CH₃(CH₂)₃—S(═O)₂—, oxetanyl, azetidinyl, pyrrolidinyl, tetrahydrofuranyl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein, the —OH, —NH₂, methyl, ethyl, n-propyl, isopropyl, n-butyl, isobutyl, sec-butyl, tert-butyl, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, cyclopropyl-O-, cyclobutyl-O-, cyclopentyl-O-, cyclohexyl-O-, —NH(CH₂)₃CH₃, CH₃(CH₂)₃S—, CH₃—S(═O)₂—, CH₃(CH₂)₃—S(═O)₂—, oxetanyl, azetidinyl, pyrrolidinyl, tetrahydrofuranyl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and idazinyl can be independently and optionally substituted with 1, 2, 3, 4 or 5 R^{x}.

4. The compound of claim 1, each R⁹ and R¹⁰is independently H, D, F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl or cyclohexyl, wherein, the —OH, —NH₂, methyl, ethyl, n-propyl, isopropyl, n-butyl, isobutyl, sec-butyl, tert-butyl, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl and cyclohexyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, —OCH₂OH and —OCH₂CH₂OH; or
R⁹ and R¹⁰,together with the carbon atom to which they are attached, form cyclopropyl, cyclobutyl, cyclopentyl, cyclohexyl, oxetanyl, azetidinyl, pyrrolidinyl, tetrahydrofuranyl, piperidinyl, piperazinyl or morpholinyl, wherein, each of the cyclopropyl, cyclobutyl, cyclopentyl, cyclohexyl, oxetanyl, azetidinyl, pyrrolidinyl, tetrahydrofuranyl, piperidinyl, piperazinyl or morpholinyl is independently unsubstituted or substituted with 1, 2, 3, 4 or 5 substituents selected from F, Cl, Br, I, —NO₂, CN, —OH, —NH₂, oxo, methyl, ethyl, n-propyl, isopropyl, trifluoromethyl, difluoromethyl, methoxy, ethoxy, isopropoxy, trifluoromethoxy, —OCH₂OH and —OCH₂CH₂OH;
R¹¹ is H, D, methyl, ethyl, n-propyl, isopropyl, n-butyl, isobutyl, sec-butyl, tert-butyl, CH₃CH₂—C(═O)—, CH₃CH₂—O—C(═O)—, —CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, cyclopropyl, cyclobutyl, cyclopentyl or cyclohexyl, wherein, each of the methyl, ethyl, n-propyl, isopropyl, n-butyl, isobutyl, sec-butyl, tert-butyl, CH₃CH₂—C(═O)—, CH₃CH₂—O—C(═O)—, —CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, cyclopropyl, cyclobutyl, cyclopentyl and cyclohexyl is independently unsubstituted or substituted with 1, 2, 3, 4 or 5 substituents selected from F, Cl, Br, I, —NO₂, CN, —OH, —NH₂, oxo, methyl, ethyl, n-propyl, isopropyl, trifluoromethyl, difluoromethyl, methoxy, ethoxy, isopropoxy, trifluoromethoxy, —OCH₂OH, —OCH₂CH₂OH, cyclopropyl, cyclobutyl, cyclopentyl and cyclohexyl.

5. The compound of claim 1, each of R^{a}, R^{c}, R^{d} and R^{e} is independently H, D, F, Cl, Br, I, —NO₂, CN, —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl or heteroaryl consisting of 5-10 atoms, wherein, the —OH, —NH₂, C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl and heteroaryl consisting of 5-10 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy;
R^{b} is H, D, C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl or heteroaryl consisting of 5-10 atoms, wherein, the C₁₋₄ alkyl, C₁₋₄ haloalkyl, C₂₋₄ alkenyl, C₂₋₄ alkynyl, C₁₋₄ alkoxy, C₃₋₆ cycloalkyl, heterocyclyl consisting of 3-6 atoms, C₆₋₁₀ aryl and heteroaryl consisting of 5-10 atoms can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ haloalkoxy and C₁₋₃ hydroxyalkoxy;
each R^{x} is independently F, Cl, Br, I, —NO₂, CN, —OH, —NH₂, C₁₋₄ alkyl, C₁₋₄ haloalkyl, C₂₋₄ alkenyl, C₂₋₄ alkynyl, C₁₋₄ alkoxy, C₁₋₄ alkylamino or C₃₋₆ cycloalkyl, wherein, the —OH, —NH₂, C₁₋₄ alkyl, C₁₋₄ haloalkyl, C₂₋₄ alkenyl, C₂₋₄ alkynyl, C₁₋₄ alkoxy, C₁₋₄ alkylamino and C₃₋₆ cycloalkyl can be independently and optionally substituted with 1, 2, 3, 4 or 5 substituents selected from F, Cl, Br, I, —NO₂, CN, —OH, —NH₂, C₁₋₃ alkyl, C₁₋₃ haloalkyl, C₁₋₃ alkoxy, C₁₋₃ alkylamino, C₁₋₃ haloalkoxy and C₁₋₃ hydroxyalkoxy.

6. The compound of claim 1, each of R^{a}, R^{c}, R^{d} and R^{e} is independently H, D, F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein, the —OH, —NH₂, methyl, ethyl, n-propyl, isopropyl, n-butyl, isobutyl, sec-butyl, tert-butyl, CH₂F, —CH₂Cl, —CHF₂, -—CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and pyridazinyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, -OCH₂OH and -OCH₂CH₂OH;
R^{b} is H, D, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, -CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl or pyridazinyl, wherein, the methyl, ethyl, n-propyl, isopropyl, n-butyl, isobutyl, sec-butyl, tert-butyl, CH₂F, CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, cyclopropyl, cyclobutyl, cyclopentyl, cyclohexyl, oxetanyl, azetidinyl, pyrrolidinyl, tetrahydrofuryl, piperidinyl, piperazinyl, morpholinyl, phenyl, naphthyl, benzimidazolyl, pyrrolyl, pyrazolyl, imidazolyl, triazolyl, tetrazolyl, furyl, thienyl, thiazolyl, oxazolyl, pyridyl, pyrimidinyl, pyrazinyl and pyridazinyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, trifluoromethoxy, -OCH₂OH and -OCH₂CH₂OH;
each R^{x} is independently F, Cl, Br, I, —NO₂, CN, —OH, —NH₂, methyl, ethyl, n-propyl, isopropyl, n-butyl, isobutyl, sec-butyl, tert-butyl, CH₂F, —CH₂Cl, —CHF₂, —CHCl₂, —CF₃, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, —N(CH₃)₂, cyclopropyl, cyclobutyl, cyclopentyl or cyclohexyl, wherein, the —OH, NH₂, methyl, ethyl, n-propyl, isopropyl, n-butyl, isobutyl, sec-butyl, tert-butyl, CH₂F, —CH₂Cl, —CHF₂, —CHCl₂, —CH₂CH₂F, —CH₂CH₂Cl, —CH₂CHF₂, —CH₂CHCl₂, —CHFCH₂F, —CHClCH₂Cl, —CH₂CF₃, —CH(CF₃)₂, —CF₂CH₂CH₃, —CH₂CH₂CH₂F, —CH₂CH₂CHF₂, —CH₂CH₂CF₃, vinyl, propenyl, allyl, ethynyl, propargyl, 1-propynyl, but-1-yne-4-yl, but-2-yne-1-yl, but-1-yne-1-yl, methoxy, ethoxy, 1-propoxy, 2-propoxy, 1-butoxy, 2-methyl-1-propoxy, 2-butoxy, 2-methyl-2-propoxy, —N(CH₃)₂, cyclopropyl, cyclobutyl, cyclopentyl and cyclohexyl can be independently and optionally substituted with 1, 2, 3, 4 or 5 substituents selected from F, Cl, Br, I, —NO₂, CN, —OH, —NH₂, methyl, ethyl, n-propyl, isopropyl, trifluoromethyl, difluoromethyl, methoxy, ethoxy, isopropoxy, —N(CH₃)₂—, trifluoromethoxy, —OCH₂OH and —OCH₂CH₂OH.

7. The compound of claim 1, which is a stereoisomer, a geometric isomer, a tautomer, an N-oxide, a hydrate, a solvate, a metabolite, a pharmaceutically acceptable salt or a prodrug thereof: 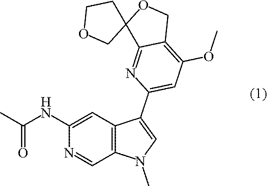 [SMILES: COc1cc(-c2cn(C)c3cnc(NC(C)=O)cc23)nc2c1COC21CCOC1] 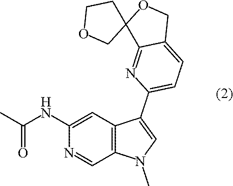 [SMILES: CC(=O)Nc1cc2c(-c3ccc4c(n3)C3(CCOC3)OC4)cn(C)c2cn1] 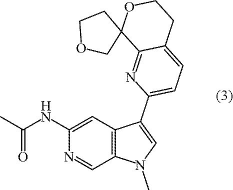 [SMILES: CC(=O)Nc1cc2c(-c3ccc4c(n3)C3(CCOC3)OCC4)cn(C)c2cn1] 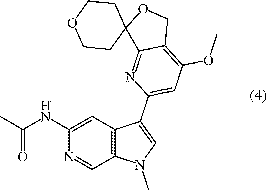 [SMILES: COc1cc(-c2cn(C)c3cnc(NC(C)=O)cc23)nc2c1COC21CCOCC1] 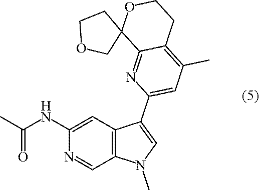 [SMILES: CC(=O)Nc1cc2c(-c3cc(C)c4c(n3)C3(CCOC3)OCC4)cn(C)c2cn1] 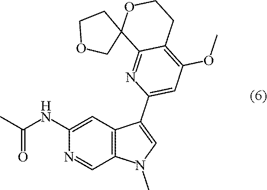 [SMILES: COc1cc(-c2cn(C)c3cnc(NC(C)=O)cc23)nc2c1CCOC21CCOC1] 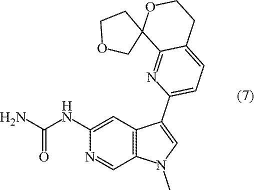 [SMILES: Cn1cc(-c2ccc3c(n2)C2(CCOC2)OCC3)c2cc(NC(N)=O)ncc21] 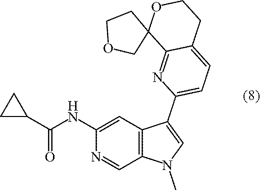 [SMILES: Cn1cc(-c2ccc3c(n2)C2(CCOC2)OCC3)c2cc(NC(=O)C3CC3)ncc21] 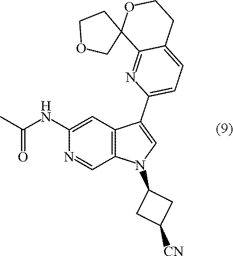 [SMILES: CC(=O)Nc1cc2c(-c3ccc4c(n3)C3(CCOC3)OCC4)cn([C@H]3C[C@@H](C#N)C3)c2cn1] 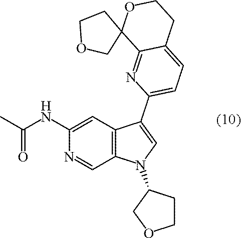 [SMILES: CC(=O)Nc1cc2c(-c3ccc4c(n3)C3(CCOC3)OCC4)cn([C@@H]3CCOC3)c2cn1] 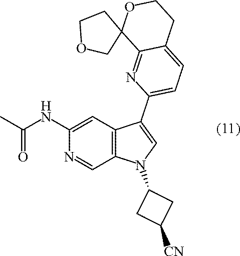 [SMILES: CC(=O)Nc1cc2c(-c3ccc4c(n3)C3(CCOC3)OCC4)cn([C@H]3C[C@H](C#N)C3)c2cn1] 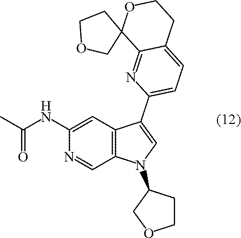 [SMILES: CC(=O)Nc1cc2c(-c3ccc4c(n3)C3(CCOC3)OCC4)cn([C@H]3CCOC3)c2cn1] 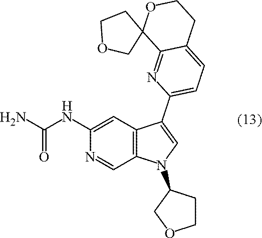 [SMILES: NC(=O)Nc1cc2c(-c3ccc4c(n3)C3(CCOC3)OCC4)cn([C@H]3CCOC3)c2cn1] 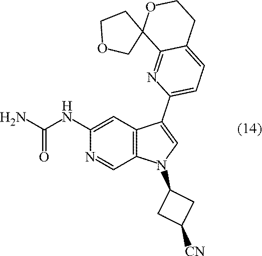 [SMILES: N#C[C@H]1C[C@@H](n2cc(-c3ccc4c(n3)C3(CCOC3)OCC4)c3cc(NC(N)=O)ncc32)C1] 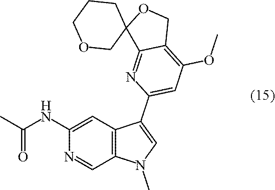 [SMILES: COc1cc(-c2cn(C)c3cnc(NC(C)=O)cc23)nc2c1COC21CCCOC1] 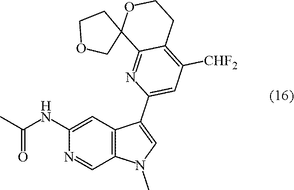 [SMILES: CC(=O)Nc1cc2c(-c3cc(C(F)F)c4c(n3)C3(CCOC3)OCC4)cn(C)c2cn1] 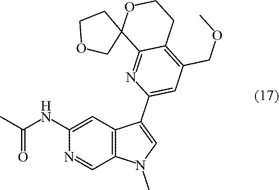 [SMILES: COCc1cc(-c2cn(C)c3cnc(NC(C)=O)cc23)nc2c1CCOC21CCOC1] 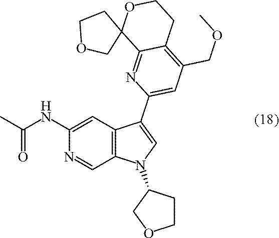 [SMILES: COCc1cc(-c2cn([C@@H]3CCOC3)c3cnc(NC(C)=O)cc23)nc2c1CCOC21CCOC1] 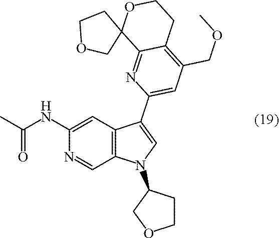 [SMILES: COCc1cc(-c2cn([C@H]3CCOC3)c3cnc(NC(C)=O)cc23)nc2c1CCOC21CCOC1] 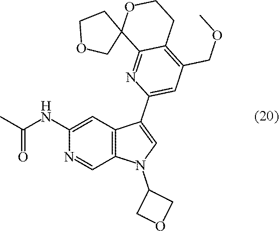 [SMILES: COCc1cc(-c2cn(C3COC3)c3cnc(NC(C)=O)cc23)nc2c1CCOC21CCOC1] 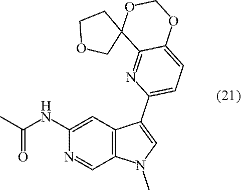 [SMILES: CC(=O)Nc1cc2c(-c3ccc4c(n3)C3(CCOC3)OCO4)cn(C)c2cn1] 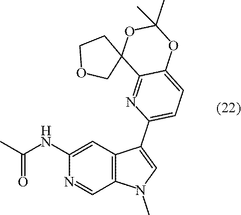 [SMILES: CC(=O)Nc1cc2c(-c3ccc4c(n3)C3(CCOC3)OC(C)(C)O4)cn(C)c2cn1] 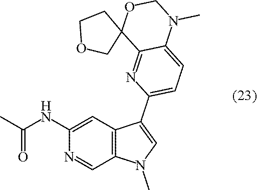 [SMILES: CC(=O)Nc1cc2c(-c3ccc4c(n3)C3(CCOC3)OCN4C)cn(C)c2cn1] 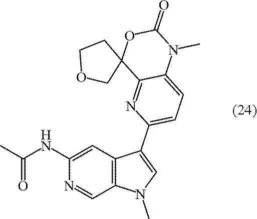 [SMILES: CC(=O)Nc1cc2c(-c3ccc4c(n3)C3(CCOC3)OC(=O)N4C)cn(C)c2cn1] 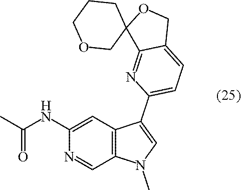 [SMILES: CC(=O)Nc1cc2c(-c3ccc4c(n3)C3(CCCOC3)OC4)cn(C)c2cn1] 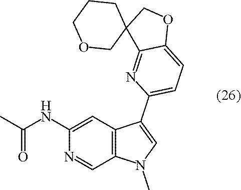 [SMILES: CC(=O)Nc1cc2c(-c3ccc4c(n3)C3(CCCOC3)CO4)cn(C)c2cn1] 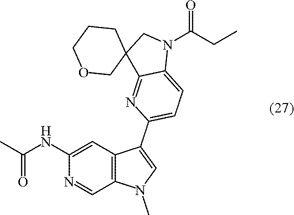 [SMILES: CCC(=O)N1CC2(CCCOC2)c2nc(-c3cn(C)c4cnc(NC(C)=O)cc34)ccc21] 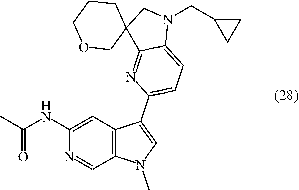 [SMILES: CC(=O)Nc1cc2c(-c3ccc4c(n3)C3(CCCOC3)CN4CC3CC3)cn(C)c2cn1] 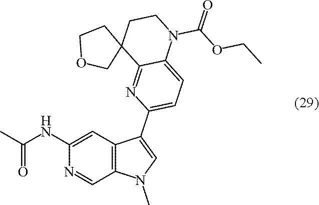 [SMILES: CCOC(=O)N1CCC2(CCOC2)c2nc(-c3cn(C)c4cnc(NC(C)=O)cc34)ccc21] 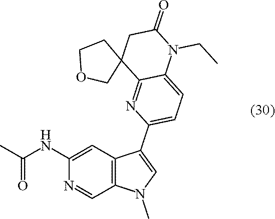 [SMILES: CCN1C(=O)CC2(CCOC2)c2nc(-c3cn(C)c4cnc(NC(C)=O)cc34)ccc21] 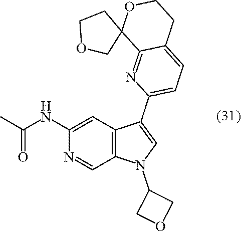 [SMILES: CC(=O)Nc1cc2c(-c3ccc4c(n3)C3(CCOC3)OCC4)cn(C3COC3)c2cn1] 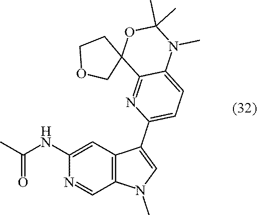 [SMILES: CC(=O)Nc1cc2c(-c3ccc4c(n3)C3(CCOC3)OC(C)(C)N4C)cn(C)c2cn1] 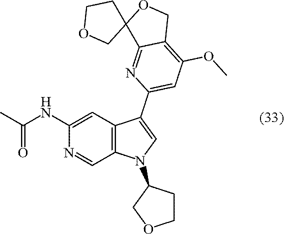 [SMILES: COc1cc(-c2cn([C@H]3CCOC3)c3cnc(NC(C)=O)cc23)nc2c1COC21CCOC1] 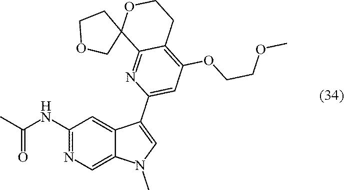 [SMILES: COCCOc1cc(-c2cn(C)c3cnc(NC(C)=O)cc23)nc2c1CCOC21CCOC1] 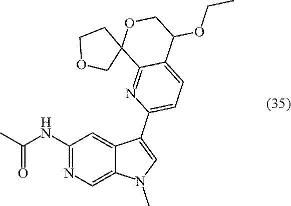 [SMILES: CCOC1COC2(CCOC2)c2nc(-c3cn(C)c4cnc(NC(C)=O)cc34)ccc21] 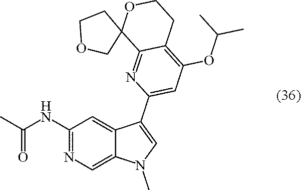 [SMILES: CC(=O)Nc1cc2c(-c3cc(OC(C)C)c4c(n3)C3(CCOC3)OCC4)cn(C)c2cn1] 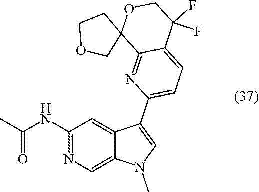 [SMILES: CC(=O)Nc1cc2c(-c3ccc4c(n3)C3(CCOC3)OCC4(F)F)cn(C)c2cn1] 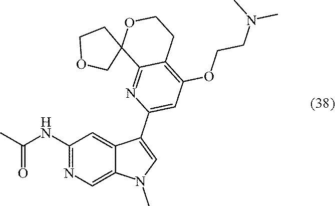 [SMILES: CC(=O)Nc1cc2c(-c3cc(OCCN(C)C)c4c(n3)C3(CCOC3)OCC4)cn(C)c2cn1] 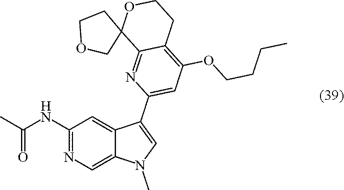 [SMILES: CCCCOc1cc(-c2cn(C)c3cnc(NC(C)=O)cc23)nc2c1CCOC21CCOC1] 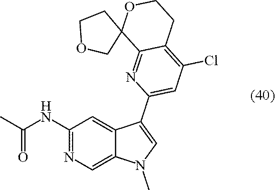 [SMILES: CC(=O)Nc1cc2c(-c3cc(Cl)c4c(n3)C3(CCOC3)OCC4)cn(C)c2cn1] 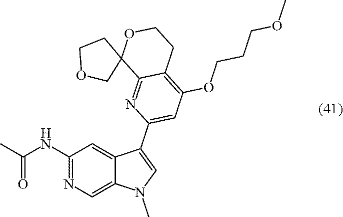 [SMILES: COCCCOc1cc(-c2cn(C)c3cnc(NC(C)=O)cc23)nc2c1CCOC21CCOC1] 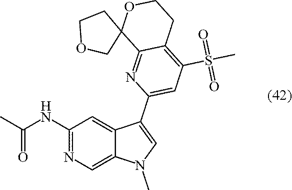 [SMILES: CC(=O)Nc1cc2c(-c3cc(S(C)(=O)=O)c4c(n3)C3(CCOC3)OCC4)cn(C)c2cn1] 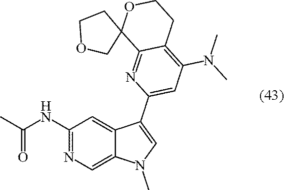 [SMILES: CC(=O)Nc1cc2c(-c3cc(N(C)C)c4c(n3)C3(CCOC3)OCC4)cn(C)c2cn1] 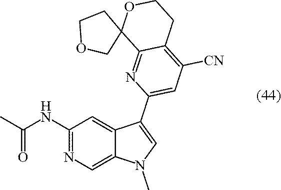 [SMILES: CC(=O)Nc1cc2c(-c3cc(C#N)c4c(n3)C3(CCOC3)OCC4)cn(C)c2cn1] 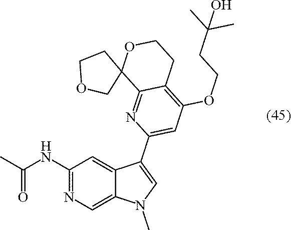 [SMILES: CC(=O)Nc1cc2c(-c3cc(OCCC(C)(C)O)c4c(n3)C3(CCOC3)OCC4)cn(C)c2cn1] 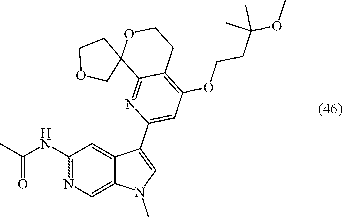 [SMILES: COC(C)(C)CCOc1cc(-c2cn(C)c3cnc(NC(C)=O)cc23)nc2c1CCOC21CCOC1] 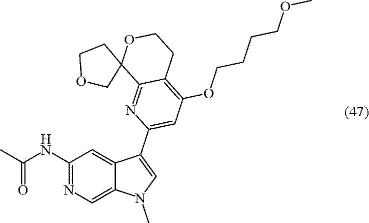 [SMILES: COCCCCOc1cc(-c2cn(C)c3cnc(NC(C)=O)cc23)nc2c1CCOC21CCOC1] 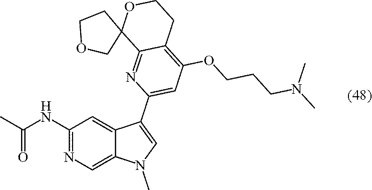 [SMILES: CC(=O)Nc1cc2c(-c3cc(OCCCN(C)C)c4c(n3)C3(CCOC3)OCC4)cn(C)c2cn1] 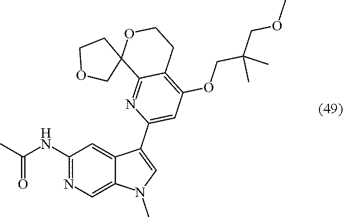 [SMILES: COCC(C)(C)COc1cc(-c2cn(C)c3cnc(NC(C)=O)cc23)nc2c1CCOC21CCOC1] 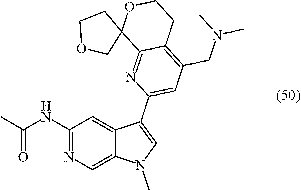 [SMILES: CC(=O)Nc1cc2c(-c3cc(CN(C)C)c4c(n3)C3(CCOC3)OCC4)cn(C)c2cn1] 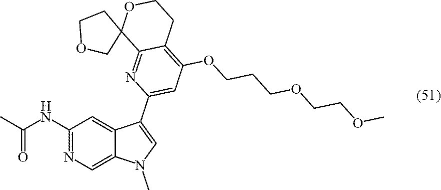 [SMILES: COCCOCCCOc1cc(-c2cn(C)c3cnc(NC(C)=O)cc23)nc2c1CCOC21CCOC1] 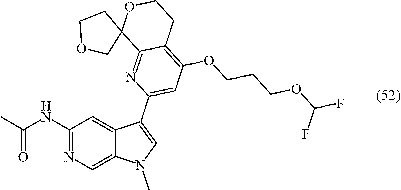 [SMILES: CC(=O)Nc1cc2c(-c3cc(OCCCOC(F)F)c4c(n3)C3(CCOC3)OCC4)cn(C)c2cn1] 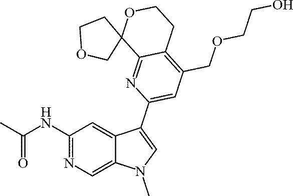 [SMILES: CC(=O)Nc1cc2c(-c3cc(COCCO)c4c(n3)C3(CCOC3)OCC4)cn(C)c2cn1] 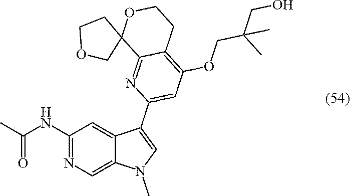 [SMILES: CC(=O)Nc1cc2c(-c3cc(OCC(C)(C)CO)c4c(n3)C3(CCOC3)OCC4)cn(C)c2cn1] 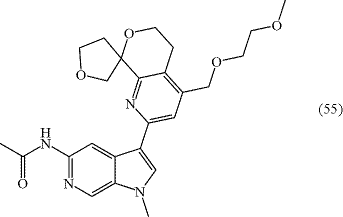 [SMILES: COCCOCc1cc(-c2cn(C)c3cnc(NC(C)=O)cc23)nc2c1CCOC21CCOC1] 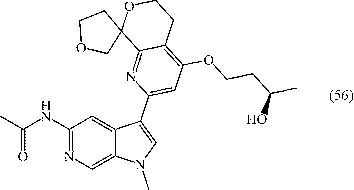 [SMILES: CC(=O)Nc1cc2c(-c3cc(OCC[C@@H](C)O)c4c(n3)C3(CCOC3)OCC4)cn(C)c2cn1] 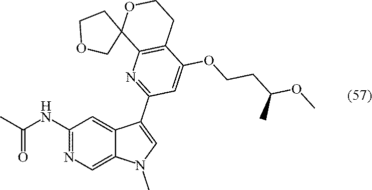 [SMILES: CO[C@@H](C)CCOc1cc(-c2cn(C)c3cnc(NC(C)=O)cc23)nc2c1CCOC21CCOC1] 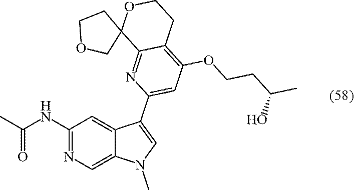 [SMILES: CC(=O)Nc1cc2c(-c3cc(OCC[C@H](C)O)c4c(n3)C3(CCOC3)OCC4)cn(C)c2cn1] 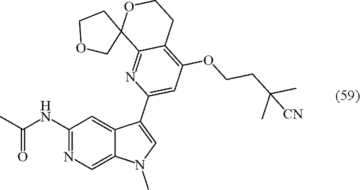 [SMILES: CC(=O)Nc1cc2c(-c3cc(OCCC(C)(C)C#N)c4c(n3)C3(CCOC3)OCC4)cn(C)c2cn1] 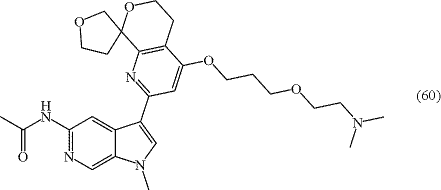 [SMILES: CC(=O)Nc1cc2c(-c3cc(OCCCOCCN(C)C)c4c(n3)C3(CCOC3)OCC4)cn(C)c2cn1] 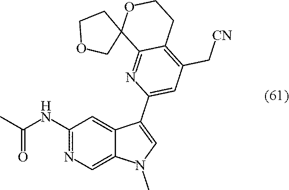 [SMILES: CC(=O)Nc1cc2c(-c3cc(CC#N)c4c(n3)C3(CCOC3)OCC4)cn(C)c2cn1] 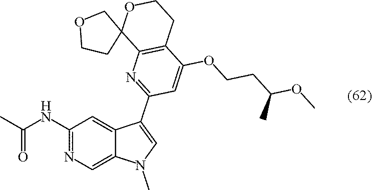 [SMILES: CO[C@@H](C)CCOc1cc(-c2cn(C)c3cnc(NC(C)=O)cc23)nc2c1CCOC21CCOC1] 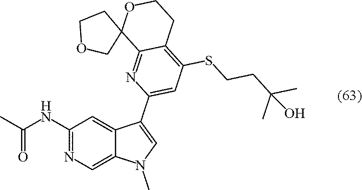 [SMILES: CC(=O)Nc1cc2c(-c3cc(SCCC(C)(C)O)c4c(n3)C3(CCOC3)OCC4)cn(C)c2cn1] 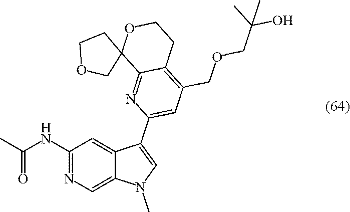 [SMILES: CC(=O)Nc1cc2c(-c3cc(COCC(C)(C)O)c4c(n3)C3(CCOC3)OCC4)cn(C)c2cn1] 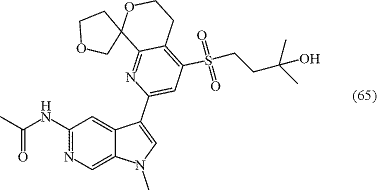 [SMILES: CC(=O)Nc1cc2c(-c3cc(S(=O)(=O)CCC(C)(C)O)c4c(n3)C3(CCOC3)OCC4)cn(C)c2cn1] 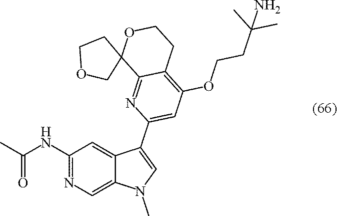 [SMILES: CC(=O)Nc1cc2c(-c3cc(OCCC(C)(C)N)c4c(n3)C3(CCOC3)OCC4)cn(C)c2cn1] 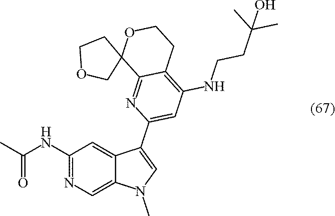 [SMILES: CC(=O)Nc1cc2c(-c3cc(NCCC(C)(C)O)c4c(n3)C3(CCOC3)OCC4)cn(C)c2cn1] 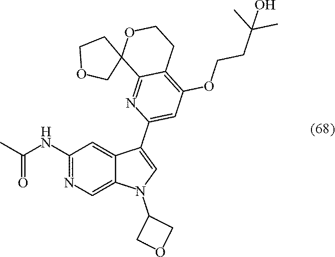 [SMILES: CC(=O)Nc1cc2c(-c3cc(OCCC(C)(C)O)c4c(n3)C3(CCOC3)OCC4)cn(C3COC3)c2cn1] 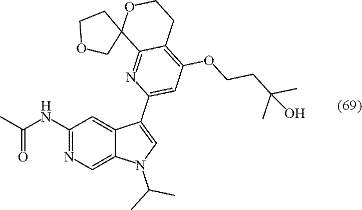 [SMILES: CC(=O)Nc1cc2c(-c3cc(OCCC(C)(C)O)c4c(n3)C3(CCOC3)OCC4)cn(C(C)C)c2cn1]
.

8. A pharmaceutical composition comprising the compound of claim 1, further comprising at least one of pharmaceutically acceptable adjuvants, excipients, carriers and vehicles.

9. The pharmaceutical composition of claim 8, which further comprises other therapeutic agents, the other therapeutic agents are selected from at least one of corticosteroids, rolipram, carvestatin, cytokine inhibitory anti-inflammatory drugs, interleukin-10, glucocorticoid, salicylate, nuclear translocation inhibitor, steroid antiviral agent, antiproliferative agent, antimalarial, TNF-a inhibitor, or combinations thereof.

10. (canceled)

11. (canceled)

12. A method of preventing, handling, treating and relieving TYK2-mediated diseases in a subject comprising administering to the subject a therapeutically effective amount of the compound of claim 1.

13. The method of claim 12, wherein the TYK2-mediated disease is pancreatitis, asthma, allergy, adult respiratory distress syndrome, chronic obstructive pulmonary disease, glomerulonephritis, rheumatoid arthritis, systemic lupus erythematosus, cutaneous lupus erythematosus, lupus nephritis, discoid lupus erythematosus, scleroderma, chronic thyroiditis, Graves disease, autoimmune gastritis, diabetes mellitus, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn’s disease, psoriasis, graft-versus-host disease, endotoxin-induced inflammatory response, tuberculosis, atherosclerosis, muscle degeneration, cachexia, psoriatic arthritis, Knight’s syndrome, gout, traumatic arthritis, rheumatoid arthritis, acute synovitis, pancreatic β-cell disease, disease characterized by massive neutrophil infiltration, rheumatoid spondylitis, gouty arthritis, cerebral malaria, chronic pulmonary inflammatory disease, silicosis, pulmonary sarcoidosis, bone resorption disease, allograft rejection, fever due to infection, myalgia due to infection, cachexia secondary to infection, keloid formation, scar tissue formation, fever, influenza, osteoporosis, osteoarthritis, acute myelogenous leukemia, chronic myelogenous leukemia, metastatic melanoma, Kaposi’s sarcoma, multiple myeloma, sepsis, septic shock, Shigellosis, Alzheimer’s disease, Parkinson’s disease, cerebral ischemia or neurodegenerative disease caused by traumatic injury, angiogenic disorders, viral diseases, CMV retinitis, AIDS, ARC, herpes, stroke, myocardial ischemia, ischemia in stroke heart attack, organ hypoxia, vascular proliferation, cardiac reperfusion injury, renal reperfusion injury, thrombosis, cardiac hypertrophy, coagulation-induced enzymatic platelet aggregation, endotoxemia, toxic shock syndrome, a disease state associated with prostaglandin endoperoxidase synthase-2, or pemphigus vulgaris.

14. (canceled)

15. (canceled)

16. A method of preventing, handling, treating and relieving TYK2-mediated diseases in a subject comprising administering to the subject a therapeutically effective amount of the pharmaceutical composition of claim 8.

17. The method of claim 16, wherein the TYK2-mediated disease is pancreatitis, asthma, allergy, adult respiratory distress syndrome, chronic obstructive pulmonary disease, glomerulonephritis, rheumatoid arthritis, systemic lupus erythematosus, cutaneous lupus erythematosus, lupus nephritis, discoid lupus erythematosus, scleroderma, chronic thyroiditis, Graves disease, autoimmune gastritis, diabetes mellitus, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn’s disease, psoriasis, graft-versus-host disease, endotoxin-induced inflammatory response, tuberculosis, atherosclerosis, muscle degeneration, cachexia, psoriatic arthritis, Knight’s syndrome, gout, traumatic arthritis, rheumatoid arthritis, acute synovitis, pancreatic β-cell disease, disease characterized by massive neutrophil infiltration, rheumatoid spondylitis, gouty arthritis, cerebral malaria, chronic pulmonary inflammatory disease, silicosis, pulmonary sarcoidosis, bone resorption disease, allograft rejection, fever due to infection, myalgia due to infection, cachexia secondary to infection, keloid formation, scar tissue formation, fever, influenza, osteoporosis, osteoarthritis, acute myelogenous leukemia, chronic myelogenous leukemia, metastatic melanoma, Kaposi’s sarcoma, multiple myeloma, sepsis, septic shock, Shigellosis, Alzheimer’s disease, Parkinson’s disease, cerebral ischemia or neurodegenerative disease caused by traumatic injury, angiogenic disorders, viral diseases, CMV retinitis, AIDS, ARC, herpes, stroke, myocardial ischemia, ischemia in stroke heart attack, organ hypoxia, vascular proliferation, cardiac reperfusion injury, renal reperfusion injury, thrombosis, cardiac hypertrophy, coagulation-induced enzymatic platelet aggregation, endotoxemia, toxic shock syndrome, a disease state associated with prostaglandin endoperoxidase synthase-2, or pemphigus vulgaris.
